SEQUENCE LISTING (77 sequences)

SEQ ID NO: 1 - DNA, 29899 bp, synthetic construct
  misc_feature 1..29899: note = sequence comprising mutated NSP1
  source 1..29899: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 2 - DNA, 28843 bp, synthetic construct
  misc_feature 1..28843: note = sequence comprising Delta6-8
  source 1..28843: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 3 - DNA, 29899 bp, synthetic construct
  misc_feature 1..29899: note = OTS45678
  source 1..29899: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 4 - DNA, 28843 bp, synthetic construct
  misc_feature 1..28843: note = sequence comprising NSP1mut Delta6-8
  source 1..28843: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 5 - DNA, 28819 bp, synthetic construct
  misc_feature 1..28819: note = sequence comprising NSP1_OTS4578_delta6-8_bristoldel
  source 1..28819: mol_type = other DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactatagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 6 - RNA, 28819 nt, synthetic construct
  source 1..28819: mol_type = other RNA
  misc_feature 1..28819: note = OTS 4-5-6-7-8 Nsp1 (K164A/H165A).delORF6-8.FCS
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactgcagctagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactatagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaa

SEQ ID NO: 7 - DNA, 29891 bp, Coronavirus SARS-CoV-2
  source 1..29891: mol_type = genomic DNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaa

SEQ ID NO: 8 - protein, 8 aa, Coronavirus SARS-CoV-2
VNFNFNGL

SEQ ID NO: 9 - DNA, 2528 bp, synthetic construct
  source 1..2528: mol_type = other DNA
ttcggatgctcgaactgcacctcatggtcatgttatggttgagttggtagcagaattggaaggcattcagtacggtcgttcaggtgagacattaggtgtcttagtccctcatgtgggcgaaataccagtggcttaccgcaaggttttattacgtaagaacggtaataaaggagctggtggccatagttacggcgccgatttaaagtcatttgacttaggcgacgagttaggcactgatccttatgaagattttcaagaaaactggaacactaaacattcgtcaggtgttacccgtgaattgatgcgtgagttaaacggaggggcatacactcgctatgtcgataacaacttctgtggccctgatggctaccctttagagtgcattaaagacttattagcacgtgctggtaaagcttcatgcactttgtccgaacaattggactttattgacactaagaggggtgtatactgctgccgtgaacatgagcatgaaattgcttggtacacggaacgttcagaaaagtcgtatgaattgcagacaccttttgaaattaaattggcaaagaaatttgacaccttcaatggggaatgtccaaattttgtatttcccttaaattcgataatcaagactattcaaccaagggttgaaaagaaaaagttagatggctttatgggtagaattcgatcagtctatccagttgcgtcaccaaatgaatgcaaccaaatgtgcctttcaactttgatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcattcacttgccgaataccataatgaatcaggcttgaaaaccattttacgtaagggtggtcgcactattgcctttggaggctgtgtgttctcatatgttggttgccataacaagtgtgcctattgggttccacgtgcttcggctaacataggttgtaaccatacaggtgttgttggagaaggttcggaaggtttaaatgacaacttattagaaatattgcaaaaagagaaagtcaacatcaatattgttggtgactttaaattaaatgaagagatcgccattattttggcatcattttcagcttcgacatcagcttttgtggaaactgtgaaaggtttggattataaagcattcaaacaaattgttgaatcgtgtggtaattttaaagttacaaaaggaaaagctaaaaaaggtgcctggaatattggtgaacagaaatcaatattgtcacctttgtatgcatttgcatcagaggctgctcgtgttgtacgatcaattttctcgcgcactttagaaactgctcaaaattcagtgcgtgttttacagaaggccgctataacaatattagatggaatttcacagtattcattgagattgattgatgctatgatgttcacatcagatttggctactaacaatttagttgtaatggcctacattacaggtggtgttgttcagttgacttcgcagtggttaactaacatctttggcactgtttatgaaaaattgaaacccgtcttagattggttagaagagaagtttaaggaaggtgtagagtttttaagagacggttgggaaattgttaaatttatctcaacctgtgcttgtgaaattgtcggtggacaaattgtcacctgtgcaaaggaaattaaggagtcagttcagacattctttaagttagtaaataaatttttggctttgtgtgctgactcaatcattattggtggagctaaattaaaagccttgaatttaggtgaaacatttgtcacgcactcaaagggattgtacagaaagtgtgttaaatcgagagaagaaactggcttattgatgcctttaaaagccccaaaagaaattatcttcttagagggagaaacattacccacagaagtgttaacagaggaagttgtcttgaaaactggtgatttacaaccattagaacaacctacttcagaagctgttgaagctccattggttggtacaccagtttgtattaacgggttaatgttgttggaaatcaaagacacagaaaagtactgtgcccttgcacctaatatgatggtaacaaacaataccttcacattgaaaggcggtgcaccaacaaaggttacttttggtgatgacactgtgatagaagtgcaaggttacaagtcagtgaatatcacttttgaattagatgaaaggattgataaagtattaaatgagaagtgctcagcctatacagttgaattgggtacagaagtaaatgagttcgcctgtgttgtggcagatgctgtcataaaaactttgcaaccagtatcagaattacttacaccattgggcattgatttagatgagtggtcaatggctacatactacttatttgatgagtcaggtgagtttaaat

SEQ ID NO: 10 - DNA, 2944 bp, synthetic construct
  source 1..2944: mol_type = other DNA
gtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagttggtagcagaattggaaggcattcagtacggtcgttcaggtgagacattaggtgtcttagtccctcatgtgggcgaaataccagtggcttaccgcaaggttttattacgtaagaacggtaataaaggagctggtggccatagttacggcgccgatttaaagtcatttgacttaggcgacgagttaggcactgatccttatgaagattttcaagaaaactggaacactaaacattcgtcaggtgttacccgtgaattgatgcgtgagttaaacggaggggcatacactcgctatgtcgataacaacttctgtggccctgatggctaccctttagagtgcattaaagacttattagcacgtgctggtaaagcttcatgcactttgtccgaacaattggactttattgacactaagaggggtgtatactgctgccgtgaacatgagcatgaaattgcttggtacacggaacgttcagaaaagtcgtatgaattgcagacaccttttgaaattaaattggcaaagaaatttgacaccttcaatggggaatgtccaaattttgtatttcccttaaattcgataatcaagactattcaaccaagggttgaaaagaaaaagttagatggctttatgggtagaattcgatcagtctatccagttgcgtcaccaaatgaatgcaaccaaatgtgcctttcaactttgatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcattcacttgccgaataccataatgaatcaggcttgaaaaccattttacgtaagggtggtcgcactattgcctttggaggctgtgtgttctcatatgttggttgccataacaagtgtgcctattgggttccacgtgcttcggctaacataggttgtaaccatacaggtgttgttggagaaggttcggaaggtttaaatgacaacttattagaaatattgcaaaaagagaaagtcaacatcaatattgttggtgactttaaattaaatgaagagatcgccattattttggcatcattttcagcttcgacatcagcttttgtggaaactgtgaaaggtttggattataaagcattcaaacaaattgttgaatcgtgtggtaattttaaagttacaaaaggaaaagctaaaaaaggtgcctggaatattggtgaacagaaatcaatattgtcacctttgtatgcatttgcatcagaggctgctcgtgttgtacgatcaattttctcgcgcactttagaaactgctcaaaattcagtgcgtgttttacagaaggccgctataacaatattagatggaatttcacagtattcattgagattgattgatgctatgatgttcacatcagatttggctactaacaatttagttgtaatggcctacattacaggtggtgttgttcagttgacttcgcagtggttaactaacatctttggcactgtttatgaaaaattgaaacccgtcttagattggttagaagagaagtttaaggaaggtgtagagtttttaagagacggttgggaaattgttaaatttatctcaacctgtgcttgtgaaattgtcggtggacaaattgtcacctgtgcaaaggaaattaaggagtcagttcagacattctttaagttagtaaataaatttttggctttgtgtgctgactcaatcattattggtggagctaaattaaaagccttgaatttaggtgaaacatttgtcacgcactcaaagggattgtacagaaagtgtgttaaatcgagagaagaaactggcttattgatgcctttaaaagccccaaaagaaattatcttcttagagggagaaacattacccacagaagtgttaacagaggaagttgtcttgaaaactggtgatttacaaccattagaacaacctacttcagaagctgttgaagctccattggttggtacaccagtttgtattaacgggttaatgttgttggaaatcaaagacacagaaaagtactgtgcccttgcacctaatatgatggtaacaaacaataccttcacattgaaaggcggtgcaccaacaaaggttacttttggtgatgacactgtgatagaagtgcaaggttacaagtcagtgaatatcacttttgaattagatgaaaggattgataaagtattaaatgagaagtgctcagcctatacagttgaattgggtacagaagtaaatgagttcgcctgtgttgtggcagatgctgtcataaaaactttgcaaccagtatcagaattacttacaccattgggcattgatttagatgagtggtcaatggctacatactacttatttgatgagtcaggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccag

SEQ ID NO: 11 - DNA, 2515 bp, synthetic construct
  source 1..2515: mol_type = other DNA
ttcggatgctcgaactgcacctcatggtcatgttatggttgagttggtagcagaattggaaggcattcagtacggtcgttcaggtgagacattaggtgtcttagtccctcatgtgggcgaaataccagtggcttaccgcaaggttttattacgtaagaacggtaataaaggagctggtggccatagttacggcgccgatttaaagtcatttgacttaggcgacgagttaggcactgatccttatgaagattttcaagaaaactggaacactaaacattcgtcaggtgttacccgtgaattgatgcgtgagttaaacggaggggcatacactcgctatgtcgataacaacttctgtggccctgatggctaccctttagagtgcattaaagacttattagcacgtgctggtaaagcttcatgcactttgtccgaacaattggactttattgacactaagaggggtgtatactgctgccgtgaacatgagcatgaaattgcttggtacacggaacgttcagaaaagtcgtatgaattgcagacaccttttgaaattaaattggcaaagaaatttgacaccttcaatggggaatgtccaaattttgtatttcccttaaattcgataatcaagactattcaaccaagggttgaaaagaaaaagttagatggctttatgggtagaattcgatcagtctatccagttgcgtcaccaaatgaatgcaaccaaatgtgcctttcaactttgatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcattcacttgccgaataccataatgaatcaggcttgaaaaccattttacgtaagggtggtcgcactattgcctttggaggctgtgtgttctcatatgttggttgccataacaagtgtgcctattgggttccacgtgcttcggctaacataggttgtaaccatacaggtgttgttggagaaggttcggaaggtttaaatgacaacttattagaaatattgcaaaaagagaaagtcaacatcaatattgttggtgactttaaattaaatgaagagatcgccattattttggcatcattttcagcttcgacatcagcttttgtggaaactgtgaaaggtttggattataaagcattcaaacaaattgttgaatcgtgtggtaattttaaagttacaaaaggaaaagctaaaaaaggtgcctggaatattggtgaacagaaatcaatattgtcacctttgtatgcatttgcatcagaggctgctcgtgttgtacgatcaattttctcgcgcactttagaaactgctcaaaattcagtgcgtgttttacagaaggccgctataacaatattagatggaatttcacagtattcattgagattgattgatgctatgatgttcacatcagatttggctactaacaatttagttgtaatggcctacattacaggtggtgttgttcagttgacttcgcagtggttaactaacatctttggcactgtttatgaaaaattgaaacccgtcttagattggttagaagagaagtttaaggaaggtgtagagtttttaagagacggttgggaaattgttaaatttatctcaacctgtgcttgtgaaattgtcggtggacaaattgtcacctgtgcaaaggaaattaaggagtcagttcagacattctttaagttagtaaataaatttttggctttgtgtgctgactcaatcattattggtggagctaaattaaaagccttgaatttaggtgaaacatttgtcacgcactcaaagggattgtacagaaagtgtgttaaatcgagagaagaaactggcttattgatgcctttaaaagccccaaaagaaattatcttcttagagggagaaacattacccacagaagtgttaacagaggaagttgtcttgaaaactggtgatttacaaccattagaacaacctacttcagaagctgttgaagctccattggttggtacaccagtttgtattaacgggttaatgttgttggaaatcaaagacacagaaaagtactgtgcccttgcacctaatatgatggtaacaaacaataccttcacattgaaaggcggtgcaccaacaaaggttacttttggtgatgacactgtgatagaagtgcaaggttacaagtcagtgaatatcacttttgaattagatgaaaggattgataaagtattaaatgagaagtgctcagcctatacagttgaattgggtacagaagtaaatgagttcgcctgtgttgtggcagatgctgtcataaaaactttgcaaccagtatcagaattacttacaccattgggcattgatttagatgagtggtcaatggctacatactacttatttgatgagtca

SEQ ID NO: 12 - DNA, 2402 bp, synthetic construct
  source 1..2402: mol_type = other DNA
accagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactc

SEQ ID NO: 13 - DNA, 2992 bp, synthetic construct
  source 1..2992: mol_type = other DNA
atcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtattgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatcagaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaac

SEQ ID NO: 14 - DNA, 2331 bp, synthetic construct
  source 1..2331: mol_type = other DNA
ttgaagtcagaggacgcgcagggaatggataatttagcctgcgaagatttaaaaccagtctcagaagaagtagtggaaaatcctaccatacagaaagacgttttagagtgtaatgtgaaaactaccgaagttgtaggagacattatattaaaaccagcaaataattcattaaaaattacagaagaggttggccacacagatttaatggctgcttatgtagacaattcatcacttactattaagaaacctaatgaattatcaagagtattaggtttgaaaaccttagctactcatggtttagctgctgttaattcagtcccttgggatactatagctaattatgctaagccttttttaaacaaagttgtttcaacaactactaacatagttacacggtgtttaaaccgtgtttgtactaattatatgccttatttctttactttattgttacaattgtgtacttttactagatcaacaaattcaagaattaaagcatcaatgccgactactatagcaaagaatactgttaagtcagtcggtaaattttgtttagaggcttcatttaattatttgaagtcacctaatttttcaaaattgataaatattataatttggtttttattattatcagtttgcttaggttcattaatctactcaaccgctgctttaggtgttttaatgtcaaatttaggcatgccttcatactgtactggttacagagaaggctatttgaactcaactaatgtcactattgcaacctactgtactggttcaataccttgttcagtttgtttatcaggtttagattcattagacacctatccttcattagaaactatacaaattaccatttcatcatttaaatgggatttaactgcttttggcttagttgcagagtggtttttggcatatattttattcactaggtttttctatgtattaggattggctgcaatcatgcaattgtttttctcgtattttgcagtacattttatttcaaattcatggttaatgtggttaataattaatttagtacaaatggccccgatttcagctatggttagaatgtacatcttctttgcatcattttattatgtatggaaatcatatgtgcatgttgtagacggttgtaattcatcaacttgtatgatgtgttacaaacgtaatagagcaacaagagtcgaatgtacaactattgttaatggtgttagaaggtcgttttatgtctatgctaatggaggtaaaggcttttgcaaattacacaattggaattgtgttaattgtgatacattctgtgctggttcaacatttatttcagatgaagttgcgagagacttgtcattacagtttaaaagaccaataaatcctactgaccagtcatcatacatcgttgattcagttacagtgaagaatggttcgatccatttatactttgataaagctggtcaaaagacttatgaaagacattcattgtcacattttgttaacttagacaacttgagagctaataacactaaaggttcattgcctattaatgttatagtttttgatggtaaatcaaaatgtgaagaatcatcagcaaaatcagcgtcagtttactacagtcagttaatgtgtcaacctatattgttattagatcaggcattagtgtcagatgttggtgattcagcggaagttgcagttaaaatgtttgatgcttacgttaatacgttttcatcaacttttaacgtaccaatggaaaaattgaaaacattagttgcaactgcagaagctgaattagcaaagaatgtgtcgttagacaatgtcttatcaacttttatttcagcagctcggcaagggtttgttgattcagatgtagaaactaaagatgttgttgaatgtttaaaattgtcacatcaatcagacatagaagttactggcgattcatgtaataactatatgttgacctataacaaagttgaaaacatgacaccccgtgacttaggtgcttgtattgactgttcagcgcgtcatattaatgcgcaggtagcaaaatcacacaacattgctttgatatggaacgttaaagatttcatgtcattgtcagaacaattacgaaaacaaatacgttcagctgctaaaaagaataacttaccttttaagttgacatgtgcaactactagacaagttgttaatgttgtaacaacaaagatagcattaaagggtggtaaaattgttaataattggttgaagcagttaattaaagttacattagtgttcttatttgttgctgctattttctatttaataacacctgttcatgtcatgtcaaaacatactgacttttcatca

SEQ ID NO: 15 - DNA, 2669 bp, synthetic construct
  source 1..2669: mol_type = other DNA
atcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttac

SEQ ID NO: 16 - DNA, 3249 bp, synthetic construct
  source 1..3249: mol_type = other DNA
gtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttcaggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttgttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttag

SEQ ID NO: 17 - DNA, 2611 bp, synthetic construct
  source 1..2611: mol_type = other DNA
aggtaagccagtaccatattgttatgataccaatgtattagaaggttcagttgcttatgaatcattacgccctgacacacgttatgtgttgatggatggctcaattattcaatttcctaacacctacttagaaggttcagttagagtggtaacaacttttgattcagagtactgtaggcacggcacttgtgaaagatcagaagctggtgtttgtgtatcaacttcaggtagatgggtattaaacaatgattattacagatcattaccaggagttttctgtggtgtagatgctgtaaatttattaactaatatgtttacaccattaattcaacctattggtgctttggacatatcagcatcaatagtagctggtggtattgtagctatcgtagtaacatgcttagcctactattttatgaggtttagaagagcttttggtgaatactcacatgtagttgcctttaatactttattattcttaatgtcattcactgtattgtgtttaacaccagtttactcattcttacctggtgtttattcagttatttacttgtacttgacattttatttaactaatgatgtttcatttttagcacatattcagtggatggttatgttcacacctttagtacctttctggataacaattgcttatatcatttgtatttcgacaaagcatttctattggttcttttcaaattacttaaagagacgtgtagtctttaatggtgtttcgttttcaacttttgaagaagctgcgttgtgcacctttttgttaaataaagaaatgtatttaaagttgcgttcagatgtgttattacctttaacgcaatataatagatacttagctttatataataagtacaagtatttttcaggagcaatggatacaacttcgtacagagaagctgcttgttgtcatttggcaaaggctttgaatgacttctcaaactcaggttcagatgttttataccaaccaccacaaacctcaatcacctcagctgttttgcagtcaggttttagaaaaatggcattcccatcaggtaaagttgagggttgtatggtacaagtaacttgtggtacaactacattaaacggtttatggttagatgacgtagtttactgtccaagacatgtgatctgcacctcagaagacatgttaaaccctaattatgaagatttattgattcgtaagtcaaatcataatttcttggtacaggctggtaatgttcaattgagggttattggacattcaatgcaaaattgtgtattaaagttaaaggttgatacagccaatcctaagacacctaagtataagtttgttcgcattcaaccaggacagactttttcagtgttagcttgttacaatggttcaccatcaggtgtttaccaatgtgctatgaggcccaatttcactattaagggttcattcttaaatggttcatgtggttcagttggttttaacatagattatgactgtgtctcattttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttttgaatcgatttaccacaactttaaatgactttaacttagtggctatgaagtacaattatgaacctttaacacaagaccatgttgacatattaggacctttatctgctcaaactggaattgccgttttagatatgtgtgcttcattaaaagaattattgcaaaatggtatgaatggacgtaccatattgggttcagctttattagaagatgaatttacaccttttgatgttgttagacaatgctcaggtgttactttccaatcagcagtgaaaagaacaatcaagggtacacaccactggttgttattgacaattttgacttcattattagttttagtccagtcaactcaatggtcattgttcttttttttgtatgaaaatgcctttttaccttttgctatgggtattattgctatgtcagcttttgcaatgatgtttgtcaaacataagcatgcatttttgtgtttgtttttgttaccttcattagccactgtagcttattttaatatggtctatatgcctgcttcatgggtgatgcgtattatgacatggttggatatggttgatacttcattgtcaggttttaagttaaaagactgtgttatgtatgcatcagctgtagtgttattaatcttaatgacagcaagaactgtgtatgatgatggtgctaggagagtgtggacattaatgaatgtcttgacattggtttataaagtttattatggtaatgctttagatcaagccatttcgatgtgggctttaataatctcagttacttcaaactactcaggtgtagttacaactgtcatgtttttggccagaggtattgtttttatgtgtgttgagtattgccctattttcttcataactggtaatacattacagtgtataatgttagtttattgtttcttaggctatttttgtacttgttactttggcttg

SEQ ID NO: 18 - DNA, 2795 bp, synthetic construct
  source 1..2795: mol_type = other DNA
actagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtca

SEQ ID NO: 19 - DNA, 3388 bp, synthetic construct
  source 1..3388: mol_type = other DNA
gatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcattaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtcagttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaa

SEQ ID NO: 20 - DNA, 2751 bp, synthetic construct
  source 1..2751: mol_type = other DNA
ttaactaacaatgttgcttttcaaactgtcaaacccggtaattttaacaaagacttctatgactttgctgtgtcaaagggtttctttaaggaaggatcatcagttgaattaaaacacttcttctttgctcaggatggtaatgctgctatctcggattatgactactatcgttataatttaccaacaatgtgtgatatcagacaattattatttgtagttgaagttgttgataagtactttgattgttacgatggtggctgtattaatgctaaccaagtcatcgtcaacaacttagacaaatcagctggttttccatttaataaatggggtaaggctagattatattatgattcaatgtcatatgaggatcaagatgcattattcgcatatacaaaacgtaatgtcatccctactataactcaaatgaatttaaagtatgccatttcagcaaagaatagagctcgcaccgtagctggtgtctcaatctgttcaactatgaccaatagacagtttcatcaaaaattattgaaatcaatagccgccactagaggagctactgtagtaattggaacatcgaaattctatggtggttggcacaacatgttaaaaactgtttattcagatgtagaaaaccctcacttaatgggttgggattatcctaaatgtgatagagccatgcctaacatgttaagaattatggcctcattagttttagctcgcaaacatacaacgtgttgttcgttgtcacaccgtttctatagattagctaatgagtgtgctcaagtattgtcagaaatggtcatgtgtggcggttcattatatgttaaaccaggtggaacctcatcaggagatgccacaactgcttatgctaattcagtttttaacatttgtcaagctgtcacggccaatgttaatgcattattatcaactgatggtaacaaaattgccgataagtatgtccgcaatttacaacacagattatatgagtgtttgtatagaaatagagatgttgacacagactttgtgaatgagttttacgcatatttgcgtaaacatttctcaatgatgatattgtcagacgatgctgttgtgtgtttcaattcgacttatgcatcacaaggtttagtggcttcgataaagaactttaagtcagttttatattatcaaaacaatgtttttatgtcagaagcaaaatgttggactgagactgacttaactaaaggacctcatgaattttgctcacaacatacaatgttagttaaacagggtgatgattatgtgtacttaccttacccagatccatcaagaatcttaggggccggctgttttgtagatgatatcgtaaaaacagatggtacattaatgattgaacggttcgtgtcattagctatagatgcttacccattaactaaacatcctaatcaggagtatgctgatgtctttcatttgtacttacaatacataagaaagttacatgatgagttaacaggacacatgttagacatgtattcagttatgttaactaatgataacacttcaaggtattgggaacctgagttttatgaggctatgtacacaccgcatacagtcttacaggctgttggggcttgtgttttatgcaattcacagacttcattaagatgtggtgcttgcatacgtagaccattcttatgttgtaaatgctgttacgaccatgtcatatcaacatcacataaattagtcttgtcagttaatccgtatgtttgcaatgctccaggttgtgatgtcacagatgtgactcaattatacttaggaggtatgtcgtattattgtaaatcacataaaccacccattagttttccattgtgtgctaatggacaagtttttggtttatataaaaatacatgtgttggttcggataatgttactgactttaatgcaattgcaacatgtgactggacaaatgctggtgattacattttagctaacacctgtactgaaagattgaagttatttgcagcagaaacgttgaaagctactgaggagacatttaaattgtcatatggtattgctactgtacgtgaagtgttgtcagacagagaattacatttatcatgggaagttggtaaacctagaccaccattaaaccgaaattatgtctttactggttatcgtgtaactaaaaactcaaaagtacaaataggagagtacacctttgaaaaaggtgactatggtgatgctgttgtttaccgaggtacaacaacttacaaattaaatgttggtgattattttgtgttgacatcacatacagtaatgccattatcagcacctacattagtgccacaagagcactatgttagaattactggcttatacccaacattgaatatctcagatgagttttcatcgaatgttgcaaattatcaaaaggttggtatgcaaaagtattcaacattacagggaccacctggtactggtaagtcacattttgctattggcttagctttgtactacccttcagctcgcatagtgtatacagcttgctcacatgccgctgttgatgcattatgtgagaaggcattaaaatatttgcctatagataaatgttcaagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgtca

SEQ ID NO: 21 - DNA, 2411 bp, synthetic construct
  source 1..2411: mol_type = other DNA
ttaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaat

SEQ ID NO: 22 - DNA, 2960 bp, synthetic construct
  source 1..2960: mol_type = other DNA
atgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattcttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcattaaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggt

SEQ ID NO: 23 - DNA, 2373 bp, synthetic construct
  source 1..2373: mol_type = other DNA
ttaacacgtaaccctgcttggagaaaagctgtctttatttcaccttataattcacagaatgctgtagcctcaaagattttgggattaccaactcaaactgttgattcatcacagggctcagaatatgactatgtcatattcactcaaaccactgaaacagctcactcatgtaatgtaaacagatttaatgttgctattaccagagcaaaagtaggcatattatgcataatgtcagatagagacttatatgacaagttgcaatttacatcattagaaattccacgtaggaatgtggcaactttacaagctgaaaatgtaacaggattgtttaaagattgttcaaaggtaatcactgggttacatcctacacaggcacctacacacttgtcagttgacactaaattcaaaactgaaggtttatgtgttgacatacctggcatacctaaggacatgacctatagaagattgatctcaatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagttaggtttttcaacaggtgttaacttagttgctgtacctacaggttatgttgatacacctaataatacagatttttcgagagtttcagctaaaccaccgcctggagatcaatttaaacacttgataccattaatgtacaaaggattaccttggaatgtagtgcgtataaagattgtacaaatgttatcagacacattaaaaaatttgtcagacagagtcgtatttgtcttatgggcacatggctttgagttgacatcaatgaagtattttgtgaaaataggacctgagcgcacctgttgtttatgtgatagacgtgccacatgcttttcgactgcttcagacacttatgcctgttggcatcattcaattggatttgattacgtctataatccgtttatgattgatgttcaacaatggggttttacaggtaacttacaatcgaaccatgatttgtattgtcaagtccatggtaatgcacatgtagcttcatgtgatgcaatcatgactaggtgtttagctgtccacgagtgctttgttaagcgtgttgactggactattgaatatcctataattggtgatgaactgaagattaatgcggcttgtagaaaggttcaacacatggttgttaaagctgcattattagcagacaaattcccagttttacacgacattggtaaccctaaagctattaagtgtgtacctcaagctgatgtagaatggaagttctatgatgcacagccttgttcagacaaagcttataaaatagaagaattattctattcatatgccacacattcagacaaattcacagatggtgtatgcttattttggaattgcaatgtcgatagatatcctgctaattcgattgtttgtagatttgacactagagtgttatcaaacttaaacttgcctggttgtgatggtggctcattgtatgtaaataaacatgcattccacacaccagcttttgataaatcagcttttgttaatttaaaacaattaccatttttctattactcagactcaccatgtgagtcacatggaaaacaagtagtgtcagatatagattatgtaccattaaagtcagctacgtgtataacacgttgcaatttaggtggtgctgtctgtagacatcatgctaatgagtacagattgtatttggatgcttataacatgatgatctcagctggcttttcgttgtgggtttacaaacaatttgatacttataacttgtggaacacttttacaagattacagtcattagaaaatgtggcttttaatgttgtaaataagggacactttgatggacaacagggtgaagtaccagtttcaatcattaataacactgtttacacaaaagttgatggtgttgatgtagaattgtttgaaaataaaacaacattacctgttaatgtagcatttgagttatgggctaagcgcaacattaaaccagtaccagaggtgaaaatattgaataatttgggtgtggacattgctgctaatactgtgatctgggactacaaaagagatgctccagcacatatatcaactattggtgtttgttcaatgactgacatagccaagaaaccaactgaaacgatttgtgcaccattgactgtcttttttgatggtagagttgatggtcaagtagacttatttagaaatgcccgtaatggtgttttaattacagaaggttcagttaaaggtttacaaccatcagtaggtcccaaacaagcttcatta

SEQ ID NO: 24 - DNA, 22 bp, synthetic construct
  source 1..22: mol_type = other DNA
gtcttatcagaggcacgtcaac

SEQ ID NO: 25 - DNA, 48 bp, synthetic construct
  source 1..48: mol_type = other DNA
ttcacgggtaacaccactgctagcagcagtgttccagttttcttgaaa

SEQ ID NO: 26 - DNA, 49 bp, synthetic construct
  source 1..49: mol_type = other DNA
ttttcaagaaaactggaacactgctgctagcagtggtgttacccgtgaa

SEQ ID NO: 27 - DNA, 25 bp, synthetic construct
  source 1..25: mol_type = other DNA
ctggtgtaagttccatctctaattg

SEQ ID NO: 28 - DNA, 20 bp, synthetic construct
  source 1..20: mol_type = other DNA
gatggcaactagcactctcc

SEQ ID NO: 29 - DNA, 52 bp, synthetic construct
  source 1..52: mol_type = other DNA
ttagtttgttcgtttagatgaaatcttactgtacaagcaaagcaatattgtc

SEQ ID NO: 30 - DNA, 56 bp, synthetic construct
  source 1..56: mol_type = other DNA
caatattgctttgcttgtacagtaagatttcatctaaacgaacaaactaaaatgtc

SEQ ID NO: 31 - DNA, 23 bp, synthetic construct
  source 1..23: mol_type = other DNA
tttggcaatgttgttccttgagg

SEQ ID NO: 32 - DNA, 23 bp, synthetic construct
  source 1..23: mol_type = other DNA
ggagtcacattaattggagaagc

SEQ ID NO: 33 - DNA, 50 bp, synthetic construct
  source 1..50: mol_type = other DNA
taggcaatgatggattgactagctatagtctgagtctgataactagcgca

SEQ ID NO: 34 - DNA, 50 bp, synthetic construct
  source 1..50: mol_type = other DNA
tgcgctagttatcagactcagactatagctagtcaatccatcattgccta

SEQ ID NO: 35 - DNA, 25 bp, synthetic construct
  source 1..25: mol_type = other DNA
tcatgttcagaaataggacttgttg

SEQ ID NO: 36 - DNA, 32 bp, synthetic construct
  source 1..32: mol_type = other DNA
acaggtacgttaatagttaatagcgtacttct

SEQ ID NO: 37 - DNA, 23 bp, synthetic construct
  source 1..23: mol_type = other DNA
acaatattgcagcagtacgcaca

SEQ ID NO: 38 - DNA, 18 bp, synthetic construct
  source 1..18: mol_type = other DNA
atccttactgcgcttcga

SEQ ID NO: 39 - DNA, 19 bp, synthetic construct
  source 1..19: mol_type = other DNA
ggtaactggtatgatttcg

SEQ ID NO: 40 - DNA, 20 bp, synthetic construct
  source 1..20: mol_type = other DNA
ctggtcaaggttaatatagg

SEQ ID NO: 41 - DNA, 19 bp, synthetic construct
  source 1..19: mol_type = other DNA
tcatacaaaccacgccagg

SEQ ID NO: 42 - DNA, 47 bp, synthetic construct
  source 1..47: mol_type = other DNA
tccttggcacccgagaattccaagtaccggcagcacaagacatctgt

SEQ ID NO: 43 - DNA, 57 bp, synthetic construct
  source 1..57: mol_type = other DNA
cgtagtgcaacaggactaagctcatabaaaaaaaaaaaaaaaaaaaaaaaaaaaaaa

SEQ ID NO: 44 - DNA, 47 bp, synthetic construct
  source 1..47: mol_type = other DNA
tccttggcacccgagaattccaatcgaagccaatccatgcacgtaca

SEQ ID NO: 45 - DNA, 57 bp, synthetic construct
  source 1..57: mol_type = other DNA
tgtcttatagcttcttcgcgggtgaabaaaaaaaaaaaaaaaaaaaaaaaaaaaaaa

SEQ ID NO: 46 - DNA, 47 bp, synthetic construct
  source 1..47: mol_type = other DNA
tccttggcacccgagaattccagactttagatcggcgccgtaactat

SEQ ID NO: 47 - DNA, 57 bp, synthetic construct
  source 1..57: mol_type = other DNA
ggccaccagctcctttattaccgttabaaaaaaaaaaaaaaaaaaaaaaaaaaaaaa

SEQ ID NO: 48 - DNA, 47 bp, synthetic construct
  source 1..47: mol_type = other DNA
tccttggcacccgagaattccactgcagcaatcaatgggcaagcttt

SEQ ID NO: 49 - DNA, 57 bp, synthetic construct
  source 1..57: mol_type = other DNA
gtcattagtataactaccaccacgcabaaaaaaaaaaaaaaaaaaaaaaaaaaaaaa

SEQ ID NO: 50 - DNA, 47 bp, synthetic construct
  source 1..47: mol_type = other DNA
tccttggcacccgagaattccaggattaacaactccggatgaaccgt

SEQ ID NO: 51 - DNA, 57 bp, synthetic construct
  source 1..57: mol_type = other DNA
cgattgtgtgaatttggacatgttcabaaaaaaaaaaaaaaaaaaaaaaaaaaaaaa

SEQ ID NO: 52 - DNA, 47 bp, synthetic construct
  source 1..47: mol_type = other DNA
tccttggcacccgagaattccacagtataaccaccaatctggtagtc

SEQ ID NO: 53 - DNA, 57 bp, synthetic construct
  source 1..57: mol_type = other DNA
atgttcagaaataggacttgttgtgabaaaaaaaaaaaaaaaaaaaaaaaaaaaaaa

SEQ ID NO: 54 - DNA, 47 bp, synthetic construct
  source 1..47: mol_type = other DNA
tccttggcacccgagaattccaagtctgaagtgaagtaactgtgtaa

SEQ ID NO: 55 - DNA, 57 bp, synthetic construct
  source 1..57: mol_type = other DNA
tacaacacagtcttttactccagatabaaaaaaaaaaaaaaaaaaaaaaaaaaaaaa

SEQ ID NO: 56 - DNA, 47 bp, synthetic construct
  source 1..47: mol_type = other DNA
tccttggcacccgagaattccatgcacaagagtagactatatatcgt

SEQ ID NO: 57 - DNA, 57 bp, synthetic construct
  source 1..57: mol_type = other DNA
aaacggaaaagcgaaaacgtttataabaaaaaaaaaaaaaaaaaaaaaaaaaaaaaa

SEQ ID NO: 58 - DNA, 47 bp, synthetic construct
  source 1..47: mol_type = other DNA
tccttggcacccgagaattccacaatattgcagcagtacgcacacaa

SEQ ID NO: 59 - DNA, 57 bp, synthetic construct
  source 1..57: mol_type = other DNA
tcgaagcgcagtaaggatggctagtabaaaaaaaaaaaaaaaaaaaaaaaaaaaaaa

SEQ ID NO: 60 - DNA, 47 bp, synthetic construct
  source 1..47: mol_type = other DNA
tccttggcacccgagaattccaagcgtcctagatggtgtccagcaat

SEQ ID NO: 61 - DNA, 57 bp, synthetic construct
  source 1..57: mol_type = other DNA
acgaagatgtccacgaaggatcacaabaaaaaaaaaaaaaaaaaaaaaaaaaaaaaa

SEQ ID NO: 62 - DNA, 47 bp, synthetic construct
  source 1..47: mol_type = other DNA
tccttggcacccgagaattccaatttgtaataagaaagcgttcgtga

SEQ ID NO: 63 - DNA, 57 bp, synthetic construct
  source 1..57: mol_type = other DNA
tgtagcaacagtgatttctttaggcabaaaaaaaaaaaaaaaaaaaaaaaaaaaaaa

SEQ ID NO: 64 - DNA, 47 bp, synthetic construct
  source 1..47: mol_type = other DNA
tccttggcacccgagaattccaatagcaattccaccggtgatccaat

SEQ ID NO: 65 - DNA, 57 bp, synthetic construct
  source 1..57: mol_type = other DNA
ttattctgtaaacagcagcaagcacabaaaaaaaaaaaaaaaaaaaaaaaaaaaaaa

SEQ ID NO: 66 - DNA, 47 bp, synthetic construct
  source 1..47: mol_type = other DNA
tccttggcacccgagaattccagggaatttaaggtcttccttgccat

SEQ ID NO: 67 - DNA, 57 bp, synthetic construct
  source 1..57: mol_type = other DNA
gttgagtgagagcggtgaaccaagaabaaaaaaaaaaaaaaaaaaaaaaaaaaaaaa

SEQ ID NO: 68 - DNA, 47 bp, synthetic construct
  source 1..47: mol_type = other DNA
tccttggcacccgagaattccaaatttccttgggtttgttctggacc

SEQ ID NO: 69 - DNA, 57 bp, synthetic construct
  source 1..57: mol_type = other DNA
acgtctgccgaaagcttgtgttacaabaaaaaaaaaaaaaaaaaaaaaaaaaaaaaa

SEQ ID NO: 70 - DNA, 47 bp, synthetic construct
  source 1..47: mol_type = other DNA
tccttggcacccgagaattccaaccaccacgaattcgtctggtagct

SEQ ID NO: 71 - DNA, 57 bp, synthetic construct
  source 1..57: mol_type = other DNA
cttcggtagtagccaatttggtcatabaaaaaaaaaaaaaaaaaaaaaaaaaaaaaa

SEQ ID NO: 72 - DNA, 47 bp, synthetic construct
  source 1..47: mol_type = other DNA
tccttggcacccgagaattccaaggatccacaagaacaacagccctt

SEQ ID NO: 73 - DNA, 56 bp, synthetic construct
  source 1..56: mol_type = other DNA
gagacaactacagcaactggtcatabaaaaaaaaaaaaaaaaaaaaaaaaaaaaaa

SEQ ID NO: 74 - DNA, 47 bp, synthetic construct
  source 1..47: mol_type = other DNA
tccttggcacccgagaattccaagtactactactctgtatggttggt

SEQ ID NO: 75 - DNA, 57 bp, synthetic construct
  source 1..57: mol_type = other DNA
aaccaacaccattagtgggttggaaabaaaaaaaaaaaaaaaaaaaaaaaaaaaaaa

SEQ ID NO: 76 - DNA, 47 bp, synthetic construct
  source 1..47: mol_type = other DNA
tccttggcacccgagaattccacctagtgatgttaatacctattggc

SEQ ID NO: 77 - DNA, 57 bp, synthetic construct
  source 1..57: mol_type = other DNA
aaatctaccaatggttctaaagccgabaaaaaaaaaaaaaaaaaaaaaaaaaaaaaa

(19) United States
(12) Patent Application Publication
(10) Pub. No.: US 2026/0124293 A1
(43) Pub. Date: May 7, 2026

(54) TREATMENT USING A ONE-TO-STOP ATTENUATED SARS-COV-2 VIRUS

(71) Applicants: UNIVERSITÄT BERN, Bern (CH); INSTITUT FÜR VIROLOGIE UND IMMUNOLOGIE (IVI), Mittelhäusern (CH); ROCKETVAX AG, Basel (CH)

(72) Inventors: Volker THIEL, Bern (CH); Nadine EBERT, Worb (CH); Bettina Salome TRUEB, Bern (CH); Güliz Tuba BARUT, Bern (CH); Annika KRATZEL, Bern (CH); Jörg JORES, Köniz (CH); Fabien LABROUSSAA, Bellevaux (FR); Martin BEER, Neuenkirchen (DE); Donata HOFFMANN, Mesekenhagen (DE); Jacob SCHÖN, Greifswald (DE); Nico Joel HALWE, Greifswald (DE); Lorenz ULRICH, Limbach bei Homburg (Saar) (DE)

(21) Appl. No.: 19/117,226

(22) PCT Filed: Oct. 12, 2023
(86) PCT No.: PCT/EP2023/078406

§ 371 Date: Mar. 31, 2025

(30) Foreign Application Priority Data
Oct. 12, 2022 (EP) 22201198.3
Mar. 28, 2023 (WO) PCT/EP2023/058069
Jul. 13, 2023 (EP) 23185420.9

(51) Int. Cl. A61K 39/215; A61K 39/00; A61P 37/04; C07K 14/005

(52) U.S. Cl. CPC A61K 39/215; A61P 37/04; C07K 14/005; A61K 2039/53; C12N 2770/20022; C12N 2770/20034; C12N 2770/20071

(57) ABSTRACT

The invention relates to pharmaceutical product comprising a polynucleotide for use in the prevention or treatment of a SARS-CoV-2 virus infection wherein said SARS-CoV-2 virus is not a Wuhan wild-type SARS-CoV-2 virus. The polynucleotide encodes an attenuated human coronavirus or a fragment thereof, wherein the polynucleotide comprises at least 20 one-to-stop codons, wherein a one-to-stop codon is i) a different but synonymous codon compared to the corresponding codon in a natural human coronavirus genome and ii) differs by one nucleotide from a STOP codon.

### RELATED APPLICATIONS

This application is a 35 U.S.C. § 371 filing of International Patent Application No. PCT/EP2023/078406, filed Oct. 12, 2023, which claims priority to European Patent Application No. 23185420.9, filed Jul. 13, 2023, International Patent Application No. PCT/EP2023/058069, filed Mar. 28, 2023, and European Patent Application No. 22201198.3, filed Oct. 12, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Mar. 28, 2025, is named 763303_VOS9-029US_ST26.xml and is 318,198 bytes in size.

The invention relates to pharmaceutical product comprising a polynucleotide for use in the prevention or treatment of a SARS-CoV-2 virus infection wherein said SARS-CoV-2 virus is not a Wuhan wild-type SARS-CoV-2 virus.

Severe acute respiratory syndrome coronavirus 2 (SARS-CoV-2) emerged in December 2019 as the causative agent of coronavirus disease 2019 (COVID-19). The virus is highly transmissible among humans. It has spread rapidly around the world within a matter of weeks and the world is still battling with the ongoing COVID-19 pandemic.

The rapid development and availability of vaccines are crucial in combating many viruses and bacteria. The production of suitable vaccines is a multi-stage, complex process and is not always successful despite often high investments. Typically, the development of a suitable vaccine takes years. These long development times consist of a major problem, especially with regard to new emerging pathogens, or mutated pathogens, as from an epidemiological point of view it is only possible to react too late, if at all, to the emergence of new diseases. In contrast, the analysis, identification and further detection of new or heavily mutated pathogens are now possible within weeks or even days, which is a huge improvement over the last century.

In this context, viruses are of special interest, as they harbor high mutation rates causing the spread from other species to humans. Rapid spreading of these viruses makes them a major challenge for modern medicine. The usual time between the detection/identification of a newly emerging virus and the development of a vaccine is typically years. In a few cases, with sufficient prior knowledge, experimental vaccines could be provided within months. However, this period is much longer than the typical time until thousands or millions of people are infected. Such rapid spread is also a direct consequence of the high mobility of today's society.

Ideally, immediately after the identification of a new virus, a vaccine would be available in sufficient quantity and of the highest quality and would allow for a nationwide vaccination of all persons who have somehow come close to the initial outbreak site of the new virus. Furthermore, an ideal method for such a vaccine would be capable of reacting to the evolution and adaptation of the virus. Such an ideal production possibility seems utopian to the person skilled in the art today.

In the recent past, in particular, the corona pandemic has dramatically increased the relevance of developing suitable tools for vaccine production. There is unanimous agreement that the development of a vaccine against the coronavirus SARS-CoV-2 is the only proven means of containing the pandemic and the associated global crisis in the long term.

The emergence of the severe acute respiratory syndrome coronavirus 2 (SARS-CoV-2) led not only to global spread, but also to the evolution of various concerning virus variants (https://www.ecdc.europa.eu/en/covid-19/variants-concern). Despite the rapid development of vaccines, the current vaccines primarily target the spike protein antigen, providing limited protection against infection and viral transmission. Consequently, SARS-CoV-2 can evade immunity through spike gene mutations, hindering consistent interruption of infection chains. Therefore, there is an urgent need for more robust and adaptable vaccination strategies.

SARS-CoV-2 variant strains are often more contagious or pathogenic than the original wild-type SARS-CoV-2 strain. Such new emerging SARS-CoV-2 strains may lead to a reduced efficiency of first-generation vaccines that were developed against the wild-type SARS-CoV-2 strain. Further, it is unclear whether a vaccination against SARS-CoV-2 to protective immune responses in case a SARS-CoV-2 infection occurs after a long period.

Thus, there is a need to provide a vaccine against variants of coronavirus SARS-CoV-2 and vaccines having a long term effect.

The above technical problem is solved by the embodiments disclosed herein and as defined in the claims. Accordingly, the invention relates to, inter alia, the following embodiments:
1. A polynucleotide encoding an attenuated human coronavirus or a fragment thereof, wherein the polynucleotide comprises at least 20 one-to-stop codons, wherein a one-to-stop codon is
   i) a different but synonymous codon compared to a corresponding codon in a natural human coronavirus genome or a fragment thereof; and
   ii) differs by only one nucleotide from a STOP codon.
2. The polynucleotide of embodiment 1, wherein the fragment of the polynucleotide when combined with corresponding human coronavirus parts encodes a coronavirus particle that induces an immune response after immunization of mice with 5000 PFU coronavirus particle after 15 days and an increased immune response upon challenge with WT human coronavirus after 21 days measured after 35 days.
3. A method for producing a polynucleotide of embodiment 1 or 2, the method comprising the steps of:
   a) providing the CDS of a natural human coronavirus genome, a fragment or cDNA clone thereof; and
   b) modifying the natural human coronavirus genome, the fragment or the retro-transcribed cDNA sequence of the cDNA clone, respectively, wherein said modification comprises replacing at least 20 codons in the natural human coronavirus genome, the fragment or the retro-transcribed cDNA sequence, by at least 20 one-to-stop codons, wherein a one-to-stop codon is
      i) a different but synonymous codon compared to a corresponding codon in the natural human coronavirus genome, the fragment or the retro-transcribed cDNA sequence; and
      ii) differs by only one nucleotide from a STOP codon.
4. The polynucleotide of embodiment 1 or 2 or the method of embodiment 3, wherein the natural human coronavirus genome or a fragment thereof is
   a) a SARS-CoV-2 sequence comprised in or consisting of a sequence as defined by SEQ ID NO: 7 or
   b) a SARS-CoV-2 sequence being 80% identical to a sequence comprised in or consisting of sequence as defined by SEQ ID NO: 7, preferably a SARS-CoV-2 sequence being 80% identical to a sequence comprised in or consisting of sequence as defined by SEQ ID NO: 7 which maintains the ability to encode one or more SARS-CoV-2 virus proteins.
5. The polynucleotide of any one of the embodiments 1, 2 or 4 or the method of embodiment 3 or 4, wherein the fragment has a minimum length of 500 nucleotides.
6. The polynucleotide of any one of the embodiments 1, 2, 4 or 5 or the method of any one of the embodiments 3 to 5, wherein the human coronavirus is SARS-CoV-2 and wherein at least one of the one-to-stop codons is comprised in a sequence part or fragment corresponding to a sequence part of ORF1ab of the natural SARS-CoV-2, a sequence part encoding a structure protein of the natural SARS-CoV-2 or a sequence part encoding an accessory protein of the natural SARS-CoV-2.
7. The polynucleotide of embodiment 6 or the method of embodiment 6, wherein at least one of the one-to-stop codons is comprised in a sequence part or fragment corresponding to a sequence part of ORF1ab of the natural SARS-CoV-2.
8. The polynucleotide of embodiment 7 or the method of embodiment 7, wherein at least one of the one-to-stop codons is comprised in a sequence part or fragment corresponding to an Nsp2 to Nsp15 encoding sequence part of the natural SARS-CoV-2 genome.
9. The polynucleotide of embodiment 8 or the method of embodiment 8, wherein at least one of the one-to-stop codons is comprised in a sequence part or fragment corresponding to an Nsp2 to Nsp7 or an Nsp13 to Nsp15 encoding sequence part of the natural SARS-CoV-2 genome.
10.1. The polynucleotide of embodiment 8 or 9 or the method of embodiment 8 or 9, wherein the one-to-stop codons comprise at least one one-to-stop codon having a position selected from Table 1 corresponding to a position on the natural SARS-CoV-2 genome.
10.2. The polynucleotide of embodiment 8 or 9 or the method of embodiment 8 or 9, wherein the one-to-stop codons comprise at least one one-to-stop codon having a position selected from Table 1 or supplementary Table 3 corresponding to a position on the natural SARS-CoV-2 genome.
11. The polynucleotide of any one of embodiments 1, 2, 4 to 10 or the method of any one of embodiments 3 to 10, wherein the amino acids encoded by the at least 20 one-to-stop codons consist of Leu, Ser, Arg and/or Gly.
12. The polynucleotide of embodiment 11 or the method of embodiment 11, wherein the amino acids encoded by the one-to-stop codons consist of Leu and/or Ser.
13. The polynucleotide of any one of embodiments 1, 2, 4 to 12 or the method of any one of embodiments 3 to 12, wherein the at least 20 one-to-stop codons are at least 50 one-to-stop codons.
14. The polynucleotide of any one of embodiments 1, 2, 4 to 13, wherein the human coronavirus is SARS-CoV-2 and wherein the polynucleotide comprises no sequence encoding a protein having an Nsp1 functionality of the natural SARS-CoV-2 or a sequence encoding a protein having a reduced Nsp1 functionality compared to the Nsp1 of a natural SARS-CoV-2, preferably wherein the polynucleotide comprises a sequence encoding a protein having a reduced Nsp1 functionality compared to the Nsp1 of a natural SARS-CoV-2, and polynucleotide comprises a mutation compared to the Nsp1 encoding sequence of natural SARS-CoV-2, wherein the mutation is K164A and/or H165A.
15. The polynucleotide of any one of embodiments 1, 2, 4 to 14, wherein the human coronavirus is SARS-CoV-2 and wherein the polynucleotide comprises no sequence encoding a protein having the functionality of a protein encoded by the ORF6 gene of the natural SARS-CoV-2 or a sequence encoding a protein having a reduced functionality of a protein encoded by the ORF6 gene of the natural SARS-CoV-2.
16. The polynucleotide of any one of embodiments 1, 2, 4 to 15, wherein the human coronavirus is SARS-CoV-2 and wherein the polynucleotide comprises no sequence encoding a protein having the functionality of a protein encoded by the ORF7a gene of the natural SARS-CoV-2 or a sequence encoding a protein having a reduced functionality of a protein encoded by the ORF7a gene of the natural SARS-CoV-2.
17. The polynucleotide of any one of embodiments 1, 2, 4 to 16, wherein the human coronavirus is SARS-CoV-2 and wherein the polynucleotide comprises no sequence encoding a protein having the functionality of a protein encoded by the ORF7b gene of the natural SARS-CoV-2 or a sequence encoding a protein having a reduced functionality of a protein encoded by the ORF7b gene of the natural SARS-CoV-2.
18. The polynucleotide of any one of embodiments 1, 2, 4 to 17, wherein the human coronavirus is SARS-CoV-2 and wherein the polynucleotide comprises no sequence encoding a protein having the functionality of a protein encoded by the ORF8 gene of the natural SARS-CoV-2 or a sequence encoding a protein having a reduced functionality of a protein encoded by the ORF8 gene of the natural SARS-CoV-2.
19. The polynucleotide of any one of embodiments 1, 2, 4 to 18, wherein the human coronavirus is SARS-CoV-2 and wherein the polynucleotide comprises a sequence part encoding a spike protein, wherein the spike protein comprises a modified or removed cleavage site compared to the cleavage site of the spike protein of the natural SARS-CoV-2.
20. The polynucleotide according to embodiment 19, wherein the polynucleotide consists of or comprises a sequence as defined SEQ ID NO: 6.
21. A vector comprising the polynucleotide of any one of the embodiments 1, 2, 4 to 20.
22. A genetically modified cell comprising the polynucleotide of any one of embodiments 1, 2, 4 to 20.
23. A method for production of an attenuated virus, the method comprising a step of culturing the genetically modified cell of embodiment 22.
24. An attenuated virus comprising the polynucleotide of any one of embodiments 1, 2, 4 to 20.
25. A pharmaceutical product comprising the vector of embodiment 21, the genetically modified cell of embodiment 22 and/or the attenuated virus of embodiment 24 for use as a medicament.
26. A pharmaceutical product comprising the vector of embodiment 21, the genetically modified cell of embodiment 22 and/or the attenuated virus of embodiment 24 for use in treatment and/or prevention of a human coronavirus infection, preferably a SARS-CoV-2 infection.
27. The pharmaceutical product for use of embodiment 25 to 26, wherein the pharmaceutical product further comprises a mutagen.
28. A method of treatment and/or prevention comprising the step of: Administering a pharmaceutical product in a therapeutically effective amount to a subject, wherein the pharmaceutical product comprises the vector of embodiment 21, the genetically modified cell of embodiment 22 and/or the attenuated virus of embodiment 24.
29. The method of embodiment 28, wherein the treatment and/or prevention is a treatment and/or prevention of a human coronavirus infection, preferably a SARS-CoV-2 infection.
30. The method of embodiment 28 or 29, wherein the method further comprises administering a mutagen in a therapeutically effective amount to a subject.
31. The pharmaceutical product for use of embodiment 27 or the method of embodiment 30, wherein the mutagen is 5-Fluorouracil or Molnupiravir.
32. The polynucleotide of the invention, wherein said polynucleotide encodes an attenuated human coronavirus or a fragment thereof, wherein the polynucleotide comprises at least 20 one-to-stop codons, wherein a one-to-stop codon is i) a different but synonymous codon compared to the corresponding codon in a natural human coronavirus genome and ii) differs by one nucleotide from a STOP codon.
33. The polynucleotide of the invention, wherein the natural human coronavirus genome is a natural SARS-CoV-2 genome, preferably a) a SARS-CoV-2 sequence comprised in or consisting of a sequence as defined by SEQ ID NO: 7 or b) a SARS-CoV-2 sequence being 80% identical to a sequence comprised in or consisting of sequence as defined by SEQ ID NO: 7, preferably a SARS-CoV-2 sequence being 80% identical to a sequence comprised in or consisting of sequence as defined by SEQ ID NO: 7 which maintains the ability to encode one or more SARS-CoV-2 virus proteins.
34. The polynucleotide of the invention, wherein at least one of the one-to-stop codons is in a sequence encoding non-structural proteins; preferably the natural human coronavirus genome is a natural SARS-CoV-2 genome, and at least one of the one-to-stop codons is in a sequence corresponding to ORF1ab in the natural SARS-CoV-2 genome.
34. The polynucleotide of the invention, wherein at least one of the one-to-stop codons is in a sequence corresponding to an Nsp1 to Nsp15, preferably Nsp3 to Nsp15 encoding sequence in the natural SARS-CoV-2 genome.
36. The polynucleotide of the invention, wherein at least one of the one-to-stop codons is in a sequence corresponding to an Nsp3 to Nsp7 or an Nsp12 to Nsp15 encoding sequence in the natural SARS-CoV-2 genome.
37. The polynucleotide of the invention, wherein the natural human coronavirus genome is a natural SARS-CoV-2 genome, and wherein at least one of the one-to-stop codons has a CDS codon number corresponding to a CDS codon number as indicated in Table 1 or supplementary Table 3 for SEQ ID NO: 7.
38. The polynucleotide of the invention, wherein at least one of the one-to-stop codons is in a sequence corresponding to an Nsp3 to Nsp7 or an Nsp12 to Nsp15 encoding sequence in the natural SARS-CoV-2 genome and at least one of the one-to-stop codons has a CDS codon number corresponding to a CDS codon number as indicated in Table 1 or supplementary Table 3 for SEQ ID NO: 7.
39. The polynucleotide of the invention, wherein the one-to-stop codons are defined by CDS codon numbers corresponding each to a CDS codon number from 2023 to 6614 as indicated in Table 1 or supplementary Table 3 for SEQ ID NO: 7;
   preferably, the one-to-stop codons are defined by codon changes and CDS codon numbers corresponding each to a CDS codon number from 2023 to 6614 as indicated in Table 1 or supplementary Table 3 for SEQ ID NO: 7.
40. The polynucleotide of the invention, wherein the polynucleotide consists of or comprises a sequence as defined in SEQ ID NO: 3-6 or 9-23, preferably SEQ ID NO: 4-6, more preferably SEQ ID NO: 5 or 6.
41. A pharmaceutical product comprising a polynucleotide for use in the prevention or treatment of a SARS-CoV-2 virus infection,
   wherein said polynucleotide encodes an attenuated human coronavirus or a fragment thereof, wherein the polynucleotide comprises at least 20 one-to-stop codons, wherein a one-to-stop codon is i) a different but synonymous codon compared to the corresponding codon in a natural human coronavirus genome and ii) differs by one nucleotide from a STOP codon, and wherein said SARS-CoV-2 virus is not a Wuhan wild-type SARS-CoV-2 virus.
42. The pharmaceutical product for use according to embodiment 41, wherein said SARS-CoV-2 virus is a variant of the Wuhan wild-type SARS-CoV-2 virus.
43. The pharmaceutical product for use according to embodiment 42, wherein said variant is of lineage B, preferably B.1, more preferably B.1.1 or B.1.617, again more preferably B.1.1.529 or B.1.617.
44. The pharmaceutical product for use according embodiment 42 or 43, wherein the variant is selected from the group comprising, or preferably consisting of, Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), Mu (lineage B.1.621) and a missense variant of a Wuhan wild-type SARS-CoV-2 virus, wherein the genome of said missense variant comprises at least one missense mutation;
   preferably the variant is selected from the group comprising, or preferably consisting of, Alpha (lineage B.1.1.7), B.1.1.7 with E484K, Beta (lineage B.1.351), Delta (lineage B.1.617.2), Omicron (B.1.1.529), Epsilon (lineages B.1.429, B.1.427, CAL.20C), Eta (lineage B.1.525), Iota (lineage B.1.526), Kappa (lineage B.1.617.1), Mu (lineage B.1.621) and a missense variant of a Wuhan wild-type SARS-CoV-2 virus comprising at least one missense mutation;
   more preferably the variant is Delta (lineage B.1.617.2), Omicron (B.1.1.529) or a missense variant of a Wuhan wild-type SARS-CoV-2 virus, wherein the genome of said missense variant comprises at least one missense mutation; and
   again more preferably the variant is Delta (B.1.617.2), Omicron BA.2, Omicron BA.5 or a variant of a Wuhan wild-type SARS-CoV-2 virus, wherein the genome of said missense variant comprises at least one missense mutation.
45. The pharmaceutical product for use according to embodiment 44, wherein said missense mutation is in an ORF encoding a SARS-CoV-2 spike protein, preferably said missense mutation is D614G.
46. The pharmaceutical product for use according to embodiments 42-45, wherein the variant is selected from the group comprising, or preferably consisting of, Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), and Mu (lineage B.1.621);
   preferably the variant is selected from the group comprising, or preferably consisting of, Alpha (lineage B.1.1.7), B.1.1.7 with E484K, Beta (lineage B.1.351), Delta (lineage B.1.617.2), Omicron (B.1.1.529), Epsilon (lineages B.1.429, B.1.427, CAL.20C), Eta (lineage B.1.525), Iota (lineage B.1.526), Kappa (lineage B.1.617.1), and Mu (lineage B.1.621);
   more preferably the variant is Delta (lineage B.1.617.2) or Omicron (B.1.1.529); and
   again more preferably the variant is Delta (B.1.617.2), Omicron BA.2 or Omicron BA.5.
47. The pharmaceutical product for use according to the any one of the preceding embodiments 41-46, wherein the pharmaceutical product is administered intranasally or intramuscularly.
48. The pharmaceutical product for use according to any one of the preceding embodiments 41-47, wherein the natural human coronavirus genome is a natural SARS-CoV-2 genome, preferably
   a) a SARS-CoV-2 sequence comprised in or consisting of a sequence as defined by SEQ ID NO: 7 or
   b) a SARS-CoV-2 sequence being 80% identical to a sequence comprised in or consisting of sequence as defined by SEQ ID NO: 7, preferably a SARS-CoV-2 sequence being 80% identical to a sequence comprised in or consisting of sequence as defined by SEQ ID NO: 7 which maintains the ability to encode one or more SARS-CoV-2 virus proteins.
49. The pharmaceutical product for use according to any one of the preceding embodiments 41-48, wherein at least one of the one-to-stop codons is in a sequence encoding non-structural proteins; preferably the natural human coronavirus genome is a natural SARS-CoV-2 genome, and at least one of the one-to-stop codons is in a sequence corresponding to ORF1ab in the natural SARS-CoV-2 genome.
50. The pharmaceutical product for use according to embodiment 49, wherein at least one of the one-to-stop codons is in a sequence corresponding to an Nsp1 to Nsp15, preferably Nsp3 to Nsp15 encoding sequence in the natural SARS-CoV-2 genome.
51.1 The pharmaceutical product for use according to embodiment 49 or 50, wherein at least one of the one-to-stop codons is in a sequence corresponding to an Nsp3 to Nsp7 or an Nsp12 to Nsp15 encoding sequence in the natural SARS-CoV-2 genome.
51.2 The pharmaceutical product for use according to embodiment 49 or 50, wherein the one-to-stop codons are in sequences corresponding to Nsp3 to Nsp7 and Nsp12 to Nsp15 encoding sequences in the natural SARS-CoV-2 genome.
52. The pharmaceutical product for use according to any one of the preceding embodiments 41-51, wherein the natural human coronavirus genome is a natural SARS-CoV-2 genome, and wherein at least one of the one-to-stop codons has a CDS codon number corresponding to a CDS codon number as indicated in Table 1 or supplementary Table 3 for (or relative to) SEQ ID NO: 7.
53.1 The pharmaceutical product for use according to embodiment 52, wherein at least one of the one-to-stop codons is in a sequence corresponding to an Nsp3 to Nsp7 or an Nsp12 to Nsp15 encoding sequence in the natural SARS-CoV-2 genome and at least one of the one-to-stop codon location is defined by a CDS codon number corresponding to a CDS codon number as indicated in Table 1 or supplementary Table 3 for (or relative to) SEQ ID NO: 7.
53.2 The pharmaceutical product for use according to embodiment 52, wherein at least one of the one-to-stop codons is in a sequence corresponding to an Nsp3 to Nsp7 or an Nsp12 to Nsp15 encoding sequence in the natural SARS-CoV-2 genome and at least one of the one-to-stop codon location is defined by a codon change and CDS codon number corresponding to a CDS codon number as indicated in Table 1 or supplementary Table 3 for (or relative to) SEQ ID NO: 7.
54. The pharmaceutical product for use according to embodiment 52 or 53, wherein the one-to-stop codon locations are defined by CDS codon numbers, each corresponding to a CDS codon number from 2023 to 6614 as indicated in Table 1 or supplementary Table 3 for (or relative to) SEQ ID NO: 7;
   preferably, the one-to-stop codons are defined by codon changes and CDS codon numbers, each corresponding to a CDS codon number from 2023 to 6614 as indicated in Table 1 or supplementary Table 3 for or relative to SEQ ID NO: 7.
55. The pharmaceutical product for use according to any one of the preceding embodiments 41-54, wherein the polynucleotide consists of or comprises a sequence as defined in SEQ ID NO: 3-6 or 9-23, preferably SEQ ID NO: 3-6, more preferably SEQ ID NO: 4-6, again more preferably SEQ ID NO: 5 or 6.
56. The pharmaceutical product of the invention and according to any one of the preceding embodiments comprising the polynucleotide of the invention, vector of the invention, genetically modified cell of the invention and/or attenuated virus of the invention, for use in the prevention or treatment of a corona virus infection in a human subject,
   wherein said polynucleotide encodes an attenuated human coronavirus or a fragment thereof, wherein the polynucleotide comprises at least 20 one-to-stop codons, wherein a one-to-stop codon is i) a different but synonymous codon compared to a corresponding codon in a natural human coronavirus genome or a fragment thereof; and ii) differs by only one nucleotide from a STOP codon, and
   wherein said human subject is challenged by corona virus infection.
57. The pharmaceutical product for use according to embodiment 56, wherein the corona virus infection is a SARS-CoV-2 virus infection.
58. The pharmaceutical product for use according to embodiment 56 or 57, wherein said human subject is challenged by a SARS-CoV-2 virus more than 21 days after vaccination with the pharmaceutical product of the invention comprising the polynucleotide, vector, genetically modified cell and/or attenuated virus according to the invention.
59.1 The pharmaceutical product for use according to embodiments 56-58, wherein the human subject is at increased risk of developing severe COVID-19.
59.2 The pharmaceutical product for use according to embodiments 56-58, wherein the human subject is at increased risk of developing severe COVID-19 or acute respiratory distress syndrome.
60. The pharmaceutical product for use according to embodiments 56-59, wherein said SARS-CoV-2 virus infection is a severe COVID-19 infection or an acute respiratory distress syndrome, preferably said SARS-CoV-2 virus infection is a severe COVID-19 infection.

### SUMMARY OF THE INVENTION

The inventors developed a safe and effective live-attenuated SARS-CoV-2 vaccines (LAVs, herein also called OTS mutants) based on the one-to-stop (OTS) approach. By introducing synonymous codon changes into the open-reading-frame (ORF) lab, the inventors maintained identical amino acid sequences to the wild-type virus while increasing the probability of premature termination codons. This compromises viral fitness and pathogenicity, contributing to attenuation.

The inventors demonstrated that the level of attenuation can be adjusted by enriching specific regions of the viral genome with one-to-stop codons. Through stepwise modifications, the inventors achieved significant attenuation in mice, resulting in 100% survival in a lethal SARS-CoV-2 animal model. Furthermore, the inventors disarmed the virus by introducing changes in specific genes known to interfere with antiviral cellular responses.

To enhance safety and antigenicity, non-structural protein 1 (NSP1) can be modified and specific ORFs, preferably 6 to 8 and the polybasic spike S1/S2 cleavage site can be deleted. By deleting these ORFs, the inventors promote early interferon responses, enhance LAV attenuation, and improve immunogenicity. Furthermore, the inventors removed the PRRAR motif from the polybasic spike S1/S2 cleavage site. Several vaccine candidates were generated using the OTS approach, and their attenuation levels were adaptable based on the extent of genome modification. Enriching OTS codons increased vulnerability to mutagenic drugs.

The combination of Nsp1 (K164A/H165A) mutations and ORF6-8 knockout resulted in a fully protective LAV candidate named OTS-206 against severe disease from various virus variants. OTS-206, showed full attenuation in animal models and provided protection against both wild-type SARS-CoV-2 and the Omicron BA.2 variant. Importantly, OTS-206 immunization led to faster clearance of the Delta variant compared to mRNA vaccines and resolved innate immune responses more rapidly. In addition, using a prime-boost scheme, the inventors observed long-term immunity for up to five months following OTS-206 immunization. The overall protection against the Delta variant was at least comparable to mRNA vaccines, suggesting that live-attenuated vaccines could serve as second-generation vaccines to boost preexisting immunity.

Additionally, OTS-228, which included an extra deletion of the furin cleavage site, successfully blocked LAV transmission without compromising its protective potential. A single intranasal dose of OTS-228 provided robust protection against severe pathology, prevented virus replication in the lungs, completely blocked transmission of the wild-type virus and significantly reduced transmission of the Omicron BA.2 and BA.5 variants. These results highlight the potential of live-attenuated vaccines like OTS-228 to provide broad and long-lasting immunity against SARS-CoV-2 and future variants.

Through in vitro and pre-clinical animal model assessments, the inventors demonstrated that OTS mutants of the invention possess exceptional safety profiles and are at least as efficient as current mRNA vaccines. They induce protective immunity against the original SARS-CoV-2 strain as well as recent variants such as Omicron BA.2 and BA.5. In summary, the OTS mutants of the invention offer promising solutions for robust and adaptable SARS-CoV-2 vaccination strategies. They elicit strong protective immune responses, prevent severe disease, and reduce viral shedding and breakthrough infections.

Accordingly, in one embodiment, the invention relates to a polynucleotide encoding an attenuated human coronavirus, preferably SARS-CoV-2, or to a fragment thereof, wherein the polynucleotide comprises at least 20 one-to-stop codons, wherein a one-to-stop codon is i) a different but synonymous codon compared to a corresponding codon in a natural human coronavirus genome, preferably natural SARS-CoV-2 genome, or a fragment thereof; and ii) differs by only one nucleotide from a STOP codon.

The term “polynucleotide”, as used herein, refers to a nucleic acid that includes at least 60 nucleic acid monomer units (e.g., nucleotides), typically more than 100 monomer units, and more typically greater than 200 monomer units. Polynucleotides are optionally prepared by any suitable method, including, but not limited to, isolation of an existing or natural sequence, DNA replication or amplification, reverse transcription, cloning and restriction digestion of appropriate sequences, or direct chemical synthesis by methods known in the art. The term “nucleic acid” refers to any kind of deoxyribonucleotide (e.g., DNA, cDNA, . . . ) or ribonucleotide (e.g. RNA, mRNA, . . . ) polymer or a combination of deoxyribonucleotide and ribonucleotide (e.g. DNA/RNA) polymer, in linear or circular conformation, and in either single—or double-stranded form. These terms can encompass known analogs of natural nucleotides, as well as nucleotides that are modified in the base, sugar and/or phosphate moieties (e.g. phosphorothioate backbones). In general, an analog of a particular nucleotide has the same base-pairing specificity, i.e., an analog of A will base-pair with T.

The term “attenuated human coronavirus”, as used herein, refers to a human coronavirus that, in comparison to a natural human coronavirus, provokes less and/or less severe or even no symptoms in a host organism after the host organism has been confronted (infected) with the attenuated virus. At the same time, the live attenuated virus induces an immune response of the host to the attenuated virus that is at least partially protective against a wild-type virus infection and/or at least one symptom thereof. In certain embodiments the human coronavirus is a beta coronavirus such as a beta coronavirus selected from the group consisting of: MERS-CoV, SARS-CoV-1, and SARS-CoV-2, preferably SARS-CoV-2.

The term “fragment”, as used herein, refers to a sequence encoding fewer proteins and/or proteins with fewer amino acids in length than the natural human coronavirus (preferably SARS-CoV-2) genome. In some embodiments, the fragment can be used to be assembled with natural human coronavirus (preferably SARS-CoV-2) sequence parts to form a sequence that encodes an attenuated human coronavirus (preferably SARS-CoV-2). In certain embodiments, the “fragment” described herein is a plurality of sequences that together encode at least 60%, at least 70%, at least 80%, at least 90%, at least 95% or 100% of the natural human coronavirus (preferably SARS-CoV-2) genome. In certain embodiments, the fragment has a length sufficient to encode a peptide that is able to induce an immune response in a human subject.

In certain embodiments the fragment of the polynucleotide described herein when combined with corresponding human coronavirus parts encodes a coronavirus particle that induces an immune response after immunization of mice with 5000 PFU coronavirus particle after 15 days.

In certain embodiments the fragment of the polynucleotide described herein when combined with corresponding human coronavirus parts encodes a coronavirus particle that induces an immune response after immunization of mice with 5000 PFU coronavirus particle after 15 days and an increased immune response upon challenge with WT human coronavirus after 21 days measured after 35 days.

In certain embodiments the fragment of the polynucleotide described herein when combined with corresponding human coronavirus parts encodes a coronavirus particle that increases the percentage of S-Tet+ CD8+ T cells upon challenge with WT human coronavirus after 21 days measured after 26 days.

In certain embodiments the fragment of the polynucleotide described herein when combined with corresponding human coronavirus parts encodes a coronavirus particle that induces an immune response after immunization of mice with 5000 PFU coronavirus particle after 15 days and increases the percentage of S-Tet+ CD8+ T cells upon challenge with WT human coronavirus after 21 days measured after 26 days.

The “corresponding human coronavirus parts” as used herein, refers to the parts of the virus genome that is missing in the fragment. The skilled person is aware how to combine virus genome fragments. For example, coronavirus particles may be produced combining the fragment sequence with sequence parts encoding the missing proteins of the virus to a complete or substantially complete sequence that encodes the coronavirus particle. Alternatively, the coronavirus particle may be produced by a trans complementing cell line. The skilled person may use any alignment method to identify which is the closest related human corona virus and which sequence part(s) is/are corresponding human coronavirus part(s).

The “coronavirus particle” is protein-complex encoded in the combination of the fragment alone or the fragment and the corresponding coronavirus sequence parts, typically comprising a virus envelope, preferably more than half of all structural proteins, more preferably all structural proteins.

The induced and/or increased immune response is preferably measured by measurement of neutralizing antibody titers in serum of the mice in a neutralization assay, more preferably with a threshold of 20 VNT100 is considered to be an “induced immune response” (see FIG. 18).

An increase in the percentage of S-Tet+ CD8+ T cells is preferably measured by tetramer staining (see FIG. 18).

The skilled person is aware which animal is sensitive to the respective coronavirus and may replace the mouse with a different animal in the above described measurement setup. Depending on the type of coronavirus, the skilled person may choose for example hamsters, rats, guinea pigs, ferrets, monkeys or domestic pigs depending on the sensitivity of the WT virus instead of mice. Additionally the skilled person may make appropriate changes to the experimental setup such as the dose and timepoints. Furthermore, the animal may be genetically modified to increase sensitivity to the WT virus.

In certain embodiments, the fragment described herein has a length of at least 500, at least 1000, at least 2000, at least 3000, at least 4000, at least 5000, at least 6000, at least 7000, at least 8000, at least 9000, at least 10000, at least 15000, at least 20000 or at least 25000 nucleotides.

The term “STOP codon”, as used herein, refers to any STOP codon known in the art. In some embodiments, the STOP codon(s) is/are at least one selected from the group of UAA (RNA), UAG (RNA), UGA (RNA), TAA (DNA), TAG (DNA) and TGA (DNA).

Two codons are considered “different” herein if they differ in their nucleotides and/or nucleotide order.

Codons are considered “synonymous” herein if they code for the same amino acid or for similar amino acids, preferably if they code for the same amino acid. “Similar amino acids” in the context of synonymous codons are amino acids that can be replaced and wherein the replacement does not or not substantially alter the antigenicity of the protein of which they are part. In a preferred embodiment, synonymous codons are two codons that code for the same amino acid.

For example, the CUU codon, which codes for Leu, is replaced by the codon UUA, which also codes for Leu, but which (contrary to the CUU codon) differs by only one nucleotide from a STOP codon (i.e., from the STOP codon UAA). One-to-stop codon modifications in the polynucleotide of the invention induce differences from the wild-type (e.g., infectious) human coronavirus genome or clone by nucleotide sequence, but not by amino acid sequence (at least not before the first replication cycle).

Alternatively or complementarily, more particularly complementarily, the means of the application may involve the replacement of codon(s), which codes(code) for Thr or Ala, by codon(s) which codes(code) for Ser and differs (differ) by only one nucleotide from a STOP codon. For example, the ACA codon, which codes for Thr, may be replaced by the UCA codon, which codes for Ser, which in turn differs from the UAA STOP codon by only one nucleotide. Such codon replacement modifies the amino acid sequence of the encoded protein and therefore is selected to not (substantially) modify the antigenicity of this protein. The polynucleotide of the invention may additionally comprise further types of near to stop codons.

In some embodiments, the polynucleotide has further modifications of different nature (i.e. modifications other than one-to-stop modifications) and/or deletions that influence the amino acid sequence in the desired manner.

The term “natural human coronavirus”, as used herein, refers to any known human coronavirus preferably SARS-CoV-2 or variants derived thereof. The natural human coronavirus “genome” described herein refers to the genome itself or to a cDNA clone thereof. The natural human coronavirus genome is preferably a natural SARS-CoV-2 genome. In some embodiments, the natural SARS-CoV-2 genome described herein is the genome of a variant selected from the group of Alpha, Beta, Gamma, Delta, Omicron, Lambda, Mu, Epsilon, Zeta, Eta, Theta and Iota, preferably Omicron. In some embodiments, the natural SARS-CoV-2 genome described herein is the genome of a variant selected from the group of Alpha, Beta, Gamma, Delta, Omicron Lineage B.1.1.529, Omicron Lineage BA.2, Lambda, Mu, Epsilon, Zeta, Eta, Theta and Iota. In some embodiments, the natural SARS-CoV-2 genome described herein is the genome of a variant derived from a variant selected from the group of Delta, Omicron Lineage B.1.1.529 and Omicron Lineage BA.2. In some embodiments, the natural SARS-CoV-2 genome described herein is the genome of the Omicron Lineage. The skilled person is aware, how to retrieve the corresponding sequences. In certain embodiments, the SARS-CoV-2 genome described herein is a sequence encoding at least 50%, at least 60%, at least 70%, at least 80%, at least 90%, at least 95% or 100% of all SARS-CoV-2 proteins. In certain embodiments, the SARS-CoV-2 genome described herein is a sequence described in the GISAID dataset describing SARS-CoV-2 variants (Khare, S., et al (2021) GISAID's Role in Pandemic Response. China CDC Weekly, 3(49): 1049-1051). Preferably the GISAID dataset describing SARS-CoV-2 variants comprising 15295201 genome sequence submissions on Mar. 28, 2023, more preferably the GISAID dataset describing SARS-CoV-2 variants on Oct. 12, 2022, even more preferably the GISAID dataset describing SARS-CoV-2 variants on Mar. 28, 2022. In some embodiments, the natural SARS-CoV-2 genome described herein is a sequence with the accession number MT108784 (SEQ ID NO: 7). The SARS-CoV-2 sequence continues to mutate. The skilled person is aware how to distinguish future mutations from other viruses. In certain embodiments, a sequence being 80%, 85%, 90%, 95%, 97%, 98%, 99% or 99.5% identical to the SARS-CoV-2 genome sequence(s) described herein is considered to be a natural SARS-CoV-2 genome, if it maintains the ability to encode one or more SARS-CoV-2 virus proteins. In some embodiments, the natural SARS-CoV-2 genome is a SARS-CoV-2 genome comprising at least one mutation selected from the group of del 69-70, RSYLTPGD246-253N, N440K, G446V, L452R, Y453F, S477G/N, E484Q, E484K, F490S, N501Y, N501S, D614G, Q677P/H, P681H and P681R. In some embodiments, the natural SARS-CoV-2 genome is a SARS-CoV-2 genome comprising at least one mutation selected from the group consisting of del 69-70, RSYLTPGD246-253N, N440K, G446V, L452R, Y453F, S477G/N, E484Q, E484K, F490S, N501Y, N501S, D614G, Q677P/H, P681H, P681R and A701V.

As such, the natural human coronavirus (preferably SARS-CoV-2) genome or fragment thereof serves as a reference sequence for the polynucleotide of the invention.

The term “corresponding” in the context of a codon in relation to the natural human coronavirus (preferably SARS-CoV-2) genome or a fragment thereof refers to the position of the codon.

The skilled person is aware of how to determine a position of a corresponding codon for example using alignment techniques, for instance, using publicly available computer software such as BLAST, BLAST-2, ALIGN or Megalign (DNASTAR) software. Those skilled in the art can determine appropriate parameters for aligning sequences and determining positions, including any algorithms needed to achieve maximal alignment over the full length of the sequences being compared.

The inventors found that the human coronavirus (preferably SARS-CoV-2) virus can be attenuated by replacing codons with synonymous one-to-stop codons. These replacements do not result in changes on protein level and induce therefore an identical or similar immune response as the original virus. The presence of one-to-stop codons reduces the fitness of the virus by increasing the likelihood of a mutation to result in a STOP codon at a critical position. The inventors found that a certain number of one-to-stop codons is required to achieve a substantial attenuation of a human coronavirus, preferably SARS-CoV-2.

Accordingly, the invention is at least in part based on the finding that an attenuated human coronavirus can safely and efficiently be achieved by a polynucleotide having a certain number of one-to-stop codons.

Furthermore, the specific one-to-stop codon replacement enables more positions in the genome for specific and targeted replacements than other attenuation methods such as codon pair deoptimization. As such, the balance between attenuation and immunogenicity can be better optimized than with previous methods. Furthermore, the one-to-stop codons also allow for a targeted attenuation that can be regulated by the location and number of one-to-stop codons as well as by the presence of a mutagen.

In certain embodiments, the invention relates to a method for producing a polynucleotide of the invention, the method comprising the steps of: a) providing the CDS of a natural human coronavirus (preferably SARS-CoV-2) genome, a fragment or cDNA clone thereof; and b) modifying the natural human coronavirus (preferably SARS-CoV-2) genome, the fragment or the retro-transcribed cDNA sequence of the cDNA clone, respectively, wherein said modification comprises replacing at least 20 codons in the natural human coronavirus (preferably SARS-CoV-2) genome, the fragment or the retro-transcribed cDNA sequence, by at least 20 one-to-stop codons, wherein a one-to-stop codon is i) a different but synonymous codon compared to a corresponding codon in the natural human coronavirus (preferably SARS-CoV-2) genome, the fragment or the retro-transcribed cDNA sequence; and ii) differs by only one nucleotide from a STOP codon.

The term “CDS” of a natural human coronavirus (preferably SARS-CoV-2) genome, as used herein, refers to the coding sequence of the natural human coronavirus (preferably SARS-CoV-2) genome

The step of “modifying”, described herein, refers to altering a sequence. This alteration can be achieved by any method known in the art including resynthesis, meganucleases and Crispr.

The replacement can be achieved by removing the sequence part (e.g. the codon) from a polynucleotide and inserting the desired sequence part and/or by resynthesizing the sequence with the desired sequence part.

The inventors found that replacing certain codons in the CDS of a natural human coronavirus (preferably SARS-CoV-2) genome enables attenuation of the fitness of the encoded human coronavirus (preferably SARS-CoV-2) if enough codons are replaced.

Accordingly, the invention is at least in part based on the finding that a polynucleotide encoding an attenuated human coronavirus (preferably SARS-CoV-2) can be produced by replacing a certain number of codons with one-to-stop codons.

In certain embodiments, the invention relates to the polynucleotide of the invention or the method of the invention, wherein at least one of the one-to-stop codons is comprised in a sequence part or fragment corresponding to a sequence part of ORF1ab of the natural SARS-CoV-2, a sequence part encoding a structure protein of the natural SARS-CoV-2 or a sequence part encoding an accessory protein of the natural SARS-CoV-2. The term “ORF1ab”, as used herein, refers to Open reading frame (ORF) 1 a and/or b of the natural SARS-CoV-2 genome or an ORF of a SARS-CoV-2 genome corresponding to the ORF1ab of SEQ ID NO: 7.

The terms “sequence part encoding an accessory gene” and “sequence encoding an accessory gene”, as used herein, refers to accessory protein ORFs 3a, 3b, 6, 7a, 7b, 8, 9b, 9c, and/or 10.

The term “structure protein”, as used herein, refers to the SARS-CoV-2 protein S, E, M and/or N.

ORF1ab, accessory genes and structure proteins comprise information that is relevant for the fitness and reproducibility of SARS-CoV-2. The inventors found that one-to-stop codons in these sequence parts are particularly effective in attenuating SARS-CoV-2. Without being bound by theory, a mutation to a STOP codon in these areas will substantially reduce or eliminate the virus's ability to reproduce.

Accordingly, the invention is at least in part based on the finding that one-to-stop codons in the sequence parts encoding for ORF1ab, accessory genes and structural proteins are particularly effective in attenuating the SARS-CoV-2.

In certain embodiments, the invention relates to the polynucleotide of the invention or the method of the invention, wherein at least one of the one-to-stop codons is comprised in a sequence part or fragment corresponding to a sequence part of ORF1ab of the natural SARS-CoV-2. In certain embodiments, at least 50%, preferably at least 70%, more preferably at least 80%, again more preferably at least 90%, again more preferably at least 95%, again more preferably at least 99%, more preferably 100% of the one-to-stop codons are in a sequence corresponding to a sequence of ORF1ab of the natural SARS-CoV-2 genome.

ORF1ab is particularly relevant for the fitness and reproducibility of SARS-CoV-2. The inventors found that one-to-stop codons in these sequence parts are particularly effective in attenuating SARS-CoV-2. Without being bound by theory, a mutation to a STOP codon in this area will substantially reduce or eliminate the virus's ability to reproduce.

Accordingly, the invention is at least in part based on the finding that one-to-stop codons in the sequence parts encoding for ORF1ab are particularly effective in attenuating the SARS-CoV-2.

In certain embodiments, the invention relates to the polynucleotide of the invention or the method of the invention, wherein at least one of the one-to-stop codons is comprised in a sequence part or fragment corresponding to an Nsp2 to Nsp15 encoding sequence part of the natural SARS-CoV-2 genome. In certain embodiments, the invention relates to the polynucleotide of the invention or the method of the invention, wherein at least one of the one-to-stop codons is comprised in a sequence corresponding to an Nsp1 to Nsp15, preferably Nsp3 to Nsp15 encoding sequence of the natural SARS-CoV-2 genome. In certain embodiments, at least 50%, preferably at least 70%, more preferably at least 80%, again more preferably at least 90%, again more preferably at least 95%, again more preferably at least 99%, more preferably 100% of the one-to-stop codons are in a sequence or fragment corresponding to an Nsp1 to Nsp15, preferably Nsp3 to Nsp15 encoding sequence of the natural SARS-CoV-2 genome.

Accordingly, the invention is at least in part based on the finding that one-to-stop codons in the sequence parts encoding for Nsp2 to Nsp15 are particularly effective in attenuating the SARS-CoV-2.

In certain embodiments, the invention relates to the polynucleotide of the invention or the method of the invention, wherein at least one of the one-to-stop codons is comprised in a sequence part or fragment corresponding to an Nsp2 to Nsp7 encoding sequence part of the natural SARS-CoV-2 genome. In certain embodiments, at least 50%, preferably at least 70%, more preferably at least 80%, again more preferably at least 90%, again more preferably at least 95%, again more preferably at least 99%, more preferably 100% of the one-to-stop codons is in a sequence corresponding to an Nsp2 to Nsp7 encoding sequence of the natural SARS-CoV-2 genome. In certain embodiments, at least one of the one-to-stop codons is comprised in a sequence corresponding to an Nsp3 to Nsp7 encoding sequence of the natural SARS-CoV-2 genome. In certain embodiments, at least 50%, preferably at least 70%, more preferably at least 80%, again more preferably at least 90%, again more preferably at least 95%, again more preferably at least 99%, more preferably 100% of the one-to-stop codons is in a sequence corresponding to an Nsp3 to Nsp7 encoding sequence of the natural SARS-CoV-2 genome.

In certain embodiments, at least one of the one-to-stop codons is comprised in a sequence corresponding to (i) an Nsp2 to Nsp7, preferably an Nsp3 to Nsp7 and (ii) an Nsp12 to Nsp15 encoding sequence of the natural SARS-CoV-2 genome. In certain embodiments, the least 50%, preferably at least 70%, more preferably at least 80%, again more preferably at least 90%, again more preferably at least 95%, again more preferably at least 99%, more preferably 100% of the one-to-stop codons is in a sequence corresponding to (i) an Nsp2 to Nsp7, preferably an Nsp3 to Nsp7 and (ii) an Nsp12 to Nsp15 encoding sequence of the natural SARS-CoV-2 genome.

In certain embodiments, the invention relates to the polynucleotide of the invention or the method of the invention, wherein at least one of the one-to-stop codons is comprised in a sequence part or fragment corresponding to an Nsp13 to Nsp15 encoding sequence part of the natural SARS-CoV-2 genome. In certain embodiments, at least 50%, preferably at least 70%, more preferably at least 80%, again more preferably at least 90%, again more preferably at least 95%, again more preferably at least 99%, more preferably 100% of the one-to-stop codons is in a sequence or fragment corresponding to an Nsp13 to Nsp15 encoding sequence of the natural SARS-CoV-2 genome. In certain embodiments, at least one of the one-to-stop codons is in a sequence corresponding to an Nsp12 to Nsp15 encoding sequence of the natural SARS-CoV-2 genome. In certain embodiments, the least 50%, preferably at least 70%, more preferably at least 80%, again more preferably at least 90%, again more preferably at least 95%, again more preferably at least 99%, more preferably 100% of the one-to-stop codons is in a sequence corresponding to an Nsp12 to Nsp15 encoding sequence of the natural SARS-CoV-2 genome.

Accordingly, the invention is at least in part based on the finding that one-to-stop codons in certain sequence parts are particularly effective in attenuating the SARS-CoV-2.

In certain embodiments, the invention relates to the polynucleotide of the invention or the method of the invention, wherein the one-to-stop codon(s) comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 one-to-stop codon having a position selected from Table 1 corresponding to a position on the natural SARS-CoV-2 genome, preferably to SEQ ID NO: 7. In certain embodiments, the invention relates to the polynucleotide of the invention or the method of the invention, wherein at least 10%, at least 20%, at least 30%, at least 40%, at least 50%, at least 60%, at least 70%, at least 80%, or at least 90% of the one-to-stop codons in the polynucleotide of the invention have a position selected from Table 1 corresponding to a position on the natural SARS-CoV-2 genome, preferably to SEQ ID NO: 7. In certain embodiments, the invention relates to the polynucleotide of the invention or the method of the invention, wherein the one-to-stop codon(s) comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, or at least 19 one-to-stop codon having a position selected from Table 1 or supplementary Table 3, corresponding to a position on the natural SARS-CoV-2 genome, preferably to SEQ ID NO: 7. In certain embodiments, the invention relates to the polynucleotide of the invention or the method of the invention, wherein at least 10%, at least 20%, at least 30%, at least 40%, at least 50%, at least 60%, at least 70%, at least 80%, or at least 90% of the one-to-stop codons in the polynucleotide of the invention have a position selected from Table 1 or supplementary Table 3, corresponding to a position on the natural SARS-CoV-2 genome, preferably to SEQ ID NO: 7.

In certain embodiments, at least one, preferably at least 10%, more preferably at least 20%, again more preferably at least 30%, again more preferably at least 40%, again more preferably at least 50%, again more preferably at least 60%, again more preferably at least 70%, again more preferably at least 80%, or again more preferably at least 90% of the one-to-stop codons in the polynucleotide of the invention have a CDS codon number corresponding to a CDS codon number, as indicated in Table 1 or Supplementary Table 3 for SEQ ID NO: 7. In certain embodiments, at least one of the one-to-stop codons has a CDS codon number corresponding to a CDS codon number of SEQ ID NO: 7, as indicated in Table 1 or Supplementary Table 3. In certain embodiments, any of the one-to-stop codons has a CDS codon number corresponding to a CDS codon number, as indicated in Table 1 or Supplementary Table 3 for SEQ ID NO: 7.

In certain embodiments, at least one, preferably any of the one-to-stop codons is in a sequence corresponding to an Nsp3 to Nsp7 or Nsp12 to Nsp15 encoding sequence in the natural SARS-CoV-2 genome, and at least one, preferably any of the one-to-stop codons has a CDS codon number corresponding to a CDS codon number, as indicated in Table 1 or Supplementary Table 3 for SEQ ID NO: 7. In certain embodiments, at least one, preferably any of the one-to-stop codons is in a sequence corresponding to an Nsp3 to Nsp7 or Nsp12 to Nsp15 encoding sequence in the natural SARS-CoV-2 genome, and at least one, preferably at least 10%, more preferably at least 20%, again more preferably at least 30%, again more preferably at least 40%, again more preferably at least 50%, again more preferably at least 60%, again more preferably at least 70%, again more preferably at least 80%, or again more preferably at least 90% of the one-to-stop codons in the polynucleotide of the invention have a CDS codon number corresponding to a CDS codon number, as indicated in Table 1 or Supplementary Table 3 for SEQ ID NO: 7.

Preferably, the one-to-stop codons are defined by CDS codon numbers corresponding each to a CDS codon number from 2023 to 6614, as indicated in Table 1 or Supplementary Table 3 for SEQ ID NO: 7. In certain embodiments, at least one, preferably at least 10%, more preferably at least 20%, again more preferably at least 30%, again more preferably at least 40%, again more preferably at least 50%, again more preferably at least 60%, again more preferably at least 70%, again more preferably at least 80%, or again more preferably at least 90% of the one-to-stop codons is defined by a CDS codon number corresponding to a CDS codon number from 2023 to 6614 as indicated in Table 1 or supplementary Table 3 for SEQ ID NO: 7. In certain embodiments, at least one, preferably any of the one-to-stop codons is defined by a CDS codon number corresponding to a CDS codon number from 2023 to 6614 as indicated in Table 1 or supplementary Table 3 for SEQ ID NO: 7.

In certain embodiments, at least one of the one-to-stop codons is defined (i) by a CDS codon number from 2023 to 6614 as indicated in Table 1 or supplementary Table 3 for SEQ ID NO: 7, and (ii) by a codon change as indicated for the corresponding CDS codon number in Table 1 or supplementary Table 3 for SEQ ID NO: 7. In certain embodiments, at least one, preferably at least 10%, more preferably at least 20%, again more preferably at least 30%, again more preferably at least 40%, again more preferably at least 50%, again more preferably at least 60%, again more preferably at least 70%, again more preferably at least 80%, or again more preferably at least 90% of the one-to-stop codons are defined (i) by a CDS codon number from 2023 to 6614 as indicated in Table 1 or supplementary Table 3 for SEQ ID NO: 7, and (ii) by a codon change as indicated for the corresponding CDS codon number in Table 1 or supplementary Table 3 for SEQ ID NO: 7. In certain embodiments, each (100%) of the one-to-stop codons is defined (i) by a CDS codon number from 2023 to 6614 as indicated in Table 1 or supplementary Table 3 for SEQ ID NO: 7, and (ii) by a codon change as indicated for the corresponding CDS codon number in Table 1 or supplementary Table 3 for SEQ ID NO: 7.

More preferably, the one-to-stop codons are defined (i) by CDS codon numbers, wherein each CDS codon number corresponds to a CDS codon number between 2023 and 6614 relative to SEQ ID NO: 7, as indicated in Table 1 or Supplementary Table 3 and (ii) by codon changes, wherein for each of the CDS codon numbers from 2023 to 6614, the codon changes are as indicated in Table 1 or Supplementary Table 3.

### OTS Fragments

In certain embodiments, the polynucleotide comprises a sequence, wherein said sequence comprise said at least 20 one-to-stop codons, wherein at least one, a portion of, or any of the one-to-stop (OTS) codons is defined by a CDS codon number corresponding to a CDS codon number between 88 and 911 as indicated in Table 1 or Supplementary Table 3 relative to SEQ ID NO: 7 (mentioned herein as OTS Nsp1-3). More preferably, the polynucleotide comprises a sequence, wherein said sequence comprise said at least 20 one-to-stop codons, wherein at least one, a portion of, or any of the OTS codons is defined by a codon change and CDS codon number corresponding to a codon change and CDS codon number between 88 and 911 as indicated in Table 1 or Supplementary Table 3 relative to SEQ ID NO: 7 (mentioned herein as Fg2).

In certain embodiments, the polynucleotide comprises a sequence, wherein said sequence comprise said at least 20 one-to-stop codons, wherein at least one, a portion of, or any of the one-to-stop (OTS) codons is defined by a CDS codon number corresponding to a CDS codon number between 2028 and 2804 as indicated in Table 1 or Supplementary Table 3 relative to SEQ ID NO: 7 (mentioned herein as OTS Nsp3-4). More preferably, the polynucleotide comprises a sequence, wherein said sequence comprise said at least 20 one-to-stop codons, wherein at least one, a portion of, or any of the OTS codons is defined by a codon change and CDS codon number corresponding to a codon change and CDS codon number between 2028 and 2804 as indicated in Table 1 or Supplementary Table 3 relative to SEQ ID NO: 7 (mentioned herein as Fg4).

In certain embodiments, the polynucleotide comprises a sequence, wherein said sequence comprise said at least 20 one-to-stop codons, wherein at least one, a portion of, or any of the one-to-stop (OTS) codons is defined by a CDS codon number corresponding to a CDS codon number between 2926 and 3796 as indicated in Table 1 or Supplementary Table 3 relative to SEQ ID NO: 7 (mentioned herein as OTS Nsp4-6). More preferably, the polynucleotide comprises a sequence, wherein said sequence comprise said at least 20 one-to-stop codons, wherein at least one, a portion of, or any of the OTS codons is defined by a codon change and CDS codon number corresponding to a codon change and CDS codon number between 2926 and 3796 as indicated in Table 1 or Supplementary Table 3 relative to SEQ ID NO: 7 (mentioned herein as Fg5).

In certain embodiments, the polynucleotide comprises a sequence, wherein said sequence comprise said at least 20 one-to-stop codons, wherein at least one, a portion of, or any of the one-to-stop (OTS) codons is defined by a CDS codon number corresponding to a CDS codon number between 4793 and 5709 as indicated in Table 1 or Supplementary Table 3 relative to SEQ ID NO: 7 (mentioned herein as OTS Nsp12-13). More preferably, the polynucleotide comprises a sequence, wherein said sequence comprise said at least 20 one-to-stop codons, wherein at least one, a portion of, or any of the OTS codons is defined by a codon change and CDS codon number corresponding to a codon change and CDS codon number between 4793 and 5709 as indicated in Table 1 or Supplementary Table 3 relative to SEQ ID NO: 7 (mentioned herein as Fg7).

In certain embodiments, the polynucleotide comprises a sequence, wherein said sequence comprise said at least 20 one-to-stop codons, wherein at least one, a portion of, or any of the one-to-stop (OTS) codons is defined by a CDS codon number corresponding to a CDS codon number between 5824 and 6614 as indicated in Table 1 or Supplementary Table 3 relative to SEQ ID NO: 7 (mentioned herein as OTS Nsp13-15). More preferably, the polynucleotide comprises a sequence, wherein said sequence comprise said at least 20 one-to-stop codons, wherein at least one, a portion of, or any of the OTS codons is defined by a codon change and CDS codon number corresponding to a codon change and CDS codon number between 5824 and 6614 as indicated in Table 1 or Supplementary Table 3 relative to SEQ ID NO: 7 (mentioned herein as Fg8).

In certain embodiments, the polynucleotide of the invention comprises OTS Nsp1-3 or Fg2. In certain embodiments, the polynucleotide of the invention comprises OTS Nsp3-4 or Fg4. In certain embodiments, the polynucleotide of the invention comprises OTS Nsp4-6 or Fg5. In certain embodiments, the polynucleotide of the invention comprises OTS Nsp12-13 or Fg7. In certain embodiments, the polynucleotide of the invention comprises OTS Nsp13-15 or Fg8.

In certain embodiments, the polynucleotide of the invention comprises OTS Nsp3-4 and OTS Nsp4-6. In certain embodiments, the polynucleotide of the invention comprises Fg4 and Fg5.

In more preferred embodiment, the polynucleotide of the invention comprises OTS Nsp12-13 and OTS Nsp13-15. In an also preferred embodiment, the polynucleotide of the invention comprises Fg7 and Fg8. In more preferred embodiment, the polynucleotide of the invention comprises OTS Nsp3-4, OTS Nsp4-6, OTS Nsp12-13 and OTS Nsp13-15. In another also more preferred embodiment, the polynucleotide of the invention comprises Fg4, Fg5, Fg7 and Fg8.

In more preferred embodiment, the polynucleotide of the invention comprises OTS Nsp3-4, OTS Nsp4-6, OTS Nsp12-13 and OTS Nsp13-15.

In another also more preferred embodiment, the polynucleotide of the invention comprises (i) either Fg4, Fg5, Fg7 and Fg8 or OTS Nsp3-4, OTS Nsp4-6, OTS Nsp12-13 and OTS Nsp13-15; and (ii) a mutated Nsp1 gene comprising at least one mutation, preferably said mutated Nsp1 comprises amino acid exchanges at position(s) that correspond(s) to position(s) K164 and/or H165 of or relative to SEQ ID NO: 7, more preferably, said exchange(s) correspond(s) to exchange(s) K164A and/or H165A in or relative to SEQ ID NO: 7 (Nsp1K164A,H165A).

In another also more preferred embodiment, the polynucleotide of the invention comprises (i) either Fg4, Fg5, Fg7 and Fg8 or OTS Nsp3-4, OTS Nsp4-6, OTS Nsp12-13 and OTS Nsp13-15; and (ii) a mutated Nsp1 gene comprising at least one mutation, preferably said mutated Nsp1 comprises amino acid exchanges at position(s) that correspond(s) to position(s) K164 and/or H165 of or relative to SEQ ID NO: 7, more preferably, said exchange(s) correspond(s) to exchange(s) K164A and/or H165A in or relative to SEQ ID NO: 7 (Nsp1K164A,H165A), and (iii) deletion or mutation, preferably deletion ORF6 to ORF8 or parts thereof.

In another also more preferred embodiment, the polynucleotide of the invention comprises (i) either Fg4, Fg5, Fg7 and Fg8 or OTS Nsp3-4, OTS Nsp4-6, OTS Nsp12-13 and OTS Nsp13-15; and (ii) a mutated Nsp1 gene comprising at least one mutation, preferably said mutated Nsp1 comprises amino acid exchanges at position(s) that correspond(s) to position(s) K164 and/or H165 of or relative to SEQ ID NO: 7, more preferably, said exchange(s) correspond(s) to exchange(s) K164A and/or H165A in or relative to SEQ ID NO: 7 (Nsp1K164A,H165A), (iii) deletion or mutation, preferably deletion of ORF6 to ORF8 or parts thereof; and (iv) a deletion of the furin cleavage site (FCS) in a region corresponding to S1/S2 of SEQ ID NO: 7, preferably, said FCS deletion is a deletion of 24 nucleotides corresponding to nucleotides 23598-23622 of SEQ ID NO: 7.

In another also more preferred embodiment, the polynucleotide of the invention comprises (i) either Fg4, Fg5, Fg7 and Fg8 or OTS Nsp3-4, OTS Nsp4-6, OTS Nsp12-13 and OTS Nsp13-15; and (ii) a mutated Nsp1 gene comprising at least one mutation, (iii) deletion or mutation of ORF6 to ORF8 or parts thereof; and (iv) a deletion of the furin cleavage site (FCS) in a region corresponding to S1/S2 of SEQ ID NO: 7.

In another also more preferred embodiment, the polynucleotide of the invention comprises (i) either Fg4, Fg5, Fg7 and Fg8 or OTS Nsp3-4, OTS Nsp4-6, OTS Nsp12-13 and OTS Nsp13-15; and (ii) a mutated Nsp1 gene comprising at least one mutation, said mutated Nsp1 comprises amino acid exchanges at position(s) that correspond(s) to position(s) K164 and/or H165 of or relative to SEQ ID NO: 7, preferably, said exchange(s) correspond(s) to exchange(s) K164A and/or H165A in or relative to SEQ ID NO: 7 (Nsp1K164A,H165A), (iii) deletion of ORF6 to ORF8 or parts thereof; and (iv) a deletion of the furin cleavage site (FCS) in a region corresponding to S1/S2 of SEQ ID NO: 7, said FCS deletion is a deletion of 24 nucleotides corresponding to nucleotides 23598-23622 of SEQ ID NO: 7.

In another preferred embodiment, the polynucleotide of the invention comprises at least a sequence selected from the group consisting at of SEQ ID NO: 9-18. In another preferred embodiment, the polynucleotide of the invention comprises SEQ ID NO: 9 or 10. In another preferred embodiment, the polynucleotide of the invention comprises SEQ ID NO: 11 or 12. In another preferred embodiment, the polynucleotide of the invention comprises SEQ ID NO: 13 or 14. In another preferred embodiment, the polynucleotide of the invention comprises SEQ ID NO: 15 or 16. In another preferred embodiment, the polynucleotide of the invention comprises SEQ ID NO: 17 or 18. In another preferred embodiment, the polynucleotide of the invention comprises SEQ ID NO: 11, 13 15 and 17. In another preferred embodiment, the polynucleotide of the invention comprises SEQ ID NO: 12, 14, 16, and 18.

In another preferred embodiment, the polynucleotide of the invention comprises (i) SEQ ID NO: 11, 13 15 and 17 or SEQ ID NO: 12, 14, 16, and 18, and (ii) a mutated Nsp1 gene comprising at least one mutation, said mutated Nsp1 comprises amino acid exchanges at position(s) that correspond(s) to position(s) K164 and/or H165 of or relative to SEQ ID NO: 7, preferably, said exchange(s) correspond(s) to exchange(s) K164A and/or H165A in or relative to SEQ ID NO: 7 (Nsp1K164A,H165A), (iii) deletion of ORF6 to ORF8 or parts thereof; and (iv) a deletion of the furin cleavage site (FCS) in a region corresponding to S1/S2 of SEQ ID NO: 7, said FCS deletion is a deletion of 24 nucleotides corresponding to nucleotides 23598-23622 of SEQ ID NO: 7.

In another preferred embodiment, the polynucleotide of the invention comprises (i) SEQ ID NO: 11, 13 15 and 17 or SEQ ID NO: 12, 14, 16, and 18, and (ii) a mutated Nsp1 gene comprising at least one mutation, (iii) deletion or mutation of ORF6 to ORF8 or parts thereof; and (iv) a deletion of the furin cleavage site (FCS) in a region corresponding to S1/S2 of SEQ ID NO: 7.

Sequences comprised in the polynucleotide of the invention can overlap, can be separated by a peptide linker or consecutively linked to each other.

Accordingly, the invention is at least in part based on the finding that one-to-stop codons in certain positions are particularly effective in attenuating the SARS-CoV-2.

In certain embodiments, the invention relates to the polynucleotide of the invention or the method of the invention, wherein the amino acids encoded by the at least 20 one-to-stop codons consist of Leu, Ser, Arg and/or Gly.

In certain embodiments, the invention relates to the polynucleotide of the invention or the method of the invention, wherein the amino acids encoded by the one-to-stop codons consist of Leu and/or Ser. Leu and Ser allow many combinations to design one-to-stop codons.

Accordingly, the invention is at least in part based on the finding that certain amino acids are encoded by codons that are particularly effective one-to-stop codons.

In certain embodiments, the invention relates to the polynucleotide of the invention or the method of the invention, wherein the at least 20 one-to-stop codons are at least 20, at least 25, at least 30, at least 35, at least 40, at least 45, at least 50, at least 55, at least 60; at least 65, at least 70, at least 75, at least 80, at least 85, at least 90, at least 95, at least 100, at least 105, at least 110, at least 115, at least 120, one-to-stop codons.

In certain embodiments, the invention relates to the polynucleotide of the invention or the method of the invention, wherein the at least 20 one-to-stop codons are at least 150, preferably at least 180, more preferably at least 200, again more preferably at least 220, again more preferably at least 250, again more preferably at least 280, again more preferably at least 300, again more preferably at least 320.

In certain embodiments, the invention relates to the polynucleotide of the invention or the method of the invention, wherein the polynucleotide comprises at least 20, at least 50, at least 100, at least 150, at least 200, at least 250, at least 300, at least 350, at least 400, at least 450, at least 500, at least 550 mutation.

Accordingly, the invention is at least in part based on the finding that the attenuation of human coronavirus (preferably SARS-CoV-2) is substantial with a certain number of one-to-stop codons.

The inventors found that combining two fragments comprising one-to-stop codons particularly attenuates the encoded SARS-CoV-2 virus.

In certain embodiments, the invention relates to the polynucleotide of the invention or the method of the invention, wherein at least 20, at least 25, at least 30, at least 35, at least 40, at least 45, at least 50, at least 55 or at least 60 one-to-stop codons are comprised in one fragment.

### Nsp1

In certain embodiments, the invention relates to the polynucleotide of the invention, wherein the polynucleotide comprises no sequence encoding a protein having an Nsp1 functionality of the natural SARS-CoV-2 or a sequence encoding a protein having a reduced Nsp1 functionality compared to the Nsp1 of the natural SARS-CoV-2.

In a preferred embodiment, the polynucleotide comprises a mutated Nsp1 gene, wherein preferably the mutated Nsp1 gene encodes a protein comprising at least one mutation. Preferably, the polynucleotide comprises a mutated Nsp1 gene comprising at least two, more preferably exactly two amino acid exchanges as compared to natural SARS-CoV-2 gene. Preferably, said amino acid exchanges is/are at position(s) that correspond(s) to position(s) K164 and/or H165 of or relative to SEQ ID NO: 7. More preferably, said least two or exactly two amino acid exchange(s) correspond(s) to exchange(s) K164A and/or H165A in or relative to SEQ ID NO: 7 (Nsp1K164A,H165A). In a very preferred embodiment, said mutated Nsp1 comprises mutations corresponding to A755G, A756C (K164A), C758G, A759C (H165A) in or relative to SEQ ID NO: 7.

The functions of Nsp1 are characterized (see, e.g., Min, Yuan-Qin, et al. Frontiers in microbiology (2020): 2393) and include inhibition of host mRNA translation and induction of inflammatory cytokines. Reduced or eliminated Nsp1 functionality, therefore results in reduced host (cell) stress induced by the attenuated virus. Therefore, without being bound by theory, the one-to-stop mechanism attenuates Sars-CoV-2s reproducibility and infectiousness, while the reduced Nsp1 functionality reduces the side-effects induced by the attenuated Sars-CoV-2, and increases host cell responses to infections since cellular translation is not blocked.

Accordingly, the invention is at least in part based on the finding that the combination of one-to-stop codon attenuation and reduced or modified Nsp1 have a synergistic effect.

In certain embodiments, the invention relates to the polynucleotide of the invention, wherein the polynucleotide comprises no sequence encoding a protein having the functionality of a protein encoded by the ORF6 gene of the natural SARS-CoV-2 or a sequence encoding a protein having a reduced functionality of a protein encoded by the ORF6 gene of the natural SARS-CoV-2.

In certain embodiments, the invention relates to the polynucleotide of the invention, wherein the polynucleotide comprises no sequence encoding a protein having the functionality of a protein encoded by the ORF7a gene of the natural SARS-CoV-2 or a sequence encoding a protein having a reduced functionality of a protein encoded by the ORF7a gene of the natural SARS-CoV-2.

In certain embodiments, the invention relates to the polynucleotide of the invention, wherein the polynucleotide comprises no sequence encoding a protein having the functionality of a protein encoded by the ORF7b gene of the natural SARS-CoV-2 or a sequence encoding a protein having a reduced functionality of a protein encoded by the ORF7b gene of the natural SARS-CoV-2.

In certain embodiments, the invention relates to the polynucleotide of the invention, wherein the polynucleotide comprises no sequence encoding a protein having the functionality of a protein encoded by the ORF8 gene of the natural SARS-CoV-2 or a sequence encoding a protein having a reduced functionality of a protein encoded by the ORF8 gene of the natural SARS-CoV-2.

In certain embodiments, the invention relates to the polynucleotide of the invention, wherein the polynucleotide comprises no sequence encoding a protein having the functionality of a protein encoded by: a) the ORF8 gene and the ORF6 gene, b) the ORF8 gene and the ORF7a gene, c) the ORF8 gene and the ORF7b gene, d) the ORF6 gene and the ORF7a gene, e) the ORF6 gene and the ORF7b gene, or f) the ORF7a gene and the ORF7b gene of the natural SARS-CoV-2 or a sequence encoding a protein having a reduced functionality of a protein encoded by the respective gene combination a)-f) of the natural SARS-CoV-2.

In certain embodiments, the invention relates to the polynucleotide of the invention, wherein the polynucleotide comprises no sequence encoding a protein having the functionality of a protein encoded by: a) the ORF8 gene and the ORF6 gene and the ORF7a gene, b) the ORF8 gene and the ORF6 gene and the ORF7b gene, c) the ORF7b gene and the ORF6 gene and the ORF7a gene, or d) the ORF8 gene and the ORF7b gene and the ORF7a gene of the natural SARS-CoV-2 or a sequence encoding a protein having a reduced functionality of a protein encoded by the respective gene combination a)-d) of the natural SARS-CoV-2.

In certain embodiments, the invention relates to the polynucleotide of the invention, wherein the polynucleotide comprises no sequence encoding a protein having the functionality of a protein encoded by the ORF8 gene and the ORF6 gene and the ORF7a gene and the ORF7b gene of the natural SARS-CoV-2 or a sequence encoding a protein having a reduced functionality of a protein encoded by the ORF8 gene and the ORF6 gene and the ORF7a gene and the ORF7b gene of the natural SARS-CoV-2.

In certain embodiments, ORF6, ORF7a, ORF7b, ORF8, parts thereof or a combination thereof has been deleted or mutated in the polynucleotide of the invention. Preferably, a region from ORF6 to ORF8 or parts thereof, preferably a region from ORF6 to ORF8 has been deleted or mutated in the polynucleotide of the invention. Preferably, said mutation may is not silent, i.e. it changes the corresponding amino acid sequence of the protein. Preferably said mutation results in a non-functional or no protein. More preferably, preferably a region starting at the beginning of or within ORF6 and ending within or at the end of ORF8 has been deleted.

In certain embodiments, the polynucleotide of the invention does not encode a protein encoded by ORF6, ORF7a, ORF7b or ORF8, or does not encode a functional protein encoded by ORF6, ORF7a, ORF7b or ORF8, of the natural human SARS-CoV-2 genome. In certain embodiments, ORF6, ORF7a, ORF7b or ORF8 have partly or completely been deleted in the polynucleotide of the invention (Deletion ORF6-ORF8). In certain embodiments, nucleotides corresponding to nucleotides at positions 27,192 to 28,247 in or relative to SEQ ID NO: 7 have been deleted (delORF6-ORF8). Deletion of nucleotides at positions 27,192 to 28,247 in SEQ ID NO: 7 is demonstrated in SEQ ID NO: 2. The functions of ORF6 and ORF8 are characterized and include immune-evasive mechanisms and are involved in virus host interactions. Reduced or eliminated functionality of the ORF6 gene, ORF7a gene, ORF7b gene, and/or ORF8 gene, therefore can result in reliable recognition by the immune system or impaired virus host interactions of the attenuated virus. Therefore, without being bound by theory, the one-to-stop mechanism attenuates Sars-CoV-2s reproducibility and infectiousness, while the absence or reduced functionality of the protein(s) expressed by the ORF6 gene, ORF7a gene, ORF7b gene, and ORF8 gene enhances recognition by the immune system and/or impairs virus host interactions of the attenuated SARS-CoV-2 and/or reduces the required dose of the attenuated SARS-CoV-2 to induce a certain immune response.

Accordingly, the invention is at least in part based on the finding that the combination of one-to-stop codon attenuation and ORF6, ORF7a gene, ORF7b gene, and/or ORF8 deletion or modification have a synergistic effect.

### ΔPRRAR

In certain embodiments, the invention relates to the polynucleotide of the invention, wherein the polynucleotide comprises a sequence encoding a spike protein, wherein the spike protein comprises a modified or removed cleavage site compared to the cleavage site of the spike protein of the natural SARS-CoV-2.

In certain embodiments, the polynucleotide of the invention encodes a spike protein, wherein the spike protein comprises a modified or removed furin cleavage site as compared to the cleavage site of the spike protein of the natural human SARS-CoV-2.

In certain embodiments, the polynucleotide of the invention comprises a polybasic S1/S2 furin cleavage site (PCS) deletion (ΔPRRAR) or modification, preferably a deletion of the furin cleavage site (FCS) in a region corresponding to S1/S2 of SEQ ID NO: 7. Preferably, ΔPRRAR is a deletion of 24 nucleotides corresponding to nucleotides 23598-23622 of SEQ ID NO: 7. This results in deletion of 8 amino acids corresponding to aa 679-686 in a protein encoded by SEQ ID NO: 7.

The inventors found, that upon production of the attenuated SARS-CoV-2, the virus tends to mutate in the host cells and modify the cleavage site or remove the cleavage site in the spike protein. By starting with a sequence comprising a modified or removed cleavage site in the starting sequence, the sequence gets replicated more uniformly and/or more efficiently.

The inventors found, that upon infection with the attenuated SARS-CoV-2, virus transmission to co-housed animals was absent or reduced when an attenuated SARS-CoV-2 was used that lacks the cleavage site in the spike protein.

The inventors found that replication of an attenuated SARS-CoV-2 lacking the cleavage site in the spike protein was still efficient in mucosal tissues of the upper respiratory tract, while replication in the lungs was reduced.

Accordingly, the invention is at least in part based on the finding that the combination of one-to-stop codon attenuation, Nsp1K164A,H165A and deletion or modification of S1/S2 furin cleavage site and ORF6, ORF7a gene, ORF7b gene, and/or ORF8 have a synergistic effect.

Accordingly, the invention is at least in part based on the finding that modifying or removing the cleavage site of the spike protein improves the production of an attenuated SARS-CoV-2 virus, reduces transmission, and reduces replication in the lower respiratory tract.

In certain embodiments, the invention relates to a polynucleotide according to the invention, wherein the polynucleotide consists of or comprises a sequence as defined SEQ ID NO: 3-6.

In certain embodiments, the invention relates to a vector comprising the polynucleotide of the invention.

The term “vector”, as used herein, refers to a nucleic acid molecule that is designed for being incorporated and expressed by a cell or for transfer between different host cells. A cloning or expression vector may comprise elements, for example, regulatory and/or post-transcriptional regulatory elements and a promoter. A vector may include sequences that allow direct autonomous replication in a cell or may include sequences sufficient to allow integration into host cell DNA. In some embodiments, the vector described herein is a vector selected from the group of plasmids (e.g., DNA plasmids or RNA plasmids), shuttle vectors, transposons, cosmids, artificial chromosomes (e.g. bacterial, yeast, human), and viral vectors.

In some embodiments, the vector described herein is used in combination with at least one transfection enhancer, e.g., a transfection enhancer selected from the group of oligonucleotides, lipoplexes, polymersomes, polyplexes, dendrimers, inorganic nanoparticles and cell-penetrating peptides.

Transduction of host cells by the vector of the invention can be achieved by stable or transient transduction (see, e.g., Stepanenko, A. A., and Heng, H. H., 2017, Mutation Research/Reviews in Mutation Research, 773, 91-103).

In certain embodiments, the invention relates to a genetically modified cell comprising the polynucleotide of the invention.

The term “genetically modified cell”, as used herein, refers to a cell modified by means of genetic engineering. The term as used herein “engineered” and other grammatical forms thereof may refer to one or more changes of nucleic acids, such as nucleic acids within the genome of an organism.

In some embodiments, the genetically modified cell described herein is a host cell for the production of an attenuated human coronavirus (preferably SARS-CoV-2) or for amplification of the polynucleotide of the invention. The term “host cell”, as used herein, refers to a cell into which exogenous nucleic acid has been introduced, including the progeny of such a cell. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein.

In some the host cell described herein comprises at least one cell type selected from the group of Vero, VeroE6, VeroE6-TMPRSS2, A549-hACE2, HEK293, MDCK, Chinese hamster ovary (CHO), BHK-21, SF9, MRC 5, Per.C6, PMK, and WI-38.

In some embodiments, the genetically modified cell is a cell for use in cell therapy.

In certain embodiments, the invention relates to a method for production of an attenuated virus, the method comprising a step of culturing the genetically modified cell of the invention.

Methods for culturing cells are known in the art (see, e.g., Celis, Julio E., ed. Cell biology: a laboratory handbook. Vol. 1. Elsevier, 2005).

In certain embodiments, the invention relates to an attenuated virus comprising the polynucleotide of the invention.

In some embodiments, the attenuated virus described herein further comprises structural proteins of SARS-CoV-2, preferably all structural proteins of SARS-CoV-2.

In certain embodiments, the invention relates to a pharmaceutical product comprising the vector of the invention, the genetically modified cell of the invention and/or the attenuated virus of the invention.

In certain embodiments, the invention relates to a pharmaceutical product comprising the vector of the invention, the genetically modified cell of the invention and/or the attenuated virus of the invention for use as a medicament.

The term “pharmaceutical product”, as used herein, refers to a preparation which is in such form as to permit the biological activity of an active ingredient contained therein to be effective, and which contains no additional components which are unacceptably toxic to a subject to which the formulation would be administered.

The terms “use as a medicament” or “treatment” (and grammatical variations thereof such as “treat” or “treating”), as used herein, refer to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis.

In some embodiments, the pharmaceutical product comprises auxiliary substances like carriers and/or adjuvants, e.g., for enhancing an immune response of a patient. In some embodiments, the adjuvants described herein are at least one selected from the group of potassium alum; aluminum hydroxide; aluminum phosphate; calcium phosphate hydroxide; aluminum hydroxyphosphate sulfate; paraffin oil; propolis; killed bacteria of the species Bordetella pertussis or Mycobacterium bovis; plant saponins from Quillaja, soybean, and/or Polygala senega; cytokines IL-1, IL-2, and/or IL-12; as well as Freund's complete adjuvant. In some embodiments, the pharmaceutical product described herein comprises the vector of the invention and vector stabilizers and/or nanoparticles such as LNPs.

The dose is chosen such that the pharmaceutical product is well tolerated by the patient but evokes an immune response that gives desired medical effect, such as protection against infection or against a severe progression of an infection. In an embodiment, the dose is the lowest protective dose, the highest tolerable dose or lies between the lowest protective dose and the highest tolerable dose.

In some embodiments, the pharmaceutical product comprises the vector of the invention in a dose of at least 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, or more, vector genomes per kilogram (vg/kg) of the weight of the subject.

In some embodiments, the pharmaceutical product comprises the attenuated virus of the invention in a dose between 1*10³ and 1*10⁸ plaque-forming units (PFU) or focus-forming units (FFU), in particular between 1*10⁴ and 1*10⁷ PFU or FFU, in particular between 1*10⁵ and 1*10⁶ PFU or FFU, of the attenuated virus.

Various factors can influence the dose used for a particular application. For example, the frequency of administration, duration of treatment, preventive or therapeutic purpose, the use of multiple treatment agents, route of administration, previous therapy, patient's clinical history, the discretion of the attending physician and severity of the disease, disorder and/or condition may influence the required dose to be administered.

As with the dose, various factors can influence the actual frequency of administration used for a particular application. For example, the dose, duration of treatment, use of multiple treatment agents, route of administration, and severity of the disease, disorder and/or condition may require an increase or decrease in administration frequency.

In some cases, an effective duration for administering the pharmaceutical product of the invention (and any additional therapeutic agent) can be any duration that reduces the severity, or occurrence, of symptoms of the disease, disorder and/or condition to be treated without producing significant toxicity to the subject. Multiple factors can influence the actual effective duration used for a particular treatment. For example, an effective duration can vary with the frequency of administration, effective amount, use of multiple treatment agents, route of administration, and severity of the disease, disorder and/or condition being treated.

In some embodiments, the pharmaceutical product is administered to the patient at once. In some embodiments, the pharmaceutical product is administered to the patient at least two times, wherein the second administration is separated from the first administration by a first time period, herein also called prime/boost vaccination. In this context, the first time period lies in a range of from 2 weeks to 36 months, in particular of from 3 weeks to 30 months, in particular of from 4 weeks to 24 months, in particular of from 5 weeks to 21 months, in particular of from 6 weeks to 18 months, in particular of from 7 weeks to 15 months, in particular of from 8 weeks to 12 months, in particular of from 9 weeks to 10 months, in particular of from 10 weeks to 8 months, in particular of from 12 weeks to 6 months, in particular of from 13 weeks to 4 months.

In an embodiment, the pharmaceutical product is administered to the patient temporally offset to administering a different vaccine (such as, e.g., a vector-based vaccine, an mRNA-based vaccine, a protein-based vaccine) to the patient, i.e., after or before vaccinating the patient with the different vaccine. In this context, the administration of the pharmaceutical product is offset to the administration of the different vaccine by a second time period. In this context, the second time period lies in a range of from 2 weeks to 36 months, in particular of from 3 weeks to 30 months, in particular of from 4 weeks to 24 months, in particular of from 5 weeks to 21 months, in particular of from 6 weeks to 18 months, in particular of from 7 weeks to 15 months, in particular of from 8 weeks to 12 months, in particular of from 9 weeks to 10 months, in particular of from 10 weeks to 8 months, in particular of from 12 weeks to 6 months, in particular of from 13 weeks to 4 months.

In certain embodiments, the invention relates to a pharmaceutical product comprising the vector of the invention, the genetically modified cell of the invention and/or the attenuated virus of the invention for use in treatment and/or prevention of a human coronavirus infection, preferably a SARS-CoV-2 infection.

In certain embodiments, the invention relates to a pharmaceutical product comprising the vector of the invention, the genetically modified cell of the invention and/or the attenuated virus of the invention for use in treatment and/or prevention of a symptom of human coronavirus infection, preferably SARS-CoV-2 infection.

Symptoms of a SARS-CoV-2 infection include, without limitation, cough, fatigue, difficulty breathing, chills, joint or muscle pain, expectoration, sputum production, dyspnoea, myalgia, arthralgia or sore throat, headache, nausea, vomiting, diarrhea, sinus pain, stuffy nose, reduced or altered sense of smell or taste, lack of appetite, loss of weight, stomach pain, conjunctivitis, skin rash, lymphoma, apathy, and somnolence, preferably fever, cough, fatigue, difficulty breathing, chills, joint or muscle pain, expectoration, sputum production, dyspnoea, myalgia, arthralgia, sore throat, headache, nausea, vomiting, diarrhea, sinus pain, stuffy nose and reduced or altered sense of smell or taste.

In certain embodiments, the pharmaceutical product of the invention is administered intranasally or intramuscularly. The pharmaceutical product is preferably administered in a single dose or in two doses. Preferably the two doses are administered in a prime/boost administration.

The inventors found that the means and methods described herein can be used to induce an immune response that is useful in the treatment and/or prevention of a human coronavirus (preferably SARS-CoV-2) infection. In certain embodiments, the pharmaceutical product described herein is a vaccine and/or a vaccine booster.

In certain embodiments, the invention relates to the pharmaceutical product for use of the invention, wherein the pharmaceutical product further comprises a mutagen.

### Prevention or Treatment of a SARS-CoV-2

In a certain embodiment, the invention relates to a pharmaceutical product comprising the polynucleotide of the invention for use in the prevention or treatment of a SARS-CoV-2 virus infection, wherein said SARS-CoV-2 virus is not a SARS-CoV-2 Wuhan wild-type virus.

The pharmaceutical product comprises the polynucleotide of the invention, the vector of the invention comprising the polynucleotide, the genetically modified cell of the invention comprising the polynucleotide and/or the attenuated virus of the invention comprising the polynucleotide of the invention.

In a certain embodiment, the invention relates to a method for prevention or treatment of a SARS-CoV-2 virus infection, wherein said SARS-CoV-2 virus is not a SARS-CoV-2 Wuhan wild-type virus, said method comprises the step of administering the pharmaceutical product of the invention in a therapeutically effective amount to a subject, wherein the pharmaceutical product comprises the polynucleotide of the invention, the vector of the invention comprising the polynucleotide, the genetically modified cell of the invention comprising the polynucleotide and/or the attenuated virus of the invention comprising the polynucleotide.

In one embodiment, said SARS-CoV-2 virus is not a Wuhan wild-type SARS-CoV-2 virus. Preferably, SARS-CoV-2 Wuhan wild-type is defined to include or is more preferably defined to be Wuhan/IPBCAMS-WH-01/2019 or Wuhan/Hu-1/2019 (hereinafter Hu-1 wild-type strain).

In another embodiment, the SARS-CoV-2 virus is not a wild-type of the SARS-CoV-2 Wuhan-Hu-1 strain.

In a certain embodiment, said SARS-CoV-2 virus used in the prevention or treatment of a SARS-CoV-2 virus infection is a variant of a SARS-CoV-2 Wuhan wild-type or a variant of a SARS-CoV-2 Wuhan-Hu-1 wild-type strain. In another embodiment, said SARS-CoV-2 virus is a variant of SARS-CoV-2 WT BetaCoV/Wuhan/IVDC-HB-01/2019, Acc. No. MT108784. In another embodiment, said SARS-CoV-2 virus is a variant of SARS-CoV-2 WT BetaCoV/Wuhan/IVDC-HB-01/2019, Acc. No. MT108784.

The term “variant of a SARS-CoV-2” as used herein refers to a SARS-CoV-2 genome that contains one or more mutations as compared to the parent SARS-CoV-2 genome, e.g., the SARS-CoV-2 Wuhan wild-type, more preferably the SARS-CoV-2 Wuhan-Hu-1 strain. A variant of a SARS-CoV-2 Wuhan wild-type is derived from or originates from a SARS-CoV-2 Wuhan wild-type. The term “lineage” as used herein refers to a group of related viruses, preferably SARS-CoV-2 viruses with a common ancestor. The term lineage excludes Wuhan wild-type SARS-CoV-2 virus, preferably SARS-CoV-2 Wuhan-Hu-1 strain.

The of lineages of SARS-CoV-2 mentioned herein are preferably according to the Pango nomenclature system (https://libguides.mskcc.org/SARS2/lineages, Jun. 4, 2023; O'Toole A et al., BMC Genomics, vol. 23 (121), 2022; Rambaut A et al., 2020, Nature Microbiology, 5 (11), pp. 1403-1407). The term “missense mutations” as used herein refers to a change in at least one amino acid in a protein, arising from a point mutation in a single nucleotide.

In a certain embodiment, the variant of a Wuhan wild-type SARS-CoV-2 virus is of a lineage selected from the group consisting of A.1-A.6, B1, B2, B.3-B.7, B.9, B.10, and B.13-B.16, preferably B1, B2, B.3-B.7, B.9, B.10, and B.13-B.16. In another preferred embodiment, the variant of a Wuhan wild-type SARS-CoV-2 virus is of the lineage B (Pekar J E et al., Science, 2022, vol. 377(6609), pp. 960-966), preferably B.1, more preferably B.1.1 or B.1.617, again more preferably B.1.1.529 or B.1.617.

In a preferred embodiment, the variant of a Wuhan wild-type SARS-CoV-2 virus is selected from the group consisting of Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), Mu (lineage B.1.621) and a missense variant of a Wuhan wild-type SARS-CoV-2 virus comprising at least one, preferably 1-3, more preferably exactly one missense mutation. In a preferred embodiment, the variant of a Wuhan wild-type SARS-CoV-2 virus is selected from the group consisting of Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), and Mu (lineage B.1.621).

In a preferred embodiment, the variant of a Wuhan wild-type SARS-CoV-2 virus is selected from the group consisting of Alpha (lineage B.1.1.7), B.1.1.7 with E484K, Beta (lineage B.1.351), Delta (lineage B.1.617.2), Omicron (B.1.1.529), Epsilon (lineages B.1.429, B.1.427, CAL.20C), Eta (lineage B.1.525), Iota (lineage B.1.526), Kappa (lineage B.1.617.1), Mu (lineage B.1.621) and a variant of a Wuhan wild-type SARS-CoV-2 virus comprising at least one, preferably 1-3, more preferably exactly one missense mutation. In a preferred embodiment, the variant of a Wuhan wild-type SARS-CoV-2 virus is selected from the group consisting of Alpha (lineage B.1.1.7), B.1.1.7 with E484K, Beta (lineage B.1.351), Delta (lineage B.1.617.2), Omicron (B.1.1.529), Epsilon (lineages B.1.429, B.1.427, CAL.20C), Eta (lineage B.1.525), Iota (lineage B.1.526), Kappa (lineage B.1.617.1), and Mu (lineage B.1.621).

In another again more preferred embodiment, the variant of a Wuhan wild-type SARS-CoV-2 virus is selected from the group consisting of Alpha (lineage B.1.1.7), B.1.1.7 with E484K, Omicron (B.1.1.529), Delta (lineage B.1.617.2), Kappa (lineage B.1.617.1) and a missense variant of a Wuhan wild-type SARS-CoV-2 virus comprising at least one, preferably 1-3, more preferably exactly one missense mutation. In another again more preferred embodiment, the variant of a Wuhan wild-type SARS-CoV-2 virus is selected from the group consisting of Alpha (lineage B.1.1.7), B.1.1.7 with E484K, Omicron (B.1.1.529), Delta (lineage B.1.617.2), and Kappa (lineage B.1.617.1) variants.

In another preferred embodiment, the variant of a wild-type SARS-CoV-2 virus is selected from the group consisting of Omicron (B.1.1.529), Delta (lineage B.1.617.2), and a missense variant of a Wuhan wild-type SARS-CoV-2 virus comprising at least one, preferably 1-3, more preferably exactly one missense mutation. In another preferred embodiment, the variant of a wild-type SARS-CoV-2 virus is Omicron (B.1.1.529) or Delta (lineage B.1.617.2).

In another preferred embodiment, the variant of a Wuhan wild-type SARS-CoV-2 virus is Delta (B.1.617.2), Omicron BA.2, Omicron BA.5 and a missense variant of a Wuhan wild-type SARS-CoV-2 virus comprising at least one, preferably 1-3, more preferably exactly one missense mutation. In another preferred embodiment, the variant of a Wuhan wild-type SARS-CoV-2 virus is Delta (B.1.617.2), Omicron BA.2, and Omicron BA.5.

Preferably said missense mutation is located in or is in a region of a SARS-CoV-2 virus genome encoding a spike protein, preferably said at least one or exactly one missense mutation is D614G, such as in SARS-CoV-2 D614G (BetaCoV/Germany/BavPat1/2020, Acc. No. EPI_ISL_406862).

In another preferred embodiment, the variant of a Wuhan wild-type SARS-CoV-2 virus is Delta (B.1.617.2). In another preferred embodiment, the variant of a Wuhan wild-type SARS-CoV-2 virus is Omicron (B.1.1.529), preferably Omicron BA.2 or Omicron BA.5, e.g. Acc. No. ON545852 or Acc. No. EPI_ISL_12268493.2. In another preferred embodiment, the variant of a Wuhan wild-type SARS-CoV-2 virus comprises at least one, preferably 1-3, more preferably exactly one missense mutation, wherein preferably said at least one or exactly one missense mutation is D614G, e.g., as in Acc. No. EPI_ISL_406862.

In another preferred embodiment, the variant of a wild-type SARS-CoV-2 virus is selected from the group consisting of SARS-CoV-2 WT D614G, SARS-CoV-2 Omicron BA.2, SARS-CoV-2 Omicron BA.5 and SARS-CoV-2 VOC Delta (B.1.617.2). In another preferred embodiment, the variant of a Wuhan wild-type SARS-CoV-2 virus is Delta (B.1.617.2), SARS-CoV-2 Omicron BA.2 (SARS-CoV-2/human/NLD/EMC-BA2-1/2022, Acc. No. ON545852), SARS-CoV-2 Omicron BA.5 (hCoV-19/South Africa/CERI-KRISP-K040013/2022, Acc. No. EPI_ISL_12268493.2) or a variant of a Wuhan wild-type SARS-CoV-2 virus comprising at least one missense mutation, wherein preferably said missense mutation is D614G. In another preferred embodiment, the variant of a Wuhan wild-type SARS-CoV-2 virus is Delta (B.1.617.2), SARS-CoV-2 Omicron BA.2 (SARS-CoV-2/human/NLD/EMC-BA2-1/2022, Acc. No. ON545852), SARS-CoV-2 Omicron BA.5 (hCoV-19/South Africa/CERI-KRISP-K040013/2022, Acc. No. EPI_ISL_12268493.2). In another preferred embodiment, the variant of a Wuhan wild-type SARS-CoV-2 virus is Delta (B.1.617.2), SARS-CoV-2 Omicron BA.2 (SARS-CoV-2/human/NLD/EMC-BA2-1/2022, Acc. No. ON545852), SARS-CoV-2 Omicron BA.5 (hCoV-19/South Africa/CERI-KRISP-K040013/2022, Acc. No. EPI_ISL_12268493.2) and SARS-CoV-2 D614G (BetaCoV/Germany/BavPat1/2020, Acc. No. EPI_ISL_406862). In another preferred embodiment, the variant of a wild-type SARS-CoV-2 virus SARS-CoV-2 D614G, SARS-CoV-2 Omicron (B.1.1.529) or SARS-CoV-2 VOC Delta (B.1.617.2).

In a certain embodiment, the invention relates to the pharmaceutical product of the invention comprising the polynucleotide of the invention, vector of the invention, genetically modified cell of the invention and/or attenuated virus of the invention, for use in the prevention or treatment of a corona virus infection in a human subject.

In a certain embodiment, the invention relates to a method for prevention or treatment of a corona virus infection in a human subject, said method comprises the step of administering the pharmaceutical product of the invention in a therapeutically effective amount to a human subject, wherein the pharmaceutical product comprises the polynucleotide of the invention, vector of the invention, the genetically modified cell of the invention and/or the attenuated virus of the invention.

In a preferred embodiment, the corona virus infection is a SARS-CoV-2 virus infection.

The pharmaceutical product of the invention provides long term protection and induces long-term immunity against SARS-CoV-2 infection. Protection and immunity is provided for at least 174 days after vaccination. Especially, protection against lung pathology, such as lung injury is provided.

The pharmaceutical product of the invention provides long term protection characterized by lower amounts of viral RNA in samples of respiratory organs, especially lung and nose, tested in subjects challenged 174 days after vaccination when compared to subjects challenged 57 days after vaccination. Thus, in a certain embodiment, the invention relates to the pharmaceutical product of the invention for use in the prevention or treatment of a corona virus infection, preferably a SARS-CoV-2 virus infection, in a human subject, wherein said human subject has lower amounts of viral RNA in samples of respiratory organs, especially lung and nose, when challenged 2 months or more after vaccination when compared to subjects challenged less than two months after vaccination. In another embodiment, said human subject has lower amounts of viral RNA in samples of respiratory organs, especially lung and nose, when challenged at least 58 days, preferably at least 86 days, more preferably at least 114 days, again more preferably at least 142 days, again more preferably at least 170 days after vaccination when compared to subjects challenged 57 days or less after vaccination. In another embodiment, said human subject has lower amounts of viral RNA in samples of respiratory organs, especially lung and nose, when challenged between 58-200 days, preferably between 86-200 days, more preferably between 114-200 days, again more preferably between 142-200 days, again more preferably between 170-200 days after vaccination when compared to subjects challenged 57 days or less after vaccination. In another embodiment, said human subject has lower amounts of viral RNA in samples of respiratory organs, especially lung and nose, when challenged between 58-250 days, preferably between 86-250 days, more preferably between 114-250 days, again more preferably between 142-250 days, again more preferably between 170-250 days after vaccination when compared to subjects challenged 57 days or less after vaccination. In another embodiment, said human subject has lower amounts of viral RNA in samples of respiratory organs, especially lung and nose, when challenged between 58-300 days, preferably between 86-300 days, more preferably between 114-300 days, again more preferably between 142-300 days, again more preferably between 170-300 days after vaccination when compared to subjects challenged 57 days or less after vaccination. Infectious virus titers from the samples are determined using TCID50 assays as described herein.

In a certain embodiment, the human subject is challenged by a SARS-CoV-2 virus more than 21 days, preferably more than 28 days, more preferably more than 35 days, again more preferably more than 42 days, again more preferably more than 56 days, again more preferably more than 70 days, again more preferably more than 84 days, again more preferably more than 98 days, again more preferably more than 112 days, again more preferably more than 126, again more preferably more than 140, again more preferably more than 154, again more preferably more than 168, again more preferably more than 174 days after vaccination.

Said human subject is preferably challenged by a wildtype SARS-CoV-2 virus or a variant thereof. Preferably, the variant is selected from the group consisting of Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), Mu (lineage B.1.621) and a missense variant comprising at least one missense mutation. More preferably, the variant is Delta (lineage B.1.617.2), Omicron (B.1.1.529) or a variant comprising at least one missense mutation, wherein preferably said missense mutation is D614G.

K18-hACE2 mice as used in the examples provides a model for studying features of severe COVID-19 in humans and acute respiratory distress syndrome (inter alia Nat Immunol 21, 1327-1335 (2020) and DOI: 10.1101/2020.08.11.246314).

In a preferred embodiment, said human subject is at increased risk of developing severe COVID-19 or acute respiratory distress syndrome.

In a preferred embodiment, said human subject is at increased risk of developing severe COVID-19.

In a certain preferred embodiment, the population of human subjects to be at increased risk of developing severe COVID-19 is defined as in the German Health Update (GEDA) 2019/2020-EHIS (Journal of Health Monitoring, 2021 6(S2), DOI 10.25646/7859, especially Table 1).

In another preferred embodiment, the term “a subject at risk for a severe COVID-19”, as used herein, refers to a subject having at least one, at least two, at least three, at least four or at least five risk factor(s) to develop severe COVID-19. The risk factor to develop severe COVID-19 are preferably selected from the group consisting of age above 50 years, Immunocompromised or weakened immune system, cancer, chronic kidney disease, chronic liver disease, chronic lung disease, cystic fibrosis, dementia, Alzheimer's disease, diabetes, Down syndrome, spinal cord injury, heart condition, hypertension, HIV infection, mood disorder, BMI above 25 kg/m², sickle cell disease, thalassemia, smoker, organ or blood stem cell transp1 ant receiver/donor, stroke, cerebrovascular disease, substance use disorder, tuberculosis, COPD and asthma.

In a preferred embodiment, said SARS-CoV-2 virus infection is severe COVID-19 or an acute respiratory distress syndrome. In a preferred embodiment, said SARS-CoV-2 virus infection is severe COVID-19. In another preferred embodiment, said human subject has severe COVID-19.

The term “severe COVID-19” or “severe COVID-19 infection” includes subjects, preferably human subjects that (1) had a confirmed positive COVID-19 test utilizing the polymerase chain reaction method from a nasopharyngeal swab sample and that (2) show a certain value of a second parameter to indicate and/or predict disease severity.

In a preferred embodiment, said second parameter is an SpO₂<94% on room air at sea level, a ratio of arterial partial pressure of oxygen to fraction of inspired oxygen (PaO₂/FiO₂)<300 mm Hg, a respiratory rate >30 breaths/min, or lung infiltrates >50%.

In another preferred embodiment, said second parameter is a serum level of C-reactive protein (CRP). Preferably, the serum CRP level indicative for severe Covid-19 is at least 18 mg/L, preferably at least 20 mg/L (Tan et al., J Med Virol. 2020; 92:856-862, DOI: 10.1002/jmv.25871; Chen et al., Ann Clin Microbiol Antimicrob 2020; 19:18. DOI: 10.1186/s12941-020-00362-2.). In another preferred embodiment, the serum CRP level indicative for severe Covid-19 is at least 30 mg/L, preferably at least 40 mg/L.

CRP is measured using ERM-DA472/IFCC and ERM-DA474/IFCC secondary reference materials as common calibrators or traceability to WHO 1^{st} International Standard 85/506 is assured through an alternative way. Thereby, the comparability of CRP results, allowing the application to different populations of common decisional cut-offs, when available (Aloisio et al., Clinical Chemistry and Laboratory Medicine (CCLM), 2023, DOI: 10.1515/cclm-2023-0276).

Preferably, CRP is measured by using the immunoturbidimetric assay on the Alinity c platform (Abbott Diagnostics) traceable to the ERM-DA472/IFCC reference material, shown to assure a good analytical performance for the clinical application of the measurements (Aloisio et al., 2023).

Preferably, the term “severe COVID-19 infection” includes human subjects that having respiratory failure, septic shock, or multiple organ dysfunction.

In a preferred embodiment, said SARS-CoV-2 virus infection is an acute respiratory distress syndrome. In another preferred embodiment, said human subject has an acute respiratory distress syndrome.

The term “acute respiratory distress syndrome” or “ARDS”, as used herein, refers to an acute respiratory condition that is characterized by a PaO₂/FiO₂ ratio of less than 3 mmHg, preferably less than 200 mmHg, more preferably less than 100 mmHg. An “acute” respiratory condition is a respiratory condition of acute onset, within 4 weeks, 3 weeks, 2 weeks or 1 week of an apparent clinical insult, preferably with the progression of respiratory symptoms. In certain embodiments, the acute respiratory distress syndrome described herein additionally comprises at least one characteristic selected from the group of inflammation, bilateral opacities on chest imaging, a positive end-expiratory pressure of more than 5 cm H₂O, O₂ saturation below 92% and respiratory failure.

In a preferred embodiment, said pharmaceutical product is administered intranasally to a human subject. In a preferred embodiment, said pharmaceutical product is administered via a prime/boost vaccination. In a preferred embodiment, the polynucleotide encompassed by the pharmaceutical product of the invention consists of or comprises a sequence as defined SEQ ID NOs: 3, 4, 5 or 6, preferably SEQ ID NOs: 4, 5 or 6.

In a certain embodiment, the invention relates to a method of treatment and/or prevention comprising the step of Administering a pharmaceutical product in a therapeutically effective amount to a subject, wherein the pharmaceutical product comprises the vector of the invention, the genetically modified cell of the invention and/or the attenuated virus of the invention.

In a certain embodiment, the invention relates to the method of treatment and/or prevention of the invention, wherein the treatment and/or prevention is a treatment and/or prevention of a human coronavirus (preferably SARS-CoV-2) infection.

In a certain embodiment, the invention relates to the method for treatment and/or prevention of the invention, wherein the method further comprises administering a mutagen in a therapeutically effective amount to a subject.

In certain embodiments, the invention relates to a combination of a mutagen with a polynucleotide encoding an attenuated virus or a fragment thereof, wherein the polynucleotide comprises at least 20 one-to-stop codons, wherein a one-to-stop codon is i) a different but synonymous codon compared to a corresponding codon in a natural virus genome or a fragment thereof; and ii) differs by only one nucleotide from a STOP codon. The attenuated virus is preferably a human coronavirus, more preferably a beta coronavirus, even more preferably SARS-CoV-2.

The combination may be administered simultaneously or sequentially. As such the administration of the mutagen described herein can occur prior to, simultaneously, and/or following, administration of the polynucleotide described herein. In certain embodiments, the combination described herein is in a composition for simultaneous administration or in several separate compositions for simultaneous or sequential administration. The mutagen and the polynucleotide described herein can be administered by the same administration route (e.g., parenteral) or by different administration routes (e.g. oral administration for the mutagen und parenteral administration for the polynucleotide described herein). In a preferred embodiment, the mutagen described herein is administered repeatedly, preferably more often than the polynucleotide described herein.

The attenuation encoded in the polynucleotide can therefore be enhanced by the mutagen. The mutagen may therefore be used in subjects where a non-typical (e.g. stronger side effects, more in vivo proliferation than usual) immune response is expected or observed. In certain embodiments, the combination of the mutagen and the polynucleotide described herein is administered to a subject with an altered immune system function. The immune system function alteration can be induced, without limitation by a disease or disorder (such as infection, autoimmune disease, cancer, immunodeficiency (acquired or congenital) or obesity) and/or by an immunomodulatory treatment (e.g., DMARDs, IMiDs and/or oncological treatment). Alternatively, the immune response to an attenuated virus can be measured and when reaching a certain threshold may be stopped or tampered by administration of the mutagen.

The mutagen may also be equivalently combined with the attenuated virus of the invention, the host cell of the invention, or the vector of the invention instead of the polynucleotide described herein. In certain embodiments, the mutagen described herein is an RNA-nucleotide analog. In certain embodiments, the mutagen described herein is 5-fluorouracil or molnupiravir (molnupiravir).

As such, the invention is at least in part based on the finding, that the attenuation of a one-to-stop attenuated virus can be regulated by a mutagen.

All embodiments of the polynucleotide can be combined in any desired way and can be transferred either individually or in any arbitrary combination to the attenuated human coronavirus (preferably SARS-CoV-2), to the pharmaceutical composition, its use, to the method of treatment, to the vector, to the host cell, and to the method of producing a virus.

“a,” “an,” and “the” are used herein to refer to one or to more than one (i.e., to at least one, or to one or more) of the grammatical object of the article. “or” should be understood to mean either one, both, or any combination thereof of the alternatives. “and/or” should be understood to mean either one, or both of the alternatives.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

The terms “include” and “comprise” are used synonymously. The term “preferably” means one option out of a series of options not excluding other options. “e.g.” means one example without restriction to the mentioned example. By “consisting of” is meant including, and limited to, whatever follows the phrase “consisting of.”

Reference throughout this specification to “one embodiment”, “an embodiment”, “a particular embodiment”, “a related embodiment”, “a certain embodiment”, “an additional embodiment”, “some embodiments”, “a specific embodiment” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Unless otherwise defined, all technical and scientific terms used herein have the same meaning as commonly understood by one of ordinary skill in the art to which this invention pertains.

Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

### BRIEF DESCRIPTION OF FIGURES

FIG. 1: Schematic illustration of generation of recombinant SARS-CoV-2 using “transformation-associated recombination” (TAR) cloning is yeast, subsequent generation of in vitro transcribed RNA resembling the recombinant SARS-CoV-2 RNA genome, and subsequent assessment of the virus phenotype.

FIG. 2: SARS-CoV-2 genome; Modular “One-to-stop” (OTS) cloning strategy

FIG. 3: SARS-CoV-2-OTS replication in primary airway epithelial cultures. Virus titer (Tissue culture infectious dose 50%; TCID50) was determined at 0 (inoculum), 1, 24, 48, 72, 96 hours post-infection in apical washes. A: OTS-Clones: 96-hour kinetics on hNEC at 33° C.; B: OTS-Clones: 96-hour kinetics on hNEC at 37° C.

FIG. 4: OTS8, OTS4-5 were assessed for attenuation: A: body weight, B: clinical score, C: Histopathological score, D: viral copies, E: Virus titer

FIG. 5: OTS2, OTS7, and OTS7-8 were assessed for attenuation: A: body weight, B: clinical score, C: Histopathological score, D: viral copies, E: Virus titer

FIG. 6: OTS4-5 and OTS7-8 attenuation and protection. Mice were immunized with OTS4-5, OTS-7-8. At day 7 half of the mice were euthanized for analysis. Challenge with pathogenic wild-type virus was done at 21 days post immunization.
A: Pre-challenge survival B: Post-challenge survival (note of A and B that at day 7 post immunization 50% of mice were euthanized for analysis), C: Pre-challenge weight D: Post-challenge weight E: Pre-challenge score, F: Post-challenge score G: viral copies 7 days post immunization: Animals with high clinical score and body weight loss H: viral copies at day 26 (day 5 post challenge), I: viral copies at day 35 (14 days post challenge), J: Pre-challenge viral copies oropharyngeal swabs, K: Post-challenge viral copies oropharyngeal swabs, L: viral titer at 5 days post challenge M: viral titer at 14 days post challenge

FIG. 7: OTS4-5 and OTS7-8 attenuation and protection, A: Neutralizing Antibody Assay against Wuhan WT: neutralizing antibody titers, B: Spike-specific CD8+ T cells: T cell responses, C Histopathological score,

FIG. 8: OTS4-5 and OTS4-5-7-8 were assessed for attenuation: A: survival, B: clinical score, C: body weight, D: Swabs, E-G: RNA, H-I: PFU

FIG. 9: Construct overview

FIG. 10: Naive Syrian hamsters (also ferr/mice) with one-to-stop 4-5/7-8 construct, P=Nasal washing; A: intra nasal inoculation: 5000 PFU/hamster, OTS4-5/7-8 inoculated N=10, WT inoculated control N=4, OTS4-5/7-8 contact N=4; Co-housing: Co-housing of the contact groups; Necropsy 1: Necropsy of half of inoculated and control group; Necropsy 2: Necropsy of 5 inoculated and contacts; B: intra nasal inoculation: 5000 PFU/hamster, OTS4-5/7-8 inoculated N=8, OTS4-5/7-8 contact N=3; Challenge: Challenge of inoculated and N=4 naive control with WT 5000 PFU/hamster and co-housing of the contact groups; Necropsy: Necropsy of inoculated and contacts. Can apply for 5 dpc necropsy.

FIG. 11: A: Hamster survival; B: Relative body weight

FIG. 12: genome copies

FIG. 13: Humoral immune response (RBD-ELISA-Data) of OTS inoculated and direct contact animals. FCS deletion prevent transmission of final OTS to naïve contact animals.

FIG. 14: Tissue specific gene copies 5 days post inoculation with WT or final OTS.

FIG. 15: Humoral immune response (RBD-ELISA-Data) at 14 dpc. Final OTS (SEQ ID NO: 6) prevent transmission of challenge virus to naïve contact animals.

FIG. 16: A: 5-FU: Cells: VeroET cells; Pre-treatment for 30 min; Infection with MOI: 0.1 for 1 h with ID3 and ID194; Remove inoculum and add DMEM+drug in concentration ranging from 40-280 uM; Harvesting and TCID50 24h pi (hours post infection); B: Molnupiravir: Cells: VeroET cells; Pre-treatment for 30 min; Infection with MOI: 0.1 for 1 h with ID3 and ID194; Remove inoculum and add DMEM+drug in concentration ranging from 0.1-10 uM; Harvesting and TCID50 24h pi

FIG. 17: Human bronchial epithelial cell (hBEC) cultures were infected with SARS-CoV-2 WT, as well as SARS-CoV-2 with OTS codons in either Fragment 2, 7 or 8 (OTS2, 7, 8). Viral titers are shown until 96 hours post infection in TCID50/ml. OTS2 is significantly attenuated at 72 and 96 hpi.

FIG. 18: Assessment of immune responses. A: Experimental design to assess virus-specific immune responses. Mice were immunized by infection with attenuated SARS-CoV-2 OTS4-5, OTS7-8, OTS4-5-7-8, OTS-206 or were mock infected. Challenge with wt SARS-CoV-2 was performed 21 days later. B: Determination of SARS-CoV-2 neutralizing antibody titers in serum obtained from mice at days 15 (pre-challenge) and days 35 (post-challenge) by virus neutralization assay. C: Determination of SARS-CoV-2-specific CD8+T-cell responses at days 15 (pre-challenge) and days 26 (post-challenge; dpc) by tetramer staining (H-2K(b) SARS-CoV-2 spike epitope 539-546 (VNFNFNGL) SEQ ID NO: 8).

FIG. 19: OTS constructs show in vitro replication kinetics comparable to WT SARS-CoV-2 but are more sensitive to treatment with mutagenic drugs. a, Schematic overview of the mutations introduced to SARS-CoV-2 genome to generate OTS codons. Fragments 4, 5, 7, and 8, which are used for TAR cloning of recombinant SARS-CoV-2 clones have been modified to enrich the number of one-to-stop codons. The number of codons and nucleotides that have been changed are indicated for each fragment. For the OTS-206 construct, two additional point mutations were introduced in nsp1 (K164A/H165A) and open reading frames ORF6 to ORF8 were deleted. OTS-228 has an additional deletion in the spike S1/S2 PCS. b, The plaque sizes of viruses at 2 dpi normalized to means size of WT. Sizes of 10 plaques/wells from one biological replicate in 6-well plates were measured in Adobe Illustrator. Each circle in the violin plot represents one plaque size. Statistical significance was determined using ordinary one-way ANOVA and p-values were adjusted using Tukey's multiple-comparison test. Due to the variation in plaque sizes no statistically significant difference between the average plaque sizes of OTS and SARS-CoV-2D614G WT viruses was found. c, Vero E6/TMPRSS2 cells (n=3) and d, Human nasal (NECs) and e, bronchial epithelial cell (hBECs) (n=6 (3 replicates from 2 donors)) cultures were infected with 0.1MOI of the SARS-CoV-2 WT and OTS viruses from the apical side and incubated at 33° C. (hNECs) and 37° C. (Vero E6/TMPRSS2 and hBECs) for 1 h. After 1 h, supernatant was discarded and the cells were washed 3 times with PBS, and the third wash was kept for analysis. Following the addition of new sera on the cells, they were incubated 33° C. (hNECs) and 37° C. (Vero E6/TMPRSS2 and hBECs). Samples were collected on designated time points post-infection. Infectious particle titers were assessed by TCID50 assays on VeroE6/TMPRSS2 cells. Each line in the graphs shows the titers obtained from one individual sample. Statistical significances in the titer differences of OTS viruses vs WT on given times were determined using two-way ANOVA and p-values were adjusted using Tukey's multiple-comparison test; *P<0.05, **P<0.01, ***P<0.001, ****P<0.0001. f, g, Vero E6/TMPRSS2 cells were treated with 5-Fluorouracil (5-FU) [40-280 uM] and Molnupiravir [0.1-10 uM] for 30 mins, then were infected with 0.1 MOI of SARS-CoV-2 WT or OTS4-5-7-8. After 1h, cells were washed and new medium was added including 5-FU and Molnupiravir with concentration ranging from 40-280 uM. Supernatant were harvested after 24 hours and titers were assessed by TCID50 assays on VeroE6/TMPRSS2 cells. Graphs represent results from two independent experiments with three replicates. Statistical significance was assessed by unpaired, nonparametric multiple t-test with Mann-Whitney test (compared ranks); *P<0.05, **P<0.01, ***P<0.001, ****P<0.0001. cf. FIG. 23.

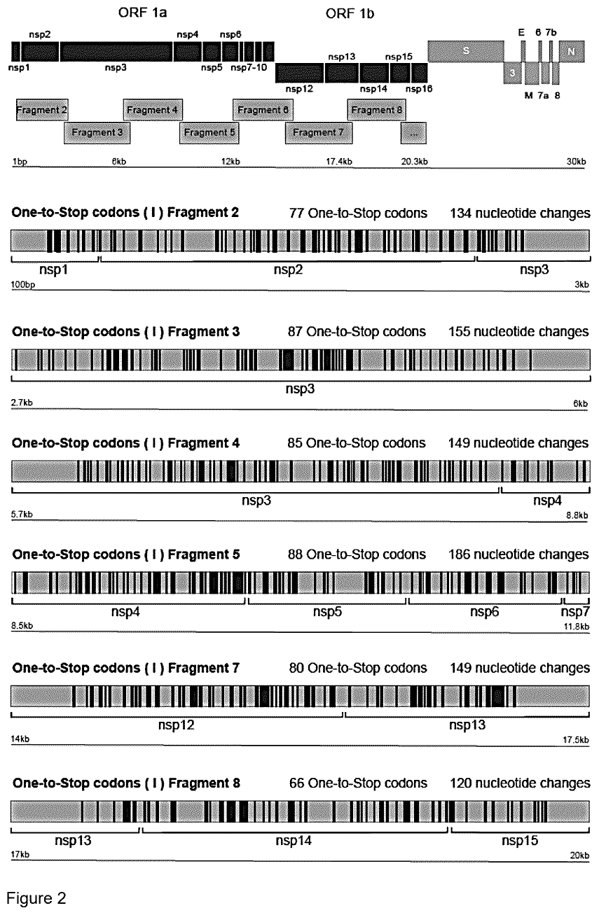

FIG. 20: Immunization with OTS constructs lead to full protection against SARS-CoV-2 challenge infection. a, Experimental setup of intranasal inoculation of K18-hACE2 mice (7-16 week-old, n=12 mice/group) with OTS4-5, OTS7-8, OTS4-5-7-8 and OTS-206 (5′000 PFU/mice), and subsequent challenge infection with WT SARS-CoV-2 (5′000 PFU/mice) at 21 dpi. At 21 dpi, naïve mice (n=12 mice) were introduced and challenged with the same amount of WT virus. b, Pre-challenge survival (%) of OTS-construct-inoculated mice. Increased number of OTS modifications correlate with an increased level of survival post-inoculation. c, Pre-challenge body weight loss of OTS-construct-inoculated mice. Reduced body weight loss was observed with increased number of OTS modifications and complete absence of body weight loss for the OTS-206. d, e, All OTS constructs provide full protection against SARS-CoV-2 wild-type challenge in terms of d, survival and e, body weight loss. f, Clinical scores post-challenge. Only naïve mice challenged with WT virus presented high clinical scores. Each circle and triangle represent a mouse. g, Viral genome copies per mL of nose and lung samples (n=12 mice/group) at 5-6 dpc and h, 14 dpc were quantified using probe-specific RT-qPCR. i, Infectious virus titers from the lung and nose samples (n=12 mice/group) were determined using plaque assays in VeroE6/TMPRSS2 cells. j, k, Histopathological scores and immunohistochemical analysis specific for SARS-CoV-2 nucleocapsid protein (k) of lung sections in OTS-construct-inoculated and naïve mice at 5 dpc following challenge infection with WT SARS-CoV-2 (magnification 50×). 1, Experimental setup of intranasal inoculation of Syrian hamsters (n=8 mice/group) with OTS4-5 or OTS7-8 and subsequently challenged with WT SARS-CoV-2. Nasal washings and organ samples were collected on the indicated days post-challenge. m, n, Post-challenge survival (%) and body weight loss of OTS-construct-inoculated and naïve hamsters. None of the pre-immunized animals succumbed to the disease or had to be euthanized, nor did they lose weight. o, p, Viral genome copies in the nasal washings and the upper/lower respiratory tissues were quantified using probe-specific RT-qPCR. Viral genome load was reduced in nasal washings over time and (p) only low levels of viral genome were detectable in lungs of pre-immunized hamsters at 14 dpc. q, Experimental setup of intranasal inoculation of Syrian hamsters (n=8 mice/group) with OTS-206 and subsequently challenged with BA.2 VOC. r, s, Post-challenge survival (%) and body weight loss of OTS-206-inoculated and naïve hamsters. All pre-immunized hamsters survived the challenge infection and (s) only the naïve animals lost weight following the challenge infection. t, u, Viral genome copies in the nasal washings and the upper/lower respiratory tissues were quantified using probe-specific RT-qPCR. Viral genome load in conchae samples were significantly reduced, while no virus could be detected in the lung samples of OTS-206 immunized hamsters. Body weight loss data of mice and hamsters are presented as mean±s.d. from the indicated number of biological replicates from a single experiment. The color key in FIG. 2a applies to FIG. 2a to j. Statistical significance of weight changes of WT- or OTS-inoculated vs mock mice was determined using two-way ANOVA (Tukey's multiple comparison test) (panels c and e), presented in color codes shown in FIG. 2a. Statistical significance of differences in gEq/ml and histopathological scores were determined by ordinary one-way ANOVA (panels g, h and j), for panels o, p two-way ANOVA (Tukey's multiple comparison test) and for panels t, u, uncorrected Fisher's LSD, individual variances computed for each comparison was used. The comparisons not marked with asterisks did not show statistical significance; *P<0.05, **P<0.01, ***P<0.001, ****P<0.0001. cf. FIG. 26

FIG. 21: OTS-206 demonstrates comparable efficacy to mRNA-vaccines and inducing long-term immunity in K18-hACE2 mice. a, Experimental setup of spatial transcriptomics analysis. K18-hACE2 (7-15 weeks old, n=8 mice/group) mice were vaccinated intramuscularly (i.m.) with a single dose of 1 μg mRNA vaccine Spikevax (Moderna) or intranasally (i.n.) with 5′000 TCID50 of OTS-206. After 28 days, mice were challenged i.n. with 104 TCID50 of SARS-CoV-2 Delta VOC, and lungs were harvested 2- or 5-days post-challenge (dpc). Mock-challenged animals were inoculated i.n. with DMEM. b, Immunohistochemistry of whole lung sections for SARS-CoV-2 nucleocapsid. Positively stained cells are brown, and asterisks have been used to highlight positive areas. c, Quantification of the percentage of lung cells stained for nucleocapsid (N) by immunohistochemistry (IHC). d, e, SARS-CoV-2 gene counts normalized across conditions. ORF10 was removed because it was not detected in our samples. Representative spatial expression profiles on the right, here SARS-CoV-2 gene counts (N, ORF1ab, M, E, S, ORF3a) are summed. f, Pathway activity scores are inferred from perturbation data. Comparison of gene expression signatures in the capture spots with perturbation signatures constructed from the expression changes of the top 100 genes in perturbation experiments. The JAK-STAT pathway shows significantly increased activity. This was also evident in the violin plots, which show the underlying distribution of pathway scores in each capture spot. g, K18-hACE2 transgenic mice (7-15 weeks old, n=8 mice/group) were immunized (prime & boost) either intramuscularly with a single dose of 1 μg of mRNA-Vaccine Spikevax (Modema), or intranasally with 5′000 PFU of OTS-206. At 57 dpi a group of mice was intranasally inoculated with 104 TCID50 of SARS-CoV-2D614G, or SARS-CoV-2 Delta VOC (h-j). The rest of the immunized mice were kept for approximately 5 months and then intranasally inoculated with 104 TCID50 of SARS-CoV-2D614G (k, m). h, k, During infection, mice were regularly monitored for body weight changes, and clinical symptoms. Each line in the body weight loss graphs represents a mouse. Six days post-challenge, mice were euthanized, and organ samples were collected for evaluation of infectious virus titers, viral genome copy numbers, and pathology. i, 1, Infectious virus titers from the nose and lung samples were determined using TCID50 assays in VeroE6/TMPRSS2 cells. j, m, Histopathological scores were given to evaluate the severity of the lung pathology. Statistical significance of weight changes of WT- or OTS-inoculated vs mock mice was determined using two-way ANOVA (Tukey's multiple comparison test) (panels h and k), and ordinary one-way ANOVA was used to for panels j and m; *P<0.05, **P<0.01, ***P<0.001, ****P<0.0001. Data were obtained from one experiment. Each data point represents one biological replicate. Infectious viral particle concentrations and genome copies from tissue samples, as well as the immunohistochemical analysis specific for SARS-CoV-2 nucleocapsid protein are shown in FIG. 28. Body weight changes, clinical scores, and histopathological scores of the lungs of all K18-hACE2 mice experiments are shown in FIG. 33.

FIG. 22: OTS-228 shows significantly reduces transmission, protects against and limits transmission of SARS-CoV-2 VOC challenge infections. a, Schematic representation of the deleted polybasic cleavage site (CS) in S1/S2 junction in OTS-228 spike region compared to WT and OTS-206. b, The plaque sizes of viruses at 2 dpi normalized to mean size of WT. Sizes of 10 plaques/wells from one biological replicate in 6-well plates were measured in Adobe Illustrator. Each circle in the violin plot represents one plaque size. Statistical significance was determined using ordinary one-way ANOVA and p-values were adjusted using Tukey's multiple-comparison test. Only significant differences are shown: black asterisks indicate the comparison to WT, orange asterisks indicate the comparison to OTS-206. Reduced plaque sizes were observed when the CS was deleted. c, Human nasal and bronchial epithelial cell (hNECs and hBECs) (n=3 donors) cultures were infected with 5×104 PFU of the indicated viruses from the apical side and incubated at 33 and 37° C. for 1 h, respectively. After 1 h, supernatant was discarded and the cells were washed 3 times with HBSS, and the third wash was kept for analysis. Then, hNECs and hBECs were incubated at 33° C. or 37° C., respectively. Samples were collected on designated time points post-infection. Infectious particle titers were assessed by TCID50 assays on VeroE6/TMPRSS2 cells. Each line in the graphs shows the mean titer obtained from 6 replicates of one individual sample. Statistical significances in the titer differences of OTS viruses vs WT on given times were determined using two-way ANOVA and p-values were adjusted using Tukey's multiple-comparison test; *P<0.05, **P<0.01, ***P<0.001, ****P<0.0001. d, Experimental setup of OTS-228 attenuation experiment in Syrian hamsters. Hamsters (n=10) were intranasally inoculated with 103.6 TCID50 of OTS-228. Naïve direct contact animals were added one day post-vaccination (dpv). e, Survival (%) and f, body weight change of immunized and contact hamsters post-vaccination. Body weight was significant different between OTS-228 and mock-group (unpaired t-test with Welch's correction, p=<0,0001). g, Viral genome copy numbers in nasal washing and h, organ samples of 5 dpv and 21 dpv were quantified using probe-specific RT-qPCR. i, Serum samples of 5 and 21 dpv were analyzed by SARS-CoV-2RBD-ELISA. j, Serum samples which reacted positive in the SARS-CoV-2RBD-ELISA, were analyzed by virus neutralization assay (capacity to neutralize 100 TCID50) against ancestral (B.1) SARS-CoV-2 as well as against Omicron BA.2 and BA.5 variants. k, Experimental setup of Omicron BA.5 challenge infection of OTS-228-vaccinated Syrian hamsters. Hamsters (n=8) were intranasally inoculated with 103.6 TCID50 of OTS-228. Naïve direct contact animals were added one day post-challenge(dpc) infection with 103.9 TCID50 of BA.5. l, Survival (%) and m, body weight change of OTS-228-immunized and contact hamsters post-BA.5-challenge. n, Viral genome copy numbers in nasal washing and o, organ samples of 5 dpc and 14 dpc were quantified using probe-specific RT-qPCR. p, Serum samples of 5 and 14 dpc were analyzed by SARS-CoV-2RBD-ELISA. r, Serum samples which reacted positive in the SARS-CoV-2RBD-ELISA were analyzed by virus neutralization assay (capacity to neutralize 100 TCID50) against ancestral (B.1) as well as against Omicron BA.2 and BA.5 variants. Statistical significance was determined using two-way ANOVA and p-values were adjusted using Tukey's multiple comparison test; *P<0.05, **P<0.01, ***P<0.001, ****P<0.0001.

FIG. 23: OTS constructs show comparable replication kinetics to WT in vitro, but higher sensitivity to treatment with antivirals. a, Schematic overview of the mutations introduced to SARS-CoV-2 genome to generate OTS codons. Fragments 2, 4, 5, 7, and 8, which are used for TAR cloning of recombinant SARS-CoV-2 clones have been modified to enrich the number of one-to-stop codons. The number of codons and nucleotides that have been changed are indicated for each fragment. For the OTS-206 construct, two additional point mutations were introduced in nsp1 (K164A/H165A) and open reading frames ORF6 to ORF8 were deleted. The number of codons and nucleotides that have been changed in each fragment are listed in Supplementary Table 3. b, Representative pictures of the plaque sizes of viruses in 6-well plates 2 dpi. c, Vero E6/TMPRSS2 cells (n=3) and d, human bronchial epithelial cell (hBECs) (n=6 (3 replicates from 2 donors)) cultures were infected with 0.1 MOI of the SARS-CoV-2 WT and OTS viruses from the apical side and incubated at 37° C. for 1 h. After 1 h, supernatant was discarded and the cells were washed 3 times with PBS, and the third wash was kept for analysis. Following the addition of new sera on the cells, they were incubated 37° C. Samples were collected on 6, 18, 24 and 48 h post-infection. Infectious particle titers were assessed by TCID50 assays on VeroE6/TMPRSS2 cells. Each line in the graphs shows the titers obtained from one individual sample. Statistical significance was determined using two-way ANOVA and p-values were adjusted using Tukey's multiple comparison test; *P<0.05, **P<0.01, ***P<0.001, ****P<0.0001.

FIG. 24: Attenuation of OTS2, OTS7, OTS8, OTS4-5 and OTS7-8 in K18-hACE2 mice. a, Experimental setup of comparison of OTS2, OTS7, OTS8 to WT infection in short-term. K18-hACE2 mice (7-16 week-old, n=4 mice/group) were infected with 5′000 PFU of either OTS2, OTS7, OTS8 and SARS-CoV-2 WT virus, or only with medium for 5 days. b, c, Mice were monitored for body weight change and clinical symptoms over the 5-day course of infection. On 5 dpi, mice were euthanized and samples from the nose, lungs, brain and olfactory bulbs are collected for evaluation of infectious virus titers, viral genome copy numbers, and pathology. d, Infectious virus titers from the nose, lung and brain samples were determined using plaque assays in VeroE6 cells. e, genome copy numbers (genome equivalence per ml, gEq/mL) in the nose, lung, brain and olfactory bulb samples of mice infected with different viruses were quantified using probe-specific RT-qPCR. f, Histopathological lung score was given for characterization and comparison of the severity of lung lesions. g, Hematoxylin and eosin stain (left panel) and immunohistochemical analysis specific for SARS-CoV-2 nucleocapsid protein (right panel) of lung and brain sections (n=4 per group) (magnification 50×). h, Experimental setup of comparison of OTS4-5, OTS7-8 to WT infection in short-term. K18-hACE2 mice (7-16 week-old, n=4 mice/group) were infected with 5′000 PFU of either OTS4-5, OTS7-8 and SARS-CoV-2 WT virus, or only with medium for 5 days. i, j, Mice were monitored for body weight change and clinical symptoms over the 5-day course of infection. On 5 dpi, mice were euthanized and samples from the nose, lungs, brain and olfactory bulbs are collected for evaluation of infectious virus titers, viral genome copy numbers, and pathology. k, Infectious virus titers from the nose, lung and brain samples were determined using plaque assays in VeroE6 cells. 1, m, genome copy numbers (genome equivalence per ml, gEq/mL) in the nose, lung, brain and olfactory bulb samples of mice infected with different viruses were quantified using probe-specific RT-qPCR. n, Histopathological lung score was given for characterization and comparison of the severity of lung lesions. o, Hematoxylin and eosin stain (left panel) and immunohistochemical analysis specific for SARS-CoV-2 nucleocapsid protein (right panel) of lung and brain sections (n=4 per group) (magnification 50×). Consolidated lung areas are highlighted with an asterisk, and perivascular and peribronchiolar lymphohistiocytic inflammation highlighted with an arrowhead. Viruses were visualized in the lungs of the infected animals by immunohistochemistry by anti-N SARS-CoV Antibody (Rockland). Statistical significance was determined using one-way or two-way ANOVA (a-d) and P values were adjusted using Tukey's multiple-comparison test; *P<0.05, **P<0.01, ***P<0.001, ****P<0.0001. Data were obtained from one experiment. Each data point represents one biological replicate. Body weight changes, clinical scores and histopathological score of the lungs of all K18-hACE2 mice experiments are shown in FIG. 33.

FIG. 25: Safety study of OTS4-5, OTS7-8 and OTS-206 in Syrian hamster model. a, Experimental setup of intranasal inoculation of Syrian hamsters with OTS4-5, OTS7-8, or OTS-206 SARS-CoV-2. b, c, Body weight changes of inoculated and contact hamsters in percent. d, e, Virus genome copy numbers in nasal washings of donor and contact hamsters. f, g, Virus genome copy numbers in the organ samples of donors at 5 and 21 dpi. h, Virus genome copy numbers in the organ samples of contact hamsters at 21 dpi. i, Serum samples of 5 and 21 dpi analyzed by SARS-CoV-2RBD-ELISA. j, Serum samples that reacted positive in the ELISA, were analyzed in addition by live virus neutralization assay (capacity to neutralize 100 TCID50) against ancestral WT SARS-CoV-2. Statistical significance was determined using two-way ANOVA and p-values were adjusted using uncorrected Fisher's LSD, with individual variances computed for each comparison. *P<0.05, **P<0.01, ***P<0.001, ****P<0.0001. k, Pneumonia-induced pulmonary atelectasis 5 dpi given in % affected area 1, Histopathology, lung whole slide images showing atelectasis, hematoxylin-eosin stain, bar 2.5 mm. m, Virus antigen score, 0=no antigen, 1=focal, 2=multifocal, 3=coalescing, 4=diffuse. n, Virus antigen, representative immunohistochemistry for SARS-CoV nucleocapsid protein detection, mainly in type-1 pneumocytes, bar 100 μm.

FIG. 26: Immunization with OTS4-5, OTS7-8, OTS4-5-7-8, and OTS-206 protects K18-hACE2 mice and Syrian hamsters from infection with SARS-CoV-2 Wuhan WT. a, K18-hACE2 transgenic mice (7-16-weeks-old, n=8 mice/group) were immunized with 5′000 PFU of either OTS viruses and SARS-CoV-2 WT virus, or only with medium (mock). b, Mice were monitored for clinical symptoms over the course of infection and c, oropharyngeal swabs were taken on the indicated days. On day 15 post-immunization blood samples were taken to have pre-challenge serum samples. 21-dpi, mice were challenged with 5′000 PFU of SARS-CoV-2 WT, and mice were euthanized on 5-days and 14-days post-challenge (dpc) (26 and 35 dpi, respectively). On 5 and 14 dpc, mice were euthanized and samples from the nose, lungs, brain, and olfactory bulbs are collected for evaluation of infectious virus titers, viral genome copy numbers, and pathology. d, f, Infectious virus titers from the brain, lung and nose samples were determined using plaque assays in VeroE6 cells. e, g, h, genome copy numbers (genome equivalence per ml, gEq/mL) in the post-challenge samples of mice infected with different viruses were quantified using probe-specific RT-qPCR. i, Hematoxylin and eosin stain of lung sections (n=4 per group and time point). Consolidated lung areas corresponding to interstitial pneumonia are highlighted with an asterisk, perivascular and peribronchiolar cuffings are highlighted with an arrowhead and tertiary lymphoid follicle formations with an arrow. No viral antigen was detected by immunohistochemistry by anti-N SARS-CoV Antibody (Rockland) in the immunized mice samples. Magnification 50×. j, Sera collected on 15 dpi (pre-challenge) and 5 and 14 dpc (post-challenge) were tested against SARS-CoV-2 Wuhan WT virus in a serum neutralization test. k, Whole blood cells collected on 15 dpi (pre-challenge) and 5 and 14 dpc (post-challenge) were labeled with Alexa fluor 647 labeled tetramer against SARS-CoV-2 Spike 539-546 (VNFNFNGL). Statistical significance was determined using one-way or two-way ANOVA and P values were adjusted using Tukey's multiple-comparison test; *P<0.05, **P<0.01, ***P<0.001, ****P<0.0001. Data were obtained from one experiment. Each data point represents one biological replicate. Body weight changes, clinical and histopathological scores of the lungs of all K18-hACE2 mice experiments are shown in FIG. 33. l, Sera samples of OTS4-5 or OTS7-8 vaccinated and subsequently SARS-CoV-2 WT challenged Syrian hamsters (FIG. 21), as well as sera of co-housed contact animals, were analyzed by SARS-CoV-2-RBD specific ELISA, confirming all samples, including the naïve contact animals positive, 14 days post-challenge. m, For those samples it was confirmed that they have virus neutralizing capacity too, while mock, OTS4-5 contact, and OTS7-8 contact animals had only low titers of around 32, OTS4-5 and OTS7-8 vaccinated and subsequently challenged animals had titers in average of 1406 (OTS4-5) and 2055 (OTS7-8). n, Organ samples of OTS-206 vaccinated and subsequently SARS-CoV-2 Omicron BA.2 challenged Syrian hamsters (FIG. 2r) were analyzed by RT-qPCR. Only residual amount of challenge virus genome was detectable in individual lung samples of mock group animals and in the conchae samples at 14 dpc. o, Serological evaluation by SARS-CoV-2-RBD specific ELISA confirmed transmission of BA.2 challenge virus to the naïve contact animals for the mock vaccinated as well as for the OTS-206 vaccinated animals. p, Comparing the live virus neutralizing capacity, revealed substantial neutralizing titers of the OTS-206 vaccinated animals against ancestral SARS-CoV-2 and Omicron BA.2 VOC, while the post BA-2 challenge seroconverted mock and contact animals only exhibit minimal neutralizing capacity against the BA.2 variant. BA.2 challenge: p Pneumonia-induced pulmonary atelectasis 5 dpi given in % affected area q, Histopathology, lung whole slide images showing atelectasis, hematoxylin-eosin stain, bar 2.5 mm. r, Virus antigen score, 0=no antigen, 1=focal, 2=multifocal, 3=coalescing, 4=diffuse. s, Virus antigen, immunohistochemistry for SARS-CoV nucleocapsid protein detection, mainly in type-1 pneumocytes, bar 100 μm.

FIG. 27: Spatial transcriptomics shows that OTS-206 vaccination induces similar activation of genes related to the immune response to viral infection and reduced inflammatory response. a, Pearson's correlation coefficients were calculated between total SARS-CoV-2 gene counts and all host genes to determine spatial correlations. These values are plotted against each other on the x and y axis for the OTS and mRNA 2 dpc samples to show that the spatial gene expression signatures are very similar, as their correlation coefficients are nearly identical. b, Top 20 spatially most correlated genes in the lungs of infected mice vaccinated with OTS-206 or mRNA vaccine. c, Changes in proinflammatory cytokine expression between conditions. d, Spatial JAK-STAT pathway activity in the lung. We can see the co-occurrence between SARS-CoV-2 transcripts from d, and the increased JAK-STAT activity. Spatial transcriptomics samples (n=11): OTS 2dpc—2, OTS 5dpc—2, mRNA 2dpc—2, mRNA 5dpc—3, mRNA mock—1, OTS mock—1.

FIG. 28: OTS-206 demonstrates comparable efficacy to mRNA-vaccines and inducing long-term immunity in K18-hACE2 mice. a, K18-hACE2 transgenic mice (7-15 weeks old, n=8 mice/group) were immunized (prime & boost) either intramuscularly with a single dose of 1 μg of mRNA-Vaccine Spikevax (Moderna), or intranasally with 5′000 PFU of OTS-206. At 57 dpi a group of mice was intranasally inoculated with 104 TCID50 of SARS-CoV-2D614G, or SARS-CoV-2 Delta VOC (c-d). The rest of the immunized mice were kept for approximately 5 months and then intranasally inoculated with 104 TCID50 of SARS-CoV-2D614G (e-h). b, During immunization, mice were regularly monitored for body weight changes. Each line in the body weight loss graphs represents a mouse. Six days post-challenge, mice were euthanized and organ samples were collected for evaluation of infectious virus titers, viral genome copy numbers, and pathology. c, e, Genome copy numbers (genome equivalence per ml, gEq/mL) in nose, lung, brain, olfactory bulb and oropharyngeal swab samples of mice infected with different viruses were quantified using probe-specific RT-qPCR. d, f Infectious virus titers from the brain samples were determined using plaque assays in VeroE6 cells. g, h Sera collected on 6 dpc (post-challenge) were tested against SARS-CoV-2 Wuhan WT virus in a serum neutralization test. i, Immunohistochemical analysis specific for SARS-CoV-2 nucleocapsid protein (magnification 50×). Statistical significance between non-immunized and immunized mice was determined using unpaired nonparametric t-test (Mann Whitney test) (panels c-h); *P<0.05, **P<0.01, ***P<0.001, ****P<0.0001. Data were obtained from one experiment. Each data point represents one biological replicate. Body weight changes, clinical scores and histopathological score of the lungs of all K18-hACE2 mice experiments are shown in FIG. 33. FIG. 29: OTS-228 shows significantly reduces transmission, protects against and limits transmission of SARS-CoV-2 VOC challenge infections. a, Vero E6/TMPRSS2 cells were infected with 0.1MOI of the indicated viruses and incubated at 37° C. for 1 h. After 1 h, supernatant was discarded and the cells were washed 3 times with PBS, and the third wash was kept for analysis. Following the addition of new sera on the cells, they were incubated 37° C. Samples were collected on designated time points post-infection. Infectious particle titers were assessed by TCID50 assays on VeroE6/TMPRSS2 cells. Each line in the graphs shows one replicate of samples. Statistical significances in the titer differences of OTS viruses vs WT on given times were determined using two-way ANOVA and p-values were adjusted using Tukey's multiple-comparison test; *P<0.05, **P<0.01, ***P<0.001, ****P<0.0001.

FIG. 30: SARS-CoV-2 WT challenge infection of OTS-228 immunized hamsters. (a) Experimental setup. (b) Survival post-challenge infection. (c) Relative body weight in percent. (d) Virus genome copy numbers in nasal washing and (e) organ samples of 5 dpc. (f) Serum samples of 5 and 14 dpc were analyzed by SARS-CoV-2RBD-ELISA. (g) Serum samples that reacted positively in the ELISA, were analyzed in addition by live virus neutralization assay (capacity to neutralize 100 TCID50) against ancestral (B.1) SARS-CoV-2 as well as against Omicron BA.2 and BA.5 variants. Whenever calculated the statistical significance was determined using ordinary one-way ANOVA with p-values adjusted by Fisher's LSD test, calculated p-values are as indicated. *P<0.05, **P<0.01, ***P<0.001, ****P<0.0001.

FIG. 31: SARS-CoV-2 BA.2 challenge infection of OTS-228 immunized hamsters. (a) Experimental setup. (b) Survival post-challenge infection. (c) Relative body weight in percent. (d) Virus genome copy numbers in nasal washing and (e) organ samples of 5 dpc. (f) Serum samples of 5 and 14 dpc were analyzed by SARS-CoV-2RBD-ELISA. (g) Serum samples that reacted positively in the ELISA, were analyzed in addition by live virus neutralization assay (capacity to neutralize 100 TCID50) against ancestral (B.1) SARS-CoV-2 as well as against Omicron BA.2 and BA.5 variants. Whenever calculated the statistical significance was determined using ordinary one-way ANOVA with p-values adjusted by Fisher's LSD test, calculated p-values are as indicated. *P<0.05, **P<0.01, ***P<0.001, ****P<0.0001.

FIG. 32: Omicron BA.5 challenge of OTS-228 vaccinated hamsters. Virus genome copy numbers in organ samples 14 dpc. Whenever calculated the statistical significance was determined using ordinary one-way Anova with p-values adjusted by Fisher's LSD test, calculated p-values are as indicated.

FIG. 33: Body weight changes, clinical score and histopathological scores of k18-hACE2 mice FIG. 34: Immunization with OTS4-5, OTS7-8, OTS4-5-7-8 and OTS-206 protects K18-hACE2 mice from an infection with SARS-CoV-2 Wuhan WT. Gating strategy for the flow cytometry analysis. Blood was collected from mock and OTS- or WT-infected mice, and red blood cells were lyzed as explained in Materials and Methods section. Antibody mixes including the following antibodies were mixed with the cells and incubated for 30 min in dark on ice: anti-mouse anti-CD8-FITC (biolegend), anti-mouse anti-CD45-PerCP (biolegend), anti-mouse anti-CD3e-PE (biolegend), either MHC-I tetramer against SARS-CoV-2 spike (H-2K, SARS-CoV-2 S 539-546, VNFNFNGL) (NIH), or negative control (Influenza A NP, NIH). In addition, a fluorescence minus one (FMO) control without the tetramer or negative control antibody, as well as single antibody staining were prepared as flow cytometry control and compensation groups. Cells were washed two times with PBS, centrifuged at 350×g, 4° C. for 5 min. Finally, PBS+4% paraformaldehyde (PFA) (company) was added on the cells to fix them to take out the samples out of BSL3 for flow cytometry acquisition in FACS Canto II (BD Bioscience) using the DIVA software.

FIG. 35: Lung histopathology and virus antigen detection of OTS-228 vaccinated hamsters and after WT, Omicron BA.2, BA.5 challenge. a, Pneumonia-induced pulmonary atelectasis given in % affected area b, Histopathology, lung whole slide images showing atelectasis in control animals only, hematoxylin-eosin stain, bar 2.5 mm. c, No virus antigen was found after challenge. Virus antigen score, 0=no antigen, 1=focal, 2=multifocal, 3=coalescing, 4=diffuse. d, Virus antigen, representative immunohistochemistry for SARS-CoV nucleocapsid protein detection in control animals only, mainly in type-1 pneumocytes (green arrow), bar 100 μm. e, WT challenge led to perivascular (2/5) (green arrow) and peribronchial (2/5) inflammatory infiltrates, partially with necrotizing bronchitis and immune cell rolling at the vascular endothelium (green asterisk). Bar 100 μm.f, BA.5 challenge was associated with peribronchial (4/5) and perivascular (5/5) inflammatory infiltrates as well as vasculitis (1/5). Bar 100 μm. g, BA.2 challenge led to perivascular (3/5) and/or peribronchial (3/5, green arrow), infiltrates as well as necrotizing bronchitis (2/5), 100 μm.

### EXAMPLES

Aspects of the present invention are additionally described by way of the following illustrative non-limiting examples that provide a better understanding of embodiments of the present invention and of its many advantages. The following examples are included to demonstrate preferred embodiments of the invention. It should be appreciated by those of skill in the art that the techniques disclosed in the examples which follow represent techniques used in the present invention to function well in the practice of the invention, and thus can be considered to constitute preferred modes for its practice. However, those of skill in the art should appreciate, in light of the present disclosure that many changes can be made in the specific embodiments which are disclosed and still obtain a like or similar result without departing from the spirit and scope of the invention.

### Example 1

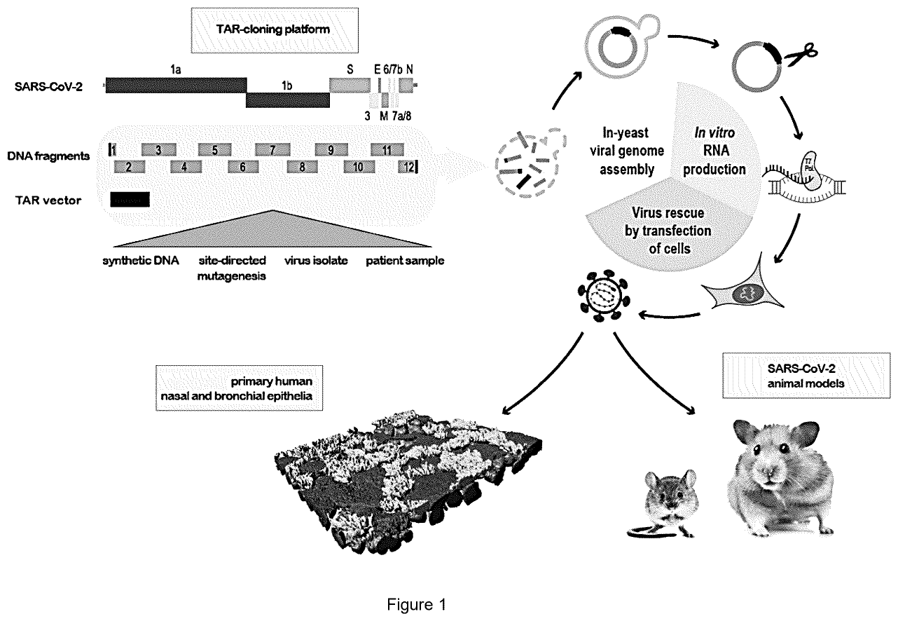

Generation of recombinant SARS-CoV-2 was done using “transformation-associated recombination” (TAR) cloning is yeast (12 overlapping DNA fragments spanning the entire SASRS-CoV-2 genome), subsequent generation of in vitro transcribed RNA resembling the recombinant SARS-CoV-2 RNA genome, and rescue of infectious recombinant viruses following transfection of in vitro transcribed RNA into BHK-SARS-N cells (Thi Nhu Thao, Tran, et al., 2020, Nature 582.7813: 561-565.; and FIG. 1).

Recombinant viruses were characterized in vitro in VeroE6 and VeroE6-TMPRSS2 cells, and primary human airway epithelial cultures. In vivo viruses were assessed in various animal models including K18-hACE2-mice, hACE2-KI-mice and Syrian hamsters (FIG. 1)

Cloning: A set of synthetic DNA fragments were designed to contain an enriched number of OTS codons encoding for Leu or Ser (see Table 1 or supplementary Table 3). Fragments 2-5, 7-8 (see FIG. 2) were selected since these encode for the viral replicase gene product and increased appearance of stop codons in this region of the genome were considered to be most effective in generating attenuated viruses.

The constructs were cloned and analyzed further.

### SARS-CoV-2-OTS Replication in Primary Airway Epithelial Cultures:

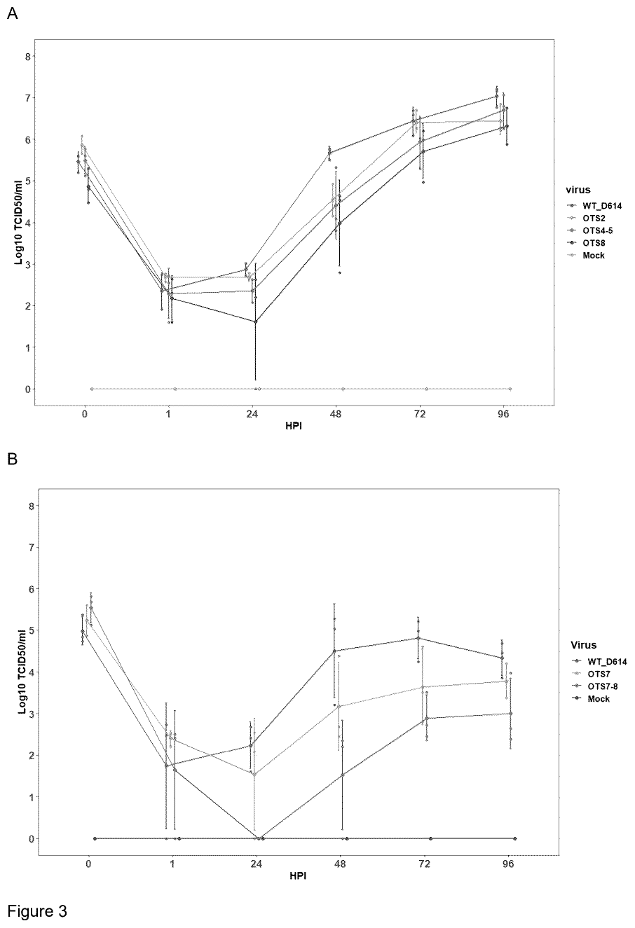

Virus titer was determined at 0 (inoculum), 1, 24, 48, 72, 96 hours post infection in apical washes. (FIG. 3)

### Assessment of Attenuation and Protection in K18-hACE2-Mice:

Based on the replication kinetics determined in primary human epithelial cultures the following experiments were conducted in vivo.

### Assessment of Attenuation:

K18-hACE2-mice were infected intranasally with 5000 PFU. Oropharyngeal swabs were taken daily. Organs were taken at days 2 and 5/6 post infection. Viral RNA was quantified by qRT-PCR and viral titers were determined by plaque assay (to determine PFUs). Clinical scores and body weight were determined daily.

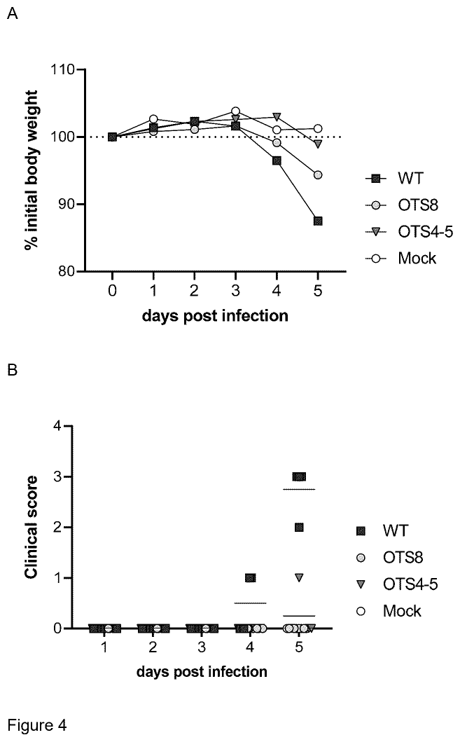

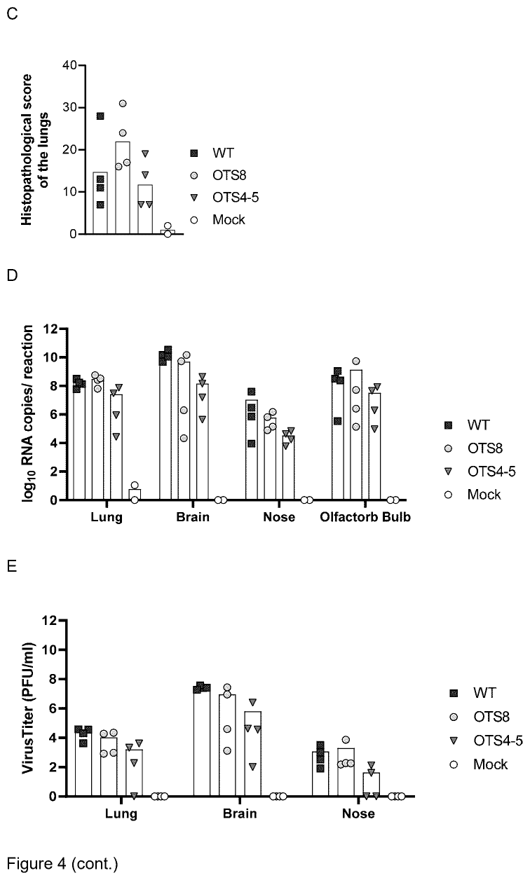

OTS8, OTS4-5 were assessed for attenuation (FIG. 4).

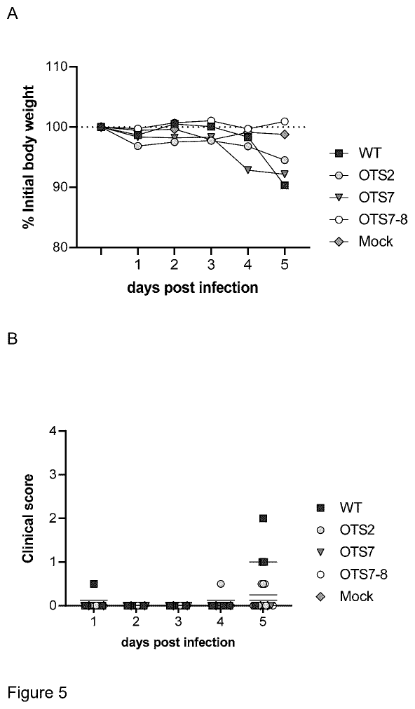

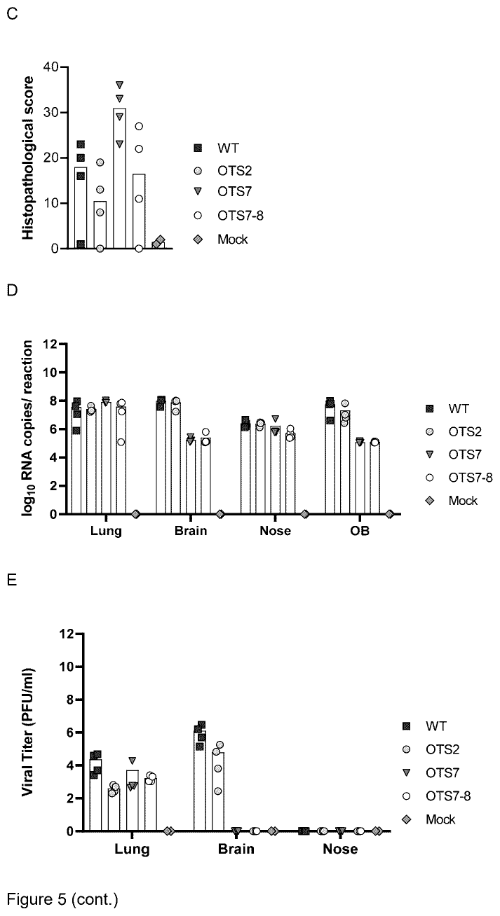

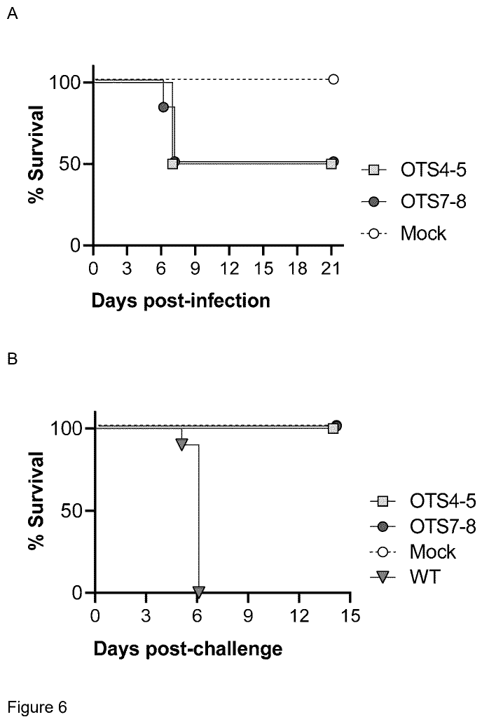

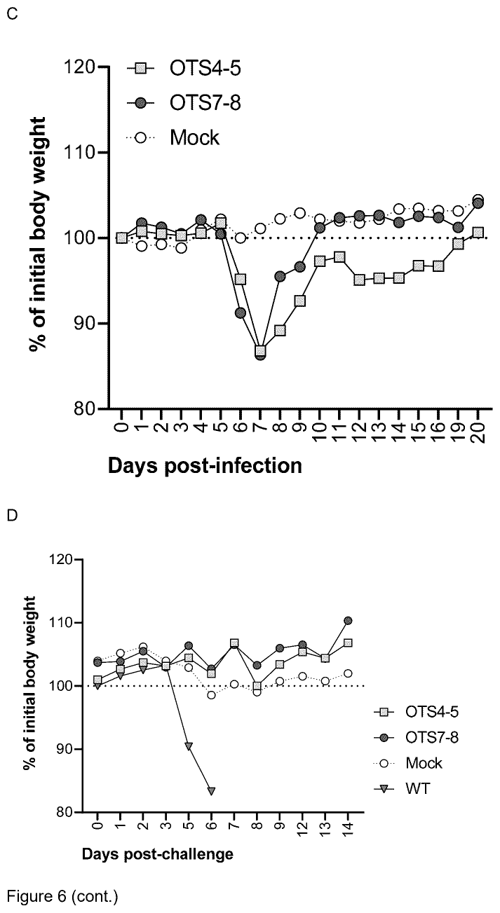

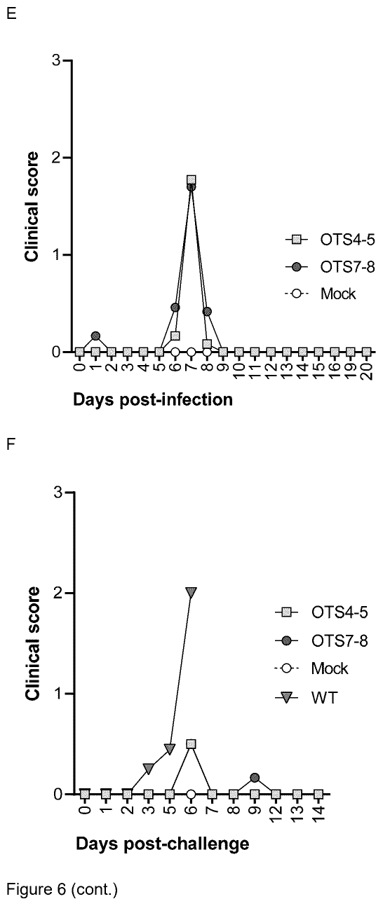

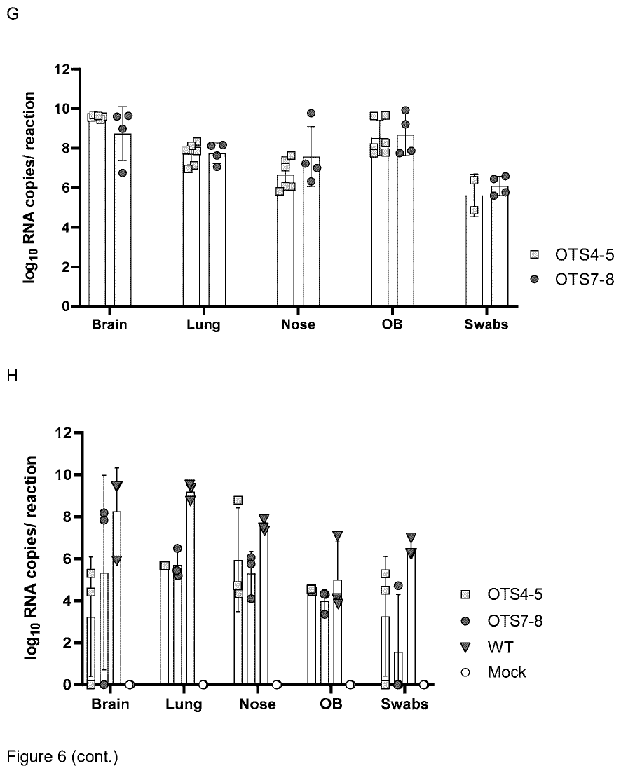

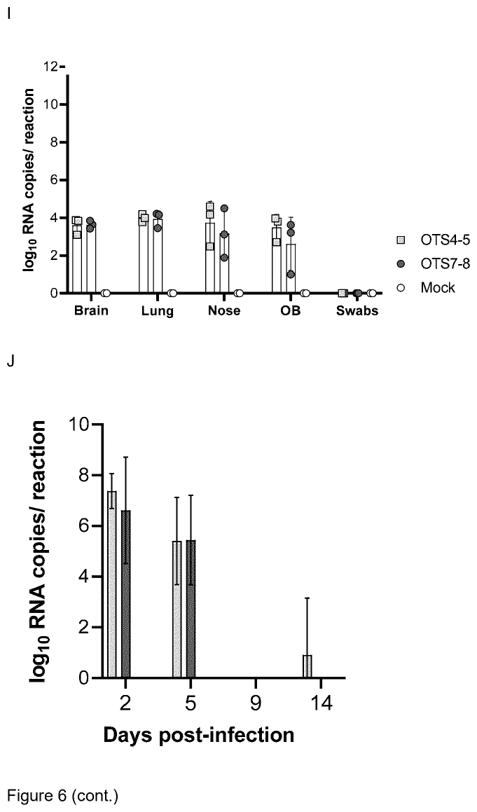

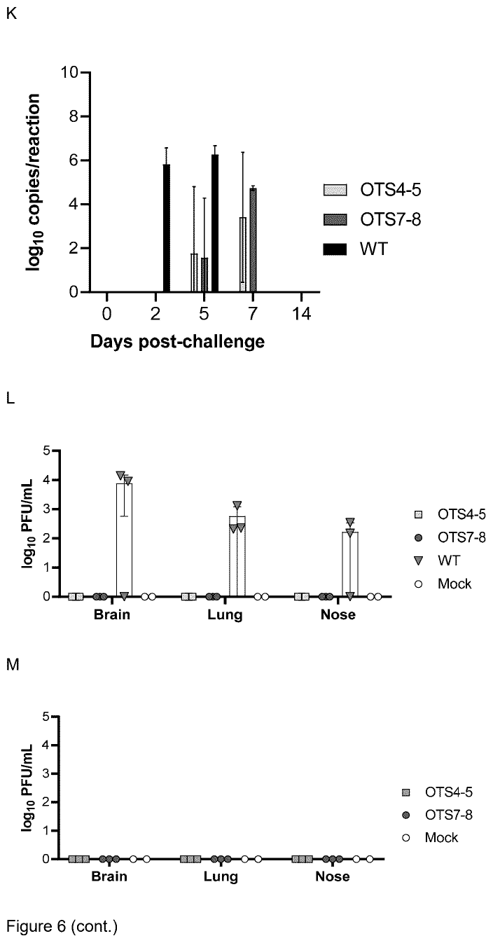

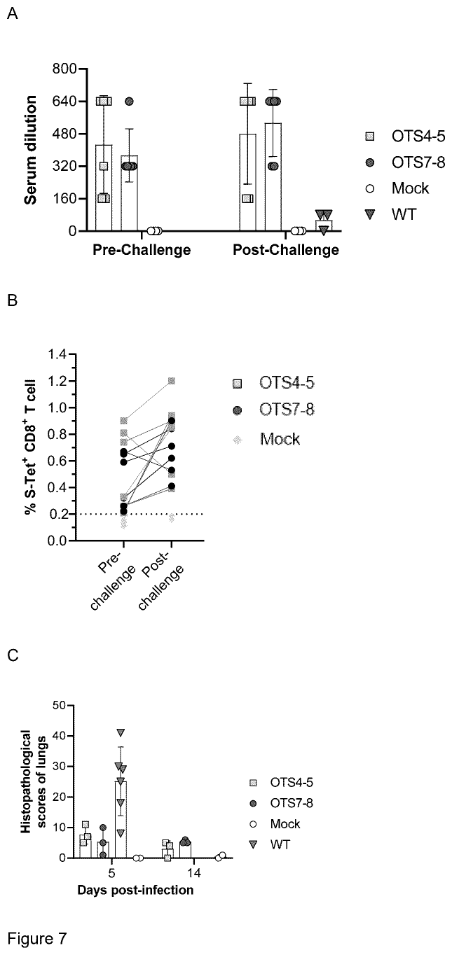

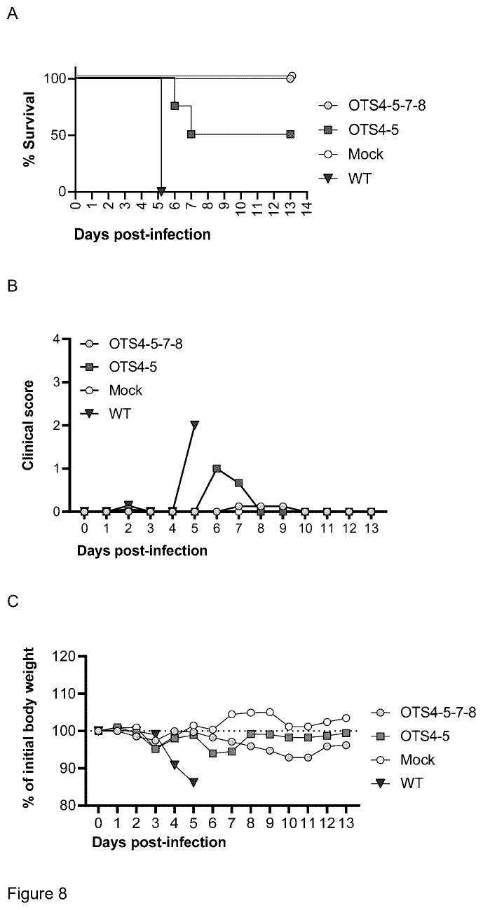

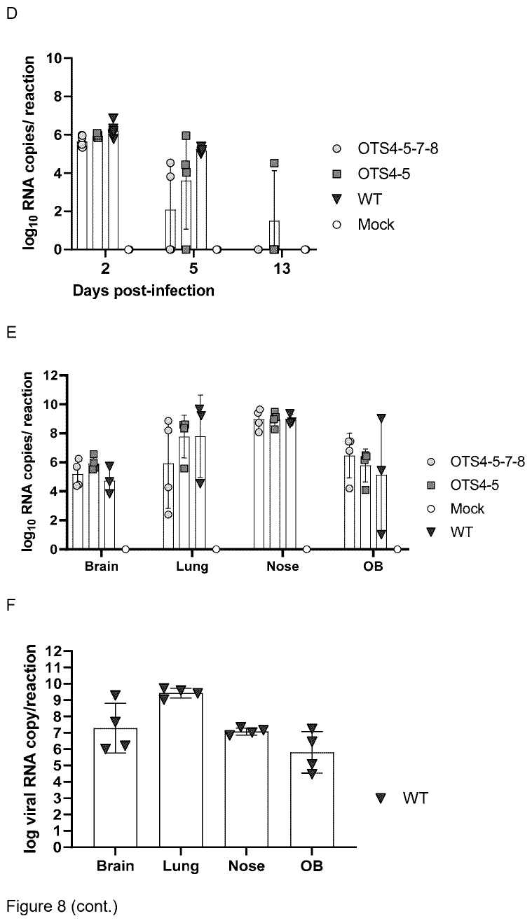

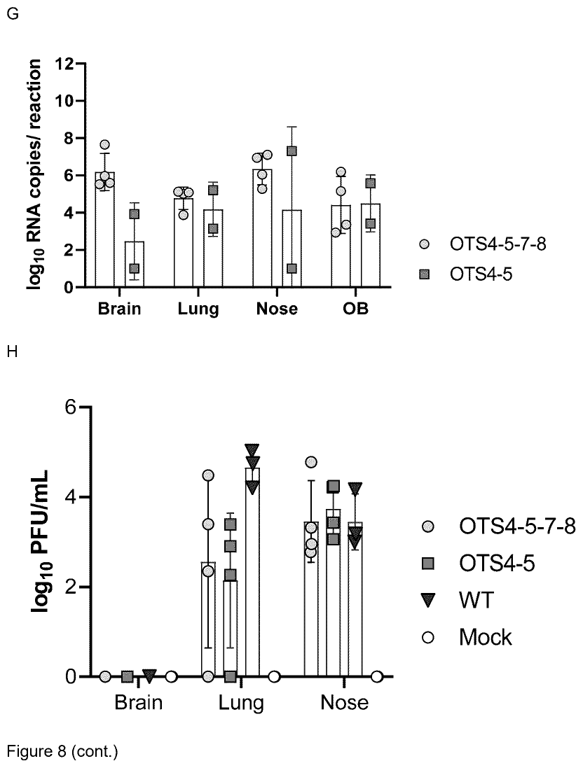

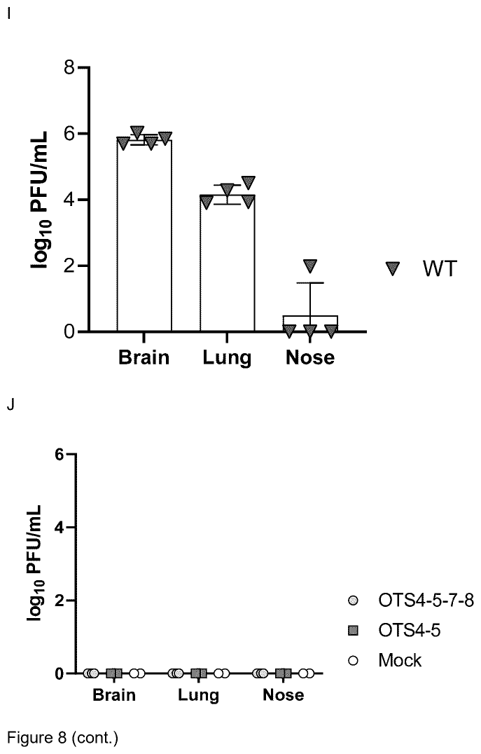

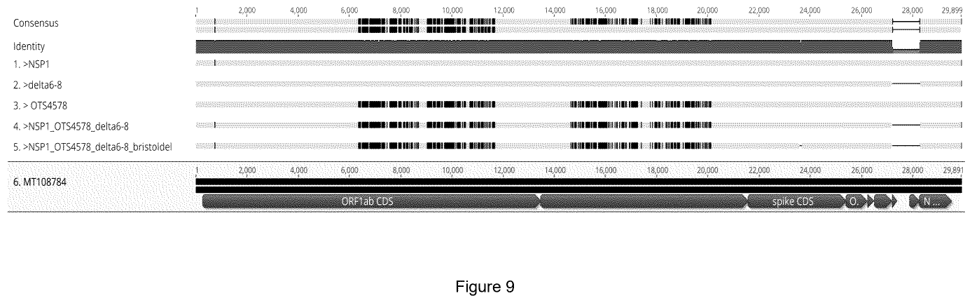

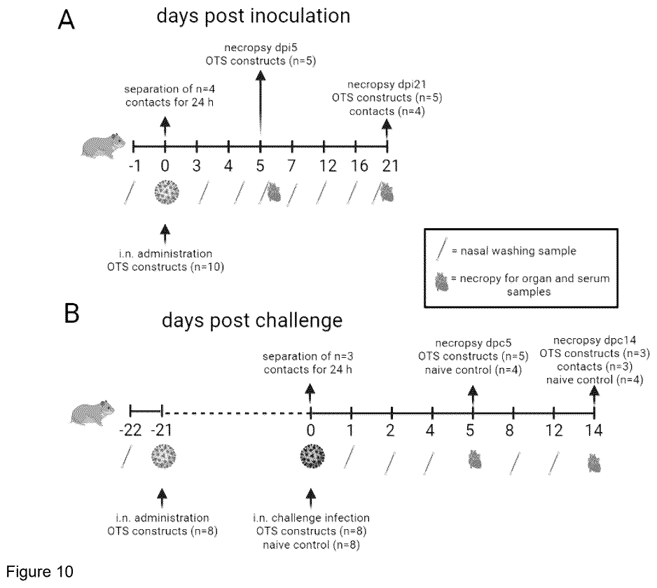

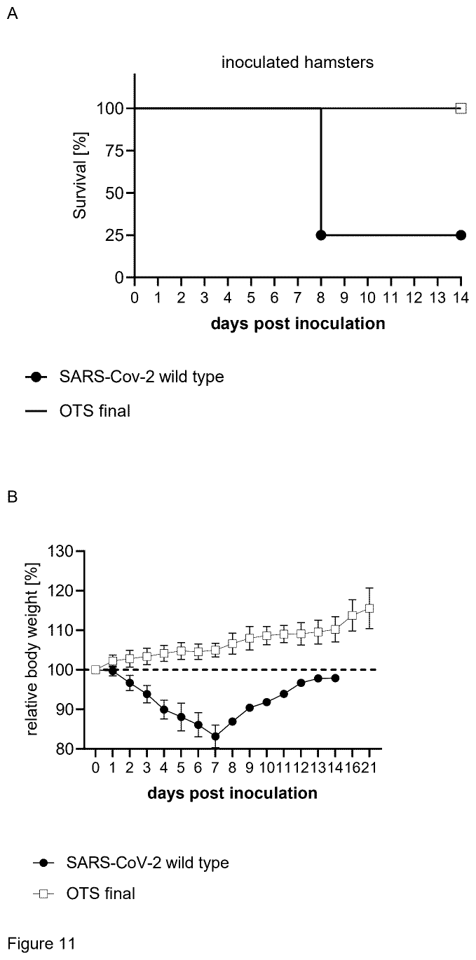

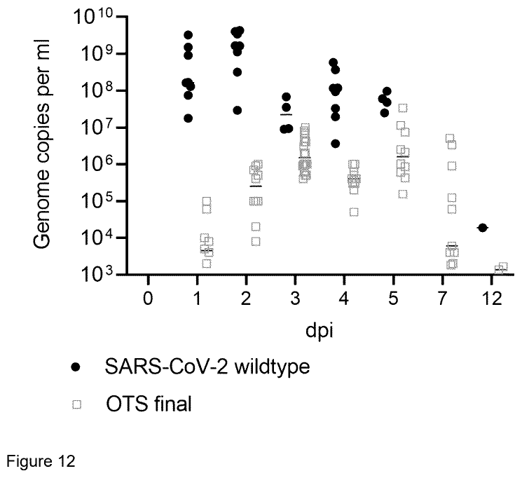

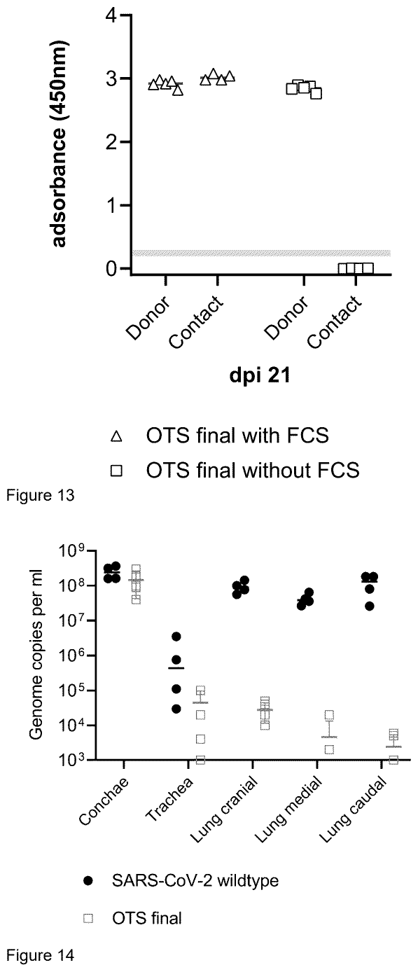

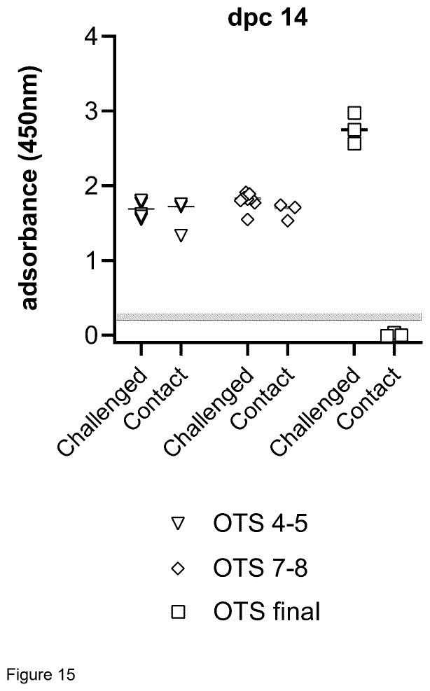

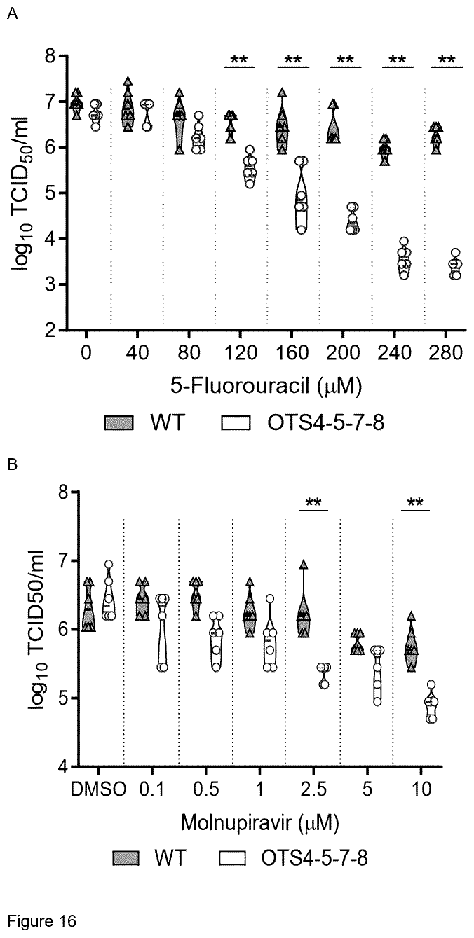

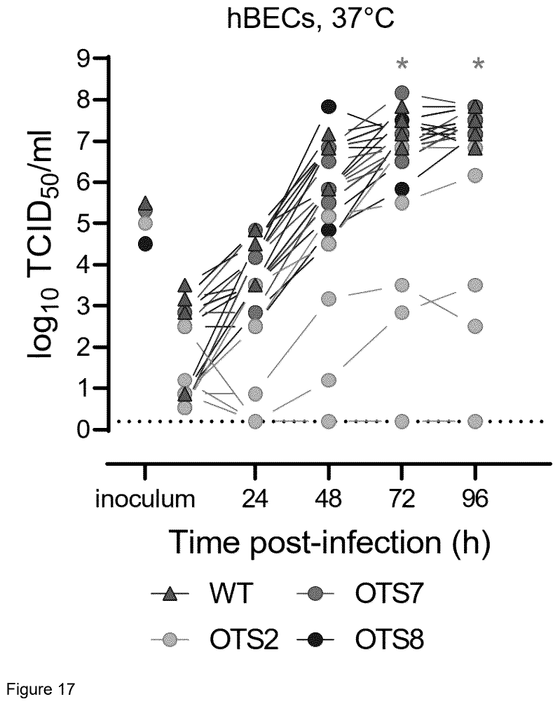

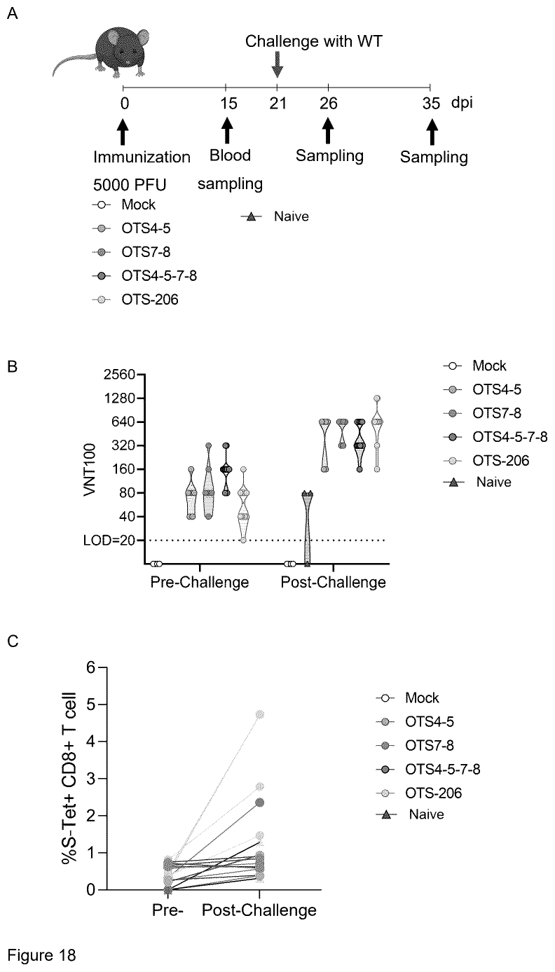

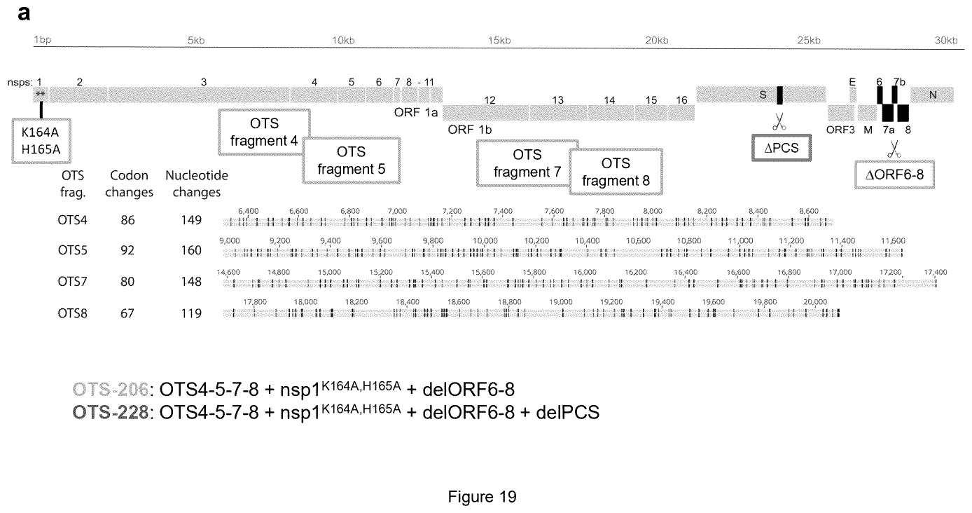

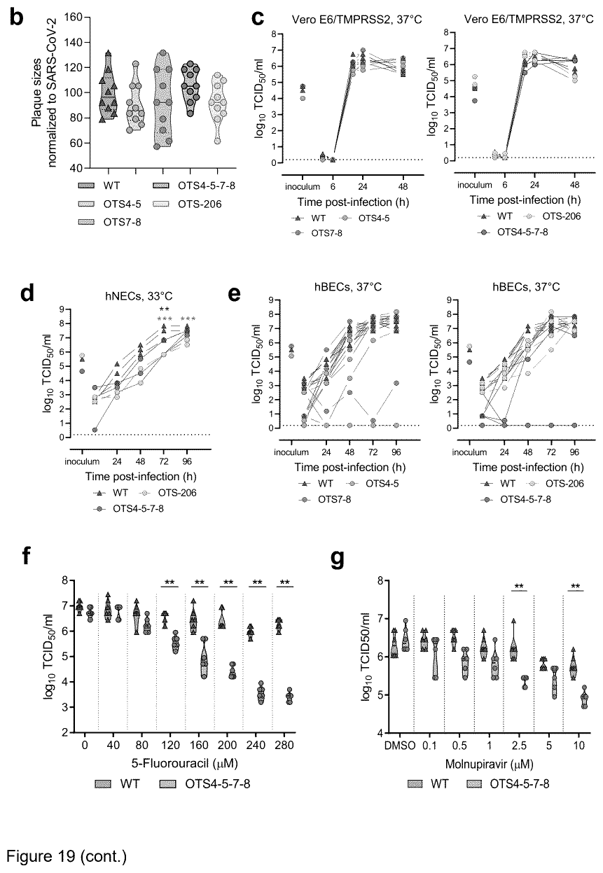

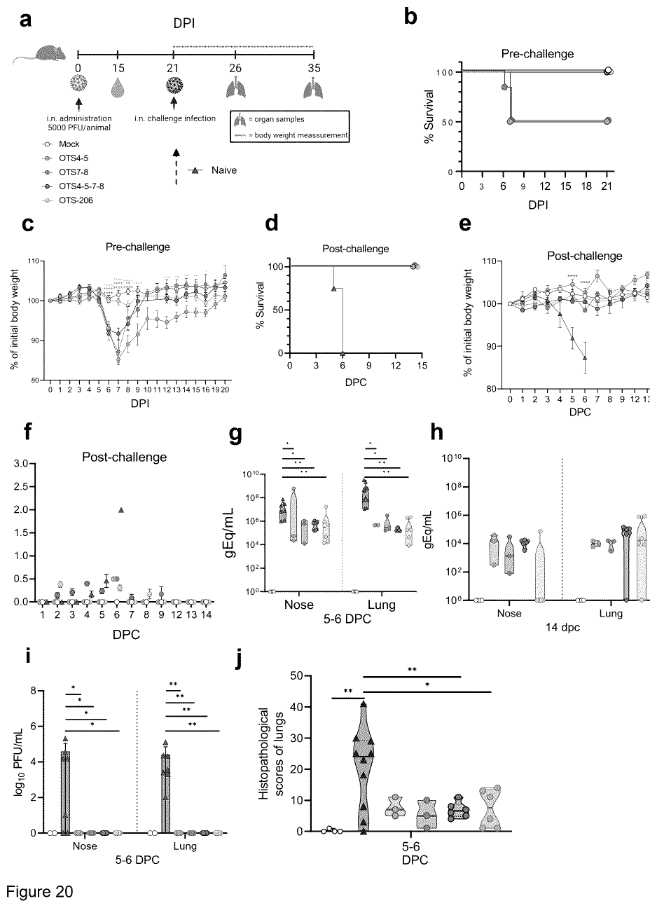

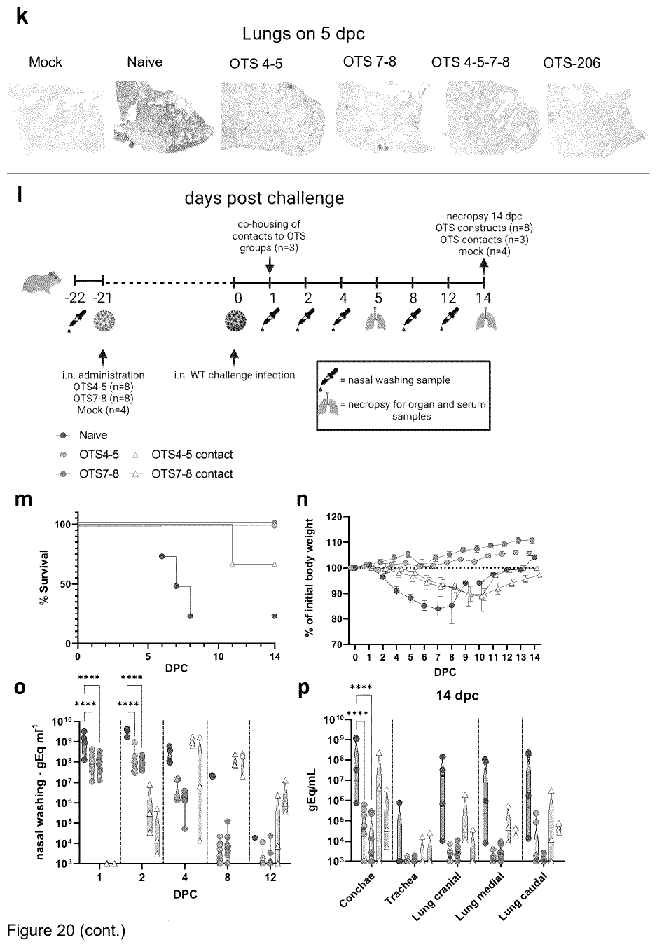

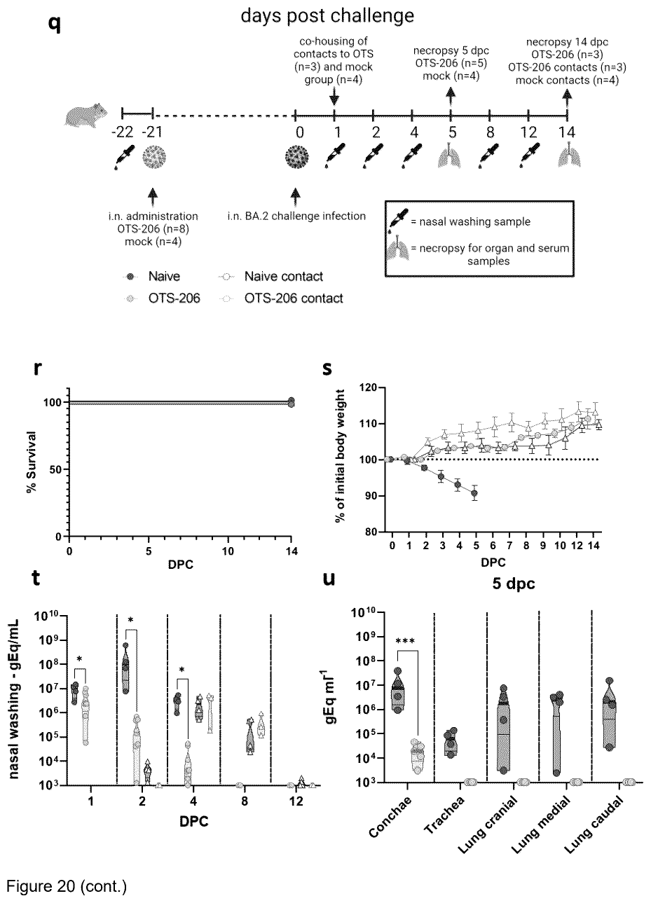

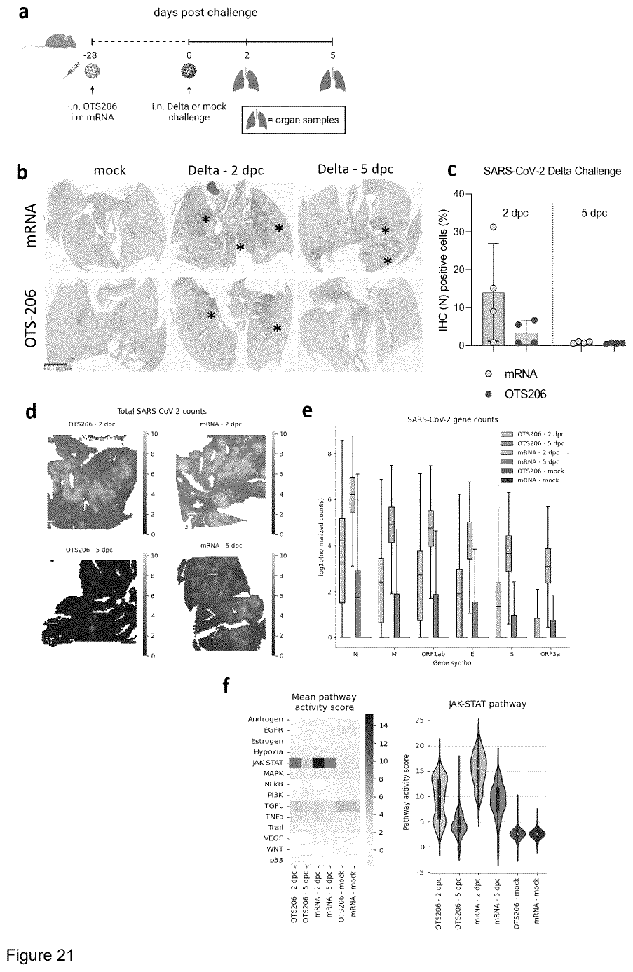

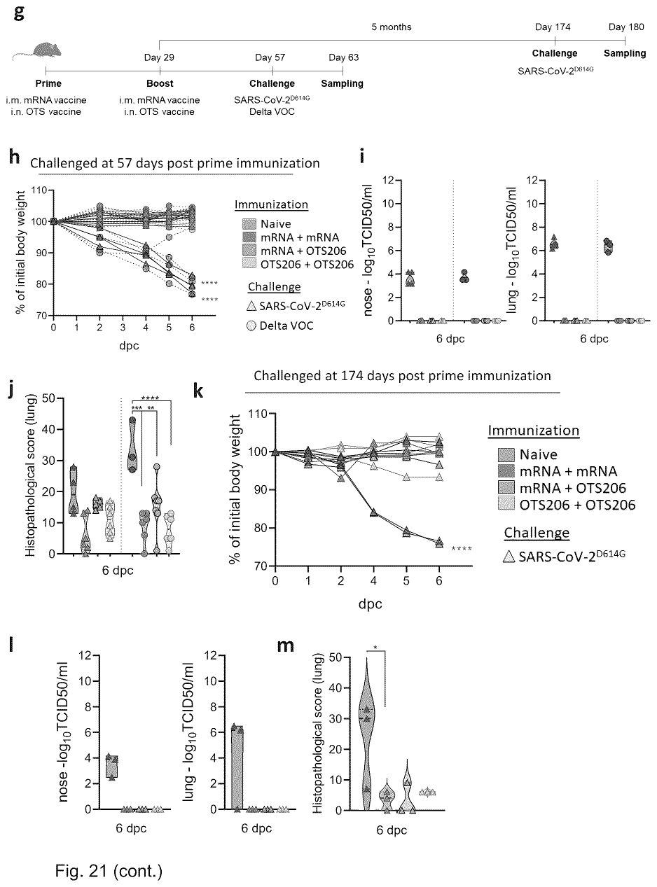

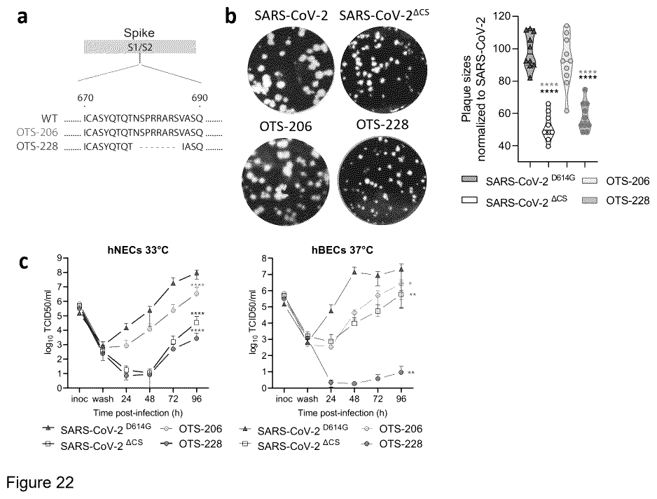

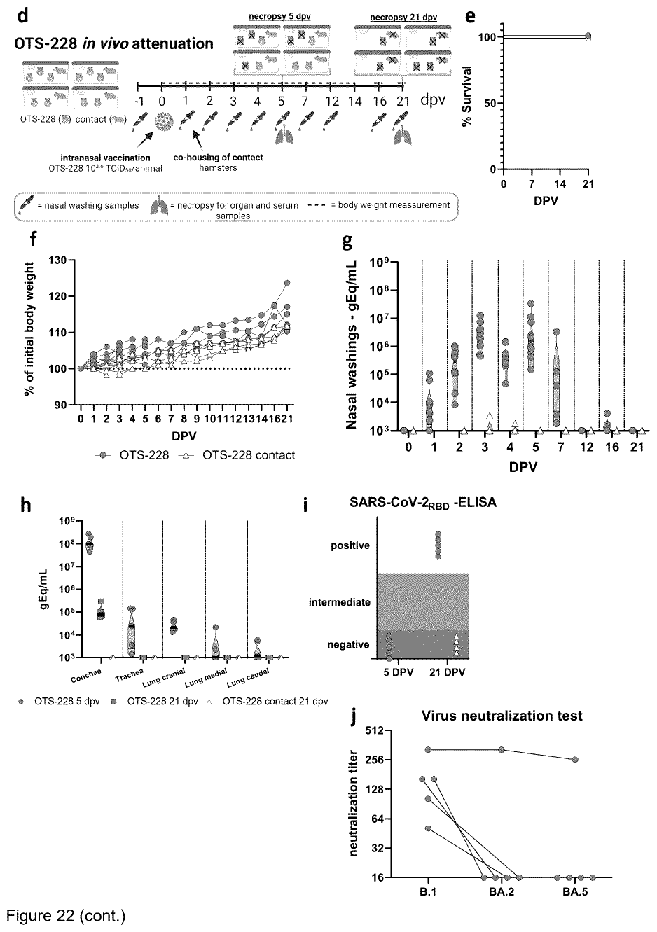

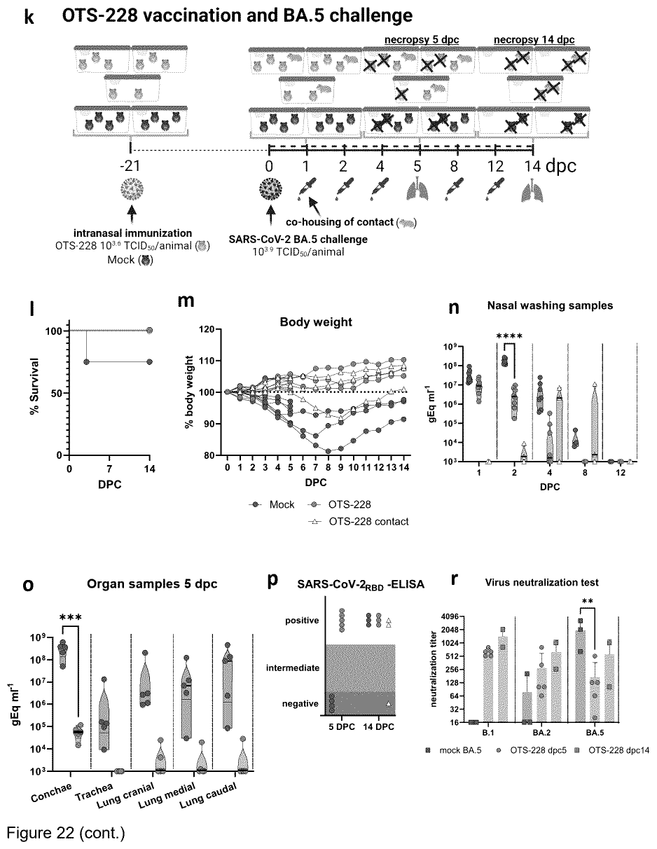

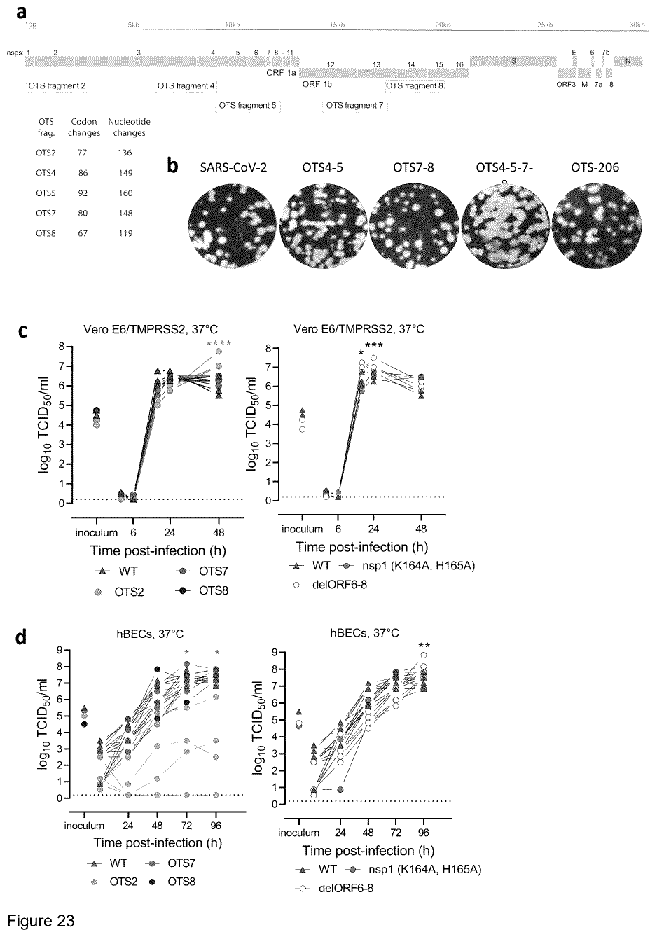

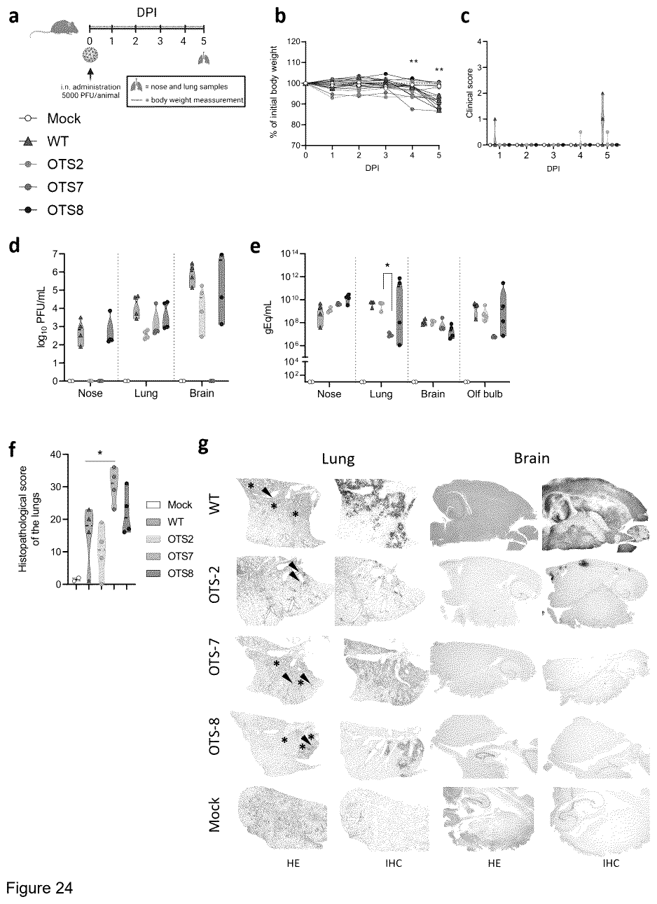

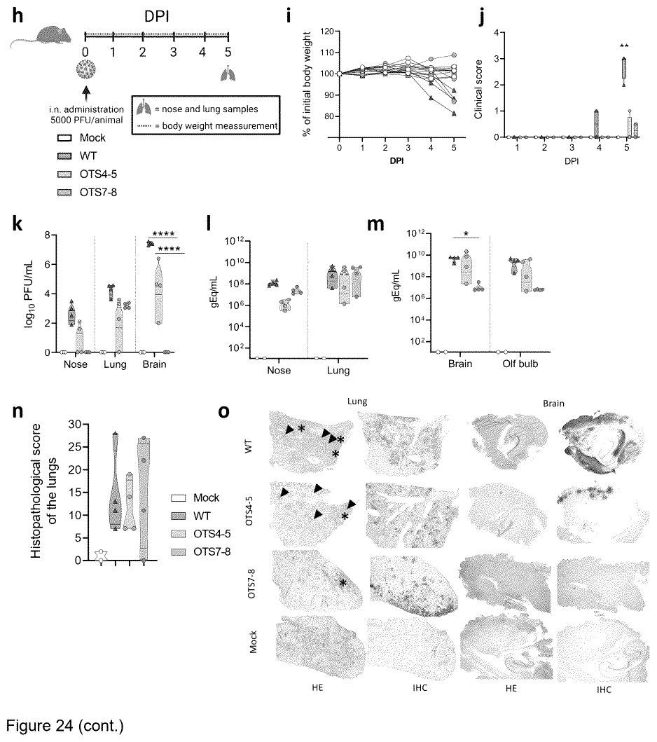

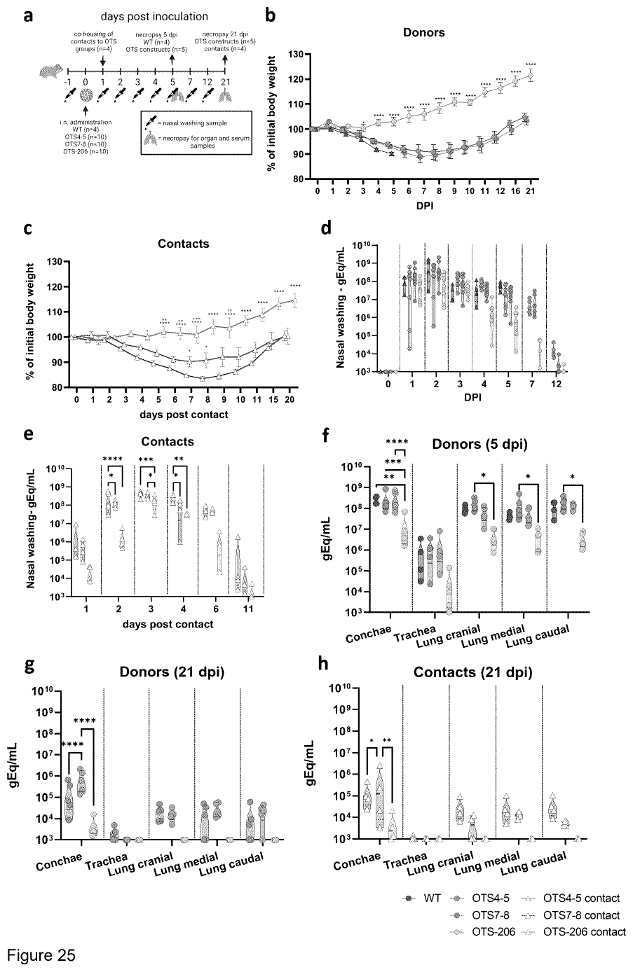

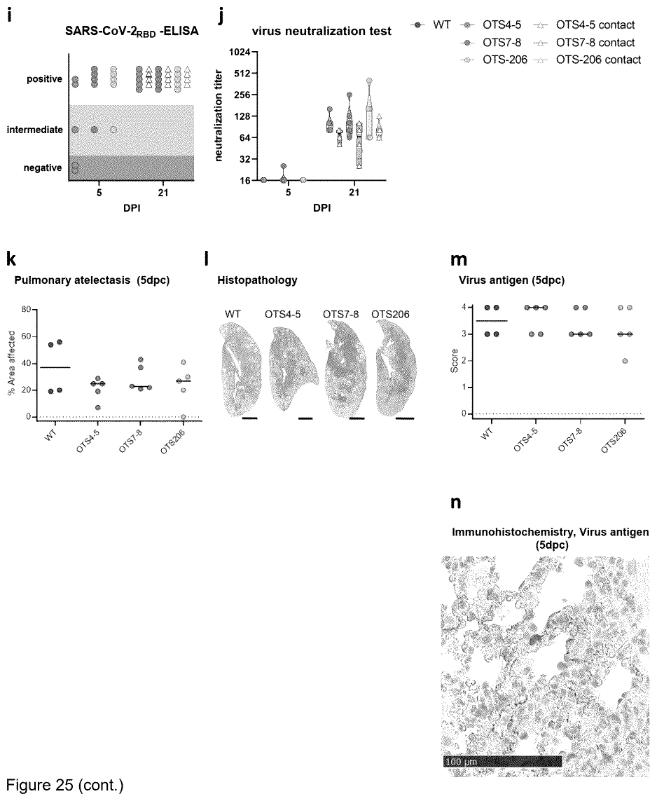

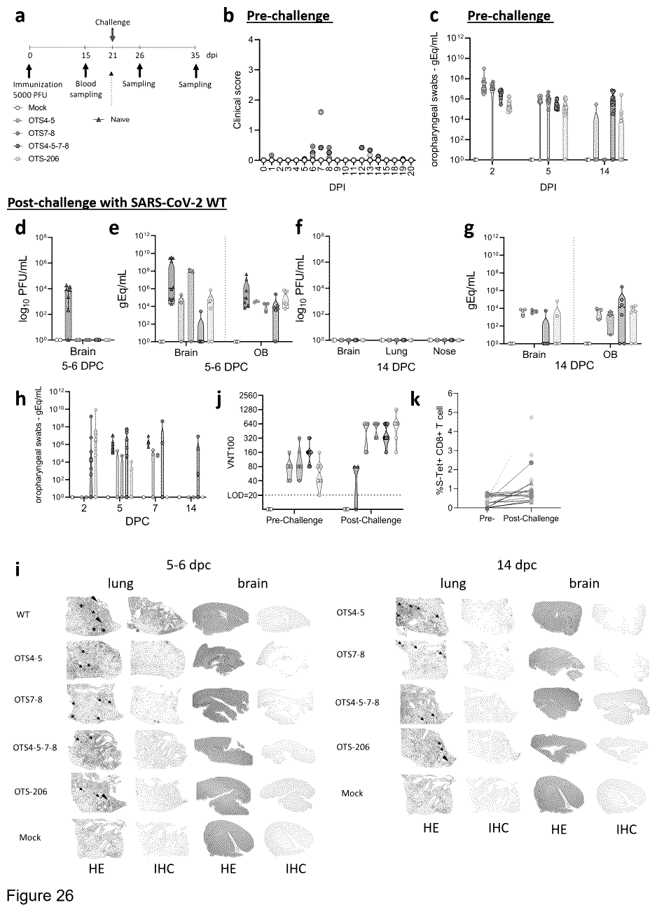

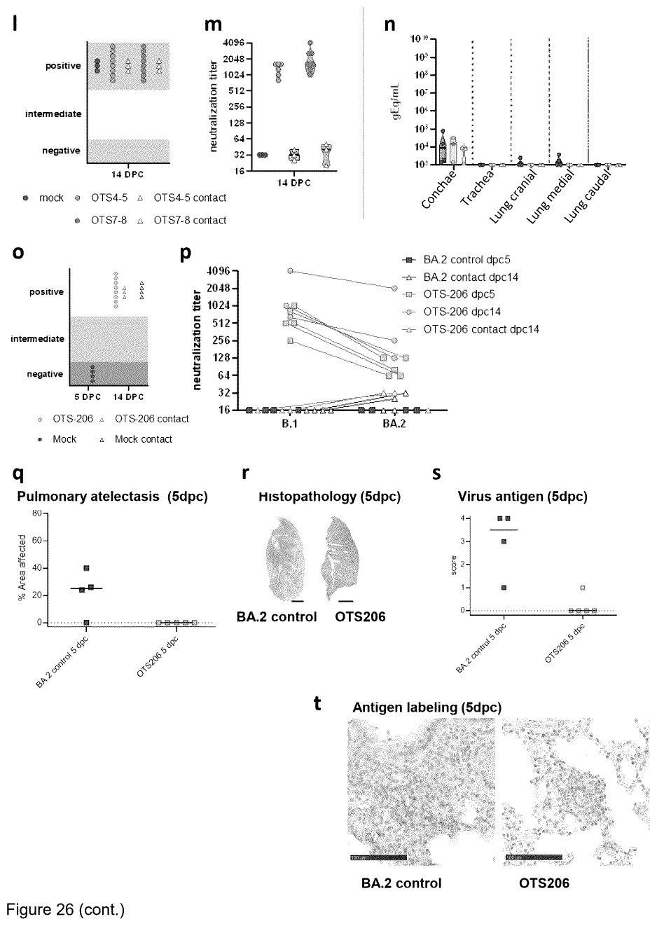

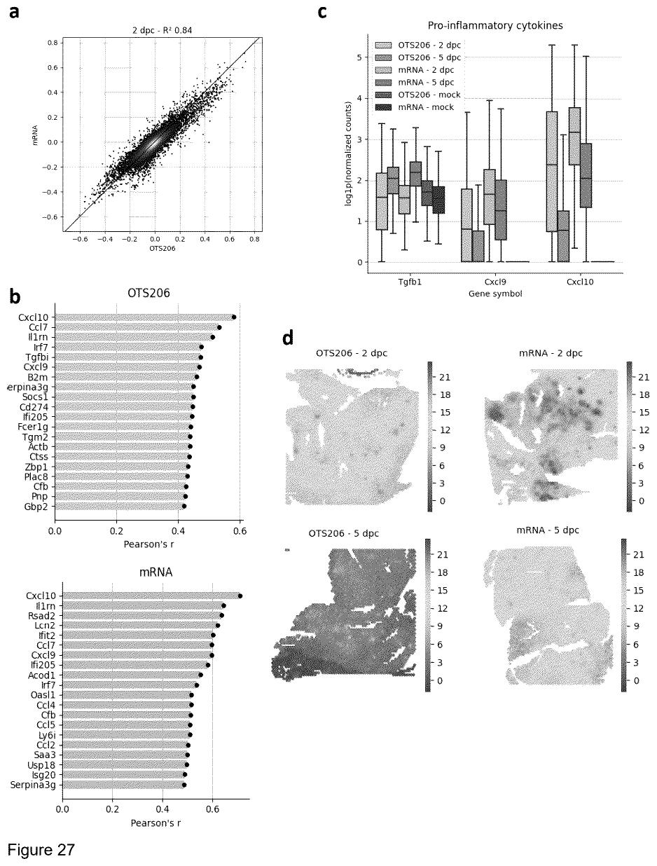

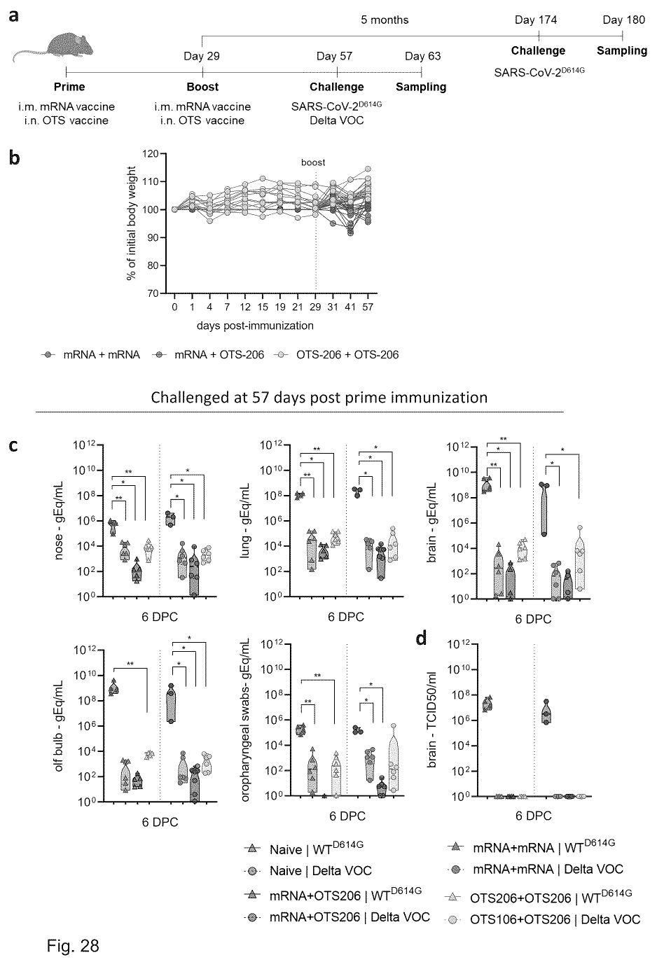

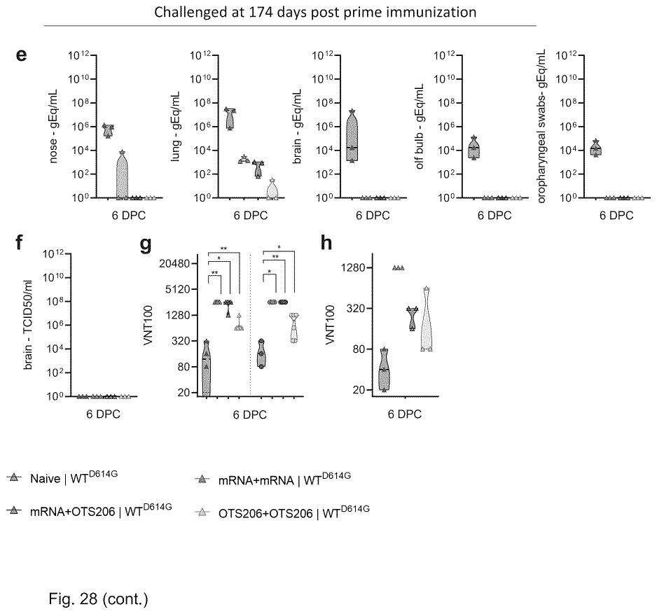

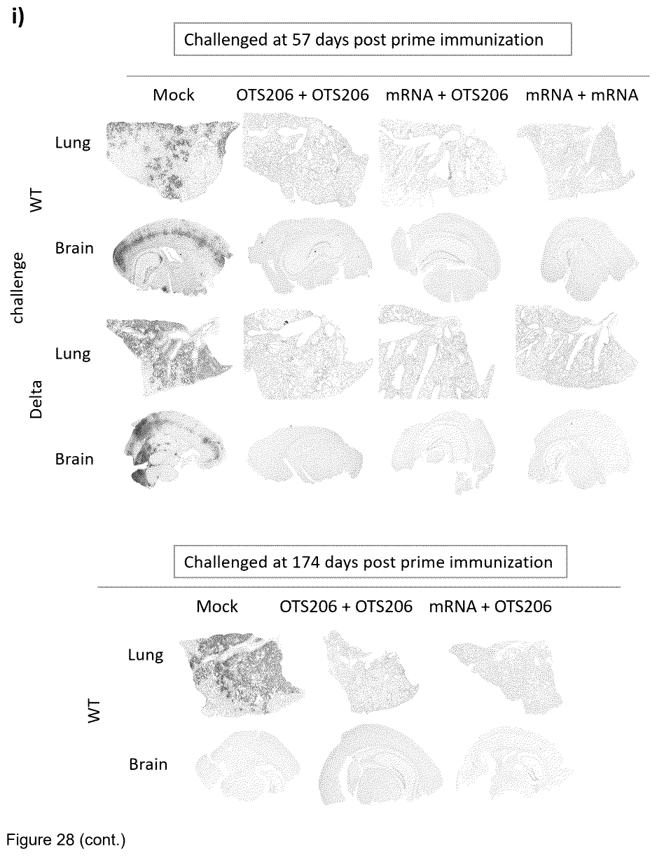

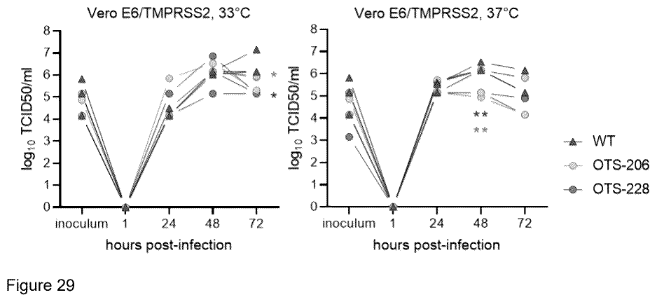

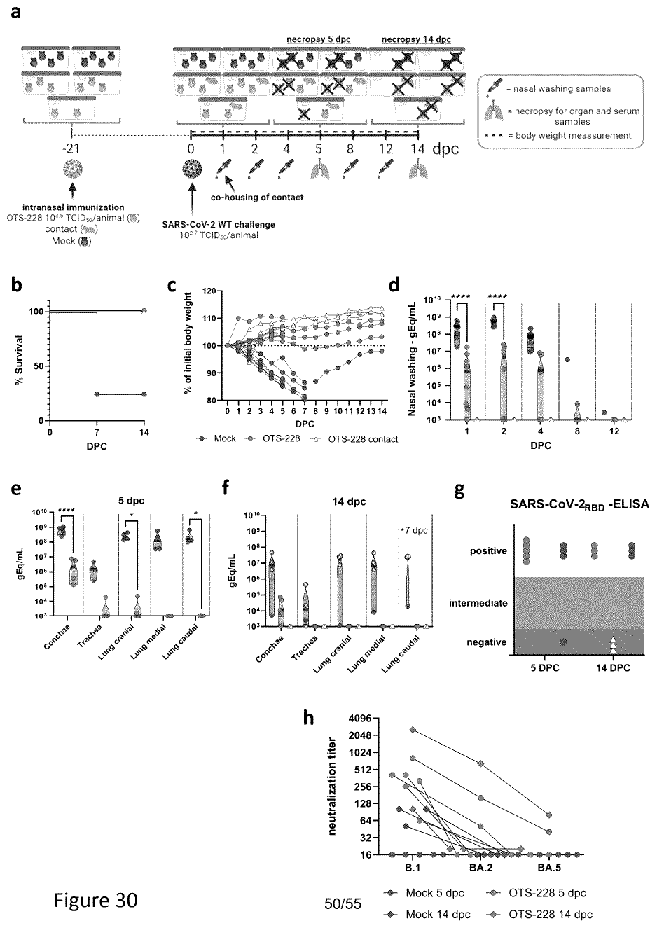

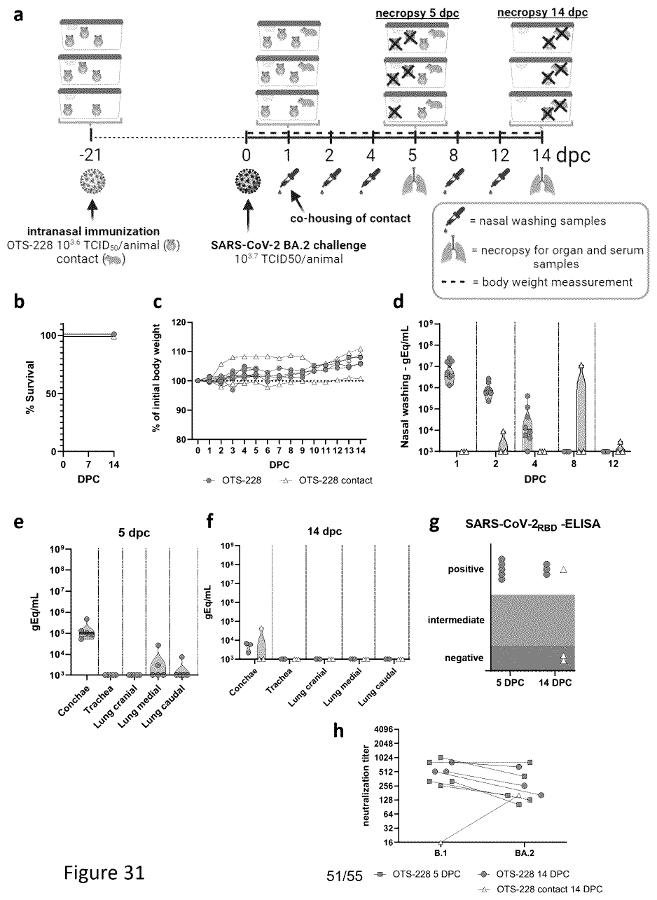

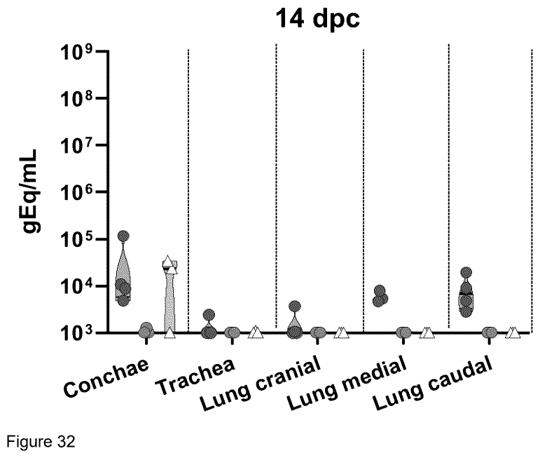

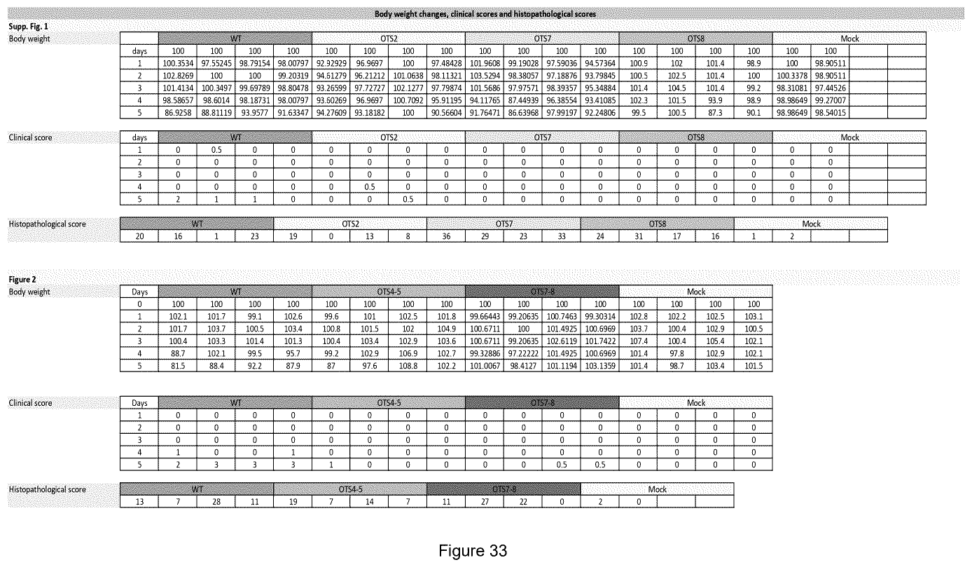

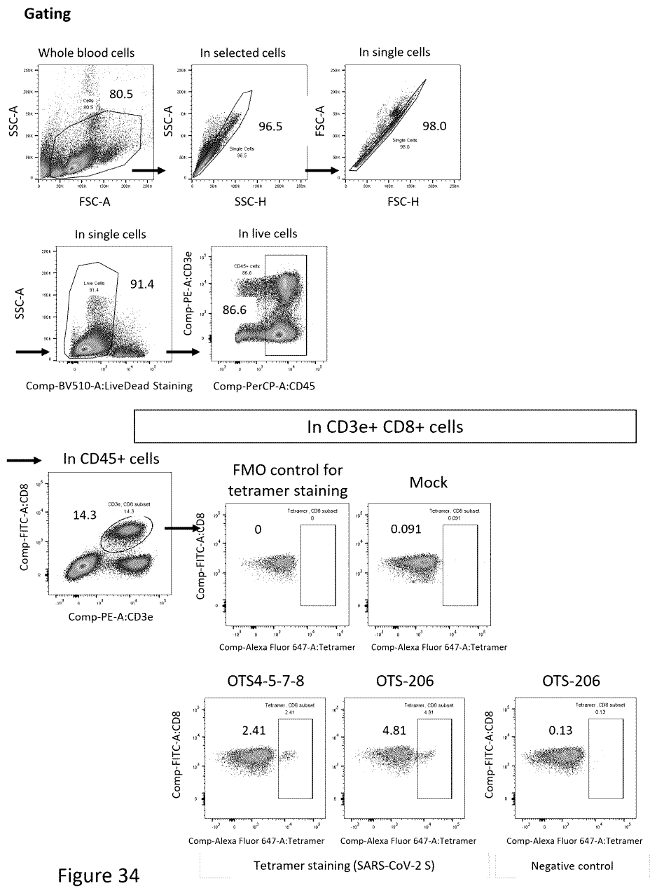

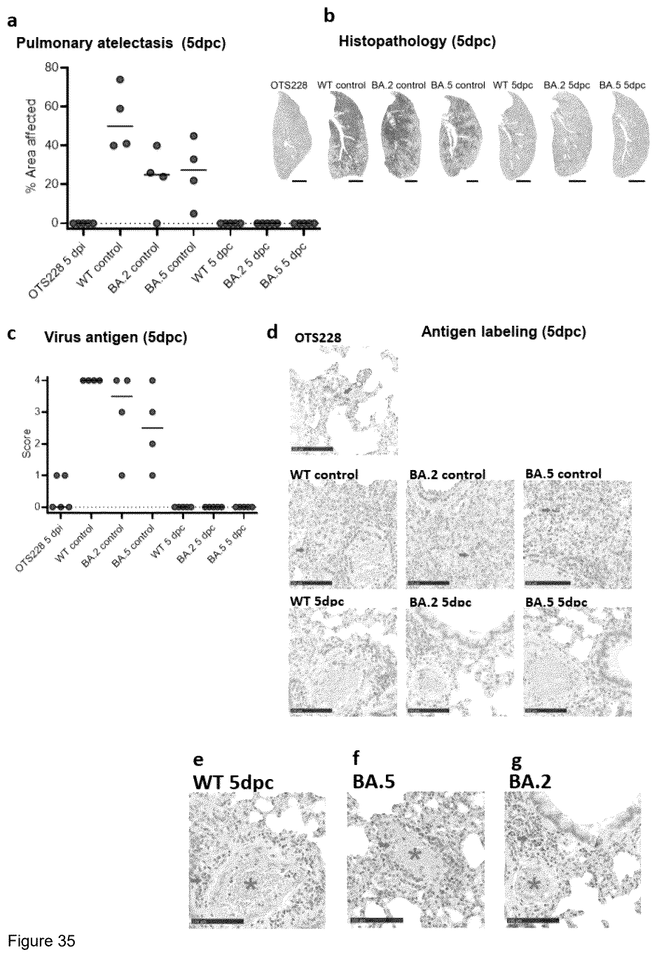

OTS2, OTS7, OTS7-8 were assessed for attenuation (FIG. 5).

### Assessment of Attenuation and Protection:

K18-hACE2-mice were infected intranasally with 5000 PFU. Oropharyngeal swabs were taken daily. Organs were taken at days 2 and 5/6 post infection. Viral RNA was quantified by qRT-PCR and viral titers were determined by plaque assay (to determine PFUs). Clinical scores and body weight were determined daily.

Challenge: >21 days post infection mice were challenged with wt SARS-CoV-2 (5000 PFU) and monitored for additional 15 days. Body weight and clinical scores were detected daily. Viral RNA load, virus titers were determined at 5 and 14/15 days post challenge. Swabs were taken 3-4 times per week. Antibody titers and CD8 T-cell responses were determined at the indicated time points.

OTS4-5 and OTS7-8 were analyzed for attenuation and protection (FIG. 6, 7, 8).

TABLE 1

| | | SARS- | OTS | | OTS | | OTS | | OTS | | OTS | | OTS | | OTS | |
|---|---|---|---|---|---|---|---|---|---|---|---|---|---|---|---|---|
| | | CoV-2 | Fragment 2 | | Fragment 2/3 | | Fragment 4/5 | | Fragment 7 | | Fragment 8 | | Fragment 7/8 | | Fragment 4/5/7/8 | |
| Genome | | WT | OTS | Nucleotide | OTS | Nucleotide | OTS | Nucleotide | OTS | Nucleotide | OTS | Nucleotide | OTS | Nucleotide | OTS | Nucleotide |
| Annotation | Genome | WT | codons | changes | codons | changes | codons | changes | codons | changes | codons | changes | codons | changes | codons | changes |
| peptide | Changes | codons | 77 | 136 | 164 | 291 | 191 | 335 | 80 | 149 | 66 | 120 | 146 | 269 | 337 | 604 |
| NSP1 | Leu | CTT | CTT | 0 | TTA | 2 | CTT | 0 | CTT | 0 | CTT | 0 | CTT | 0 | CTT | 0 |
| | 46 | | | | | | | | | | | | | | | |
| | Leu | | | | | | | | | | | | | | | |
| NSP1 | Leu | CTT | CTT | 0 | TTA | 2 | CTT | 0 | CTT | 0 | CTT | 0 | CTT | 0 | CTT | 0 |
| | 64 | | | | | | | | | | | | | | | |
| | Leu | | | | | | | | | | | | | | | |
| NSP1 | Leu | CTG | TTG | 1 | TTG | 1 | CTG | 0 | CTG | 0 | CTG | 0 | CTG | 0 | CTG | 0 |
| | 88 | | | | | | | | | | | | | | | |
| | Leu | | | | | | | | | | | | | | | |
| NSP1 | Leu | CTC | TTG | 2 | TTG | 2 | CTC | 0 | CTC | 0 | CTC | 0 | CTC | 0 | CTC | 0 |
| | 92 | | | | | | | | | | | | | | | |
| | Leu | | | | | | | | | | | | | | | |
| NSP1 | Ser | AGT | TCA | 3 | TCA | 3 | AGT | 0 | AGT | 0 | AGT | 0 | AGT | 0 | AGT | 0 |
| | 100 | | | | | | | | | | | | | | | |
| | Ser | | | | | | | | | | | | | | | |
| NSP1 | Leu | CTT | TTA | 2 | TTA | 2 | CTT | 0 | CTT | 0 | CTT | 0 | CTT | 0 | CTT | 0 |
| | 104 | | | | | | | | | | | | | | | |
| | Leu | | | | | | | | | | | | | | | |
| NSP1 | Leu | CTT | TTA | 2 | TTA | 2 | CTT | 0 | CTT | 0 | CTT | 0 | CTT | 0 | CTT | 0 |
| | 107 | | | | | | | | | | | | | | | |
| | Leu | | | | | | | | | | | | | | | |
| NSP1 | Leu | CTT | TTA | 2 | TTA | 2 | TTC | 0 | TTC | 0 | TTC | 0 | TTC | 0 | TTC | 0 |
| | 122 | | | | | | | | | | | | | | | |
| | Leu | | | | | | | | | | | | | | | |
| NSP1 | Leu | CTT | TTA | 2 | TTA | 2 | TTC | 0 | TTC | 0 | TTC | 0 | TTC | 0 | TTC | 0 |
| | 123 | | | | | | | | | | | | | | | |
| | Leu | | | | | | | | | | | | | | | |
| NSP1 | Leu | CTA | TTA | 1 | TTA | 1 | CTA | 0 | CTA | 0 | CTA | 0 | CTA | 0 | CTA | 0 |
| | 140 | | | | | | | | | | | | | | | |
| | Leu | | | | | | | | | | | | | | | |
| NSP1 | Leu | CTT | TTA | 2 | TTA | 2 | CTT | 0 | CTT | 0 | CTT | 0 | CTT | 0 | CTT | 0 |
| | 149 | | | | | | | | | | | | | | | |
| | Leu | | | | | | | | | | | | | | | |
| NSP1 | Ser | AGC | TCG | 3 | TCG | 3 | AGC | 0 | AGC | 0 | AGC | 0 | AGC | 0 | AGC | 0 |
| | 166 | | | | | | | | | | | | | | | |
| | Ser | | | | | | | | | | | | | | | |
| NSP1 | Ser | AGT | TCA | 3 | TCA | 3 | AGT | 0 | AGT | 0 | AGT | 0 | AGT | 0 | AGT | 0 |
| | 167 | | | | | | | | | | | | | | | |
| | Ser | | | | | | | | | | | | | | | |
| NSP1 | Leu | CTC | TTG | 2 | TTG | 2 | CTC | 0 | CTC | 0 | CTC | 0 | CTC | 0 | CTC | 0 |
| | 173 | | | | | | | | | | | | | | | |
| | Leu | | | | | | | | | | | | | | | |
| NSP1 | Leu | CTT | TTA | 2 | TTA | 2 | CTT | 0 | CTT | 0 | CTT | 0 | CTT | 0 | CTT | 0 |
| | 177 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 198 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 204 | | | | | | | | | | | | | | | |
| | Leu | | | | | | | | | | | | | | | |
| NSP2 | Leu | CTA | TTA | 1 | TTA | 1 | CTA | 0 | CTA | 0 | CTA | 0 | CTA | 0 | CTA | 0 |
| | 205 | | | | | | | | | | | | | | | |
| | Leu | | | | | | | | | | | | | | | |
| NSP2 | Leu | CTG | TTG | 1 | TTG | 1 | CTG | 0 | CTG | 0 | CTG | 0 | CTG | 0 | CTG | 0 |
| | 219 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 245 | | | | | | | | | | | | | | | |
| | Ser | | | | | | | | | | | | | | | |
| NSP2 | Ser | AGC | TCG | 3 | TCG | 3 | AGC | 0 | AGC | 0 | AGC | 0 | AGC | 0 | AGC | 0 |
| | 248 | | | | | | | | | | | | | | | |
| | Ser | | | | | | | | | | | | | | | |
| NSP2 | Ser | TCC | TCG | 1 | TCG | 1 | TCC | 0 | TCC | 0 | TCC | 0 | TCC | 0 | TCC | 0 |
| | 279 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 293 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 302 | | | | | | | | | | | | | | | |
| | Ser | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 320 | | | | | | | | | | | | | | | |
| | Leu | | | | | | | | | | | | | | | |
| NSP2 | Ser | AGT | TCA | 3 | TCA | 3 | AGT | 0 | AGT | 0 | AGT | 0 | AGT | 0 | AGT | 0 |
| | 383 | | | | | | | | | | | | | | | |
| | Ser | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 391 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 397 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 412 | | | | | | | | | | | | | | | |
| | Ser | | | | | | | | | | | | | | | |
| NSP2 | Ser | AGC | TCG | 3 | TCG | 3 | AGC | 0 | AGC | 0 | AGC | 0 | AGC | 0 | AGC | 0 |
| | 428 | | | | | | | | | | | | | | | |
| | Ser | | | | | | | | | | | | | | | |
| NSP2 | Ser | TCC | TCG | 1 | TCG | 1 | TCC | 0 | TCC | 0 | TCC | 0 | TCC | 0 | TCC | 0 |
| | 443 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 446 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 450 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 451 | | | | | | | | | | | | | | | |
| | Leu | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 454 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 469 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 479 | | | | | | | | | | | | | | | |
| | Ser | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 481 | | | | | | | | | | | | | | | |
| | Ser | | | | | | | | | | | | | | | |
| NSP2 | Ser | TCC | TCG | 1 | TCG | 1 | TCC | 0 | TCC | 0 | TCC | 0 | TCC | 0 | TCC | 0 |
| | 483 | | | | | | | | | | | | | | | |
| | Ser | | | | | | | | | | | | | | | |
| NSP2 | Ser | AGT | TCA | 3 | TCA | 3 | AGT | 0 | AGT | 0 | AGT | 0 | AGT | 0 | AGT | 0 |
| | 485 | | | | | | | | | | | | | | | |
| | Ser | | | | | | | | | | | | | | | |
| NSP2 | Ser | TCC | TCG | 1 | TCG | 1 | TCC | 0 | TCC | 0 | TCC | 0 | TCC | 0 | TCC | 0 |
| | 505 | | | | | | | | | | | | | | | |
| | Ser | | | | | | | | | | | | | | | |
| NSP2 | Leu | CTG | TTG | 1 | TTG | 1 | CTG | 0 | CTG | 0 | CTG | 0 | CTG | 0 | CTG | 0 |
| | 530 | | | | | | | | | | | | | | | |
| | Leu | | | | | | | | | | | | | | | |
| NSP2 | Ser | AGT | TCA | 3 | TCA | 3 | AGT | 0 | AGT | 0 | AGT | 0 | AGT | 0 | AGT | 0 |
| | 531 | | | | | | | | | | | | | | | |
| | Ser | | | | | | | | | | | | | | | |
| NSP2 | Leu | CTT | TTG | 2 | TTG | 2 | CTT | 0 | CTT | 0 | CTT | 0 | CTT | 0 | CTT | 0 |
| | 533 | | | | | | | | | | | | | | | |
| | Leu | | | | | | | | | | | | | | | |
| NSP2 | Ser | TCC | TCG | 1 | TCG | 1 | TCC | 0 | TCC | 0 | TCC | 0 | TCC | 0 | TCC | 0 |
| | 549 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 552 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 558 | | | | | | | | | | | | | | | |
| | Ser | | | | | | | | | | | | | | | |
| NSP2 | Leu | CTA | TTA | 1 | TTA | 1 | CTA | 0 | CTA | 0 | CTA | 0 | CTA | 0 | CTA | 0 |
| | 570 | | | | | | | | | | | | | | | |
| | Leu | | | | | | | | | | | | | | | |
| NSP2 | Leu | CTG | TTG | 1 | TTG | 1 | CTG | 0 | CTG | 0 | CTG | 0 | CTG | 0 | CTG | 0 |
| | 578 | | | | | | | | | | | | | | | |
| | Leu | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 580 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 588 | | | | | | | | | | | | | | | |
| | Ser | | | | | | | | | | | | | | | |
| NSP2 | Leu | CTA | TTA | 1 | TTA | 1 | CTA | 0 | CTA | 0 | CTA | 0 | CTA | 0 | CTA | 0 |
| | 595 | | | | | | | | | | | | | | | |
| | Leu | | | | | | | | | | | | | | | |
| NSP2 | Leu | CTA | TTA | 1 | TTA | 1 | CTA | 0 | CTA | 0 | CTA | 0 | CTA | 0 | CTA | 0 |
| | 613 | | | | | | | | | | | | | | | |
| | Leu | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 624 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 628 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 631 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 642 | | | | | | | | | | | | | | | |
| | Leu | | | | | | | | | | | | | | | |
| NSP2 | Ser | AGT | TCA | 3 | TCA | 3 | AGT | 0 | AGT | 0 | AGT | 0 | AGT | 0 | AGT | 0 |
| | 674 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 681 | | | | | | | | | | | | | | | |
| | Leu | | | | | | | | | | | | | | | |
| NSP2 | Ser | TCT | TCA | 1 | TCA | 1 | TCT | 0 | TCT | 0 | TCT | 0 | TCT | 0 | TCT | 0 |
| | 692 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 700 | | | | | | | | | | | | | | | |
| | Leu | | | | | | | | | | | | | | | |
| NSP2 | Ser | TCC | TCG | 1 | TCG | 1 | TCC | 0 | TCC | 0 | TCC | 0 | TCC | 0 | TCC | 0 |
| | 723 | | | | | | | | | | | | | | | |
| | Ser | | | | | | | | | | | | | | | |
| NSP2 | Leu | CTA | TTA | 1 | TTA | 1 | CTA | 0 | CTA | 0 | CTA | 0 | CTA | 0 | CTA | 0 |
| | 729 | | | | | | | | | | | | | | | |
| | Leu | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 730 | | | | | | | | | | | | | | | |
| | Leu | | | | | | | | | | | | | | | |
| NSP2 | Leu | CTA | TTA | 1 | TTA | 1 | CTA | 0 | CTA | 0 | CTA | 0 | CTA | 0 | CTA | 0 |
| | 733 | | | | | | | | | | | | | | | |
| | Leu | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 747 | | | | | | | | | | | | | | | |
| | Leu | | | | | | | | | | | | | | | |
| NSP2 | Ser | AGT | TCA | 3 | TCA | 3 | AGT | 0 | AGT | 0 | AGT | 0 | AGT | 0 | AGT | 0 |
| | 771 | | | | | | | | | | | | | | | |
| | Ser | | | | | | | | | | | | | | | |
| NSP2 | Leu | CTT | TTA | 2 | TTA | 2 | CTT | 0 | CTT | 0 | CTT | 0 | CTT | 0 | CTT | 0 |
| | 788 | | | | | | | | | | | | | | | |
| | Leu | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 791 | | | | | | | | | | | | | | | |
| | Leu | | | | | | | | | | | | | | | |
| NSP2 | Leu | CTC | TTG | 2 | TTG | 2 | CTC | 0 | CTC | 0 | CTC | 0 | CTC | 0 | CTC | 0 |
| | 815 | | | | | | | | | | | | | | | |
| | Leu | | | | | | | | | | | | | | | |
| NSP3 | Ser | AGT | TCA | 3 | TCA | 3 | AGT | 0 | AGT | 0 | AGT | 0 | AGT | 0 | AGT | 0 |
| | 838 | | | | | | | | | | | | | | | |
| | Ser | | | | | | | | | | | | | | | |
| NSP3 | Leu | CTT | TTA | 2 | TTA | 2 | CTT | 0 | CTT | 0 | CTT | 0 | CTT | 0 | CTT | 0 |
| | 845 | | | | | | | | | | | | | | | |
| | Leu | | | | | | | | | | | | | | | |
| NSP3 | Leu | CTT | TTA | 2 | TTA | 2 | CTT | 0 | CTT | 0 | CTT | 0 | CTT | 0 | CTT | 0 |
| | 853 | | | | | | | | | | | | | | | |
| | Leu | | | | | | | | | | | | | | | |
| NSP3 | Ser | TCT | TCA | 1 | TCA | 1 | TCT | 0 | TCT | 0 | TCT | 0 | TCT | 0 | TCT | 0 |
| | 858 | | | | | | | | | | | | | | | |
| | Ser | | | | | | | | | | | | | | | |
| NSP3 | Leu | CTC | TTG | 2 | TTG | 2 | CTC | 0 | CTC | 0 | CTC | 0 | CTC | 0 | CTC | 0 |
| | 864 | | | | | | | | | | | | | | | |
| | Leu | | | | | | | | | | | | | | | |
| NSP3 | Ser | TCT | TCA | 1 | TCA | 1 | TCT | 0 | TCT | 0 | TCT | 0 | TCT | 0 | TCT | 0 |
| | 887 | | | | | | | | | | | | | | | |
| | Ser | | | | | | | | | | | | | | | |
| NSP3 | Leu | CTG | TTG | 1 | TTG | 1 | CTG | 0 | CTG | 0 | CTG | 0 | CTG | 0 | CTG | 0 |
| | 893 | | | | | | | | | | | | | | | |
| | Leu | | | | | | | | | | | | | | | |
| NSP3 | Ser | AGT | TCA | 3 | TCA | 3 | AGT | 0 | AGT | 0 | AGT | 0 | AGT | 0 | AGT | 0 |
| | 901 | | | | | | | | | | | | | | | |
| | Ser | | | | | | | | | | | | | | | |
| NSP3 | Ser | TCT | TCA | 1 | TCA | 1 | TCT | 0 | TCT | 0 | TCT | 0 | TCT | 0 | TCT | 0 |
| | 911 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 923 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 966 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 969 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 984 | | | | | | | | | | | | | | | |
| | Ser | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 994 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1016 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1034 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1065 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1087 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1097 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1102 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1110 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1115 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1130 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1131 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1133 | | | | | | | | | | | | | | | |
| | Ser | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1144 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1145 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1161 | | | | | | | | | | | | | | | |
| | Ser | | | | | | | | | | | | | | | |
| NSP3 | Leu | CTC | CTC | 0 | TTG | 2 | CTC | 0 | CTC | 0 | CTC | 0 | CTC | 0 | CTC | 0 |
| | 1182 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1186 | | | | | | | | | | | | | | | |
| | Leu | | | | | | | | | | | | | | | |
| NSP3 | Ser | AGC | AGC | 0 | TCG | 3 | AGC | 0 | AGC | 0 | AGC | 0 | AGC | 0 | AGC | 0 |
| | 1189 | | | | | | | | | | | | | | | |
| | Ser | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1195 | | | | | | | | | | | | | | | |
| | Ser | | | | | | | | | | | | | | | |
| NSP3 | Leu | CTG | CTG | 0 | TTG | 1 | CTG | 0 | CTG | 0 | CTG | 0 | CTG | 0 | CTG | 0 |
| | 1243 | | | | | | | | | | | | | | | |
| | Leu | | | | | | | | | | | | | | | |
| NSP3 | Leu | CTC | CTC | 0 | TTG | 2 | CTC | 0 | CTC | 0 | CTC | 0 | CTC | 0 | CTC | 0 |
| | 1249 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1255 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1263 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1270 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1272 | | | | | | | | | | | | | | | |
| | Ser | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1313 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1346 | | | | | | | | | | | | | | | |
| | Leu | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1356 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1358 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1361 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1268 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1372 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1379 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1427 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1434 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1438 | | | | | | | | | | | | | | | |
| | Leu | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1441 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1445 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1450 | | | | | | | | | | | | | | | |
| | Leu | | | | | | | | | | | | | | | |
| NSP3 | Leu | CTC | CTC | 0 | TTG | 2 | CTC | 0 | CTC | 0 | CTC | 0 | CTC | 0 | CTC | 0 |
| | 1469 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1476 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1478 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1490 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1492 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1493 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1494 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1507 | | | | | | | | | | | | | | | |
| | Leu | | | | | | | | | | | | | | | |
| NSP3 | Ser | TCC | TCC | 0 | TCG | 1 | TCC | 0 | TCC | 0 | TCC | 0 | TCC | 0 | TCC | 0 |
| | 1510 | | | | | | | | | | | | | | | |
| | Ser | | | | | | | | | | | | | | | |
| NSP3 | Ser | TCC | TCC | 0 | TCG | 1 | TCC | 0 | TCC | 0 | TCC | 0 | TCC | 0 | TCC | 0 |
| | 1515 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1517 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1520 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1528 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1534 | | | | | | | | | | | | | | | |
| | Ser | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1539 | | | | | | | | | | | | | | | |
| | Ser | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1546 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1556 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1559 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1560 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1561 | | | | | | | | | | | | | | | |
| | Ser | | | | | | | | | | | | | | | |
| NSP3 | Leu | TTG | TTG | 0 | CTC | 2 | TTG | 0 | TTG | 0 | TTG | 0 | TTG | 0 | TTG | 0 |
| | 1579 | | | | | | | | | | | | | | | |
| | Leu | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1627 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1641 | | | | | | | | | | | | | | | |
| | Ser | | | | | | | | | | | | | | | |
| NSP3 | Leu | CTG | CTG | 0 | TTG | 1 | CTG | 0 | CTG | 0 | CTG | 0 | CTG | 0 | CTG | |
| | 1643 | | | | | | | | | | | | | | | |
| | Leu | | | | | | | | | | | | | | | 0 |
| NSP3 | Ser | TCT | TCT | 0 | TCA | 1 | TCT | 0 | TCT | 0 | TCT | 0 | TCT | 0 | TCT | 0 |
| | 1666 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1676 | | | | | | | | | | | | | | | |
| | Leu | | | | | | | | | | | | | | | |
| NSP3 | Leu | CTC | CTC | 0 | TTG | 2 | CTC | 0 | CTC | 0 | CTC | 0 | CTC | 0 | CTC | 0 |
| | 1683 | | | | | | | | | | | | | | | |
| | Leu | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1695 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1713 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1733 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1743 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1762 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1774 | | | | | | | | | | | | | | | |
| | Leu | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1775 | | | | | | | | | | | | | | | |
| | Ser | | | | | | | | | | | | | | | |
| NSP3 | Leu | CTA | CTA | 0 | TTA | 1 | CTA | 0 | CTA | 0 | CTA | 0 | CTA | 0 | CTA | 0 |
| | 1797 | | | | | | | | | | | | | | | |
| | Leu | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1816 | | | | | | | | | | | | | | | |
| | Leu | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1825 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1841 | | | | | | | | | | | | | | | |
| | Ser | | | | | | | | | | | | | | | |
| NSP3 | Leu | CTT | CTT | 0 | TTA | 2 | CTT | 0 | CTT | 0 | CTT | 0 | CTT | 0 | CTT | 0 |
| | 1853 | | | | | | | | | | | | | | | |
| | Leu | | | | | | | | | | | | | | | |
| NSP3 | Ser | TCC | TCC | 0 | TCG | 1 | TCC | 0 | TCC | 0 | TCC | 0 | TCC | 0 | TCC | 0 |
| | 2856 | | | | | | | | | | | | | | | |
| | Ser | | | | | | | | | | | | | | | |
| NSP3 | Ser | AGT | AGT | 0 | TCA | 3 | AGT | 0 | AGT | 0 | AGT | 0 | AGT | 0 | AGT | 0 |
| | 1872 | | | | | | | | | | | | | | | |
| | Ser | | | | | | | | | | | | | | | |
| NSP3 | Ser | TCT | TCT | 0 | TCA | 1 | TCT | 0 | TCT | 0 | TCT | 0 | TCT | 0 | TCT | 0 |
| | 1905 | | | | | | | | | | | | | | | |
| | Ser | | | | | | | | | | | | | | | |
| NSP3 | Leu | CTG | CTG | 0 | CTG | 0 | TTG | 1 | CTG | 0 | CTG | 0 | CTG | 0 | TTG | 1 |
| | 2028 | | | | | | | | | | | | | | | |
| | Leu | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2039 | | | | | | | | | | | | | | | |
| | Leu | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2044 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2048 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2062 | | | | | | | | | | | | | | | |
| | Leu | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2077 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2083 | | | | | | | | | | | | | | | |
| | Ser | | | | | | | | | | | | | | | |
| NSP3 | Leu | TTA | TTA | 0 | TTA | 0 | CTA | 1 | TTA | 0 | TTA | 0 | TTA | 0 | CTA | 1 |
| | 2095 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2103 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2104 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2114 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2122 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2132 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2146 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2151 | | | | | | | | | | | | | | | |
| | Ser | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2177 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2185 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2188 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2193 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2205 | | | | | | | | | | | | | | | |
| | Ser | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2211 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2224 | | | | | | | | | | | | | | | |
| | Ser | | | | | | | | | | | | | | | |
| NSP3 | Leu | CTG | CTG | 0 | CTG | 0 | TTG | 1 | CTG | 0 | CTG | 0 | CTG | 0 | TTG | 1 |
| | 2226 | | | | | | | | | | | | | | | |
| | Leu | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2235 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2237 | | | | | | | | | | | | | | | |
| | Ser | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2240 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2242 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2255 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2261 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2273 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2285 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2289 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2292 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2293 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2297 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 3303 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2313 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2333 | | | | | | | | | | | | | | | |
| | Leu | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2341 | | | | | | | | | | | | | | | |
| | Leu | | | | | | | | | | | | | | | |
| NSP3 | Ser | AGC | AGC | 0 | AGC | 0 | TCG | 3 | AGC | 0 | AGC | 0 | AGC | 0 | TCG | 3 |
| | 2352 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | |
| | 2360 | | | | | | | | | | | | | | | 3 |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2362 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2364 | | | | | | | | | | | | | | | |
| | Leu | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2371 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2396 | | | | | | | | | | | | | | | |
| | Ser | | | | | | | | | | | | | | | |
| NSP3 | Ser | TCC | TCC | 0 | TCC | 0 | TCG | 1 | TCC | 0 | TCC | 0 | TCC | 0 | TCG | 1 |
| | 2433 | | | | | | | | | | | | | | | |
| | Ser | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2447 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2462 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2466 | | | | | | | | | | | | | | | |
| | Ser | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2475 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2487 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2488 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2493 | | | | | | | | | | | | | | | |
| | Ser | | | | | | | | | | | | | | | |
| NSP3 | Ser | TCC | TCC | 0 | TCC | 0 | TCG | 1 | TCC | 0 | TCC | 0 | TCC | 0 | TCG | 1 |
| | 2500 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2503 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2517 | | | | | | | | | | | | | | | |
| | Ser | | | | | | | | | | | | | | | |
| NSP3 | Leu | CTC | CTC | 0 | CTC | 0 | TTG | 2 | CTC | 0 | CTC | 0 | CTC | 0 | TTG | 2 |
| | 2518 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2519 | | | | | | | | | | | | | | | |
| | Ser | | | | | | | | | | | | | | | |
| NSP3 | Leu | CTG | CTG | 0 | CTG | 0 | TTG | 1 | CTG | 0 | CTG | 0 | CTG | 0 | TTG | 1 |
| | 2527 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2553 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2558 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2564 | | | | | | | | | | | | | | | |
| | Leu | | | | | | | | | | | | | | | |
| NSP3 | Leu | CTG | CTG | 0 | CTG | 0 | TTG | 1 | CTG | 0 | CTG | 0 | CTG | 0 | TTG | 1 |
| | 2570 | | | | | | | | | | | | | | | |
| | Leu | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2572 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2578 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2583 | | | | | | | | | | | | | | | |
| | Ser | | | | | | | | | | | | | | | |
| NSP3 | Leu | CTC | CTC | 0 | CTC | 0 | TTG | 2 | CTC | 0 | CTC | 0 | CTC | 0 | TTG | 2 |
| | 2609 | | | | | | | | | | | | | | | |
| | Leu | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2612 | | | | | | | | | | | | | | | |
| | Leu | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2620 | | | | | | | | | | | | | | | |
| | Leu | | | | | | | | | | | | | | | |
| NSP3 | Ser | TCC | TCC | 0 | TCC | 0 | TCG | 1 | TCC | 0 | TCC | 0 | TCC | 0 | TCG | 1 |
| | 2625 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2631 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2655 | | | | | | | | | | | | | | | |
| | Leu | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2661 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2669 | | | | | | | | | | | | | | | |
| | Ser | | | | | | | | | | | | | | | |
| NSP3 | Leu | CTC | CTC | 0 | CTC | 0 | TTG | 2 | CTC | 0 | CTC | 0 | CTC | 0 | TTG | |
| | 2675 | | | | | | | | | | | | | | | 2 |
| | Leu | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2688 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2695 | | | | | | | | | | | | | | | |
| | Ser | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2706 | | | | | | | | | | | | | | | |
| | Ser | | | | | | | | | | | | | | | |
| NSP3 | Ser | TCT | TCT | 0 | TCT | 0 | TCA | 1 | TCT | 0 | TCT | 0 | TCT | 0 | TCA | 1 |
| | 2722 | | | | | | | | | | | | | | | |
| | Ser | | | | | | | | | | | | | | | |
| NSP3 | Leu | CTA | CTA | 0 | CTA | 0 | TTA | 1 | CTA | 0 | CTA | 0 | CTA | 0 | TTA | 1 |
| | 2725 | | | | | | | | | | | | | | | |
| | Leu | | | | | | | | | | | | | | | |
| NSP3 | Ser | AGT | AGT | 0 | AGT | 0 | TCA | 3 | AGT | 0 | AGT | 0 | AGT | 0 | TCA | 3 |
| | 2731 | | | | | | | | | | | | | | | |
| | Ser | | | | | | | | | | | | | | | |
| NSP3 | Leu | CTT | CTT | 0 | CTT | 0 | TTA | 2 | CTT | 0 | CTT | 0 | CTT | 0 | TTA | 2 |
| | 2760 | | | | | | | | | | | | | | | |
| | Leu | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 2778 | | | | | | | | | | | | | | | |
| | Leu | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 2781 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2797 | | | | | | | | | | | | | | | |
| | Ser | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 2804 | | | | | | | | | | | | | | | |
| | Ser | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2822 | | | | | | | | | | | | | | | |
| | Ser | | | | | | | | | | | | | | | |
| NSP4 | Ser | AGC | AGC | 0 | AGC | 0 | TCG | 3 | AGC | 0 | AGC | 0 | AGC | 0 | TCG | 3 |
| | 2839 | | | | | | | | | | | | | | | |
| | Ser | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 2844 | | | | | | | | | | | | | | | |
| | Ser | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 2890 | | | | | | | | | | | | | | | |
| | Ser | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 2902 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2926 | | | | | | | | | | | | | | | |
| | Ser | | | | | | | | | | | | | | | |
| NSP4 | Leu | CTA | CTA | 0 | CTA | 0 | TTA | 1 | CTA | 0 | CTA | 0 | CTA | 0 | TTA | 1 |
| | 2939 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2942 | | | | | | | | | | | | | | | |
| | Ser | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 2947 | | | | | | | | | | | | | | | |
| | Ser | | | | | | | | | | | | | | | |
| NSP4 | Leu | CTC | CTC | 0 | CTC | 0 | TTG | 2 | CTC | 0 | CTC | 0 | CTC | 0 | TTG | 2 |
| | 2956 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2960 | | | | | | | | | | | | | | | |
| | Ser | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 2969 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2972 | | | | | | | | | | | | | | | |
| | Ser | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2981 | | | | | | | | | | | | | | | |
| | Ser | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 2999 | | | | | | | | | | | | | | | |
| | Ser | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 3001 | | | | | | | | | | | | | | | |
| | Ser | | | | | | | | | | | | | | | |
| NSP4 | Leu | CTT | CTT | 0 | CTT | 0 | TTA | | CTT | 0 | CTT | 0 | CTT | 0 | TTA | 2 |
| | 3006 | | | | | | | 2 | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 3013 | | | | | | | | | | | | | | | |
| | Ser | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3027 | | | | | | | | | | | | | | | |
| | Leu | | | | | | | | | | | | | | | |
| NSP4 | Leu | CTA | CTA | 0 | CTA | 0 | TTA | 1 | CTA | 0 | CTA | 0 | CTA | 0 | TTA | 1 |
| | 3034 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 3046 | | | | | | | | | | | | | | | |
| | Ser | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3060 | | | | | | | | | | | | | | | |
| | Leu | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 3075 | | | | | | | | | | | | | | | |
| | Ser | | | | | | | | | | | | | | | |
| NSP4 | Leu | CTA | CTA | 0 | CTA | 0 | TTA | 1 | CTA | 0 | CTA | 0 | CTA | 0 | TTA | 1 |
| | 3084 | | | | | | | | | | | | | | | |
| | Leu | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3086 | | | | | | | | | | | | | | | |
| | Leu | | | | | | | | | | | | | | | |
| NSP4 | Leu | CTC | CTC | 0 | CTC | 0 | TGG | 2 | CTC | 0 | CTC | 0 | CTC | 0 | TGG | 2 |
| | 3092 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 3106 | | | | | | | | | | | | | | | |
| | Ser | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3116 | | | | | | | | | | | | | | | |
| | Leu | | | | | | | | | | | | | | | |
| NSP4 | Ser | TCT | TCT | 0 | TCT | 0 | TCA | 1 | TCT | | TCT | 0 | TCT | 0 | TCA | 1 |
| | 3121 | | | | | | | | | 0 | | | | | | |
| | Ser | | | | | | | | | | | | | | | |
| NSP4 | Ser | TCC | TCC | 0 | TCC | 0 | TCG | 1 | TCC | 0 | TCC | 0 | TCC | 0 | TCG | 1 |
| | 3149 | | | | | | | | | | | | | | | |
| | Ser | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 3158 | | | | | | | | | | | | | | | |
| | Ser | | | | | | | | | | | | | | | |
| NSP4 | Leu | CTA | CTA | 0 | CTA | 0 | TTA | 1 | CTA | 0 | CTA | 0 | CTA | 0 | TTA | 1 |
| | 3161 | | | | | | | | | | | | | | | |
| | Leu | | | | | | | | | | | | | | | |
| NSP4 | Ser | TCC | TCC | 0 | TCC | 0 | TCG | 1 | TCC | 0 | TCC | 0 | TCC | 0 | TCG | 1 |
| | 3171 | | | | | | | | | | | | | | | |
| | Ser | | | | | | | | | | | | | | | |
| NSP4 | Ser | AGT | AGT | 0 | AGT | 0 | TCA | 2 | AGT | 0 | AGT | 0 | AGT | 0 | TCA | 2 |
| | 3173 | | | | | | | | | | | | | | | |
| | Ser | | | | | | | | | | | | | | | |
| NSP4 | Leu | CTG | CTG | 0 | CTG | 0 | TTG | 1 | CTG | 0 | CTG | 0 | CTG | 0 | TTG | 1 |
| | 3180 | | | | | | | | | | | | | | | |
| | Leu | | | | | | | | | | | | | | | |
| NSP4 | Leu | CTA | CTA | 0 | CTA | 0 | TTA | 1 | CTA | 0 | CTA | 0 | CTA | 0 | TTA | 1 |
| | 3191 | | | | | | | | | | | | | | | |
| | LEu | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 3195 | | | | | | | | | | | | | | | |
| | Ser | | | | | | | | | | | | | | | |
| NSP4 | Leu | CTA | CTA | 0 | CTA | 0 | TTA | 1 | CTA | 0 | CTA | 0 | CTA | 0 | TTA | 1 |
| | 3198 | | | | | | | | | | | | | | | |
| | Leu | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3201 | | | | | | | | | | | | | | | |
| | Leu | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3210 | | | | | | | | | | | | | | | |
| | Leu | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 3218 | | | | | | | | | | | | | | | |
| | Ser | | | | | | | | | | | | | | | |
| NSP4 | Ser | AGC | AGC | 0 | AGC | 0 | TCG | 3 | AGC | 0 | AGC | 0 | AGC | 0 | TCG | 3 |
| | 3225 | | | | | | | | | | | | | | | |
| | Ser | | | | | | | | | | | | | | | |
| NSP4 | Leu | CTC | CTC | 0 | CTC | 0 | TTG | 2 | CTC | 0 | CTC | 0 | CTC | 0 | TTG | 2 |
| | 3234 | | | | | | | | | | | | | | | |
| | Leu | | | | | | | | | | | | | | | |
| NSP4 | Leu | CTC | CTC | 0 | CTC | 0 | TTG | 2 | CTC | 0 | CTC | 0 | CTC | 0 | TTG | 2 |
| | 3238 | | | | | | | | | | | | | | | |
| | Leu | | | | | | | | | | | | | | | |
| NSP4 | Ser | AGT | AGT | 0 | AGT | 0 | TCA | 3 | AGT | 0 | AGT | 0 | AGT | 0 | TCA | 3 |
| | 3242 | | | | | | | | | | | | | | | |
| | Ser | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 3246 | | | | | | | | | | | | | | | |
| | Ser | | | | | | | | | | | | | | | |
| NSP4 | Leu | CTT | CTT | 0 | CTT | 0 | TTA | 2 | CTT | 0 | CTT | 0 | CTT | 0 | TTA | 2 |
| | 3249 | | | | | | | | | | | | | | | |
| | Leu | | | | | | | | | | | | | | | |
| NSP4 | Ser | TCT | TCT | 0 | TCT | 0 | TCA | 1 | TCT | 0 | TCT | 0 | TCT | 0 | TCA | 1 |
| | 3256 | | | | | | | | | | | | | | | |
| | Ser | | | | | | | | | | | | | | | |
| NSP5 | Ser | AGT | AGT | 0 | AGT | 0 | TCA | 3 | AGT | 0 | AGT | 0 | AGT | 0 | TCA | 3 |
| | 3264 | | | | | | | | | | | | | | | |
| | Ser | | | | | | | | | | | | | | | |
| NSP5 | Ser | TCT | TCT | 0 | TCT | 0 | TCA | 1 | TCT | 0 | TCT | 0 | TCT | 0 | TCA | 1 |
| | 3273 | | | | | | | | | | | | | | | |
| | Ser | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3290 | | | | | | | | | | | | | | | |
| | Leu | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3293 | | | | | | | | | | | | | | | |
| | Leu | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3295 | | | | | | | | | | | | | | | |
| | Leu | | | | | | | | | | | | | | | |
| NSP5 | Ser | TCT | TCT | 0 | TCT | 0 | TCA | 1 | TCT | 0 | TCT | 0 | TCT | 0 | TCA | 1 |
| | 3309 | | | | | | | | | | | | | | | |
| | Ser | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3313 | | | | | | | | | | | | | | | |
| | Leu | | | | | | | | | | | | | | | |
| NSP5 | Leu | CTC | CTC | 0 | CTC | 0 | TTG | 2 | CTC | 0 | CTC | 0 | CTC | 0 | TTG | 2 |
| | 3321 | | | | | | | | | | | | | | | |
| | Leu | | | | | | | | | | | | | | | |
| NSP5 | Ser | TCT | TCT | 0 | TCT | 0 | TCA | 1 | TCT | 0 | TCT | 0 | TCT | 0 | TCA | 1 |
| | 3325 | | | | | | | | | | | | | | | |
| | Ser | | | | | | | | | | | | | | | |
| NSP5 | Leu | CTC | CTC | 0 | CTC | 0 | TTG | 2 | CTC | 0 | CTC | 0 | CTC | 0 | TTG | 2 |
| | 3338 | | | | | | | | | | | | | | | |
| | Leu | | | | | | | | | | | | | | | |
| NSP5 | Ser | TCT | TCT | 0 | TCT | 0 | TCA | 1 | TCT | 0 | TCT | 0 | TCT | 0 | TCA | 1 |
| | 3344 | | | | | | | | | | | | | | | |
| | Ser | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3350 | | | | | | | | | | | | | | | |
| | Leu | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3352 | | | | | | | | | | | | | | | |
| | Leu | | | | | | | | | | | | | | | |
| NSP5 | Ser | TCT | TCT | 0 | TCT | 0 | TCA | 1 | TCT | 0 | TCT | 0 | TCT | 0 | TCA | 1 |
| | 3386 | | | | | | | | | | | | | | | |
| | Ser | | | | | | | | | | | | | | | |
| NSP5 | Leu | TTA | TTA | 0 | TTA | 0 | CTT | 2 | TTA | 0 | TTA | 0 | TTA | 0 | CTT | 2 |
| | 3404 | | | | | | | | | | | | | | | |
| | Leu | | | | | | | | | | | | | | | |
| NSP5 | Ser | AGT | AGT | 0 | AGT | 0 | TCA | 3 | AGT | 0 | AGT | 0 | AGT | 0 | TCA | 3 |
| | 3410 | | | | | | | | | | | | | | | |
| | Ser | | | | | | | | | | | | | | | |
| NSP5 | Ser | TCT | TCT | 0 | TCT | 0 | TCA | 1 | TCT | 0 | TCT | 0 | TCT | 0 | TCA | 1 |
| | 3421 | | | | | | | | | | | | | | | |
| | Ser | | | | | | | | | | | | | | | |
| NSP5 | Leu | CTC | CTC | 0 | CTC | 0 | TTG | 2 | CTC | 0 | CTC | 0 | CTC | 0 | TTG | 2 |
| | 3483 | | | | | | | | | | | | | | | |
| | Leu | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3490 | | | | | | | | | | | | | | | |
| | Leu | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3495 | | | | | | | | | | | | | | | |
| | Leu | | | | | | | | | | | | | | | |
| NSP5 | Leu | CTA | CTA | 0 | CTA | 0 | TTA | 1 | CTA | 0 | CTA | 0 | CTA | 0 | TTA | 1 |
| | 3505 | | | | | | | | | | | | | | | |
| | Leu | | | | | | | | | | | | | | | |
| NSP5 | Leu | CTA | CTA | 0 | CTA | 0 | TTA | 1 | CTA | 0 | CTA | 0 | CTA | 0 | TTA | 1 |
| | 3513 | | | | | | | | | | | | | | | |
| | Leu | | | | | | | | | | | | | | | |
| NSP5 | Leu | CTT | CTT | 0 | CTT | 0 | TTA | 2 | CTT | 0 | CTT | 0 | CTT | 0 | TTA | 2 |
| | 3516 | | | | | | | | | | | | | | | |
| | Leu | | | | | | | | | | | | | | | |
| NSP5 | Leu | CTG | CTG | 0 | CTG | 0 | TTG | 1 | CTG | 0 | CTG | 0 | CTG | 0 | TTG | 1 |
| | 3535 | | | | | | | | | | | | | | | |
| | Leu | | | | | | | | | | | | | | | |
| NSP5 | Ser | AGT | AGT | 0 | AGT | 0 | TCA | 3 | AGT | 0 | AGT | 0 | AGT | 0 | TCA | 3 |
| | 3547 | | | | | | | | | | | | | | | |
| | Ser | | | | | | | | | | | | | | | |
| NSP6 | Ser | AGT | AGT | 0 | AGT | 0 | TCA | 3 | AGT | 0 | AGT | 0 | AGT | 0 | TCA | 3 |
| | 3570 | | | | | | | | | | | | | | | |
| | Ser | | | | | | | | | | | | | | | |
| NSP6 | Leu | CTC | CTC | 0 | CTC | 0 | TTG | 2 | CTC | 0 | CTC | 0 | CTC | 0 | TTG | 2 |
| | 3585 | | | | | | | | | | | | | | | |
| | Leu | | | | | | | | | | | | | | | |
| NSP6 | Leu | CTT | CTT | 0 | CTT | 0 | TTA | 2 | CTT | 0 | CTT | 0 | CTT | 0 | TTA | 2 |
| | 3581 | | | | | | | | | | | | | | | |
| | Leu | | | | | | | | | | | | | | | |
| NSP6 | Ser | AGT | AGT | 0 | AGT | 0 | TCA | 3 | AGT | 0 | AGT | 0 | AGT | 0 | TCA | 3 |
| | 3597 | | | | | | | | | | | | | | | |
| | Ser | | | | | | | | | | | | | | | |
| NSP6 | Ser | TCT | TCT | 0 | TCT | 0 | TCA | 1 | TCT | | TCT | 0 | TCT | 0 | TCA | 1 |
| | 3601 | | | | | | | | | 0 | | | | | | |
| | Ser | | | | | | | | | | | | | | | |
| NSP6 | Ser | TCT | TCT | 0 | TCT | 0 | TCA | 1 | TCT | 0 | TCT | 0 | TCT | 0 | TCA | 1 |
| | 3622 | | | | | | | | | | | | | | | |
| | Ser | | | | | | | | | | | | | | | |
| NSP6 | Leu | CTC | CTC | 0 | CTC | 0 | TTG | 2 | CTC | 0 | CTC | 0 | CTC | 0 | TTG | 2 |
| | 3636 | | | | | | | | | | | | | | | |
| | Leu | | | | | | | | | | | | | | | |
| NSP6 | Ser | TCT | TCT | 0 | TCT | 0 | TCA | 1 | TCT | 0 | TCT | 0 | TCT | 0 | TCA | 1 |
| | 3643 | | | | | | | | | | | | | | | |
| | Ser | | | | | | | | | | | | | | | |
| NSP6 | Leu | CTT | CTT | 0 | CTT | 0 | TTA | 2 | CTT | 0 | CTT | 0 | CTT | 0 | TTA | 2 |
| | 3644 | | | | | | | | | | | | | | | |
| | Leu | | | | | | | | | | | | | | | |
| NSP6 | Ser | AGT | AGT | 0 | AGT | 0 | TCA | 3 | AGT | 0 | AGT | 0 | AGT | 0 | TCA | 3 |
| | 3658 | | | | | | | | | | | | | | | |
| | Ser | | | | | | | | | | | | | | | |
| NSP6 | Ser | AGT | AGT | 0 | AGT | 0 | TCA | 3 | AGT | 0 | AGT | 0 | AGT | 0 | TCA | 3 |
| | 3673 | | | | | | | | | | | | | | | |
| | Ser | | | | | | | | | | | | | | | |
| NSP6 | Ser | TCT | TCT | 0 | TCT | 0 | TCA | 1 | TCT | 0 | TCT | 0 | TCT | 0 | TCA | 1 |
| | 3675 | | | | | | | | | | | | | | | |
| | Ser | | | | | | | | | | | | | | | |
| NSP6 | Leu | CTA | CTA | 0 | CTA | 0 | TTA | 1 | CTA | 0 | CTA | 0 | CTA | 0 | TTA | 1 |
| | 3679 | | | | | | | | | | | | | | | |
| | Leu | | | | | | | | | | | | | | | |
| NSP6 | Leu | CTA | CTA | 0 | CTA | 0 | TTA | 1 | CTA | 0 | CTA | 0 | CTA | 0 | TTA | 1 |
| | 3692 | | | | | | | | | | | | | | | |
| | Leu | | | | | | | | | | | | | | | |
| NSP6 | Leu | CTT | CTT | 0 | CTT | 0 | TTA | 2 | CTT | 0 | CTT | 0 | CTT | 0 | TTA | 2 |
| | 3694 | | | | | | | | | | | | | | | |
| | Leu | | | | | | | | | | | | | | | |
| NSP6 | Leu | CTT | CTT | 0 | CTT | 0 | TTA | 2 | CTT | 0 | CTT | 0 | CTT | 0 | TTA | 2 |
| | 3711 | | | | | | | | | | | | | | | |
| | Leu | | | | | | | | | | | | | | | |
| NSP6 | Leu | CTC | CTC | 0 | CTC | 0 | TTG | 2 | CTC | 0 | CTC | 0 | CTC | 0 | TTG | 2 |
| | 3717 | | | | | | | | | | | | | | | |
| | Leu | | | | | | | | | | | | | | | |
| NSP6 | Ser | TCC | TCC | 0 | TCC | 0 | TCG | 1 | TCC | 0 | TCC | 0 | TCC | 0 | TCG | 1 |
| | 3732 | | | | | | | | | | | | | | | |
| | Ser | | | | | | | | | | | | | | | |
| NSP6 | Leu | CTT | CTT | 0 | CTT | 0 | TTA | 2 | CTT | 0 | CTT | 0 | CTT | 0 | TTA | 2 |
| | 3736 | | | | | | | | | | | | | | | |
| | Leu | | | | | | | | | | | | | | | |
| NSP6 | Ser | TCT | TCT | 0 | TCT | 0 | TCA | 1 | TCT | 0 | TCT | 0 | TCT | 0 | TCA | 1 |
| | 3739 | | | | | | | | | | | | | | | |
| | Ser | | | | | | | | | | | | | | | |
| NSP6 | Ser | TCT | TCT | 0 | TCT | 0 | TCA | 1 | TCT | 0 | TCT | 0 | TCT | 0 | TCA | 1 |
| | 3742 | | | | | | | | | | | | | | | |
| | Ser | | | | | | | | | | | | | | | |
| NSP6 | Leu | CTT | CTT | 0 | CTT | 0 | TTA | 2 | CTT | 0 | CTT | 0 | CTT | 0 | TTA | 2 |
| | 3776 | | | | | | | | | | | | | | | |
| | Leu | | | | | | | | | | | | | | | |
| NSP6 | Leu | CTA | CTA | 0 | CTA | 0 | TTA | 2 | CTA | 0 | CTA | 0 | CTA | 0 | TTA | 2 |
| | 3781 | | | | | | | | | | | | | | | |
| | Leu | | | | | | | | | | | | | | | |
| NSP6 | Leu | CTC | CTC | 0 | CTC | 0 | TTG | 2 | CTC | 0 | CTC | 0 | CTC | 0 | TTG | 2 |
| | 3796 | | | | | | | | | | | | | | | |
| | Leu | | | | | | | | | | | | | | | |
| NSP6 | Leu | CTA | CTA | 0 | CTA | 0 | TTA | 1 | CTA | 0 | CTA | 0 | CTA | 0 | TTA | 1 |
| | 3828 | | | | | | | | | | | | | | | |
| | Leu | | | | | | | | | | | | | | | |
| NSP6 | Leu | CTC | CTC | 0 | CTC | 0 | TTG | 2 | CTC | 0 | CTC | 0 | CTC | 0 | TTG | 2 |
| | 3829 | | | | | | | | | | | | | | | |
| | Leu | | | | | | | | | | | | | | | |
| NSP6 | Ser | AGC | AGC | 0 | AGC | 0 | TCG | 3 | AGC | 0 | AGC | 0 | AGC | 0 | TCG | 3 |
| | 3834 | | | | | | | | | | | | | | | |
| | Ser | | | | | | | | | | | | | | | |
| NSP6 | Leu | CTC | CTC | 0 | CTC | 0 | TTG | 2 | CTC | 0 | CTC | 0 | CTC | 0 | TTG | |
| | 3840 | | | | | | | | | | | | | | | |
| | Leu | | | | | | | | | | | | | | | 2 |
| NSP7 | Ser | TCT | TCT | 0 | TCT | 0 | TCA | 1 | TCT | 0 | TCT | 0 | TCT | 0 | TCA | 1 |
| | 3860 | | | | | | | | | | | | | | | |
| | Ser | | | | | | | | | | | | | | | |
| NSP7 | Leu | CTC | CTC | 0 | CTC | 0 | TTG | 2 | CTC | 0 | CTC | 0 | CTC | 0 | TTG | 2 |
| | 3873 | | | | | | | | | | | | | | | |
| | Leu | | | | | | | | | | | | | | | |
| NSP7 | Leu | CTC | CTC | 0 | CTC | 0 | TTG | 2 | CTC | 0 | CTC | 0 | CTC | 0 | TTG | 2 |
| | 3879 | | | | | | | | | | | | | | | |
| | Leu | | | | | | | | | | | | | | | |
| NSP7 | Ser | TCT | TCT | 0 | TCT | 0 | TCA | 1 | TCT | 0 | TCT | 0 | TCT | 0 | TCA | 1 |
| | 3885 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 4792 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 4816 | | | | | | | | | | | | | | | |
| | Ser | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 4824 | | | | | | | | | | | | | | | |
| | Ser | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 4825 | | | | | | | | | | | | | | | |
| | Ser | | | | | | | | | | | | | | | |
| NSP 12 | Ser | AGC | AGC | 0 | AGC | 0 | AGC | 0 | TCG | 3 | AGC | 0 | TCG | 3 | TCG | 3 |
| | 4842 | | | | | | | | | | | | | | | |
| | Ser | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 4851 | | | | | | | | | | | | | | | |
| | Leu | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 4860 | | | | | | | | | | | | | | | |
| | Leu | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 4861 | | | | | | | | | | | | | | | |
| | Leu | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 4889 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 4905 | | | | | | | | | | | | | | | |
| | Leu | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 4911 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 4918 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 4935 | | | | | | | | | | | | | | | |
| | Leu | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 4940 | | | | | | | | | | | | | | | |
| | Ser | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 4952 | | | | | | | | | | | | | | | |
| | Ser | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 4955 | | | | | | | | | | | | | | | |
| | Ser | | | | | | | | | | | | | | | |
| NSP 12 | Ser | AGC | AGC | 0 | AGC | 0 | AGC | 0 | TCG | 3 | AGC | 0 | TCG | 3 | TCG | 3 |
| | 4983 | | | | | | | | | | | | | | | |
| | Ser | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 4998 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5005 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5021 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5027 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5029 | | | | | | | | | | | | | | | |
| | Leu | | | | | | | | | | | | | | | |
| NSP 12 | Ser | AGC | AGC | 0 | AGC | 0 | AGC | 0 | TCG | 3 | AGC | 0 | TCG | 3 | TCG | 3 |
| | 5038 | | | | | | | | | | | | | | | |
| | Ser | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 5055 | | | | | | | | | | | | | | | |
| | Ser | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 5064 | | | | | | | | | | | | | | | |
| | Leu | | | | | | | | | | | | | | | |
| NSP 12 | Ser | AGT | AGT | 0 | AGT | 0 | AGT | 0 | TCA | 3 | AGT | 0 | TCA | 3 | TCA | 3 |
| | 5083 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5098 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5100 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5118 | | | | | | | | | | | | | | | |
| | Leu | | | | | | | | | | | | | | | |
| NSP 12 | Leu | CTC | CTC | 0 | CTC | 0 | CTC | 0 | TTG | 2 | CTC | 0 | TTG | 2 | TTG | 2 |
| | 5122 | | | | | | | | | | | | | | | |
| | Leu | | | | | | | | | | | | | | | |
| NSP 12 | Leu | CTC | CTC | 0 | CTC | 0 | CTC | 0 | TTG | 2 | CTC | 0 | TTG | 2 | TTG | 2 |
| | 5149 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5150 | | | | | | | | | | | | | | | |
| | Ser | | | | | | | | | | | | | | | |
| NSP 12 | Ser | AGC | AGC | 0 | AGC | 0 | AGC | 0 | TCG | 3 | AGC | 0 | TCG | 3 | TCG | 3 |
| | 5159 | | | | | | | | | | | | | | | |
| | Ser | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5163 | | | | | | | | | | | | | | | |
| | Ser | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 5166 | | | | | | | | | | | | | | | |
| | Leu | | | | | | | | | | | | | | | |
| NSP 12 | Ser | AGC | AGC | 0 | AGC | 0 | AGC | 0 | TCG | 3 | AGC | 0 | TCG | 3 | TCG | 3 |
| | 5169 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5177 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5186 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5196 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5205 | | | | | | | | | | | | | | | |
| | Ser | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 5210 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5220 | | | | | | | | | | | | | | | |
| | Leu | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 5229 | | | | | | | | | | | | | | | |
| | Leu | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5245 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5252 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5260 | | | | | | | | | | | | | | | |
| | Leu | | | | | | | | | | | | | | | |
| NSP 12 | Leu | CTA | CTA | 0 | CTA | 0 | CTA | 0 | TTA | 1 | CTA | 0 | TTA | 1 | TTA | 1 |
| | 5282 | | | | | | | | | | | | | | | |
| | Leu | | | | | | | | | | | | | | | |
| NSP 12 | Ser | TCT | TCT | 0 | TCT | 0 | TCT | 0 | TCA | 1 | TCT | 0 | TCA | 1 | TCA | 1 |
| | 5295 | | | | | | | | | | | | | | | |
| | Ser | | | | | | | | | | | | | | | |
| NSP 12 | Leu | CTT | CTT | 0 | CTT | 0 | CTT | 0 | TTA | 2 | CTT | 0 | TTA | 2 | TTA | 2 |
| | 5298 | | | | | | | | | | | | | | | |
| | Leu | | | | | | | | | | | | | | | |
| NSP 13 | Leu | CTT | CTT | 0 | CTT | 0 | CTT | 0 | TTA | 2 | CTT | 0 | TTA | 2 | TTA | 2 |
| | 5330 | | | | | | | | | | | | | | | |
| | Leu | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5367 | | | | | | | | | | | | | | | |
| | Ser | | | | | | | | | | | | | | | |
| NSP 13 | Leu | CTT | CTT | 0 | CTT | 0 | CTT | 0 | TTA | 2 | CTT | 0 | TTA | 2 | TTA | 2 |
| | 5386 | | | | | | | | | | | | | | | |
| | Leu | | | | | | | | | | | | | | | |
| NSP 13 | Ser | AGC | AGC | 0 | AGC | 0 | AGC | 0 | TCG | 3 | AGC | 0 | TCG | 3 | TCG | 3 |
| | 5392 | | | | | | | | | | | | | | | |
| | Ser | | | | | | | | | | | | | | | |
| NSP 13 | Ser | AGC | AGC | 0 | AGC | 0 | AGC | 0 | TCG | 3 | AGC | 0 | TCG | 3 | TCG | 3 |
| | 5423 | | | | | | | | | | | | | | | |
| | Ser | | | | | | | | | | | | | | | |
| NSP 13 | Leu | CTC | CTC | 0 | CTC | 0 | CTC | 0 | TTG | 2 | CTC | 0 | TTG | 2 | TTG | 2 |
| | 5453 | | | | | | | | | | | | | | | |
| | Leu | | | | | | | | | | | | | | | |
| NSP 13 | Leu | CTT | CTT | 0 | CTT | 0 | CTT | 0 | TTA | 2 | CTT | 0 | TTA | 2 | TTA | 2 |
| | 5455 | | | | | | | | | | | | | | | |
| | Leu | | | | | | | | | | | | | | | |
| NSP 13 | Leu | CTC | CTC | 0 | CTC | 0 | CTC | 0 | TTG | 2 | CTC | 0 | TTG | 2 | TTG | 2 |
| | 5461 | | | | | | | | | | | | | | | |
| | Leu | | | | | | | | | | | | | | | |
| NSP 13 | Leu | CTG | CTG | 0 | CTG | 0 | CTG | 0 | TTG | 1 | CTG | 0 | TTG | 1 | TTG | 1 |
| | 5470 | | | | | | | | | | | | | | | |
| | Leu | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5471 | | | | | | | | | | | | | | | |
| | Ser | | | | | | | | | | | | | | | |
| NSP 13 | Lez5481 | CTG | CTG | 0 | CTG | 0 | CTG | 0 | TTG | 1 | CTG | 0 | TTG | 1 | TTG | 1 |
| | Leu | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5482 | | | | | | | | | | | | | | | |
| | Ser | | | | | | | | | | | | | | | |
| NSP 13 | Leu | CTT | CTT | 0 | CTT | 0 | CTT | 0 | TTA | 2 | CTT | 0 | TTA | 2 | TTA | 2 |
| | 5488 | | | | | | | | | | | | | | | |
| | Leu | | | | | | | | | | | | | | | |
| NSP 13 | Leu | CTT | CTT | 0 | CTT | 0 | CTT | 0 | TTA | 2 | CTT | 0 | TTA | 2 | TTA | 2 |
| | 5499 | | | | | | | | | | | | | | | |
| | Leu | | | | | | | | | | | | | | | |
| NSP 13 | Ser | AGT | AGT | 0 | AGT | 0 | AGT | 0 | TCA | 3 | AGT | 0 | TCA | 3 | TCA | 3 |
| | 5514 | | | | | | | | | | | | | | | |
| | Ser | | | | | | | | | | | | | | | |
| NSP 13 | Leu | CTC | CTC | 0 | CTC | 0 | CTC | 0 | TTG | 2 | CTC | 0 | TTG | 2 | TTG | 2 |
| | 5550 | | | | | | | | | | | | | | | |
| | Leu | | | | | | | | | | | | | | | |
| NSP 13 | Ser | AGT | AGT | 0 | AGT | 0 | AGT | 0 | TCA | 3 | AGT | 0 | TCA | 3 | TCA | 3 |
| | 5559 | | | | | | | | | | | | | | | |
| | Ser | | | | | | | | | | | | | | | |
| NSP 13 | Leu | CTA | CTA | 0 | CTA | 0 | CTA | 0 | TTA | 1 | CTA | 0 | TTA | 1 | TTA | 1 |
| | 5563 | | | | | | | | | | | | | | | |
| | Leu | | | | | | | | | | | | | | | |
| NSP 13 | Leu | CTC | CTC | 0 | CTC | 0 | CTC | 0 | TTG | 2 | CTC | 0 | TTG | 2 | TTG | 2 |
| | 5579 | | | | | | | | | | | | | | | |
| | Leu | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5586 | | | | | | | | | | | | | | | |
| | Ser | | | | | | | | | | | | | | | |
| NSP 13 | Ser | AGC | AGC | 0 | AGC | 0 | AGC | 0 | TCG | 3 | AGC | 0 | TCG | 3 | TCG | 3 |
| | 5587 | | | | | | | | | | | | | | | |
| | Ser | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5601 | | | | | | | | | | | | | | | |
| | Ser | | | | | | | | | | | | | | | |
| NSP 13 | Leu | CTC | CTC | 0 | CTC | 0 | CTC | 0 | TTA | 2 | CTC | 0 | TTA | 2 | TTA | 2 |
| | 5603 | | | | | | | | | | | | | | | |
| | Leu | | | | | | | | | | | | | | | |
| NSP 13 | Ser | AGT | AGT | 0 | AGT | 0 | AGT | 0 | TCA | 3 | AGT | 0 | TCA | 3 | TCA | 3 |
| | 5612 | | | | | | | | | | | | | | | |
| | Ser | | | | | | | | | | | | | | | |
| NSP 13 | Leu | CTA | CTA | 0 | CTA | 0 | CTA | 0 | TTA | 1 | CTA | 0 | TTA | 1 | TTA | 1 |
| | 5618 | | | | | | | | | | | | | | | |
| | Leu | | | | | | | | | | | | | | | |
| NSP 13 | Leu | CTC | CTC | 0 | CTC | 0 | CTC | 0 | TTG | 2 | CTC | 0 | TTG | 2 | TTG | 2 |
| | 5620 | | | | | | | | | | | | | | | |
| | Leu | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5624 | | | | | | | | | | | | | | | |
| | Ser | | | | | | | | | | | | | | | |
| NSP 13 | Ser | TCT | TCT | 0 | TCT | 0 | TCT | 0 | TCA | 1 | TCT | 0 | TCA | 1 | TCA | 1 |
| | 5633 | | | | | | | | | | | | | | | |
| | Ser | | | | | | | | | | | | | | | |
| NSP 13 | Leu | CTA | CTA | 0 | CTA | 0 | CTA | 0 | TTA | 1 | CTA | 0 | TTA | 1 | TTA | 1 |
| | 5640 | | | | | | | | | | | | | | | |
| | Leu | | | | | | | | | | | | | | | |
| NSP 13 | Ser | AGT | AGT | 0 | AGT | 0 | AGT | 0 | TCA | 3 | AGT | 0 | TCA | 3 | TCA | 3 |
| | 5654 | | | | | | | | | | | | | | | |
| | Ser | | | | | | | | | | | | | | | |
| NSP 13 | Ser | AGT | AGT | 0 | AGT | 0 | AGT | 0 | TCA | 3 | AGT | 0 | TCA | 3 | TCA | 3 |
| | 5708 | | | | | | | | | | | | | | | |
| | Ser | | | | | | | | | | | | | | | |
| NSP 13 | Leu | CTT | CTT | 0 | CTT | 0 | CTT | 0 | CTT | 0 | TTA | 2 | TTA | 2 | TTA | 2 |
| | 5823 | | | | | | | | | | | | | | | |
| | Leu | | | | | | | | | | | | | | | |
| NSP 13 | Leu | CTA | CTA | 0 | CTA | 0 | CTA | 0 | CTA | 0 | TTA | 1 | TTA | 1 | TTA | 1 |
| | 5851 | | | | | | | | | | | | | | | |
| | Leu | | | | | | | | | | | | | | | |
| NSP 13 | Ser | TCT | TCT | 0 | TCT | 0 | TCT | 0 | TCT | 0 | TCA | 1 | TCA | 1 | TCA | 1 |
| | 5878 | | | | | | | | | | | | | | | |
| | Ser | | | | | | | | | | | | | | | |
| NSP 13 | Leu | CTT | CTT | 0 | CTT | 0 | CTT | 0 | CTT | 0 | TTA | 2 | TTA | 2 | TTA | 2 |
| | 5896 | | | | | | | | | | | | | | | |
| | Leu | | | | | | | | | | | | | | | |
| NSP 13 | Ser | TCT | TCT | 0 | TCT | 0 | TCT | 0 | TCT | 0 | TCA | 1 | TCA | 1 | TCA | 1 |
| | 5900 | | | | | | | | | | | | | | | |
| | Ser | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 5904 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 5912 | | | | | | | | | | | | | | | |
| | Ser | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 5913 | | | | | | | | | | | | | | | |
| | Leu | | | | | | | | | | | | | | | |
| NSP 14 | Leu | CTC | CTC | 0 | CTC | 0 | CTC | 0 | CTC | 0 | TTG | 2 | TTG | 2 | TTG | 2 |
| | 5931 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 5936 | | | | | | | | | | | | | | | |
| | Ser | | | | | | | | | | | | | | | |
| NSP 14 | Leu | CTC | CTC | 0 | CTC | 0 | CTC | 0 | CTC | 0 | TTG | 2 | TTG | 2 | TTG | 2 |
| | 5951 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 5952 | | | | | | | | | | | | | | | |
| | Ser | | | | | | | | | | | | | | | |
| NSP 14 | Leu | CTC | CTC | 0 | CTC | 0 | CTC | 0 | CTC | 0 | TTG | 2 | TTG | 2 | TTG | 2 |
| | 5978 | | | | | | | | | | | | | | | |
| | Leu | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 5980 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6033 | | | | | | | | | | | | | | | |
| | Leu | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6036 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6041 | | | | | | | | | | | | | | | |
| | Leu | | | | | | | | | | | | | | | |
| NSP 14 | Ser | TCC | TCC | 0 | TCC | 0 | TCC | 0 | TCC | 0 | TCG | 1 | TCG | 1 | TCG | 1 |
| | 6058 | | | | | | | | | | | | | | | |
| | Ser | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6061 | | | | | | | | | | | | | | | |
| | Ser | | | | | | | | | | | | | | | |
| NSP 14 | Leu | CTC | CTC | 0 | CTC | 0 | CTC | 0 | CTC | 0 | TTA | 2 | TTA | 2 | TTA | 2 |
| | 6073 | | | | | | | | | | | | | | | |
| | Leu | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 6076 | | | | | | | | | | | | | | | |
| | Leu | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 6081 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6095 | | | | | | | | | | | | | | | |
| | Ser | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 6098 | | | | | | | | | | | | | | | |
| | Leu | | | | | | | | | | | | | | | |
| NSP 14 | Leu | CTC | CTC | 0 | CTC | 0 | CTC | 0 | CTC | 0 | TTG | 2 | TTG | 2 | TTG | 2 |
| | 6101 | | | | | | | | | | | | | | | |
| | Leu | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6102 | | | | | | | | | | | | | | | |
| | Ser | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6118 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6133 | | | | | | | | | | | | | | | |
| | Leu | | | | | | | | | | | | | | | |
| NSP 14 | Ser | TCC | TCC | 0 | TCC | 0 | TCC | 0 | TCC | 0 | TCG | 1 | TCG | 1 | TCG | 1 |
| | 6142 | | | | | | | | | | | | | | | |
| | Ser | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6154 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6177 | | | | | | | | | | | | | | | |
| | Leu | | | | | | | | | | | | | | | |
| NSP 14 | Ser | AGC | AGC | 0 | AGC | 0 | AGC | 0 | AGC | 0 | TCG | 3 | TCG | 3 | TCG | 3 |
| | 6179 | | | | | | | | | | | | | | | |
| | Ser | | | | | | | | | | | | | | | |
| NSP 14 | Leu | CTG | CTG | 0 | CTG | 0 | CTG | 0 | CTG | 0 | TTG | 1 | TTG | 1 | TTG | 1 |
| | 6183 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6195 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 620462 | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 6253 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6281 | | | | | | | | | | | | | | | |
| | Ser | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6293 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6307 | | | | | | | | | | | | | | | |
| | Leu | | | | | | | | | | | | | | | |
| NSP 14 | Ser | TCC | TCC | 0 | TCC | 0 | TCC | 0 | TCC | 0 | TCG | 1 | TCG | 1 | TCG | 1 |
| | 6320 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6330 | | | | | | | | | | | | | | | |
| | Leu | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6331 | | | | | | | | | | | | | | | |
| | Ser | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 6333 | | | | | | | | | | | | | | | |
| | Leu | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6342 | | | | | | | | | | | | | | | |
| | Ser | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6358 | | | | | | | | | | | | | | | |
| | Ser | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6372 | | | | | | | | | | | | | | | |
| | Ser | | | | | | | | | | | | | | | |
| NSP 14 | Ser | AGT | AGT | 0 | AGT | 0 | AGT | 0 | AGT | 0 | TCA | 3 | TCA | 3 | TCA | 3 |
| | 6374 | | | | | | | | | | | | | | | |
| | Ser | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6378 | | | | | | | | | | | | | | | |
| | Ser | | | | | | | | | | | | | | | |
| NSP 14 | Leu | CTA | CTA | 0 | CTA | 0 | CTA | 0 | CTA | 0 | TTA | 1 | TTA | 1 | TTA | 1 |
| | 6392 | | | | | | | | | | | | | | | |
| | Leu | | | | | | | | | | | | | | | |
| NSP 14 | Ser | TCT | TCT | 0 | TCT | 0 | TCT | 0 | TCT | 0 | TCA | 1 | TCA | 1 | TCA | 1 |
| | 6394 | | | | | | | | | | | | | | | |
| | Ser | | | | | | | | | | | | | | | |
| NSP 14 | Leu | CTC | CTC | 0 | CTC | 0 | CTC | 0 | CTC | 0 | TTG | 2 | TTG | 2 | TTG | 2 |
| | 6419 | | | | | | | | | | | | | | | |
| | Leu | | | | | | | | | | | | | | | |
| NSP 14 | Ser | AGC | AGC | 0 | AGC | 0 | AGC | 0 | AGC | 0 | TCG | 3 | TCG | 3 | TCG | 3 |
| | 6431 | | | | | | | | | | | | | | | |
| | Ser | | | | | | | | | | | | | | | |
| NSP 14 | Leu | CTC | CTC | 0 | CTC | 0 | CTC | 0 | CTC | 0 | TTG | 2 | TTG | 2 | TTG | 2 |
| | 6443 | | | | | | | | | | | | | | | |
| | Leu | | | | | | | | | | | | | | | |
| NSP 14 | Leu | CTT | CTT | 0 | CTT | 0 | CTT | 0 | CTT | 0 | TTA | 2 | TTA | 2 | TTA | 2 |
| | 6450 | | | | | | | | | | | | | | | |
| | Leu | | | | | | | | | | | | | | | |
| NSP 15 | Ser | AGT | AGT | 0 | AGT | 0 | AGT | 0 | AGT | 0 | TCA | 3 | TCA | 3 | TCA | 3 |
| | 6452 | | | | | | | | | | | | | | | |
| | Ser | | | | | | | | | | | | | | | |
| NSP 15 | Ser | TCT | TCT | 0 | TCT | 0 | TCT | 0 | TCT | 0 | TCA | 1 | TCA | 1 | TCA | 1 |
| | 6476 | | | | | | | | | | | | | | | |
| | Ser | | | | | | | | | | | | | | | |
| NSP 15 | Leu | CTT | CTT | 0 | CTT | 0 | CTT | 0 | CTT | 0 | TTA | 2 | TTA | 2 | TTA | 2 |
| | 6508 | | | | | | | | | | | | | | | |
| | Leu | | | | | | | | | | | | | | | |
| NSP 15 | Leu | CTC | CTC | 0 | CTC | 0 | CTC | 0 | CTC | 0 | TTG | 2 | TTG | 2 | TTG | 2 |
| | 6523 | | | | | | | | | | | | | | | |
| | Leu | | | | | | | | | | | | | | | |
| NSP 15 | Ser | CTC | CTC | 0 | CTC | 0 | CTC | 0 | CTC | 0 | TCA | 3 | TCA | 3 | TCA | 3 |
| | 6548 | | | | | | | | | | | | | | | |
| | Ser | | | | | | | | | | | | | | | |
| NSP 15 | Ser | TCT | TCT | 0 | TCT | 0 | TCT | 0 | TCT | 0 | TCA | 1 | TCA | 1 | TCA | 1 |
| | 6554 | | | | | | | | | | | | | | | |
| | Ser | | | | | | | | | | | | | | | |
| NSP 15 | Leu | CTC | CTC | 0 | CTC | 0 | CTC | 0 | CTC | 0 | TTG | 2 | TTG | 2 | TTG | 2 |
| | 6570 | | | | | | | | | | | | | | | |
| | Leu | | | | | | | | | | | | | | | |
| NSP 15 | Leu | CTT | CTT | 0 | CTT | 0 | CTT | 0 | CTT | 0 | TTA | 2 | TTA | 2 | TTA | 2 |
| | 6593 | | | | | | | | | | | | | | | |
| | Leu | | | | | | | | | | | | | | | |
| NSP 15 | Ser | AGT | AGT | 0 | AGT | 0 | AGT | 0 | AGT | 0 | TCA | 3 | TCA | 3 | TCA | 3 |
| | 6598 | | | | | | | | | | | | | | | |
| | Ser | | | | | | | | | | | | | | | |
| NSP 15 | Ser | TCT | TCT | 0 | TCT | 0 | TCT | 0 | TCT | 0 | TCA | 1 | TCA | 1 | TCA | 1 |
| | 6605 | | | | | | | | | | | | | | | |
| | Ser | | | | | | | | | | | | | | | |
| NSP 15 | Ser | AGT | AGT | 0 | AGT | 0 | AGT | 0 | AGT | 0 | TCA | 3 | TCA | 3 | TCA | 3 |
| | 6612 | | | | | | | | | | | | | | | |
| | Ser | | | | | | | | | | | | | | | |
| NSP 15 | Leu | CTT | CTT | 0 | CTT | 0 | CTT | 0 | CTT | 0 | TTA | 2 | TTA | 2 | TTA | 2 |
| | 6613 | | | | | | | | | | | | | | | |
| | Leu | | | | | | | | | | | | | | | |

### Example 2—Mutation of Nsp1

The inventors explored as a strategy for the development of alive-attenuated vaccine for SARS-CoV-2. The Nsp1 double mutant K164A/H165A loses its inhibition capability and the inventors' preliminary analysis of transcriptional responses to SARS-CoV-2 Nsp1 mutant infection confirms an increased host response to infection.

The inventors additionally mutated Nsp1 in two positions corresponding to K164A, H165A in SEQ Id NO: 7, and deleted accessory ORFs 6-8 as in SEQ ID NO: 2.

Deletion of the FCS region. The FCS region was deleted as described in Davidson A D, Williamson M K, Lewis S, et al., 2020, Genome Med. 2020; 12(1):68.

The inventors infected hamsters with the OTS viruses by intranasal administration of 5000 PFU/mouse, followed by a challenge infection with the ancestral SARS-CoV-2 (Wuhan wild-type (WT)) 21-days post-infection (FIG. 10).

The inventors evaluated the survival of animals inoculated with OTS viruses or SARS-CoV-2 WT (FIG. 11). 75% of the animals inoculated with SARS-CoV-2 wild-type succumbed to the disease or reached termination criteria within 8 days post-inoculation. In strong contrast, none of the animals inoculated with OTS constructs died.

Animals inoculated with SARS-CoV-2 WT, OTS4-5 and OTS7-8 viruses lost weight upon infection (mean bodyweights=84% (7 dpi; days post-infection), 91% (8 dpi) and 89% (7 dpi), respectively). In strong contrast, animals inoculated with OTS 4-5-6-7-8 Nsp1^{K164A/H165A}.delORF6-8 and OTS 4-5-6-7-8 Nsp1^{K164A/H165A}.delORF6-8.FCS (SEQ ID NO: 6 referred to as OTS final in the figures) gradually gained weight (mean bodyweight=106% (7 dpi) and 108% (8 dpi)), indicative of the lack of pathogenicity of OTS 4-5-6-7-8 Nsp1^{K164A/H165A}.delORF6-8 and OTS 4-5-6-7-8 Nsp1^{K164A/H165A}.delORF6-8.FCS in the highly sensitive Syrian hamster model (FIG. 11).

Additionally, conchae, trachea, lung (cranial, medial, caudal) samples, and nasal washing samples were collected 5 days post-infection and analyzed by an ORF1ab (Nsp12) specific RT-qPCR. By using a genome copy standard, the total amount of virus genome copies per ml (gc/ml) was calculated for each sample. Based on this information the amounts of virus genome copies were compared to each other and a fold change value was calculated (FIG. 12-14). Hamsters infected with SARS-CoV-2 WT, OTS4-5, and OTS7-8 did not differ in their virus genome loads in organs and washing samples. In contrast, OTS 4-5-6-7-8 Nsp1^{K164A}/H1^{65A}.delORF6-8 and OTS 4-5-6-7-8 Nsp1^{K164A/H165A}.delORF6-8.FCS had reduced virus genome load in the organs and in the washing samples.

The in vivo evaluation of OTS vaccine candidates OTS4-5, OTS7-8, and OTS 4-5-6-7-8 Nsp1^{K164A/H165A}.delORF6-8 and OTS 4-5-6-7-8 Nsp1^{K164A/H165A}.delORF6-8.FCS in Syrian hamsters confirms the partial attenuation of OTS4-5 and OTS7-8 and the improved properties of OTS 4-5-6-7-8 Nsp1^{K164A/H165A}.delORF6-8 and OTS 4-5-6-7-8 Nsp1^{K164A/H165A}.delORF6-8.FCS.

### Example 3

Adding a mutagen such as 5-Fluorouracil or molnupiravir reduced the number of infectious virus particles in a TCID50 virus assay. Particularly, the OTS virus is more prone to inactivation by a mutagen than WT SARS-CoV-2 (FIG. 16).

### Example 4

SARS-CoV-2 genome was reverse-engineered to increase the likelihood of generating stop codons, resulting in so-called “one-to-stop (OTS)” codons, which in turn would lead to attenuated SARS-CoV-2 variants (called herein also OTS constructs or attenuated OTS viruses) that could serve as live-attenuated vaccines (LAV). In addition, the inventors mutated Nsp1 (K164A/H165A) and deleted ORF6 to 8 to further enhance both OTS-driven attenuation and in vivo immunogenicity. To evaluate attenuation and protection, the inventors inoculated K18-hACE2 transgenic mice and Syrian hamsters with different OTS viruses and assessed protection by diverse SARS-CoV-2 challenge infections. It was demonstrated that a single intranasal administration of attenuated OTS viruses, either OTS-206 (OTS4-5-7-8.Nsp1K164A,H165A.delORF6-8), or OTS-228 (OTS.4-5-7-8.Nsp1K164A,H165A.delORF6-8.FCS), containing a polybasic cleavage site (PCS) deletion in addition to the modifications of candidate OTS-206, provided protection against SARS-CoV-2 and its variants of concern (VOCs) Omicron BA.2 and BA.5. The deletion of the PCS in the final vaccine candidate OTS-228 moreover even led to a significant reduction in virus transmission to contact animals, highlighting OTS-228 as a very promising live-attenuated vaccine candidate.

### Materials and Methods

### Cell Culture

VeroE6 (Vero C1008, ATCC) and VeroE6/TMPRSS2 cells (NIBSC Research Reagent Depository, UK) were cultured in Dulbecco's modified Eagle's medium (DMEM) supplemented with 10% (v/v) fetal bovine serum (FBS), 1% (w/v) non-essential amino acids (NEAA), 100 IU/mL penicillin, 100 μg/mL streptomycin μg/ml. BHK-21 cells expressing the N protein of SARS-CoV (BHK-SARS-N) (PLoS ONE 7(3): e32857, doi:10.1371/journal.pone.0032857) were grown in minimal essential medium (MEM) supplemented as DMEM above. Cells were maintained at 37° C. with 5% CO2, under the selection with puromycin (Vero E6/TMPRSS2) and doxycycline (BHK-SN).

VeroE6 (Collection of Cell Lines in Veterinary Medicine CCLV-RIE 0929) were cultured using a mixture of equal volumes of Eagle MEM (Hanks' balanced salts solution) and Eagle MEM (Earle's balanced salts solution) supplemented with 2 mM L-Glutamine, NEAA adjusted to 850 mg/L, NaHCO₃, 120 mg/L sodium pyruvate, 10% FBS, pH 7.2.

Generation of Infectious cDNA Clones Using Transformation-Associated Recombination Cloning and Rescue of Recombinant Viruses

The in-yeast transformation-associated recombination (TAR) cloning method, as previously described (Nature 582, 561-565 (2020), doi:10.1038/s41586-020-2294-9), was used to generate recombinant one-to-stop (OTS) SARS-CoV-2 viruses of SARS-CoV-2. Briefly, 12 overlapping DNA fragments encoding the entire SARS-CoV-2 genome (referred to as WU-Fragments 1-12), along with a TAR-vector, were homologously recombined in yeast to form the yeast artificial chromosome (YAC). WU-Fragments 2, 4, 5, 7, and 8 were recoded according to the OTS strategy to produce OTS-Fragments. The OTS strategy involves recoding all serine and leucine codons to synonymous codons that are just one further nucleotide change away from encoding a stop codon. For example, the leucine coding CUU was changed to the synonymous UUA. Consequently, the UUA codon just needs one mutation to change into the UGA stop codon.

Initially, single OTS fragments (cf. SEQ listing) were used to create infectious SARS-CoV-2 clones, namely OTS2 (WU-Fragment 2 out of the 12 WU-Fragments was replaced with OTS Fragment 2), OTS4, OTS5, OTS7, OTS8. Subsequently, clones with multiple OTS fragments were created, such as OTS4-5, OTS7-8, and OTS4-5-7-8. Supplementary Table 3 provides a detailed list of all nucleotide changes recoded in the OTS fragments (changes in OTS2 under fg 2, OTS4 under fg 4, OTS5 under fg 5, OTS7 under fg 7, OTS8 under fg 8). The recombinant SARS-CoV-2 OTS-206 infectious clone contains additional modifications, for which the inventors created WU-Fragment 2-Nsp1:K164A,H165A, and WU-Fragment 11:delORF6-8. Four point mutations were introduced into WU-Fragment 2 to create amino acid changes K164A and H165A in the Nsp1 gene, and deleted ORF6 to ORF8 from WU-Fragment 11 using PCR. Lastly, to create OTS-228, the final iteration of attenuation strategy, WU-Fragment 10 was replaced with WU-Fragment 10:delFCS, where the polybasic cleavage site in the SARS-CoV-2 spike was removed. The primers used for these modifications are listed in Supplementary Table 1. The inventors recombined the overlapping fragments encoding the recombinant viruses in yeast to create the YAC. The YACs were cleaved by EagI digestion, and in vitro transcription was performed using the T7 RiboMAX Large Scale RNA production system (Promega), as previously described (Nature 582, 561-565 (2020), doi:10.1038/s41586-020-2294-9). The resulting capped mRNA was electroporated into BHK-21 cells expressing the SARS-CoV N protein. Electroporated BHK-21 cells were then co-cultured with VeroE6/TMPRSS2 cells to produce passage 0 (p.0) of the recombinant viruses. To generate a p.1 virus stock for downstream experiments, the p.0 viruses were used to infect VeroE6/TMPRSS2 cells.

### Determination of Infectious Viral Particles, Plaque Phenotype and Foci Sizes

A complete list of viruses used in this study can be found in Supplementary Table 1. VeroE6 or VeroE6/TMPRSS2 were used to culture viruses, and the identity of all virus stocks was verified by whole-genome NGS sequencing. Infectious viral particle titers were determined by TCID50 measurement on VeroE6 or VeroE6/TMPRSS2 cells. Briefly, 2×104 cells/well were seeded in a 96-well plate one day before the titration and were then inoculated with a 10-fold serial dilution of the samples. Three to six technical replicates were performed for each sample. Cells were then incubated at 37° C. in a humidified incubator with 5% CO2. After 72 h, cells were fixed with 4% (v/v) buffered formalin (formafix) and stained with crystal violet. TCID50 was calculated according to the Spearman-Kaerber formula. The plaque sizes caused by the respective viruses in 6-well plates 2 post inoculation (dpi) were measured in Adobe Illustrator. Statistical significance was determined using ordinary one-way Anova and p-values were adjusted using Tukey's multiple-comparison test; *P<0.05, **P<0.01, ***P<0.001, ****P<0.0001.

### Genetic Stability of Recombinant OTS Viruses

To evaluate their genetic stability, OTS4-5 (10-times VeroE6), OTS7-8, (10-times VeroE6) OTS206 (15-times VeroE6/TMPRSS2) were passaged at low MOI (0.01) and sequenced by Ion Torrent Sequencing. Also, conchae samples of OTS4-5 and OTS7-8 contact animals 20 days post initial contact were sequenced. Results are shown in Supplementary Table 5.

### Ion Torrent Sequencing

Virus stocks and animal samples were sequenced using a generic metagenomics sequencing workflow as described previously (Wylezich et al. 2018, Sci Rep 8, 13108) with some modifications. For reverse-transcribing RNA into cDNA, SuperScriptIV First-Strand cDNA Synthesis System (Invitrogen, Germany) and the NEBNext Ultra II Non-Directional RNA Second Strand Synthesis Module (New England Biolabs, Germany) were used, and library quantification was done with the QIAseq Library Quant Assay Kit (Qiagen, Germany). Animal samples were treated with a myBaits panel (Daicel Arbor Biosciences) specific for SARS-CoV-2 as described (Wylezich et al. 2021, Microbiome. 2021; 9: 51). Libraries were quality-checked, quantified and sequenced using an Ion 530 chip and chemistry for 400 base pair reads on an Ion Torrent S5XL instrument (Thermo Fisher Scientific, Germany). Raw sequencing reads data were analyzed using the Genome Sequencer Software Suite (version 2.6; Roche, Mannheim, Germany https://roche.com) applying default software settings for quality filtering and mapping. The obtained genome sequences were compared with their reference genomes via alignment using MAFFT version 7.38837, as implemented in Geneious version 10.2.3 (Biomatters, Auckland, New Zealand; https://www.geneious.com). The variant analysis integrated in Geneious Prime 10.2.3 were applied (default settings, minimum variant frequency 0.02) to detect single nucleotide variants (SNV).

### Illumina Sequencing

Sequencing reads were trimmed using TrimGalore v.0.6.5 and FastQC v.0.11.9 was used to assess overall read quality. Trimmed reads for each OTS sample were then aligned to their corresponding OTS reference sequence using Bowtie2 v.2.3.4. For virus stocks, consensus sequences were generated using Samtools v.1.10 with the -d option set to 10,000. For OTS passaged samples, nucleotide variants were called using Lofreq v.2.1.5 with the -C option set to 100 and the -d option set to 10,000. The resulting VCF files were filtered using the lofreq filter command for variants called at a frequency of Q 0.1. Data analysis was performed on UBELIX, the high-performance computing (HPC) cluster at the University of Bern (http://www.id.unibe.ch/hpc).

### Virus Replication Kinetics, Fluorouracil (5-FU) and Molnupiravir Treatment

The virus replication kinetics of the OTS viruses in comparison to WT SARS-CoV-2 (Acc. No. MT108784) were determined without any treatment, as well as under fluorouracil (5-FU) (Sigma, F6627) and molnupiravir (Lucerna Chem, HY-135853-10MG) treatment conditions. VeroE6/TMPRSS2 cells were infected with 0.1 MOIs of the WT SARS-CoV-2 or OTS viruses for 1 hour. After an hour, inoculum was removed, cells were washed three times with 1×PBS and new media was added on the cells. Supernatant from wells were collected at 6-, 18-, 24-, 48- and 72 hpi (hours post infection) for the infectious virus titer determination and diluted 1:1 with virus transport medium (VTM). For the antiviral treatment condition, VeroE6/TMPRSS2 cells were pretreated for 30 minutes with 5-FU and molnupiravir, and then infected with 0.1 MOI of WT SARS-CoV-2 and OTS4-5-7-8 for 1 hour. Afterwards, inoculum was removed, cells were washed and new medium containing either 5-FU (concentration ranging from 40-280 μM), or molnupiravir (concentration ranging from 0.1-10 μM) was added on the cells for 24 hours. After 24 hours, supernatant from cells were collected and used to determine the virus titers. Infectious virus titers were assessed by standard TCID50 assays on Vero-E6/TMPRSS2 cells, as explained above.

### Well-Differentiated Primary Airway Epithelial Cells

Primary human bronchial epithelial cells (hBECs) were isolated from lung explants and human nasal epithelial cells (hNECs) were obtained commercially (Epithelix Sàrl). The generation of well-differentiated hBECs and hNECs at the air-liquid interface (ALI) was described previously with minor adjustments (Cell Rep Med. 2021 Dec. 21; 2(12):100456, doi:10.1016/j.xcrm.2021.100456). Human BECs/NECs were expanded in collagen-coated (Sigma) cell culture flasks (Costar) in PneumaCult Ex Plus medium, supplemented with 1 μM hydrocortisone, 5 μM Y-27632 (Stem Cell Technologies), 1 μM A-83-01 (Tocris), 3 μM isoproterenol (Abcam), and 100 μg/mL primocin (Invivogen) and maintained at 37° C., 5% CO2. Expanded hBECs/hNECs were seeded onto 24-well plate inserts with a pore size of 0.4 μm (Greiner Bio-One) at a density of 50′000 cells/insert, submerged into 200 μl of supplemented PneumaCult ExPlus medium on the apical side and 500 μl in the basolateral chamber. To induce the differentiation of the cells, PneumaCult ALI medium supplemented with 4 μg/mL heparin (Stem Cell Technologies), 5 μM hydrocortisone, and 100 μg/mL primocin was added to the basolateral chamber. Basal medium was replaced every 2-3 days and the cells were maintained at 37° C., 5% CO2 until ciliated cells appeared and mucus was produced. After 3 to 4 weeks post-exposure to ALI, hBECs/hNECs were considered well-differentiated. For FIG. 19d, well-differentiated commercial hNECs) were obtained commercially (Epithelix Sàrl) were obtained and consisting of a pool of 14 human donors each. Basal medium (Epithelix Sàrl) was replaced every 2-3 days and cells were maintained at 33° C., 5% CO2. To remove mucus from hBECs and hNECs, cells were washed once a week with 250 μl of pre-warmed Hank's balanced salt solution (HBSS, Gibco) for 20 min at 37° C.

### Virus Replication Kinetics on Human Primary Airway Cells

Human BECs and NECs were infected with 5×104 PFU of the OTS viruses listed or WT SARS-CoV-2 (Acc. No. MT108784) as described previously (Nat Commun. 2022 Oct. 7, 13(1):5929, doi.org/10.1038/s41467-022-33632-y). Viruses were diluted in HBSS, applied apically, and incubated for 1 hour at 37° C. or 33° C. for hBECs or hNECs, respectively. Then, the inoculum was removed, and the cells were washed three times with 100 μl of HBSS. The last wash was collected as the 1 hpi time point and diluted 1:1 with VTM. Afterwards, hBECs and hNECs were incubated in a humidified incubator with 5% CO2 at 37° C. or 33° C., respectively. For quantification of infectious viral particle release 24, 48, 72, and 96 hpi, 100 μl HBSS were applied to the apical surface 10 min prior to the respective time point, incubated, and subsequently collected. Apical washes were diluted 1:1 with VTM and stored at −80° C. until further analysis. Infectious virus titers in the apical washes were assessed by a standard TCID50 assay on VeroE6/TMPRSS2 cells.

A well-characterized SARS-CoV-2 model (J Virol. 2007, 81(2):813-21, doi.org/10.1128/jvi.02012-06; Nature 2021, 592(7852):122-127), doi:10.1038/s41586-021-03361-1) hACE2-K18Tg mice (Tg(K18-hACE2)2Prlmn) were bred at the specific pathogen-free facility of the Institute of Virology and Immunology and housed as previously described (Nature 2022, 602(7896):307-313), doi:10.1038/s41586-021-04342-0). For infection, 8- to 17-week-old female and male mice were anesthetized with isoflurane and inoculated intranasally with 20 μl per nostril. The titers of each virus used in individual experiments are given in the text and figure legends. The mice were observed for clinical symptoms, weighed and swabbed at specific time points. The clinical symptoms were scored, and the animals were euthanized before they reached the humane endpoint. On euthanasia day, swabs, serum and organs samples were harvested as mentioned in previous studies (Nature 2021, 592(7852):122-127, doi:10.1038/s41586-021-03361-1).

For the vaccination experiments, K18-hACE2 mice (7-16 weeks old) were immunized intramuscularly with a single dose of 1 μg of mRNA-Vaccine Spikevax (Moderna) or intranasally with 5′000 PFU of OTS viruses. Four weeks after prime immunization, mice were booster again either i.m. with 1 μg of mRNA-Vaccine Spikevax (Modema) or intranasally with 5′000 PFU of OTS viruses. Four weeks after the boost, the immunized mice and a group of sex- and age-matched naïve animals were challenged intranasally with the challenge virus inoculum described in the results section. Euthanasia and organ collection was performed 6 dpc as described above. All mice were monitored daily for body weight loss and clinical signs. Oropharyngeal swabs were collected daily as described before.

In addition, specific pathogen free male Syrian golden hamsters (Mesocricetus auratus) were purchased from Janvier labs, Le Genest-Saint-Isle, France. Table S1 summarized the animal numbers used for the inoculation experiments. Syrian hamsters received either 70 μl (35 μl into each nostril) of the respective OTS constructs (OTS4-5, OTS7-8, OTS-206 or OTS-228) intranasally or were challenged 3 weeks post immunization with SARS-CoV-2 WT (BetaCoV/Wuhan/IVDC-HB-01/2019, Acc. No. MT108784), SARS-CoV-2 Omicron BA.2 (SARS-CoV-2/human/NLD/EMC-BA2-1/2022, Acc. No. ON545852) or SARS-CoV-2 Omicron BA.5 (hCoV-19/South Africa/CERI-KRISP-K040013/2022, Acc. No. EPI_ISL_12268493.2). Details about OTS-viruses and challenge viruses which were used to be is found under in Supplementary Table 1. Body weight was tracked and nasal washing samples, under short term isoflurane anesthesia, were taken (flushing 200 μl PBS into each nostril and collecting the reflux into a 2 mL tube) at time points as specifically indicated for each experiment (FIG. 201, r; FIG. 22d, k; FIG. 25a; FIG. 30, FIG. 31). To obtain organ samples (nasal conchae, trachea, lung caudal, medial and cranial) animals were euthanized by an isoflurane overdose and subsequent decapitation. Serum samples were obtained during euthanasia by collecting the blood into serum separating tubes (BD Vacutainer™)

### Processing of Animal Specimens, Viral RNA and Infectious Particle Quantification

Organ samples of about 0.1 cm³ size from hamsters were homogenized in a 1 mL mixture composed of equal volumes of Hank's balanced salts MEM and Earle's balanced salts MEM containing 2 mM L-glutamine, 850 mg/L NaHCO3, 120 mg/L sodium pyruvate, and 1% penicillin-streptomycin) at 300 Hz for 2 min using a Tissuelyser II (Qiagen) and were then centrifuged to clarify the supernatant.

Nucleic acid was extracted from 100 μl of the nasal washes of hamsters after a short centrifugation step or 100 μl of organ sample supernatant using the NucleoMag Vet kit (Macherey Nagel). Nasal washings, oropharyngeal swabs and organ samples from hamsters were tested by virus-specific RT-qPCR. The RT-qPCR reaction was prepared using the qScript XLT One-Step RT-qPCR ToughMix (QuantaBio, Beverly, MA, USA) in a volume of 12.5 μl including 1 μl of the respective FAM mix and 2.5 μl of extracted RNA. The reaction was performed for 10 min at 50° C. for reverse transcription, 1 min at 95° C. for activation, and 42 cycles of 10 sec at 95° C. for denaturation, 10 sec at 60° C. for annealing and 20 sec at 68° C. for elongation. Fluorescence was measured during the annealing phase. RT-qPCRs were performed on a BioRad real-time CFX96 detection system (Bio-Rad, Hercules, USA). The primers are listed in Supplementary Table 2.

Organ samples from mice were either homogenized in 0.5 mL of RA1 lysis buffer supplemented with 1% β-mercaptoethanol and later used for RNA isolation, or in 1 ml DMEM containing gentleMACS M-tubes (Miltenyi Biotec) for the detection of infectious particles as described before (doi:10.1038/s41586-021-04342-0). RNA was isolated using the NucleoMag Vet kit (Macherey Nagel). The RT-qPCR reaction was prepared using TaqPath™ 1 Step Multiplex Master Mix kit (Thermofisher) with primers and probes targeting SARS-CoV-2 E gene, and was performed for 10 min at 45° C. for reverse transcription, 10 min at 95° C. for activation, and 45 cycles of 15 sec at 95° C. for denaturation, 30 sec at 58° C. for annealing and 30 sec at 72° C. for elongation. Fluorescence was measured during the annealing phase. RT-qPCRs were performed on a BioRad real-time CFX96 detection system (Bio-Rad, Hercules, USA). The primers are listed in Supplementary Table 2. Infectious virus titers were determined by TCID50 measurement on VeroE6 cells and were calculated according to the Spearman-Kaerber formula.

### Histopathological and Immunohistochemical Analysis

The left lung and the left hemisphere of the brain from mice were collected into 4% formalin. After fixation, both tissues were embedded in paraffin, cut at 4 μm and stained with hematoxylin and eosin (H&E) for histological evaluation. Scoring of the lung tissue pathology was done according to a previously published scoring scheme (Ulrich, L. et al. Enhanced fitness of SARS-CoV-2 variant of concern Alpha but not Beta. Nature 602, 307-313 (2022)). Immunohistochemical (IHC) analysis of the lung and the brain was performed by using a rabbit polyclonal anti-SARS-CoV nucleocapsid antibody (Rockland, 200-401-A50) in a BOND RXm immunostainer (Leica Byosystems, Germany). For that purpose, paraffin blocks were cut at 3 μm, incubated with citrate buffer for 30 min at 100° C. for antigen retrieval, and incubated with a 1:3000 dilution of the first antibody for 30 min at room temperature. Bond™ Polymer Refine Detection visualizsation kit (Leica Byosystems, Germany) was afterwards used for signal detection using DAB as chromogen and counterstaining with hematoxylin.

The left lung lobe was carefully removed, immersion-fixed in 10% neutral-buffered formalin, paraffin-embedded, and 2-3 μm sections were stained with hematoxylin and eosin (HE). Consecutive sections were processed for immunohistochemistry (IHC) used according to standardized procedures for the of avidin-biotin-peroxidase complex (ABC)-method. Briefly, endogenous peroxidase was quenched on dewaxed lung slides with 3% hydrogen peroxide in distilled water for 10 minutes at room temperature (RT). Antigen heat retrieval was performed in 10 mM citrate buffer (pH 6) for 20 minutes in a pressure cooker. Nonspecific antibody binding was blocked for 30 minutes at RT with goat normal serum, diluted in PBS (1:2). A primary anti-SARS-CoV nucleocapsid protein antibody was applied overnight at 4° C. (Rockland, 200-401-A50, 1:3000), the secondary biotinylated goat anti-mouse antibody was applied for 30 minutes at room temperature (Vector Laboratories, Burlingame, CA, USA, 1:200). Color was developed by incubation with ABC solution (Vectastain Elite ABC Kit; Vector Laboratories), followed by exposure to 3-amino-9-ethylcarbazole substrate (AEC, Dako, Carpinteria, CA, USA). The sections were counterstained with Mayer's haematoxylin and coverslipped. As negative control, consecutive sections were labelled with an irrelevant antibody (M protein of Influenza A virus, ATCC clone HB-64). An archived control slide from a SARS-CoV2 infected Syrian hamster was included in each run. All slides were scanned using a Hamamatsu S60 scanner and evaluated using the NDPview.2 plus software (Version 2.8.24, Hamamatsu Photonics, K.K. Japan) by a trained (TB) and board-certified pathologist (AB), blind to treatment. The lung tissue was evaluated using a 500×500 μm grid, and the extent of pneumonia-associated consolidation was recorded as percentage of affected lung fields. Further, the lung was examined for the presence of SARS-CoV-2-characteristic lesions described for hamsters, i.e. intra-alveolar, interstitial, peribronchial and perivascular inflammatory infiltrates, alveolar edema, necrosis of the bronchial epithelium, diffuse alveolar damage, vasculitis, activation of endothelium with immune cell rolling, as well as bronchial epithelial and pneumocyte type 2 hyperplasia. Following IHC the distribution of virus antigen was graded on an ordinal scale with scores 0=no antigen, 1=focal, affected cells/tissue <5% or up to 3 foci per tissue; 2=multifocal, 6%-40% affected; 3=coalescing, 41%-80% affected; 4=diffuse, >80% affected. The target cell was identified based on morphology.

### Serological Tests

To evaluate the virus neutralizing potential of hamster serum samples, a live virus neutralization test was done following an established standard protocol as described before (Schlottau, K. et al. SARS-CoV-2 in fruit bats, ferrets, pigs, and chickens: an experimental transmission study. The Lancet Microbel, e218-e225 (2020)). Briefly, sera were prediluted 1/16 in MEM and further diluted in log 2 steps until a final tested dilution of 1/4096. Each dilution was evaluated for its potential to prevent 100 TCID50 SARS-CoV-2/well of the respective VOC from inducing cytopathic effect in Vero E6 cells, giving the virus neutralization titer (VNT100). Following SARS-CoV-2 variants were used to test against: SARS-CoV-2 WT D614G (BetaCoV/Germany/BavPat1/2020, Acc. No. EPI_ISL_406862), SARS-CoV-2 Omicron BA.2 (SARS-CoV-2/human/NLD/EMC-BA2-1/2022, Acc. No. ON545852) or SARS-CoV-2 Omicron BA.5 (hCoV-19/South Africa/CERI-KRISP-K040013/2022, Acc. No. EPI_ISL_12268493.2).

Additionally, serum samples were tested by multispecies ELISA for sero-reactivity against the SARS-CoV-2 RBD domain (Wernike K. et al., Multi-species ELISA for the detection of antibodies against SARS-CoV-2 in animals. Transbound Emerg. Dis. 68, 1779-1785 (2021)).

Similarly, for mouse samples, serum was diluted initially at 1:20 with DMEM, and subsequently was further diluted to reach the final dilution of 1:2560. Diluted sera were first incubated with the virus in 1:1 volume ratio, and after 1h incubation, the serum-virus mixture was applied on Vero E6 cells in 96-well plates for 2-3 days incubation period. The serum dilution in which the cells were still intact was recorded as neutralization titer of the serum for the given virus.

### Spatial Transcriptomics and Gene Expression Analysis

5 μm thick formalin-fixed paraffin-embedded (FFPE) lung tissue sections were placed on Visium Spatial Gene Expression slides (10× Genomics) containing four capture areas each and processed according to the manufacturer's recommendations. In addition to the mouse transcriptome probes, the inventors designed probes for the SARS-CoV-2 virus targeting ORF1ab, ORF3a, ORF10, and the genes encoding the structural proteins spike (S), envelope (E), membrane (M), and nucleocapsid (N). The custom SARS-CoV-2 probes are listed in Supplementary Table 3 and the final concentration for each primer in the probe hybridization mix was 1.2 nM. The cDNA libraries were loaded onto the NovaSeq 6000 (Illumina) and sequenced with a minimum of 50,000 reads per covered spot. Reads contained in Illumina FASTQ files were aligned to a custom multi-species reference transcriptome generated with Space Ranger using the GRCm38 (version mm10-2020-A_build, 10× Genomics) mouse and NC_045512.2 SARS-CoV-2 references. Downstream data analysis of the mouse samples was performed using SCANPY (Wolf, F. Alexander, Philipp Angerer, and Fabian J. Theis. “SCANPY: large-scale single-cell gene expression data analysis.” Genome Biology 19 (2018): 1-5) python package. To compare host and viral gene expression levels across conditions, the counts were first normalized and then log transformed. To examine spatial correlations between total viral mRNA counts and host genes, pairwise Pearson's correlation coefficients were calculated and compared across conditions. Cellular pathway activity scores for 13 different cellular pathways were calculated using PROGENy (Schubert, Michael, et al. “Perturbation-response genes reveal signaling footprints in cancer gene expression.” Nature Communications 9.1 (2018): 20).

Statistical analysis was performed using GraphPad Prism 9 (Version 9.5.1). Unless noted otherwise, the results are expressed as mean±s.d. Specific tests are indicated in the main text or the figure legends. All experiments with infectious SARS-CoV-2 variants as well as the attenuated OTS constructs were performed in enhanced biosafety level 3 (BSL3) containment laboratories approved by relevant authorities in Switzerland and Germany. All personnel received relevant training before commencing work in BSL3 laboratories. Tetramer staining of mice blood cells All the preparation of the cells and staining was done in BSL3 conditions. Whole blood was collected in EDTA tubes with heparinized capillary tubes (Sigma-Aldrich, BR749311). After the centrifugation of the blood at 400×g for 10 min, sera were collected, heat inactivated at 56° C. and immediately stored in −80° C. In-house red blood cell lysis buffer (containing ammonium chloride, sodium bicarbonate, EDTA) was added on the rest of the blood, and the mix was incubated on ice for 10 min. Later, cold PBS was added in the tubes, and they were centrifuged at 350×g, 4° C. for 5 min, supernatant was discarded. Following the addition Live/Dead fixable aqua dead cell stain (Thermofisher), cells were incubated on ice for 10 min, then washed with cold PBS, and centrifuged at 350×g, 4° C. for 5 min. After discarding the supernatant, cells were incubated with avidin (MERCK) and FcR-blocking reagent (anti-mouse CD16/32) (Miltenyi biotec) for 20 min on ice. Subsequently, antibody mixes including the following antibodies were mixed with the cells and incubated for 30 min in dark on ice: anti-mouse anti-CD8-FITC (biolegend), anti-mouse anti-CD45-PerCP (biolegend), anti-mouse anti-CD3e-PE (biolegend), either MHC-I tetramer against SARS-CoV-2 spike (H-2K(b), SARS-CoV-2 S 539-546, VNFNFNGL) (NIH tetramer core facility), or negative control (H-2D(b) Influenza A NP 366-374 ASNENME™). In addition, a fluorescence minus one (FMO) control without the tetramer or negative control antibody, as well as single antibody staining were prepared as flow cytometry control and compensation groups. Cells were washed two times with PBS, centrifuged at 350×g, 4° C. for 5 min. Finally, PBS+4% paraformaldehyde (PFA) (in-house) was added on the cells to fix them to take out the samples out of BSL3 for flow cytometry acquisition in FACS Canto II (BD Bioscience) using the DIVA software.

SUPPLEMENTARY TABLE 1
List of viruses used for the experiments:

| | | | | |
|---|---|---|---|---|
| Abbreviation used | | | Accession | |
| in the manuscript | Name | Explanation | Number | Experiment |

Challenge viruses

| | | | | |
|---|---|---|---|---|
| WT | BetaCoV/Wuhan/IVDC-HB- | wild-type SARS-CoV-2 L | MT108784 | FIG. 2 l, p; Extended |
| | 01/2019 | Lineage B | | Data FIG. 4 l, m |
| WT^{D614G} | BetaCoV/Germany/BavPat1/ | SARS-CoV-2 WT with D614G | EPI_ISL_{—} | VNT(hamster), FIG. 3 |
| | 2020 | mutation - Lineage B.1 | 406862 | g-m, FIG. 4 b-c |
| Omicron BA.2 | SARS-CoV-2/human/NLS/ | VOC Omicron BA.2 | ON545852 | FIG. 2 q-v; Extended |
| | EMC-BA2-1/2022 | | | Data FIG. 4 o, p; |
| | | | | VNT (hamster) |
| Omicron BA.5 | hCoV-19/South Africa/CERI- | VOC Omicron BA.5 | EPI_ISL_{—} | FIG. 4 k-r; Extended |
| | KRISP-K040013/2022 | | 12268493.2 | Data FIG. 10 a-c; |
| | | | | VNT (hamster) |
| Delta (B.1.617.2) | hCoV-19/Germany/BW- | VOC Delta GK (B.1.617.2 + | EPI_ISL_{—} | FIG. 3 a-m; |
| | FR1407/2021 | AY.*) | 2535433 | Extended Data Fig. |
| | | | | 6 and 7 |

Modified viruses

| | | |
|---|---|---|
| OTS2 | | WT SARS-CoV-2 with OTS |
| | | modifications in fragment 2 |
| OTS7 | | WT SARS-CoV-2 with OTS |
| | | modifications in fragment 7 |
| OTS8 | | WT SARS-CoV-2 with OTS |
| | | modifications in fragment 8 |
| OTS4-5 | | WT SARS-CoV-2 with OTS |
| | | modifications in fragments 4 |
| | | and 5 |
| OTS7-8 | | WT SARS-CoV-2 with OTS |
| | | modifications in fragments 7 |
| | | and 8 |
| OTS4-5-7-8 | | WT SARS-CoV-2 with OTS |
| | | modifications in fragments |
| | | 4, 5, 7 and 8 |
| OTS-206 | OTS4-5-7- | OTS in fragments 4-5, 7-8; point |
| | 8.NSP1^{K164A, H165A}.delORF6-8 | mutations in NSP1 (K164A, |
| | | H165A), deletion of ORF6 to ORF8 |
| OTS-228 | OTS4-5-7- | OTS in fragments 4-5, 7-8; point |
| | 8.NSP1^{K164A, H165A}.delORF6- | mutations in NSP1 (K164A, |
| | 8.delCS | H165A), deletion of ORF6 to |
| | | ORF8, deletion of 24 nucleotides |
| | | in furin cleavage site (23598- |
| | | 23622) |
| delORF6-8 | | WT SARS-CoV-2, deletion of ORF6 |
| | | to ORF8 |
| nsp1 | | WT SARS-CoV-2, point mutations |
| | | in NSP1 (K164A, H165A) |
| SARS-CoV-2 [symbol] | | WT SARS-CoV-2, deletion of 24 |
| | | nucleotides in furin cleavage |
| | | site (23598-23622) |

[symbol]

indicates data missing or illegible when filed

SUPPLEMENTARY TABLE 2
Primers: OTS primer and modifications introduced into the SARS-CoV-2 genome
OTS primers

| | | | | Amplified |
|---|---|---|---|---|
| | | | Primer | region |
| Fragment | Primer name | Primer sequence (5′-3′) | length | (bp) |
| Fg 2 nsp1_dm1 | WU-S-F | GTCTTATCAGAGGCACGTCAAC | 22 | 405 |
| (K164A and H165A) | | | | |
| | WU-124-R | TTCACGGGTAACACCACTGCTAGCAG | 48 | |
| | | CAGTGTTCCAGTTTTCTTGAAA | | |
| | WU-135-F | TTTTCAAGAAAACTGGAACACTGCTG | 49 | 2588 |
| | | CTAGCAGTGGTGTTACCCGTGAA | | |
| | WU-6-R | CTGGTGTAAGTTCCATCTCTAATTG | 25 | |
| Fg 11 Mutant | WU-23-F | GATGGCAACTAGCACTCTCC | 20 | 1622 |
| delORF6-8 | | | | |
| | CoV2mutdel6- | TTAGTTTGTTCGTTTAGATGAAATCT | 52 | |
| | 8-R | TACTGTACAAGCAAAGCAATATTGTC | | |
| | CoVZmutdel6- | CAATATTGCTTTGCTTGTACAGTAAG | 56 | 557 |
| | 8-F | ATTTCATCTAAACGAACAAACTAAAA | | |
| | | TGTC | | |
| | WU-24-R | TTTGGCAATGTTGTTCCTTGAGG | 23 | |
| Fg 9&10 | WU-19-F | GGAGTCACATTAATTGGAGAAGC | 23 | 3488 |
| Bristol del | | | | |
| | CoV2-Sp-bristol | TAGGCAATGATGGATTGACTAGCTAT | 50 | |
| | del-R | AGTCTGAGTCTGATAACTAGCGCA | | |
| | CoV2-Sp-bristol | TGCGCTAGTTATCAGACTCAGACTAT | 50 | 2319 |
| | del-F | AGCTAGTCAATCCATCATTGCCTA | | |
| | WU-22-R | TCATGTTCAGAAATAGGACTTGTTG | 25 | |

Primers: RTqPCR primer for the detection of viral genome
RTPCR primers

| | | | | Amplified |
|---|---|---|---|---|
| | | | Primer | region |
| Gene | Primer name | Primer sequence (5′-3′) | length | (bp) |
| At IVI: | | | | |
| E (forward) | PWhSF-E-F21 | ACAGGTACGTTAATAGTTA | 32 | 116 |
| | | ATAGCGTACTTCT | | |
| E (reverse) | PWhSF-R-22 | ACAATATTGCAGCAGTACGCACA | 23 | |
| E (probe) | PWhSF-E-P23mgb | ATCCTTACTGCGCTTCGA | 18 | |
| At FLI: | | | | |
| RdRp gene/nCoV_IP4 | Reference: National Refence Center for Respiratory | | | |
| | Viruses, Institut Pasteur, Paris. | | | |
| nCoV_IP4-14059Fw | | GGTAACTGGTATGATTTCG | 19 | 107 |
| nCoV_IP4-14146Rv | | CTGGTCAAGGTTAATATAGG | 20 | |
| nCoV_IP4-14084 | | TCATACAAACCACGCCAGG[5′] | 19 | |
| Probe(+) | | Fam[3′]BHQ-1 | | |

SUPPLEMENTARY TABLE 3
Modifications introduced into the SARS-CoV-2 genome
OTS changes introduced into SARS CoV-2 genome

| | | Amino Acid | | | | CDS Codon |
|---|---|---|---|---|---|---|
| OTS | Length | Change | CDS | Change | Codon Change | Number |
| Fg 2 - OTS | 1 | — | polyprotein lab CDS | C −> T | CTG −> TTG | 88 |
| Fg 2 - OTS | 2 | — | polyprotein lab CDS | CTC −> TTG | CTC −> TTG | 92 |
| Fg 2 - OTS | 3 | — | polyprotein lab CDS | AGT −> TCA | AGT −> TCA | 100 |
| Fg 2 - OTS | 2 | — | polyprotein lab CDS | CTT −> TTA | CTT −> TTA | 104 |
| Fg 2 - OTS | 2 | — | polyprotein lab CDS | CTT −> TTA | CTT −> TTA | 107 |
| Fg 2 - OTS | 2 | — | polyprotein lab CDS | CTT −> TTA | CTT −> TTA | 122 |
| Fg 2 - OTS | 2 | — | polyprotein lab CDS | CTT −> TTA | CTT −> TTA | 123 |
| Fg 2 - OTS | 1 | — | polyprotein lab CDS | C −> T | CTA −> TTA | 140 |
| Fg 2 - OTS | 2 | — | polyprotein lab CDS | CTT −> TTA | CTT −> TTA | 149 |
| Fg 2 - OTS | 3 | — | polyprotein lab CDS | AGC −> TCG | AGC −> TCG | 166 |
| Fg 2 - OTS | 3 | — | polyprotein lab CDS | AGT −> TCA | AGT −> TCA | 167 |
| Fg 2 - OTS | 2 | — | polyprotein lab CDS | CTC −> TTG | CTC −> TTG | 173 |
| Fg 2 - OTS | 2 | — | polyprotein lab CDS | CTT −> TTA | CTT −> TTA | 177 |
| Fg 2 - OTS | 2 | — | polyprotein lab CDS | CTT −> TTA | CTT −> TTA | 198 |
| Fg 2 - OTS | 2 | — | polyprotein lab CDS | CTT −> TTA | CTT −> TTA | 204 |
| Fg 2 - OTS | 1 | — | polyprotein lab CDS | C −> T | CTA −> TTA | 205 |
| Fg 2 - OTS | 1 | — | polyprotein lab CDS | C −> T | CTG −> TTG | 219 |
| Fg 2 - OTS | 1 | — | polyprotein lab CDS | T −> A | TCT −> TCA | 245 |
| Fg 2 - OTS | 3 | — | polyprotein lab CDS | AGC −> TCG | AGC −> TCG | 248 |
| Fg 2 - OTS | 1 | — | polyprotein lab CDS | C −> G | TCC −> TCG | 279 |
| Fg 2 - OTS | 2 | — | polyprotein lab CDS | CTT −> TTA | CTT −> TTA | 293 |
| Fg 2 - OTS | 1 | — | polyprotein lab CDS | T −> A | TCT −> TCA | 302 |
| Fg 2 - OTS | 2 | — | polyprotein lab CDS | CTC −> TTG | CTC −> TTG | 320 |
| Fg 2 - OTS | 3 | — | polyprotein lab CDS | AGT −> TCA | AGT −> TCA | 383 |
| Fg 2 - OTS | 1 | — | polyprotein lab CDS | T −> A | TCT −> TCA | 391 |
| Fg 2 - OTS | 2 | — | polyprotein lab CDS | CTT −> TTA | CTT −> TTA | 397 |
| Fg 2 - OTS | 1 | — | polyprotein lab CDS | T −> A | TCT −> TCA | 412 |
| Fg 2 - OTS | 3 | — | polyprotein lab CDS | AGC −> TCG | AGC −> TCG | 428 |
| Fg 2 - OTS | 1 | — | polyprotein lab CDS | C −> G | TCC −> TCG | 443 |
| Fg 2 - OTS | 2 | — | polyprotein lab CDS | CTT −> TTA | CTT −> TTA | 446 |
| Fg 2 - OTS | 2 | — | polyprotein lab CDS | CTT −> TTA | CTT −> TTA | 450 |
| Fg 2 - OTS | 2 | — | polyprotein lab CDS | CTT −> TTA | CTT −> TTA | 451 |
| Fg 2 - OTS | 2 | — | polyprotein lab CDS | CTC −> TTG | CTC −> TTG | 454 |
| Fg 2 - OTS | 2 | — | polyprotein lab CDS | CTT −> TTA | CTT −> TTA | 469 |
| Fg 2 - OTS | 1 | — | polyprotein lab CDS | T −> A | TCT −> TCA | 479 |
| Fg 2 - OTS | 1 | — | polyprotein lab CDS | T −> A | TCT −> TCA | 481 |
| Fg 2 - OTS | 1 | — | polyprotein lab CDS | C −> G | TCC −> TCG | 483 |
| Fg 2 - OTS | 3 | — | polyprotein lab CDS | AGT −> TCA | AGT −> TCA | 485 |
| Fg 2 - OTS | 1 | — | polyprotein lab CDS | C −> G | TCC −> TCG | 505 |
| Fg 2 - OTS | 1 | — | polyprotein lab CDS | C −> T | CTG −> TTG | 530 |
| Fg 2 - OTS | 3 | — | polyprotein lab CDS | AGT −> TCA | AGT −> TCA | 531 |
| Fg 2 - OTS | 2 | — | polyprotein lab CDS | CTT −> TTG | CTT −> TTG | 533 |
| Fg 2 - OTS | 1 | — | polyprotein lab CDS | C −> G | TCC −> TCG | 549 |
| Fg 2 - OTS | 2 | — | polyprotein lab CDS | CTT −> TTA | CTT −> TTA | 552 |
| Fg 2 - OTS | 1 | — | polyprotein lab CDS | T −> A | TCT −> TCA | 558 |
| Fg 2 - OTS | 1 | — | polyprotein lab CDS | C −> T | CTA −> TTA | 570 |
| Fg 2 - OTS | 1 | — | polyprotein lab CDS | C −> T | CTG −> TTG | 578 |
| Fg 2 - OTS | 2 | — | polyprotein lab CDS | CTC −> TTG | CTC −> TTG | 580 |
| Fg 2 - OTS | 1 | — | polyprotein lab CDS | T −> A | TCT −> TCA | 588 |
| Fg 2 - OTS | 1 | — | polyprotein lab CDS | C −> T | CTA −> TTA | 595 |
| Fg 2 - OTS | 1 | — | polyprotein lab CDS | C −> T | CTA −> TTA | 613 |
| Fg 2 - OTS | 2 | — | polyprotein lab CDS | CTC −> TTG | CTC −> TTG | 624 |
| Fg 2 - OTS | 2 | — | polyprotein lab CDS | CTT −> TTA | CTT −> TTA | 628 |
| Fg 2 - OTS | 2 | — | polyprotein lab CDS | CTT −> TTA | CTT −> TTA | 631 |
| Fg 2 - OTS | 2 | — | polyprotein lab CDS | CTT −> TTA | CTT −> TTA | 642 |
| Fg 2 - OTS | 3 | — | polyprotein lab CDS | AGT −> TCA | AGT −> TCA | 674 |
| Fg 2 - OTS | [symbol] | — | polyprotein lab CDS | CTT −> TTA | CTT −> TTA | 681 |
| Fg 2 - OTS | 1 | — | polyprotein lab CDS | T −> A | TCT −> TCA | 692 |
| Fg 2 - OTS | [symbol] | — | polyprotein lab CDS | CTT −> TTA | CTT −> TTA | 700 |
| Fg 2 - OTS | 1 | — | polyprotein lab CDS | C −> G | TCC −> TCG | 723 |
| Fg 2 - OTS | 1 | — | polyprotein lab CDS | C −> T | CTA −> TTA | 729 |
| Fg 2 - OTS | [symbol] | — | polyprotein lab CDS | CTC −> TTG | CTC −> TTG | 730 |
| Fg 2 - OTS | 1 | — | polyprotein lab CDS | C −> T | CTA −> TTA | 733 |
| Fg 2 - OTS | 2 | — | polyprotein lab CDS | CTT −> TTA | CTT −> TTA | 747 |
| Fg 2 - OTS | 3 | — | polyprotein lab CDS | AGT −> TCA | AGT −> TCA | 771 |
| Fg 2 - OTS | 2 | — | polyprotein lab CDS | CTT −> TTA | CTT −> TTA | 788 |
| Fg 2 - OTS | 2 | — | polyprotein lab CDS | CTC −> TTG | CTC −> TTG | 791 |
| Fg 2 - OTS | 2 | — | polyprotein lab CDS | CTC −> TTG | CTC −> TTG | 815 |
| Fg 2 - OTS | 3 | — | polyprotein lab CDS | AGT −> TCA | AGT −> TCA | 838 |
| Fg 2 - OTS | 2 | — | polyprotein lab CDS | CTT −> TTA | CTT −> TTA | 845 |
| Fg 2 - OTS | 2 | — | polyprotein lab CDS | CTT −> TTA | CTT −> TTA | 853 |
| Fg 2 - OTS | 1 | — | polyprotein lab CDS | T −> A | TCT −> TCA | 858 |
| Fg 2 - OTS | 2 | — | polyprotein lab CDS | CTC −> TTG | CTC −> TTG | 864 |
| Fg 2 - OTS | 1 | — | polyprotein lab CDS | T −> A | TCT −> TCA | 887 |
| Fg 2 - OTS | 1 | — | polyprotein lab CDS | C −> T | CTG −> TTG | 893 |
| Fg 2 - OTS | 3 | — | polyprotein lab CDS | AGT −> TCA | AGT −> TCA | 901 |
| Fg 2 - OTS | 1 | — | polyprotein lab CDS | T −> A | TCT −> TCA | 911 |
| Total Fg 2 - OTS | 1 [symbol] | | | | Total codon changes Fg 2 = [symbol] 7 | |
| Fg 4 - OTS | 1 | — | polyprotein lab CDS | C −> T | CTG −> TTG | 2028 |
| Fg 4 - OTS | 2 | — | polyprotein lab CDS | CTT −> TTA | CTT −> TTA | 2039 |
| Fg 4 - OTS | 1 | — | polyprotein lab CDS | C −> T | CTA −> TTA | 2044 |
| Fg 4 - OTS | 1 | — | polyprotein lab CDS | T −> A | TCT −> TCA | 2048 |
| Fg 4 - OTS | 2 | — | polyprotein lab CDS | CTT −> TTA | CTT −> TTA | 2062 |
| Fg 4 - OTS | 2 | — | polyprotein lab CDS | CTT −> TTA | CTT −> TTA | 2077 |
| Fg 4 - OTS | 3 | — | polyprotein lab CDS | AGT −> TCA | AGT −> TCA | 2083 |
| Fg 4 - OTS | 1 | — | polyprotein lab CDS | C −> T | CTA −> TTA | 2095 |
| Fg 4 - OTS | 1 | — | polyprotein lab CDS | T −> A | TCT −> TCA | 2103 |
| Fg 4 - OTS | 3 | — | polyprotein lab CDS | AGT −> TCA | AGT −> TCA | 2104 |
| Fg 4 - OTS | 1 | — | polyprotein lab CDS | T −> A | TCT −> TCA | 2114 |
| Fg 4 - OTS | 2 | — | polyprotein lab CDS | CTT −> TTA | CTT −> TTA | 2122 |
| Fg 4 - OTS | 3 | — | polyprotein lab CDS | AGT −> TCA | AGT −> TCA | 2132 |
| Fg 4 - OTS | 2 | — | polyprotein lab CDS | CTT −> TTA | CTT −> TTA | 2146 |
| Fg 4 - OTS | 3 | — | polyprotein lab CDS | AGT −> TCA | AGT −> TCA | 2151 |
| Fg 4 - OTS | 1 | — | polyprotein lab CDS | C −> T | CTA −> TTA | 2177 |
| Fg 4 - OTS | 3 | — | polyprotein lab CDS | AGT −> TCA | AGT −> TCA | 2185 |
| Fg 4 - OTS | 1 | — | polyprotein lab CDS | T −> A | TCT −> TCA | 2188 |
| Fg 4 - OTS | 1 | — | polyprotein lab CDS | T −> A | TCT −> TCA | 2193 |
| Fg 4 - OTS | 3 | — | polyprotein lab CDS | AGT −> TCA | AGT −> TCA | 2205 |
| Fg 4 - OTS | 1 | — | polyprotein lab CDS | C −> T | CTA −> TTA | 2211 |
| Fg 4 - OTS | 1 | — | polyprotein lab CDS | T −> A | TCT −> TCA | 2224 |
| Fg 4 - OTS | 1 | — | polyprotein lab CDS | C −> T | CTG −> TTG | 2226 |
| Fg 4 - OTS | 1 | — | polyprotein lab CDS | C −> T | CTA −> TTA | 2235 |
| Fg 4 - OTS | 3 | — | polyprotein lab CDS | AGT −> TCA | AGT −> TCA | 2237 |
| Fg 4 - OTS | 1 | — | polyprotein lab CDS | C −> T | CTA −> TTA | 2240 |
| Fg 4 - OTS | 1 | — | polyprotein lab CDS | T −> A | TCT −> TCA | 2242 |
| Fg 4 - OTS | 1 | — | polyprotein lab CDS | T −> A | TCT −> TCA | 2255 |
| Fg 4 - OTS | 1 | — | polyprotein lab CDS | T −> A | TCT −> TCA | 2261 |
| Fg 4 - OTS | 1 | — | polyprotein lab CDS | T −> A | TCT −> TCA | 2273 |
| Fg 4 - OTS | 1 | — | polyprotein lab CDS | T −> A | TCT −> TCA | 2285 |
| Fg 4 - OTS | 3 | — | polyprotein lab CDS | AGT −> TCA | AGT −> TCA | 2289 |
| Fg 4 - OTS | 2 | — | polyprotein lab CDS | CTT −> TTA | CTT −> TTA | 2292 |
| Fg 4 - OTS | 3 | — | polyprotein lab CDS | AGT −> TCA | AGT −> TCA | 2293 |
| Fg 4 - OTS | 1 | — | polyprotein lab CDS | T −> A | TCT −> TCA | 2297 |
| Fg 4 - OTS | 1 | — | polyprotein lab CDS | T −> A | TCT −> TCA | 2303 |
| Fg 4 - OTS | 1 | — | polyprotein lab CDS | T −> A | TCT −> TCA | 2313 |
| Fg 4 - OTS | 2 | — | polyprotein lab CDS | CTT −> TTA | CTT −> TTA | 2333 |
| Fg 4 - OTS | 2 | — | polyprotein lab CDS | CTT −> TTA | CTT −> TTA | 2341 |
| Fg 4 - OTS | 3 | — | polyprotein lab CDS | AGC −> TCG | AGC −> TCG | 2352 |
| Fg 4 - OTS | 3 | — | polyprotein lab CDS | AGT −> TCA | AGT −> TCA | 2360 |
| Fg 4 - OTS | 1 | — | polyprotein lab CDS | T −> A | TCT −> TCA | 2362 |
| Fg 4 - OTS | 2 | — | polyprotein lab CDS | CTT −> TTA | CTT −> TTA | 2364 |
| Fg 4 - OTS | 2 | — | polyprotein lab CDS | CTT −> TTA | CTT −> TTA | 2371 |
| Fg 4 - OTS | 3 | — | polyprotein lab CDS | AGT −> TCA | AGT −> TCA | 2396 |
| Fg 4 - OTS | 1 | — | polyprotein lab CDS | C −> G | TCC −> TCG | 2433 |
| Fg 4 - OTS | 1 | — | polyprotein lab CDS | C −> T | CTA −> TTA | 2447 |
| Fg 4 - OTS | 3 | — | polyprotein lab CDS | AGT −> TCA | AGT −> TCA | 2462 |
| Fg 4 - OTS | 3 | — | polyprotein lab CDS | AGT −> TCA | AGT −> TCA | 2466 |
| Fg 4 - OTS | 1 | — | polyprotein lab CDS | C −> T | CTA −> TTA | 2475 |
| Fg 4 - OTS | 1 | — | polyprotein lab CDS | T −> A | TCT −> TCA | 2487 |
| Fg 4 - OTS | 1 | — | polyprotein lab CDS | T −> A | TCT −> TCA | 2488 |
| Fg 4 - OTS | 3 | — | polyprotein lab CDS | AGT −> TCA | AGT −> TCA | 2493 |
| Fg 4 - OTS | 1 | — | polyprotein lab CDS | C −> G | TCC −> TCG | 2500 |
| Fg 4 - OTS | 2 | — | polyprotein lab CDS | CTT −> TTA | CTT −> TTA | 2503 |
| Fg 4 - OTS | 1 | — | polyprotein lab CDS | T −> A | TCT −> TCA | 2517 |
| Fg 4 - OTS | 2 | — | polyprotein lab CDS | CTC −> TTG | CTC −> TTG | 2518 |
| Fg 4 - OTS | 1 | — | polyprotein lab CDS | T −> A | TCT −> TCA | 2519 |
| Fg 4 - OTS | 1 | — | polyprotein lab CDS | C −> T | CTG −> TTG | 2527 |
| Fg 4 - OTS | 1 | — | polyprotein lab CDS | T −> A | TCT −> TCA | 2553 |
| Fg 4 - OTS | 1 | — | polyprotein lab CDS | T −> A | TCT −> TCA | 2558 |
| Fg 4 - OTS | 2 | — | polyprotein lab CDS | CTT −> TTA | CTT −> TTA | 2564 |
| Fg 4 - OTS | 1 | — | polyprotein lab CDS | C −> T | CTG −> TTG | 2570 |
| Fg 4 - OTS | 1 | — | polyprotein lab CDS | C −> T | CTA −> TTA | 2572 |
| Fg 4 - OTS | 1 | — | polyprotein lab CDS | T −> A | TCT −> TCA | 2578 |
| Fg 4 - OTS | 3 | — | polyprotein lab CDS | AGT −> TCA | AGT −> TCA | 2583 |
| Fg 4 - OTS | 2 | — | polyprotein lab CDS | CTC −> TTG | CTC −> TTG | 2609 |
| Fg 4 - OTS | 1 | — | polyprotein lab CDS | C −> T | CTA −> TTA | 2612 |
| Fg 4 - OTS | 2 | — | polyprotein lab CDS | CTT −> TTA | CTT −> TTA | 2620 |
| Fg 4 - OTS | 1 | — | polyprotein lab CDS | C −> G | TCC −> TCG | 2625 |
| Fg 4 - OTS | 1 | — | polyprotein lab CDS | T −> A | TCT −> TCA | 2631 |
| Fg 4 - OTS | 2 | — | polyprotein lab CDS | CTT −> TTA | CTT −> TTA | 2655 |
| Fg 4 - OTS | 1 | — | polyprotein lab CDS | T −> A | TCT −> TCA | 2661 |
| Fg 4 - OTS | 3 | — | polyprotein lab CDS | AGT −> TCA | AGT −> TCA | 2669 |
| Fg 4 - OTS | 2 | — | polyprotein lab CDS | CTC −> TTG | CTC −> TTG | 2675 |
| Fg 4 - OTS | 2 | — | polyprotein lab CDS | CTT −> TTA | CTT −> TTA | 2688 |
| Fg 4 - OTS | 3 | — | polyprotein lab CDS | AGT −> TCA | AGT −> TCA | 2695 |
| Fg 4 - OTS | 3 | — | polyprotein lab CDS | AGT −> TCA | AGT −> TCA | 2706 |
| Fg 4 - OTS | 1 | — | polyprotein lab CDS | T −> A | TCT −> TCA | 2722 |
| Fg 4 - OTS | 1 | — | polyprotein lab CDS | C −> T | CTA −> TTA | 2725 |
| Fg 4 - OTS | 3 | — | polyprotein lab CDS | AGT −> TCA | AGT −> TCA | 2731 |
| Fg 4 - OTS | 2 | — | polyprotein lab CDS | CTT −> TTA | CTT −> TTA | 2760 |
| Fg 4 - OTS | 2 | — | polyprotein lab CDS | CTT −> TTA | CTT −> TTA | 2778 |
| Fg 4 - OTS | 2 | — | polyprotein lab CDS | CTT −> TTA | CTT −> TTA | 2781 |
| Fg 4 - OTS | 1 | — | polyprotein lab CDS | T −> A | TCT −> TCA | 2797 |
| Fg 4 - OTS | 3 | — | polyprotein lab CDS | AGT −> TCA | AGT −> TCA | 2804 |
| Total Fg 4 - OTS | 149 | | | | Total codon changes Fg 4 = [symbol] 6 | |
| Fg 5 - OTS | 1 | — | polyprotein lab CDS | T −> A | TCT −> TCA | 2926 |
| Fg 5 - OTS | 1 | — | polyprotein lab CDS | C −> T | CTA −> TTA | 293 [symbol] |
| Fg 5 - OTS | 3 | — | polyprotein lab CDS | T −> A | TCT −> TCA | 2942 |
| Fg 5 - OTS | 2 | — | polyprotein lab CDS | AGT −> TCA | AGT −> TCA | 2947 |
| Fg 5 - OTS | 1 | — | polyprotein lab CDS | CTC −> TTG | CTC −> TTG | 2956 |
| Fg 5 - OTS | 2 | — | polyprotein lab CDS | T −> A | TCT −> TCA | 2960 |
| Fg 5 - OTS | 1 | — | polyprotein lab CDS | CTT −> TTA | CTT −> TTA | 2969 |
| Fg 5 - OTS | 1 | — | polyprotein lab CDS | T −> A | TCT −> TCA | 2972 |
| Fg 5 - OTS | 1 | — | polyprotein lab CDS | T −> A | TCT −> TCA | 23 [symbol] 1 |
| Fg 5 - OTS | 3 | — | polyprotein lab CDS | T −> A | TCT −> TCA | 2999 |
| Fg 5 - OTS | 2 | — | polyprotein lab CDS | AGT −> TCA | AGT −> TCA | 3001 |
| Fg 5 - OTS | 1 | — | polyprotein lab CDS | CTT −> TTA | CTT −> TTA | 3006 |
| Fg 5 - OTS | 2 | — | polyprotein lab CDS | T −> A | TCT −> TCA | 3013 |
| Fg 5 - OTS | 1 | — | polyprotein lab CDS | CTT −> TTA | CTT −> TTA | 3027 |
| Fg 5 - OTS | 1 | — | polyprotein lab CDS | C −> T | CTA −> TTA | 3034 |
| Fg 5 - OTS | [symbol] | — | polyprotein lab CDS | T −> A | TCT −> TCA | 304 [symbol] |
| Fg 5 - OTS | 3 | — | polyprotein lab CDS | CTT −> TTA | CTT −> TTA | 3060 |
| Fg 5 - OTS | 1 | — | polyprotein lab CDS | AGT −> TCA | AGT −> TCA | 3075 |
| Fg 5 - OTS | 2 | — | polyprotein lab CDS | C −> T | CTA −> TTA | 3084 |
| Fg 5 - OTS | 2 | — | polyprotein lab CDS | CTT −> TTA | CTT −> TTA | 3086 |
| Fg 5 - OTS | [symbol] | — | polyprotein lab CDS | CTC −> TTG | CTC −> TTG | 3092 |
| Fg 5 - OTS | 1 | — | polyprotein lab CDS | T −> A | TCT −> TCA | 3106 |
| Fg 5 - OTS | 2 | — | polyprotein lab CDS | CTT −> TTA | CTT −> TTA | 3116 |
| Fg 5 - OTS | 1 | — | polyprotein lab CDS | T −> A | TCT −> TCA | 3121 |
| Fg 5 - OTS | 1 | — | polyprotein lab CDS | C −> G | TCC −> TCG | 3149 |
| Fg 5 - OTS | 3 | — | polyprotein lab CDS | AGT −> TCA | AGT −> TCA | 3158 |
| Fg 5 - OTS | 1 | — | polyprotein lab CDS | C −> T | CTA −> TTA | 3161 |
| Fg 5 - OTS | 1 | — | polyprotein lab CDS | C −> G | TCC −> TCG | 3171 |
| Fg 5 - OTS | 3 | — | polyprotein lab CDS | AGT −> TCA | AGT −> TCA | 3173 |
| Fg 5 - OTS | 1 | — | polyprotein lab CDS | C −> T | CTG −> TTG | 3180 |
| Fg 5 - OTS | 1 | — | polyprotein lab CDS | C −> T | CTA −> TTA | 3191 |
| Fg 5 - OTS | 3 | — | polyprotein lab CDS | AGT −> TCA | AGT −> TCA | 3195 |
| Fg 5 - OTS | 1 | — | polyprotein lab CDS | C −> T | CTA −> TTA | 3198 |
| Fg 5 - OTS | 2 | — | polyprotein lab CDS | CTT −> TTA | CTT −> TTA | 3201 |
| Fg 5 - OTS | 2 | — | polyprotein lab CDS | CTT −> TTA | CTT −> TTA | 3210 |
| Fg 5 - OTS | 3 | — | polyprotein lab CDS | AGT −> TCA | AGT −> TCA | 3218 |
| Fg 5 - OTS | 3 | — | polyprotein lab CDS | AGC −> TCG | AGC −> TCG | 3225 |
| Fg 5 - OTS | 2 | — | polyprotein lab CDS | CTC −> TTG | CTC −> TTG | 3234 |
| Fg 5 - OTS | 2 | — | polyprotein lab CDS | CTC −> TTG | CTC −> TTG | 3238 |
| Fg 5 - OTS | 3 | — | polyprotein lab CDS | AGT −> TCA | AGT −> TCA | 3742 |
| Fg 5 - OTS | 1 | — | polyprotein lab CDS | T −> A | TCT −> TCA | 3246 |
| Fg 5 - OTS | 2 | — | polyprotein lab CDS | CTT −> TTA | CTT −> TTA | 3249 |
| Fg 5 - OTS | 1 | — | polyprotein lab CDS | T −> A | TCT −> TCA | 3256 |
| Fg 5 - OTS | 3 | — | polyprotein lab CDS | AGT −> TCA | AGT −> TCA | 3264 |
| Fg 5 - OTS | 1 | — | polyprotein lab CDS | T −> A | TCT −> TCA | 3273 |
| Fg 5 - OTS | 2 | — | polyprotein lab CDS | CTT −> TTA | CTT −> TTA | 3290 |
| Fg 5 - OTS | 2 | — | polyprotein lab CDS | CTT −> TTA | CTT −> TTA | 329 [symbol] |
| Fg 5 - OTS | 2 | — | polyprotein lab CDS | CTT −> TTA | CTT −> TTA | 329 [symbol] |
| Fg 5 - OTS | 1 | — | polyprotein lab CDS | T −> A | TCT −> TCA | 3309 |
| Fg 5 - OTS | 2 | — | polyprotein lab CDS | CTT −> TTA | CTT −> TTA | 3313 |
| Fg 5 - OTS | 2 | — | polyprotein lab CDS | CTC −> TTG | CTC −> TTG | 3321 |
| Fg 5 - OTS | 1 | — | polyprotein lab CDS | T −> A | TCT −> TCA | 3325 |
| Fg 5 - OTS | 2 | — | polyprotein lab CDS | CTC −> TTG | CTC −> TTG | 3338 |
| Fg 5 - OTS | 1 | — | polyprotein lab CDS | T −> A | TCT −> TCA | 3344 |
| Fg 5 - OTS | 2 | — | polyprotein lab CDS | CTT −> TTA | CTT −> TTA | 3350 |
| Fg 5 - OTS | 2 | — | polyprotein lab CDS | CTT −> TTA | CTT −> TTA | 3352 |
| Fg 5 - OTS | 1 | — | polyprotein lab CDS | T −> A | TCT −> TCA | 33 [symbol] |
| Fg 5 - OTS | 2 | — | polyprotein lab CDS | CTT −> TTA | CTT −> TTA | 3404 |
| Fg 5 - OTS | 3 | — | polyprotein lab CDS | AGT −> TCA | AGT −> TCA | 3410 |
| Fg 5 - OTS | 1 | — | polyprotein lab CDS | T −> A | TCT −> TCA | 342 [symbol] |
| Fg 5 - OTS | 2 | — | polyprotein lab CDS | CTC −> TTG | CTC −> TTG | 3483 |
| Fg 5 - OTS | 2 | — | polyprotein lab CDS | CTT −> TTA | CTT −> TTA | 3490 |
| Fg 5 - OTS | 2 | — | polyprotein lab CDS | CTT −> TTA | CTT −> TTA | 3495 |
| Fg 5 - OTS | 1 | — | polyprotein lab CDS | C −> T | CTA −> TTA | 350 [symbol] |
| Fg 5 - OTS | 1 | — | polyprotein lab CDS | C −> T | CTA −> TTA | 3513 |
| Fg 5 - OTS | 2 | — | polyprotein lab CDS | CTT −> TTA | CTT −> TTA | 3516 |
| Fg 5 - OTS | 1 | — | polyprotein lab CDS | C −> T | CTG −> TTG | 3535 |
| Fg 5 - OTS | 3 | — | polyprotein lab CDS | AGT −> TCA | AGT −> TCA | 3547 |
| Fg 5 - OTS | 3 | — | polyprotein lab CDS | AGT −> TCA | AGT −> TCA | 3 [symbol] 70 |
| Fg 5 - OTS | 2 | — | polyprotein lab CDS | CTC −> TTG | CTC −> TTG | 3585 |
| Fg 5 - OTS | 2 | — | polyprotein lab CDS | CTT −> TTA | CTT −> TTA | 3591 |
| Fg 5 - OTS | 3 | — | polyprotein lab CDS | AGT −> TCA | AGT −> TCA | 3597 |
| Fg 5 - OTS | 1 | — | polyprotein lab CDS | T −> A | TCT −> TCA | 3601 |
| Fg 5 - OTS | 1 | — | polyprotein lab CDS | T −> A | TCT −> TCA | 3622 |
| Fg 5 - OTS | 2 | — | polyprotein lab CDS | CTC −> TTG | CTC −> TTG | 3636 |
| Fg 5 - OTS | 1 | — | polyprotein lab CDS | T −> A | TCT −> TCA | 3643 |
| Fg 5 - OTS | 2 | — | polyprotein lab CDS | CTT −> TTA | CTT −> TTA | 3644 |
| Fg 5 - OTS | 3 | — | polyprotein lab CDS | AGT −> TCA | AGT −> TCA | 3658 |
| Fg 5 - OTS | 3 | — | polyprotein lab CDS | AGT −> TCA | AGT −> TCA | 3673 |
| Fg 5 - OTS | 1 | — | polyprotein lab CDS | T −> A | TCT −> TCA | 367 [symbol] |
| Fg 5 - OTS | 1 | — | polyprotein lab CDS | C −> T | CTA −> TTA | 3 [symbol] |
| Fg 5 - OTS | 1 | — | polyprotein lab CDS | C −> T | CTA −> TTA | 3692 |
| Fg 5 - OTS | 2 | — | polyprotein lab CDS | CTT −> TTA | CTT −> TTA | 3694 |
| Fg 5 - OTS | 2 | — | polyprotein lab CDS | CTT −> TTA | CTT −> TTA | 3711 |
| Fg 5 - OTS | 2 | — | polyprotein lab CDS | CTC −> TTG | CTC −> TTG | 3717 |
| Fg 5 - OTS | 1 | — | polyprotein lab CDS | C −> G | TCC −> TCG | 37 [symbol] |
| Fg 5 - OTS | 2 | — | polyprotein lab CDS | CTT −> TTA | CTT −> TTA | 3736 |
| Fg 5 - OTS | 1 | — | polyprotein lab CDS | T −> A | TCT −> TCA | 3739 |
| Fg 5 - OTS | 1 | — | polyprotein lab CDS | T −> A | TCT −> TCA | 3742 |
| Fg 5 - OTS | 2 | — | polyprotein lab CDS | CTT −> TTA | CTT −> TTA | 3776 |
| Fg 5 - OTS | 1 | — | polyprotein lab CDS | C −> T | CTA −> TTA | 3781 |
| Fg 5 - OTS | 2 | — | polyprotein lab CDS | CTC −> TTG | CTC −> TTG | 3796 |
| Total Fg 5 - OTS | 1 [symbol] 0 | | | | Total codon changes Fg 5 = [symbol] | |
| Fg 7 - OTS | 2 | — | polyprotein lab CDS | CTT −> TTA | CTT −> TTA | 4793 |
| Fg 7 - OTS | 1 | — | polyprotein lab CDS | T −> A | TCT −> TCA | 4817 |
| Fg 7 - OTS | 3 | — | polyprotein lab CDS | AGT −> TCA | AGT −> TCA | 4825 |
| Fg 7 - OTS | 1 | — | polyprotein lab CDS | T −> A | TCT −> TCA | 4826 |
| Fg 7 - OTS | 3 | — | polyprotein lab CDS | AGC −> TCG | AGC −> TCG | 4843 |
| Fg 7 - OTS | 1 | — | polyprotein lab CDS | C −> T | CTA −> TTA | 4852 |
| Fg 7 - OTS | 1 | — | polyprotein lab CDS | C −> T | CTA −> TTA | 4861 |
| Fg 7 - OTS | 1 | — | polyprotein lab CDS | C −> T | CTA −> TTA | 4862 |
| Fg 7 - OTS | 1 | — | polyprotein lab CDS | C −> T | CTA −> TTA | 4890 |
| Fg 7 - OTS | 2 | — | polyprotein lab CDS | CTT −> TTA | CTT −> TTA | 4906 |
| Fg 7 - OTS | 3 | — | polyprotein lab CDS | AGT −> TCA | AGT −> TCA | 4912 |
| Fg 7 - OTS | 2 | — | polyprotein lab CDS | CTT −> TTA | CTT −> TTA | 4919 |
| Fg 7 - OTS | 2 | — | polyprotein lab CDS | CTT −> TTA | CTT −> TTA | 4936 |
| Fg 7 - OTS | 3 | — | polyprotein lab CDS | AGT −> TCA | AGT −> TCA | 4941 |
| Fg 7 - OTS | 1 | — | polyprotein lab CDS | T −> A | TCT −> TCA | 4953 |
| Fg 7 - OTS | 3 | — | polyprotein lab CDS | AGT −> TCA | AGT −> TCA | 4956 |
| Fg 7 - OTS | 3 | — | polyprotein lab CDS | AGC −> TCG | AGC −> TCG | 4984 |
| Fg 7 - OTS | 3 | — | polyprotein lab CDS | AGT −> TCA | AGT −> TCA | 4999 |
| Fg 7 - OTS | 2 | — | polyprotein lab CDS | CTT −> TTA | CTT −> TTA | 5006 |
| Fg 7 - OTS | 2 | — | polyprotein lab CDS | CTT −> TTA | CTT −> TTA | 5022 |
| Fg 7 - OTS | 2 | — | polyprotein lab CDS | CTT −> TTA | CTT −> TTA | 5028 |
| Fg 7 - OTS | 2 | — | polyprotein lab CDS | CTT −> TTA | CTT −> TTA | 5030 |
| Fg 7 - OTS | 3 | — | polyprotein lab CDS | AGC −> TCG | AGC −> TCG | 5039 |
| Fg 7 - OTS | 3 | — | polyprotein lab CDS | AGT −> TCA | AGT −> TCA | 5056 |
| Fg 7 - OTS | 1 | — | polyprotein lab CDS | C −> T | CTA −> TTA | 5065 |
| Fg 7 . OTS | 3 | — | polyprotein lab CDS | AGT −> TCA | AGT −> TCA | 5084 |
| Fg 7 - OTS | 2 | — | polyprotein lab CDS | CTT −> TTA | CTT −> TTA | 5099 |
| Fg 7 - OTS | 1 | — | polyprotein lab CDS | T −> A | TCT −> TCA | 5101 |
| Fg 7 - OTS | 2 | — | polyprotein lab CDS | CTT −> TTA | CTT −> TTA | 5119 |
| Fg 7 - OTS | 2 | — | polyprotein lab CDS | CTC −> TTG | CTC −> TTG | 5123 |
| Fg 7 - OTS | 2 | — | polyprotein lab CDS | CTC −> TTG | CTC −> TTG | 5150 |
| Fg 7 - OTS | 1 | — | polyprotein lab CDS | T −> A | TCT −> TCA | 5151 |
| Fg 7 - OTS | 3 | — | polyprotein lab CDS | AGC −> TCG | AGC −> TCG | 5160 |
| Fg 7 - OTS | 1 | — | polyprotein lab CDS | T −> A | TCT −> TCA | 5164 |
| Fg 7 - OTS | 1 | — | polyprotein lab CDS | C −> T | CTA −> TTA | 5167 |
| Fg 7 - OTS | 3 | — | polyprotein lab CDS | AGC −> TCG | AGC −> TCG | 5170 |
| Fg 7 - OTS | 2 | — | polyprotein lab CDS | CTT −> TTA | CTT −> TTA | 5178 |
| Fg 7 - OTS | 1 | — | polyprotein lab CDS | T −> A | TCT −> TCA | 5187 |
| Fg 7 - OTS | 2 | — | polyprotein lab CDS | CTT −> TTA | CTT −> TTA | 5197 |
| Fg 7 - OTS | 1 | — | polyprotein lab CDS | T −> A | TCT −> TCA | 5206 |
| Fg 7 - OTS | 1 | — | polyprotein lab CDS | C −> T | CTA −> TTA | 5211 |
| Fg 7 - OTS | 2 | — | polyprotein lab CDS | CTT −> TTA | CTT −> TTA | 5221 |
| Fg 7 - OTS | 1 | — | polyprotein lab CDS | C −> T | CTA −> TTA | 5230 |
| Fg 7 - OTS | 2 | — | polyprotein lab CDS | CTT −> TTA | CTT −> TTA | 5246 |
| Fg 7 - OTS | 1 | — | polyprotein lab CDS | T −> A | TCT −> TCA | 5253 |
| Fg 7 - OTS | 2 | — | polyprotein lab CDS | CTT −> TTA | CTT −> TTA | 5261 |
| Fg 7 - OTS | 1 | — | polyprotein lab CDS | C −> T | CTA −> TTA | 5283 |
| Fg 7 - OTS | 1 | — | polyprotein lab CDS | T −> A | TCT −> TCA | 5296 |
| Fg 7 - OTS | 2 | — | polyprotein lab CDS | CTT −> TTA | CTT −> TTA | 5299 |
| Fg 7 - OTS | 2 | — | polyprotein lab CDS | CTT −> TTA | CTT −> TTA | 5331 |
| Fg 7 - OTS | 1 | — | polyprotein lab CDS | T −> A | TCT −> TCA | 5368 |
| Fg 7 - OTS | 2 | — | polyprotein lab CDS | CTT −> TTA | CTT −> TTA | 5387 |
| Fg 7 - OTS | 3 | — | polyprotein lab CDS | AGC −> TCG | AGC −> TCG | 5393 |
| Fg 7 - OTS | 3 | — | polyprotein lab CDS | AGC −> TCG | AGC −> TCG | 5424 |
| Fg 7 - OTS | 2 | — | polyprotein lab CDS | CTC −> TTG | CTC −> TTG | 5454 |
| Fg 7 - OTS | 2 | — | polyprotein lab CDS | CTT −> TTA | CTT −> TTA | 5456 |
| Fg 7 - OTS | 2 | — | polyprotein lab CDS | CTC −> TTG | CTC −> TTG | 5462 |
| Fg 7 - OTS | 1 | — | polyprotein lab CDS | C −> T | CTG −> TTG | 5471 |
| Fg 7 - OTS | 1 | — | polyprotein lab CDS | T −> A | TCT −> TCA | 5472 |
| Fg 7 - OTS | 1 | — | polyprotein lab CDS | C −> T | CTG −> TTG | 5482 |
| Fg 7 - OTS | 1 | — | polyprotein lab CDS | T −> A | TCT −> TCA | 5483 |
| Fg 7 - OTS | 2 | — | polyprotein lab CDS | CTT −> TTA | CTT −> TTA | 5489 |
| Fg 7 - OTS | 2 | — | polyprotein lab CDS | CTT −> TTA | CTT −> TTA | 5500 |
| Fg 7 . OTS | 3 | — | polyprotein lab CDS | AGT −> TCA | AGT −> TCA | 5515 |
| Fg 7 - OTS | 1 | — | polyprotein lab CDS | C −> T | CTG −> TTG | 5551 |
| Fg 7 - OTS | 3 | — | polyprotein lab CDS | AGT −> TCA | AGT −> TCA | 5560 |
| Fg 7 - OTS | 1 | — | polyprotein lab CDS | C −> T | CTA −> TTA | 5564 |
| Fg 7 - OTS | 2 | — | polyprotein lab CDS | CTC −> TTG | CTC −> TTG | 5580 |
| Fg 7 - OTS | 1 | — | polyprotein lab CDS | T −> A | TCT −> TCA | 5587 |
| Fg 7 - OTS | 3 | — | polyprotein lab CDS | AGC −> TCG | AGC −> TCG | 5588 |
| Fg 7 - OTS | 1 | — | polyprotein lab CDS | T −> A | TCT −> TCA | 5602 |
| Fg 7 - OTS | 2 | — | polyprotein lab CDS | CTC −> TTA | CTC −> TTA | 5604 |
| Fg 7 - OTS | 3 | — | polyprotein lab CDS | AGT −> TCA | AGT −> TCA | 5613 |
| Fg 7 - OTS | 1 | — | polyprotein lab CDS | C −> T | CTA −> TTA | 5619 |
| Fg 7 - OTS | 2 | — | polyprotein lab CDS | CTC −> TTG | CTC −> TTG | 5621 |
| Fg 7 - OTS | 1 | — | polyprotein lab CDS | T −> A | TCT −> TCA | 5625 |
| Fg 7 . OTS | 1 | — | polyprotein lab CDS | T −> A | TCT −> TCA | 5634 |
| Fg 7 - OTS | 1 | — | polyprotein lab CDS | C −> T | CTA −> TTA | 5641 |
| Fg 7 - OTS | 3 | — | polyprotein lab CDS | AGT −> TCA | AGT −> TCA | 5655 |
| Fg 7 - OTS | 3 | — | polyprotein lab CDS | AGT −> TCA | AGT −> TCA | 5709 |
| Total Fg 7 - OTS | 14 [symbol] | | | | Total codon changes Fg 7 = 80 | |
| Fg 8 - OTS | 2 | — | polyprotein lab CDS | CTT −> TTA | CTT −> TTA | 5824 |
| Fg 8 - OTS | 1 | — | polyprotein lab CDS | C −> T | CTA −> TTA | 5852 |
| Fg 8 - OTS | 1 | — | polyprotein lab CDS | T −> A | TCT −> TCA | 5879 |
| Fg 8 - OTS | 2 | — | polyprotein lab CDS | CTT −> TTA | CTT −> TTA | 5897 |
| Fg 8 - OTS | 1 | — | polyprotein lab CDS | T −> A | TCT −> TCA | 5901 |
| Fg 8 - OTS | 2 | — | polyprotein lab CDS | CTT −> TTA | CTT −> TTA | 5905 |
| Fg 8 - OTS | 3 | — | polyprotein lab CDS | AGT −> TCA | AGT −> TCA | 5913 |
| Fg 8 - OTS | 2 | — | polyprotein lab CDS | CTT −> TTA | CTT −> TTA | 5914 |
| Fg 8 - OTS | 2 | — | polyprotein lab CDS | CTC −> TTG | CTC −> TTG | 5932 |
| Fg 8 - OTS | 3 | — | polyprotein lab CDS | AGT −> TCA | AGT −> TCA | 5937 |
| Fg 8 - OTS | 2 | — | polyprotein lab CDS | CTC −> TTG | CTC −> TTG | 5952 |
| Fg 8 - OTS | 3 | — | polyprotein lab CDS | AGT −> TCA | AGT −> TCA | 5953 |
| Fg 8 - OTS | 2 | — | polyprotein lab CDS | CTC −> TTG | CTC −> TTG | 5979 |
| Fg 8 - OTS | 1 | — | polyprotein lab CDS | T −> A | TCT −> TCA | 5981 |
| Fg 8 - OTS | 1 | — | polyprotein lab CDS | C −> T | CTA −> TTA | 6034 |
| Fg 8 - OTS | 1 | — | polyprotein lab CDS | T −> A | TCT −> TCA | 6037 |
| Fg 8 - OTS | 1 | — | polyprotein lab CDS | C −> T | CTA −> TTA | 6042 |
| Fg 8 - OTS | 1 | — | polyprotein lab CDS | C −> G | TCC −> TCG | 5059 |
| Fg 8 - OTS | 3 | — | polyprotein lab CDS | AGT −> TCA | AGT −> TCA | 6062 |
| Fg 8 - OTS | 2 | — | polyprotein lab CDS | CTC −> TTG | CTC −> TTG | 6074 |
| Fg 8 - OTS | 2 | — | polyprotein lab CDS | CTT −> TTA | CTT −> TTA | 6077 |
| Fg 8 - OTS | 2 | — | polyprotein lab CDS | CTT −> TTA | CTT −> TTA | 6082 |
| Fg 8 - OTS | 3 | — | polyprotein lab CDS | AGT −> TCA | AGT −> TCA | 6096 |
| Fg 8 - OTS | 2 | — | polyprotein lab CDS | CTT −> TTA | CTT −> TTA | 6099 |
| Fg 8 - OTS | 2 | — | polyprotein lab CDS | CTC −> TTG | CTC −> TTG | 6102 |
| Fg 8 - OTS | 1 | — | polyprotein lab CDS | T −> A | TCT −> TCA | 6103 |
| Fg 8 - OTS | 1 | — | polyprotein lab CDS | T −> A | TCT −> TCA | 6119 |
| Fg 8 - OTS | 1 | — | polyprotein lab CDS | C −> T | CTA −> TTA | 6134 |
| Fg 8 - OTS | 1 | — | polyprotein lab CDS | C −> G | TCC −> TCG | 6143 |
| Fg 8 - OTS | 1 | — | polyprotein lab CDS | T −> A | TCT −> TCA | 6155 |
| Fg 8 - OTS | 1 | — | polyprotein lab CDS | C −> T | CTA −> TTA | 6178 |
| Fg 8 - OTS | 3 | — | polyprotein lab CDS | AGC −> TCG | AGC −> TCG | 6180 |
| Fg 8 - OTS | 1 | — | polyprotein lab CDS | C −> T | CTG −> TTG | 6184 |
| Fg 8 - OTS | 3 | — | polyprotein lab CDS | AGT −> TCA | AGT −> TCA | 6196 |
| Fg 8 - OTS | 1 | — | polyprotein lab CDS | C −> T | CTA −> TTA | 6205 |
| Fg 8 - OTS | 2 | — | polyprotein lab CDS | CTT −> TTA | CTT −> TTA | 6254 |
| Fg 8 - OTS | 3 | — | polyprotein lab CDS | AGT −> TCA | AGT −> TCA | 6282 |
| Fg 8 - OTS | 1 | — | polyprotein lab CDS | T −> A | TCT −> TCA | 6294 |
| Fg 8 - OTS | 1 | — | polyprotein lab CDS | T −> A | TCT −> TCA | 6299 |
| Fg 8 - OTS | 1 | — | polyprotein lab CDS | C −> T | CTA −> TTA | 6308 |
| Fg 8 - OTS | 1 | — | polyprotein lab CDS | C −> G | TCC −> TCG | 6321 |
| Fg 8 - OTS | 1 | — | polyprotein lab CDS | C −> T | CTA −> TTA | 6331 |
| Fg 8 - OTS | 1 | — | polyprotein lab CDS | T −> A | TCT −> TCA | 6332 |
| Fg 8 - OTS | 2 | — | polyprotein lab CDS | CTT −> TTA | CTT −> TTA | 6334 |
| Fg 8 - OTS | 3 | — | polyprotein lab CDS | AGT −> TCA | AGT −> TCA | 6343 |
| Fg 8 - OTS | 3 | — | polyprotein lab CDS | AGT −> TCA | AGT −> TCA | 6359 |
| Fg 8 - OTS | 1 | — | polyprotein lab CDS | T −> A | TCT −> TCA | 6373 |
| Fg 8 - OTS | 3 | — | polyprotein lab CDS | AGT −> TCA | AGT −> TCA | 6375 |
| Fg 8 - OTS | 1 | — | polyprotein lab CDS | T −> A | TCT −> TCA | 6379 |
| Fg 8 - OTS | 1 | — | polyprotein lab CDS | C −> T | CTA −> TTA | 6393 |
| Fg 8 - OTS | 1 | — | polyprotein lab CDS | T −> A | TCT −> TCA | 6395 |
| Fg 8 - OTS | 2 | — | polyprotein lab CDS | CTC −> TTG | CTC −> TTG | 6420 |
| Fg 8 - OTS | 3 | — | polyprotein lab CDS | AGC −> TCG | AGC −> TCG | 6432 |
| Fg 8 - OTS | 2 | — | polyprotein lab CDS | CTC −> TTG | CTC −> TTG | 6444 |
| Fg 8 - OTS | 2 | — | polyprotein lab CDS | CTT −> TTA | CTT −> TTA | 6451 |
| Fg 8 - OTS | 3 | — | polyprotein lab CDS | AGT −> TCA | AGT −> TCA | 6453 |
| Fg 8 - OTS | 1 | — | polyprotein lab CDS | T −> A | TCT −> TCA | 6477 |
| Fg 8 - OTS | 2 | — | polyprotein lab CDS | CTT −> TTA | CTT −> TTA | 6509 |
| Fg 8 - OTS | 2 | — | polyprotein lab CDS | CTC −> TTG | CTC −> TTG | 6524 |
| Fg 8 - OTS | 1 | — | polyprotein lab CDS | T −> A | TCT −> TCA | 6549 |
| Fg 8 - OTS | 1 | — | polyprotein lab CDS | T −> A | TCT −> TCA | 6555 |
| Fg 8 - OTS | 2 | — | polyprotein lab CDS | CTC −> TTG | CTC −> TTG | 6571 |
| Fg 8 - OTS | 2 | — | polyprotein lab CDS | CTT −> TTA | CTT −> TTA | 6594 |
| Fg 8 - OTS | 3 | — | polyprotein lab CDS | AGT −> TCA | AGT −> TCA | 6599 |
| Fg 8 - OTS | 1 | — | polyprotein lab CDS | T −> A | TCT −> TCA | 6606 |
| Fg 8 - OTS | 3 | — | polyprotein lab CDS | AGT −> TCA | AGT −> TCA | 6613 |
| Fg 8 - OTS | 2 | — | polyprotein lab CDS | CTT −> TTA | CTT −> TTA | 6614 |

| | | |
|---|---|---|
| Total Fg 8 - OTS | 11 [symbol] | Total codon changes Fg [symbol] = [symbol] 7 |

[symbol]

indicates data missing or illegible when filed

SUPPLEMENTARY TABLE 4
Primers for gene expression analysis

| SARS-CoV-2 target gene | | LHS (5′-3′) | RHS (5′phosphorylated-3′) |
|---|---|---|---|
| ORF1ab | 1 | TCC TTG GCA CCC GAG AAT | CGT AGT GCA ACA GGA CTA |
| | | TCC AAG TAC CGG CAG CAC | AGC TCA TAB AAA AAA AAA |
| | | AAG ACA TCT GT | AAA AAA AAA AAA AAA AAA |
| | | | AAA |
| | 2 | TCC TTG GCA CCC GAG AAT | TGT CTT ATA GCT TCT TCG |
| | | TCC AAT CGA AGC CAA TCC | CGG GTG AAB AAA AAA AAA |
| | | ATG CAC GTA CA | AAA AAA AAA AAA AAA AAA |
| | | | AAA |
| | 4 | TCC TTG GCA CCC GAG AAT | GGC CAC CAG CTC CTT TAT |
| | | TCC AGA CTT TAG ATC GGC | TAC CGT TAB AAA AAA AAA |
| | | GCC GTA ACT AT | AAA AAA AAA AAA AAA AAA |
| | | | AAA |
| | 5 | TCC TTG GCA CCC GAG AAT | GTC ATT AGT ATA ACT ACC |
| | | TCC ACT GCA GCA ATC AAT | ACC ACG CAB AAA AAA AAA |
| | | GGG CAA GCT TT | AAA AAA AAA AAA AAA AAA |
| | | | AAA |
| ORF3a | 1 | TCC TTG GCA CCC GAG AAT | CGA TTG TGT GAA TTT GGA |
| | | TCC AGG ATT AAC AAC TCC | CAT GTT CAB AAA AAA AAA |
| | | GGA TGA ACC GT | AAA AAA AAA AAA AAA AAA |
| | | | AAA |
| | 2 | TCC TTG GCA CCC GAG AAT | ATG TTC AGA AAT AGG ACT |
| | | TCC ACA GTA TAA CCA CCA | TGT TGT GAB AAA AAA AAA |
| | | ATC TGG TAG TC | AAA AAA AAA AAA AAA AAA |
| | | | AAA |
| | 3 | TCC TTG GCA CCC GAG AAT | TAC AAC ACA GTC TTT TAC |
| | | TCC AAG TCT GAA GTG AAG | TCC AGA TAB AAA AAA AAA |
| | | TAA CTG TGT AA | AAA AAA AAA AAA AAA AAA |
| | | | AAA |
| ORF10 | | TCC TTG GCA CCC GAG AAT | AAA CGG AAA AGC GAA AAC |
| | | TCC ATG CAC AAG AGT AGA | GTT TAT AAB AAA AAA AAA |
| | | CTA TAT ATC GT | AAA AAA AAA AAA AAA AAA |
| | | | AAA |
| Envelope (E) | | TCC TTG GCA CCC GAG AAT | TCG AAG CGC AGT AAG GAT |
| | | TCC ACA ATA TTG CAG CAG | GGC TAG TAB AAA AAA AAA |
| | | TAC GCA CAC AA | AAA AAA AAA AAA AAA AAA |
| | | | AAA |
| ORF1ab | 1 | TCC TTG GCA CCC GAG | CGT AGT GCA ACA GGA CTA |
| | | AAT TCC AAG TAC CGG | AGC TCA TAB AAA AAA AAA |
| | | CAG CAC AAG ACA TCT | AAA AAA AAA AAA AAA AAA |
| | | GT | AAA |
| Membrane (M) | 1 | TCC TTG GCA CCC GAG | ACG AAG ATG TCC ACG AAG |
| | | AAT TCC AAG CGT CCT | GAT CAC AAB AAA AAA AAA |
| | | AGA TGG TGT CCA GCA | AAA AAA AAA AAA AAA AAA |
| | | AT | AAA |
| | 2 | TCC TTG GCA CCC GAG | TGT AGC AAC AGT GAT TTC |
| | | AAT TCC AAT TTG TAA | TTT AGG CAB AAA AAA AAA |
| | | TAA GAA AGC GTT CGT | AAA AAA AAA AAA AAA AAA |
| | | GA | AAA |
| | 3 | TCC TTG GCA CCC GAG | TTA TTC TGT AAA CAG CAG |
| | | AAT TCC AAT AGC AAT | CAA GCA CAB AAA AAA AAA |
| | | TCC ACC GGT GAT CCA | AAA AAA AAA AAA AAA AAA |
| | | AT | AAA |
| Nucleocapsid (N) | 1 | TCC TTG GCA CCC GAG | GTT GAG TGA GAG CGG TGA |
| | | AAT TCC AGG GAA TTT | ACC AAG AAB AAA AAA AAA |
| | | AAG GTC TTC CTT GCC | AAA AAA AAA AAA AAA AAA |
| | | AT | AAA |
| | 2 | TCC TTG GCA CCC GAG | ACG TCT GCC GAA AGC TTG |
| | | AAT TCC AAA TTT CCT | TGT TAC AAB AAA AAA AAA |
| | | TGG GTT TGT TCT GGA | AAA AAA AAA AAA AAA AAA |
| | | CC | AAA |
| | 3 | TCC TTG GCA CCC GAG | CTT CGG TAG TAG CCA ATT |
| | | AAT TCC AAC CAC CAC | TGG TCA TAB AAA AAA AAA |
| | | GAA TTC GTC TGG TAG | AAA AAA AAA AAA AAA AAA |
| | | CT | AAA |
| Spike (S) | 1 | TCC TTG GCA CCC GAG | GAG ACA ACT ACA GCA ACT |
| | | AAT TCC AAG GAT CCA | GGT CAT ABA AAA AAA AAA |
| | | CAA GAA CAA CAG CCC | AAA AAA AAA AAA AAA AAA |
| | | TT | AA |
| | 2 | TCC TTG GCA CCC GAG | AAC CAA CAC CAT TAG TGG |
| | | AAT TCC AAG TAC TAC | GTT GGA AAB AAA AAA AAA |
| | | TAC TCT GTA TGG TTG | AAA AAA AAA AAA AAA AAA |
| | | GT | AAA |
| | 3 | TCC TTG GCA CCC GAG | AAA TCT ACC AAT GGT TCT |
| | | AAT TCC ACC TAG TGA | AAA GCC GAB AAA AAA AAA |
| | | TGT TAA TAC CTA TTG | AAA AAA AAA AAA AAA AAA |
| | | GC | AAA |

SUPPLEMENTARY TABLE 5
Sequencing results of OTS viruses after in vitro and in vivo passaging:
In vitro - Mutations detected after ten or fifteen serial passages
of OTS viruses and SARS-CoV-2 WT in Vero E6 cells.

| | | Nucleotide | Nucleotide | Amino acid | |
|---|---|---|---|---|---|
| Construct | Locus | position | exchange | exchange | Frequency |
| OTS4-5 | ORF1b | 19720 | A → G | K2085E | 90% |
| p.10 | S | 21789 | C → T | T76I | 18% |
| in Vero E6 | S | 23525 | C → T | H655Y | 99% |
| | S | 23585-23599 | Deletion | del675-679 | 93% |
| | M | 26895 | C → T | H125Y | 43% |
| OTS7-8 | ORF1a | 4979 | A → G | T1572A | 19% |
| p.10 | ORF1b | 14599 | C → T | synonymous | 13% |
| in Vero E6 | ORF1b | 19741 | G → A | E2092K | 98% |
| | S | 22296 | A → G | H245R | 87% |
| | S | 23597-23617 | Deletion | del679-685 | 95% |
| OTS-228 | S | 21765-21785 | Deletion | del68-73 | 100% |
| p.15 | ORF10 | 29587 | G → A | synonymous | 13% |
| OTS-228 | ORF1ab | 7563 | C → T | S → L | 15% |
| p.15 rep.1 | ORF1ab | 7729 | T → A | synonymous | 77% |
| in VeroE6 | ORF1ab | 11460 | C → T | S → L | 61% |
| | S | 22320 | A → T | D → V | 85% |
| OTS-228 | ORF1ab | 553 | G → A | synonymous | 13% |
| p.15 rep.2 | ORF1ab | 1684 | C → T | synonymous | 43% |
| in VeroE6 | ORF1ab | 2909 | A → G | T → A | 21% |
| | ORF1ab | 7334 | C → T | H → Y | 49% |
| | ORF1ab | 7729 | A → C | synonymous | 88% |
| | ORF1ab | 8660 | C → T | H → Y | 33% |
| | S | 22320 | A → T | D → V | 92% |
| OTS-228 | ORF1ab | 3003 | A → G | E → G | 46% |
| p.15 rep.3 | ORF1ab | 7729 | A → C | synonymous | 31% |
| in VeroE6 | ORF1ab | 7932 | C → T | S → L | 20% |
| | ORF1ab | 8872 | G → T | L → F | 61% |
| | ORF1ab | 16538 | A → C | I → L | 18% |
| | S | 22320 | A → T | D → V | 88% |
| | S | 26147 | T → A | M → K | 49% |
| | 3a | 26532 | G → A | E → K | 10% |
| SARS-CoV-2 | ORF1b | 16609 | A → G | T1048A | 54% |
| WT p.15 in | ORF1b | 17432 | T → G | I1322S | 100% |
| Vero E6 | S | 23597-23626 | Deletion | del679-688 | 100% |

Mutations detected after ten serial passages of OTS-206 in
TMPRSS2-expressing Vero E6 cells in 3 replicates. Only
mutations with a frequency less than 10% are included.

| | | Nucleotide | Nucleotide | Amino acid | |
|---|---|---|---|---|---|
| Replicate | Locus | position | change | change | Frequency |
| OTS-206_1 | S | 22206 | A → G | D → G | 68.5% |
| p.10 | 3a | 25893 | T → C | N/A | 78.5% |
| OTS-206_2 | ORF1a | 2840 | G → A | A → T | 61.6% |
| p.10 | ORF1a | 7334 | C → T | H → Y | 88.6% |
| | S | 21752 | T → A | W → R | 95% |
| OTS-206_3 | ORF1a | 895 | A → G | N/A | 12% |
| p.10 | ORF1a | 4456 | C → T | N/A | 10.3% |
| | ORF1a | 6251 | A → C | N → H | 10% |
| | ORF1a | 7678 | A → C | R → S | 13.8% |
| | ORF1b | 14352 | C → T | T → I | 22.2% |
| | ORF1b | 15278 | A → G | T → A | 10.7% |
| | S | 22206 | A → G | D → G | 50.5% |
| | S | 23525 | C → T | H → Y | 13.5% |
| | 3a | 25893 | T → C | N/A | 27.8% |

In vivo - Mutations detected after animal passage of OTS4-5
and OTS7-8 in Syrian hamsters. For “OTS4-5 animal4-d21”, no
full-length genomic sequence could be obtained; the final
sequence contains N-stretches (1.4%) also localized in fragment
4 (5.5% of the 2992 nucleotides are Ns) and fragment 5
(3.4% of the 3249 nucleotides are Ns).

| | | Nucleotide | Nucleotide | Amino acid | |
|---|---|---|---|---|---|
| Construct | Locus | position | exchange | exchange | Frequency |
| OTS4-5 | ORF1a | 9442 | C → T | synonymous | 100% |
| animal4-d21 | M | 26895 | C → T | H125Y | 66% |
| | ORF6 | 27243 | A → G | I14M | 88% |
| | 5 | 23585-23599 | Deletion | del675-679 | 26% |
| OTS7-8 | ORF1b | 18394 | G → A | A1643T | 28% |
| animal18- | | | | | |
| d21 | | | | | |

In vivo - Mutations detected after in vivo replication passage
of OTS-228 in nasal conchae tissue of Syrian hamsters
(inoculated animals). For “animal3”, “animal 7” and “animal 10”,
no full-length genomic sequence could be obtained; the final
sequences contain N-stretches (“animal3” 10.5%, “animal 7”
0.2% and “animal 10” 0.6%), n.a., not analyzed due to low
coverage.

| | | Nucleotide | Nucleotide | Amino acid | |
|---|---|---|---|---|---|
| Construct | Locus | position | exchange | exchange | Frequency |
| OTS-228 | n.a. | | | | |
| animal3-d21 | | | | | |
| OTS-228 | n.a. | | | | |
| animal7-d21 | | | | | |
| OTS-228 | ORF1a | 7063 | C → A | Y2266* | 100% |
| animal10-d21 | S | 25018 | A → T | L1152F | 23% |
| OTS-228 | ORF1a | 1684 | C → T | synonymous | 29% |
| animal12-d21 | | | | | |
| OTS-228 | ORF1a | 7008 | C → T | A2248V | 100% |
| animal13-d21 | | | | | |

List of antibodies used for flow cytometry analysis

| Live/Dead Fixable Aqua | | | Thermofisher |
|---|---|---|---|
| Avidin | | | MERCK |
| FcR blockung reagent, mouse | Fc Block (CD16/32) | | Miltenyi biotec |
| Anti-mouse antibodies | Dye | Clone | Company |
| CD8- FITC | FITC | 53-6.7 | biolegend |
| CD45- PerCP | PerCP | 30-F11 | biolegend |
| CD3e- PE | PE | 145-2C11 | biolegend |
| H-2K(b) SARS-CoV-2 S | Alexa Fluor | | NIH tetramer |
| 539-546 VNFNFNGL | 647 | | core facility |
| H-2D(b) Influenza A NP | Alexa Fluor | | NIH tetramer |
| 366-374 ASNENMETM | 647 | | core facility |
| CD3- AF647 (compensation) | Alexa Fluor | 145-2C11 | biolegend |
| | 647 | | |

### Results

### Development of Improved SARS-CoV-2 LAV Candidates Using the OTS Approach

To incorporate the one-to-stop (OTS) approach into the SARS-CoV-2 genome and to generate OTS fragments and OTS mutants (called herein also OTS constructs), the inventors used the in-yeast transformation-associated recombination (TAR) cloning method (Thao, doi:10.1038/s41586-020-2294-9). Nucleotide changes were introduced to specific areas of ORF1ab using serine and leucine codons (FIG. 19a). This resulted in various recombinant SARS-CoV-2 mutants: OTS2, OTS4, OTS5, OTS7, and OTS8 (FIG. 19a, FIG. 23a, Supplementary Table 1 and 3). The inventors combined these recoded fragments to create OTS4-5, OTS7-8, and finally OTS4-5-7-8 mutants. The OTS4-5-7-8 mutant had a total of 576 mutations and 325 synonymous codon changes in the recoded ORF1ab (Supplementary Table 1 and 3).

For the subsequent OTS live attenuated vaccine (LAV) candidates OTS-206 and OTS-228, the inventors used the massively recoded ORF1ab from OTS4-5-7-8 as foundation. The OTS-206 vaccine virus combined the OTS4-5-7-8 mutations resulting in two amino acid substitutions (K164A, H165A) in the Nsp1 gene and the deletion of the accessory genes ORF6-8 (FIG. 19a). To create OTS-228, the inventors deleted the polybasic spike S1/S2 cleavage site (ΔPRRAR) from OTS-206 (FIG. 19a).

In summary, the inventors employed the TAR cloning method to introduce nucleotide changes in specific areas of ORF1ab, resulting in multiple OTS mutants. From these mutants, the inventors developed the OTS-206 by combining OTS4-5-7-8 mutations, nucleotide substitutions in Nsp1 and deletion of accessory genes. In OTS-228, the polybasic spike S1/S2 cleavage site was additionally deleted from OTS-206.

OTS Constructs are More Sensitive to Treatment with Mutagenic Drugs, but Show In Vitro Replication Kinetics Comparable to SARS-CoV-2 WT

The inventors compared plaque sizes and replication kinetics of different OTS viruses to the ancestral wild-type SARS-CoV-2 (WT) to evaluate the impact of OTS changes on phenotype and replication fitness. OTS4-5, OTS7-8, OTS4-5-7-8, and OTS-206 exhibited significant variation in plaque sizes. On average, OTS4-5, OTS7-8, and OTS-206 had smaller plaques, though not statistically significant, while OTS4-5-7-8 had larger plaques (FIG. 19b, FIG. 23b).

Replication kinetics were assessed in VeroE6/TMPRSS2 cells, human nasal epithelial cells (hNECs), and bronchial epithelial cells (hBECs). OTS4-5, OTS7-8, OTS4-5-7-8, and OTS-206 replicated similarly to WT in VeroE6/TMPRSS2 cells but displayed notable differences in hNECs and hBECs (FIG. 19c-e, FIG. 23c, d, e). In hNECs, OTS4-5-7-8 and OTS-206 exhibited reduced fitness compared to WT, with lower apical titers up to 96 hours post-infection (hpi) (FIG. 19d). Variability was observed in hBECs for OTS4-5, OTS7-8, and OTS4-5-7-8, while OTS-206 reached similar apical titers as WT at 96 hpi (FIG. 19e). Recombinant viruses with Nsp1 mutation (K164A, H165A) or deletion of accessory ORFs 6-8 (delORF6-8) served as controls for OTS-206 (FIG. 23d, c). The Nsp1 mutant displayed kinetics similar to WT in both the cell line and hBECs, while the delORF6-8 virus showed increased titers at 24 hpi in VeroE6/TMPRSS2 and 96 hpi in hBECs (FIG. 23c, d).

Furthermore, the inventors assessed the vulnerability of OTS4-5-7-8 to 5-fluorouracil (5-FU) and molnupiravir treatment, expecting increased susceptibility due to OTS modifications. OTS4-5-7-8 showed a dose-dependent decrease in viral titers compared to WT when exposed to 5-FU (FIG. 19f). Although not as dramatic as with 5-FU, OTS4-5-7-8 replicated significantly less than WT when treated with molnupiravir (FIG. 19g).

In summary, in vitro evaluation of the viruses in conditions simulating human upper respiratory epithelium (hNECs at 33° C.) and lower respiratory epithelium (hBECs at 37° C.) revealed that OTS mutations led to reduced fitness or no significant difference compared to WT SARS-CoV-2. Notably, treatment with 5-FU or molnupiravir dramatically reduced replication of OTS4-5-7-8, suggesting increased susceptibility to mutagenic treatments that enhance replication errors and the likelihood of stop codon emergence.

### Stability of OTS Modifications

The genetic stability of OTS4-5, OTS7-8, OTS-228, and WT SARS-CoV-2 after ten or fifteen passages in VeroE6 cells was assessed by next-generation sequencing (NGS). OTS4-5, OTS7-8, and WT exhibited loss of the S1/S2 cleavage site through deletion (S 679-NSPRRAR-685), a known characteristic when SARS-CoV-2 is propagated in TMPRSS2-deficient environments like VeroE6 cells (10.1038/s41586-021-03237-4). However, the S1/S2 cleavage site of OTS-206 and the ΔPRRAR deletion of OTS-228 remained unchanged when passaged on VeroE6/TMPRSS2 cells (Supplementary Table 5).

Crucially, none of the modified leucine and serine codons (OTS codons) reverted to the wild-type sequence after ten passages (OTS4-5, OTS7-8, and OTS-206) or fifteen passages (OTS-228) in either VeroE6 or VeroE6/TMPRSS2 cells. Additionally, the introduced Nsp1 mutations (K164A, H165A) in OTS-206 and OTS-228, as well as the ORF6-8 deletions, were retained during passages.

### OTS Genome Modification Influences Level of Attenuation

To assess the attenuation levels of OTS mutations, various experiments were conducted in K18-hACE2 mice and Syrian hamsters (FIG. 24a). In K18-hACE2 mice, individual OTS mutations (OTS2, OTS7, OTS8) resulted in no weight loss (FIG. 24b) or clinical signs (FIG. 24c), but infectious virus titers (FIG. 24d), genome copies (FIG. 24e), and lung pathology (FIG. 24f, g) were comparable to WT. Additionally, no detectable infectious virus progeny was found with OTS2 and OTS7 in the nasal conchae or OTS7 in the brain (FIG. 24d). Therefore, OTS mutations in multiple fragments (OTS4-5, OTS7-8) (FIG. 24h) and the OTS-206 construct, which included NSP1 mutations and ORF6-8 knockout, were tested.

In K18-hACE2 mice, WT SARS-CoV-2 and also one OTS4-5 mice were associated with weight loss (FIG. 24i), while only WT exhibit clinical signs 5 5 days post inoculation (dpi) (FIG. 24j). Infectious virus titers in the lungs, noses, and brains of OTS4-5 and OTS7-8 infected mice were lower than WT, or even completely negative for OTS7-8 nose and brain samples (FIG. 24k), although viral RNA copies were still high (FIG. 24l, m). Notably, OTS constructs, including OTS7, did not lead to infectious viruses in the brain.

In Syrian hamsters, OTS4-5, OTS7-8, and OTS-206 were compared to WT (FIG. 25a). While none of these OTS constructs induced lethality, OTS4-5 and OTS7-8 caused weight loss similar to WT, whereas OTS-206 did not induce weight loss (FIG. 25b). OTS-206 also showed reduced genome copy numbers in nasal washings (FIG. 25d) and respiratory tract tissues compared to OTS4-5 and OTS7-8 (FIG. 25f, g). Histopathology revealed characteristic lung lesions, with predominantly type I pneumocytes, and virus antigen distribution in all infected animals (FIG. 25k, l).

Transmission from both OTS-inoculated hamster groups to the naive contact animals was observed. OTS4-5 and OTS7-8 contact hamsters experienced weight loss, while OTS-206 contact animals did not (FIG. 25c). Viral RNA copies in nasal washings (FIG. 25d, e) and organs (FIG. 25h) and seropositivity (FIG. 25i, j) were detected in contact animals, confirming transmission. Importantly, sequencing of 21 dpi conchae samples of OTS4-5 and OTS7-8 contact animals, confirmed that the OTS codons remained stable after in vivo passage (Supplementary Table 5).

In summary, introducing OTS codon modifications in combinations of two OTS fragments (OTS4-5 and OTS7-8) led to modest attenuation, reducing virulence but not eliminating weight loss or viral shedding. However, when four OTS fragments were recoded, such as in the OTS-206 construct, significant attenuation was observed, with no weight loss and fewer viral genome copies. Lung lesions were still present, but the OTS genome modifications remained genetically stable after in vivo passage.

Immunization with OTS Constructs Lead to Full Protection Against SARS-CoV-2 Challenge Infection

To evaluate the immunogenicity and protective efficacy of OTS4-5-7-8 and OTS-206 compared to OTS4-5 and OTS7-8, the inventors conducted intranasal immunization of K18-hACE2 mice (FIG. 20a, FIG. 26a). Mice immunized with OTS4-5-7-8 and OTS-206 showed no significant weight loss or clinical symptoms (FIG. 26b, c), unlike those immunized with OTS4-5 or OTS7-8, which required euthanasia due to high clinical scores (FIG. 20b, c). Following immunization, all mice were challenged with wild-type (WT) SARS-CoV-2. Naïve mice in the control group reached a humane endpoint and had to be euthanized (FIG. 20d, e, f), while mice immunized with OTS4-5 and OTS7-8 displayed rapid recovery and no significant weight loss or clinical signs (FIG. 20d, e, f). The viral genome copies in the nose and lung samples of OTS-immunized mice were significantly lower than those of non-immunized mice (FIG. 20 g, h, FIG. 26e-h). No infectious virus was detected in the samples of pre-immunized and challenged mice, indicating virus clearance (FIG. 2i, FIG. 26d, f). Histopathological analysis showed mild lung pathology in mice immunized with two OTS fragments. However, mice pre-immunized with OTS-206 exhibited only minor signs of infection that resolved quickly (FIG. 26i). These findings confirmed that the OTS mutants and especially OTS-206 provided protection against lethal SARS-CoV-2 challenge and elicited neutralizing antibody responses (FIG. 26j) and SARS-CoV-2 spike-specific CD8 T-cell responses (FIG. 26k).

The protective efficacy of OTS mutants was further evaluated in Syrian hamsters. In the first experiment, hamsters were immunized with OTS4-5 or OTS7-8 and challenged with WT SARS-CoV-2 (FIG. 201). None of the immunized hamsters succumbed to the challenge infection, while 75% of the naïve control animals did (FIG. 20m). The immunized animals did not experience weight loss, in contrast to the control group (FIG. 20n). Viral genome copy numbers in nasal washing samples were significantly lower in the immunized groups (FIG. 20o). At 14 days post-challenge, viral genome loads in organ samples were barely above the threshold, indicating virus clearance (FIG. 20p). However, transmission of the challenge virus to naïve contact animals was not blocked by OTS4-5 or OTS7-8 immunization, as proven by increased lethality (FIG. 20m), body weight loss (FIG. 20n), virus genome positive nasal washing (FIG. 20o) and organ samples (FIG. 20p), as well as serological evaluation of the final serum samples (FIG. 26l, m).

In the second experiment, hamsters were immunized with OTS-206 and challenged with the SARS-CoV-2 Omicron BA.2 variant (FIG. 20q). Neither the immunized nor the naïve hamsters in direct contact showed any lethality (FIG. 20r), or weight loss, while the challenged naïve control animals continuously lost weight (FIG. 20s). Viral RNA in nasal washing samples was significantly reduced in the immunized group compared to the control group (FIG. 20t), and delayed virus transmission to contact animals for the immunized group (FIG. 20t). Analysis of organ samples showed high protection against BA.2 replication in the lung of OTS-206-immunized animals (FIG. 20u, FIG. 26n). Sera from OTS-206-immunized hamsters exhibited a high level of wild type RBD-specific (FIG. 260) and neutralizing capacity against both WT D614G and Omicron BA.2 (FIG. 26p). Although transmission of the challenge virus to direct contact animals could not be prevented, OTS-206-immunized hamsters were protected from weight loss, and pulmonary atelectasis (FIG. 26 q, r), with only marginal virus antigen detectable in lung samples (FIG. 26 s, t).

In conclusion, immunization with OTS candidates provided protection against lethal SARS-CoV-2 challenge in mice and hamsters. The vaccines elicited neutralizing antibody responses and specific CD8 T-cell responses. While OTS4-5 and OTS7-8 reduced viral loads and prevented lethality and morbidity in hamsters, they did not block transmission to naïve contact animals. OTS-206 immunization showed superior protection against weight loss, pulmonary atelectasis, and viral replication, but transmission to contact animals still occurred to a low degree.

OTS-206 Induces Long-Term Immunity and is Superior in Virus Clearance after Challenge

The inventors challenged K18-hACE2 mice 28 days after they have been immunized with a single dose of an mRNA-vaccine (monovalent Spikevax) or the OTS-206, with the most pathogenic SARS-CoV-2 VOC Delta (B.1.617.2) (FIG. 21a). To assess vaccine protection early after the heterologous challenge infection, lungs were harvested 2- or 5 dpc. Immunohistochemistry of the whole lungs showed a variable but higher abundance of nucleocapsid proteins detected in the lungs of mRNA vaccinated mice 2 dpc, and almost undetectable in both conditions 5 dpc (FIG. 21b, c). Spatial transcriptomics of the lungs focusing on SARS-CoV-2 transcripts confirmed lung immunochemistry results and showed higher viral mRNA expression per capture spot in the lung tissue for the mRNA vaccinated mice than for the OTS-206 vaccinated mice (FIG. 21d). Strikingly, different SARS-CoV-2 transcripts were detected at lower levels in OTS-206 vaccinated mice at 2 dpc compared to mRNA-vaccinated mice, and not detected anymore at 5 dpc in OTS-206 vaccinated mice (FIG. 21d, e), suggesting faster clearance of the challenge virus in OTS-206 vaccinated mice. The inventors also assessed spatial host gene transcriptional expression in the vicinity of sites of virus infection in the lungs. The inventors compared the pathway activity scores constructed from the expression changes of the top 100 genes that are involved in several cellular pathways such as MAPK, JAK-STAT, TGF-β and TNF-α (FIG. 21f). The inventors observed a consistent spatial correlation pattern between the viral and the host genes in the infected lungs for the mRNA and OTS-206 groups 2 dpc (FIG. 27a). This similarity in gene expression signatures suggests a comparable response in terms of gene activation between the two conditions. It is interesting to note that the mRNA and OTS-206 groups share 8 of the 20 host genes with the highest spatial correlation with virus RNA transcripts (FIG. 27b). The expression of pro-inflammatory cytokines that have been reported to be upregulated in SARS-CoV-2 patients (10.3390/v13061062; https://doi.org/10.1038/s41467-021-22210-3) was elevated in the mRNA vaccinated group compared to the OTS-206 group (FIG. 27c). Notably, the JAK-STAT pathway, that is crucial in processes such as innate and adaptive immune responses, cell division, hematopoiesis and tissue repair, showed significantly increased activity in the lung at sites of infection (FIG. 27d). As shown in the violin plots, which show the underlying distribution of pathway scores in each capture spot, the JAK-STAT pathway activation at 2 dpc was higher in mRNA-vaccinated mice compared to OTS-206 vaccinated mice (FIG. 21f). Most strikingly, at 5 dpc, JAK-STAT activation was almost back to baseline levels in OTS-206 vaccinated mice, demonstrating that faster clearance of heterologous SARS-CoV-2 VOC is accompanied by faster resolution of virus-induced host responses.

The inventors then immunized K18-hACE2 mice either with a homologous or heterologous prime-boost combination of mRNA vaccine (monovalent Spikevax) or OTS-206 (FIG. 21g). To compare the immediate protection, mice were challenged with WT D614G or the Delta VOC (B.1.617.2) 28-days post-boost (28 dpb), while long term protection was evaluated by challenge 5 months post-boost (5 mpb) using the WT D614G virus (FIG. 21g, FIG. 28a, b). All immunized mice, regardless of the immunization combination or the challenge virus, were protected from disease and body weight loss, when challenged 28 days post-boost or 5-month post-boost (FIG. 21h, k). No infectious virus was detected 6 dpc in nose or lung samples of the immunized animals (FIG. 21i, l). Naïve WT D614G and Delta VOC challenged mice showed similar levels of viral titers (FIG. 21i), but the histopathological score of the lungs of Delta-challenged mice were significantly higher than the WT D614G-challenged mice (FIG. 3j). Viral RNA load in organ samples and oropharyngeal swabs of all immunized groups showed a significant reduction in replication compared to the naïve control animals which were challenged with either WT or Delta VOC (FIG. 28c). Strikingly, mice challenged 174 days after vaccination showed less amount of viral RNA in the organ samples compared to the similarly immunized mice challenged 57 days after vaccination (FIG. 28e), pointing that the protection provided by the immunization did not decrease within about 5 months. This trend was also reflected in the histopathological scores of the lungs (FIG. 21j, m). Altogether, these data show the ability of OTS-206 to induce long-term protection against SARS-CoV-2 in the very sensitive K18-hACE2 mice model.

### Deletion of the Spike Polybasic Cleavage Site Blocks LAV Transmission, and Inhibits Transmission of WT SARS-CoV-2 Challenge Infections

In order to avoid transmission to naïve subjects, the inventors developed an optimized version called OTS-228 by removing the polybasic cleavage site (PCS) in the spike protein (FIG. 22a).

In vitro analysis showed that the deletion of the PCS resulted in smaller plaque sizes (FIG. 22b), no impaired replication in VeroE6/TMPRSS2 cells (FIG. 29), delayed replication kinetics in human nasal epithelial cells (hNECs), and reduced viral titers in human bronchial epithelial cells (hBECs) (FIG. 22c). The transmissive potential of OTS-228 was evaluated in a hamster model. Ten hamsters were intranasally inoculated with OTS-228, and four naïve contact animals were introduced at 1-day post-inoculation (FIG. 22d). None of the inoculated animals or contact animals experienced lethality (FIG. 22e) or weight loss (FIG. 22f). While the viral genome was detectable in nasal washing samples of the inoculated hamsters until 7 days post-inoculation to levels of 107 gc/mL and higher, contact animals exhibit only marginal amount of virus genome at two time points (3399 (3 dpi) and 1782 (4 dpi) gc/mL) (FIG. 22g). The viral RNA in samples collected from the inoculated animals at 5 days post-inoculation showed a significant reduction, except in the conchae, and viral genome was still detectable at 21 days post-inoculation (FIG. 22h). The genetic stability of the OTS-228 modifications was confirmed through deep sequencing of these conchae samples (Supplementary Data Table 1). No viral genome was detected in organ samples from the naïve contact animals at 21 days post-inoculation (FIG. 22h). Serological evaluation confirmed that all contact animals remained seronegative after 20 days of direct contact with the inoculated hamsters (FIG. 22i). The immunized animals showed neutralizing capacity against wild-type SARS-CoV-2, while one animal even exhibited neutralizing activity against the Omicron BA.2 and BA.5 variants (FIG. 22j). Histopathological analysis of the lungs from the inoculated hamsters at 5 days post-inoculation showed no signs of pneumonia-related atelectasis or characteristic SARS-CoV-2 vascular lesions (FIG. 35a-d). Some animals exhibited mild expansion of the pulmonary interstitium with macrophages, and a focal perivascular immune cell infiltration was found in one hamster.

These findings demonstrate that OTS-228 is completely attenuated and capable of inducing a broad neutralizing humoral immune response in the Syrian hamster model. Importantly, transmission to naïve direct contact animals was completely prevented, addressing a key concern associated with the previous OTS-206 vaccine candidate.

### OTS-228 Vaccination Protects Against VOC Challenge Infection and Limits Challenge Virus Transmission Events

The inventors assessed the protective efficacy of the OTS-228 vaccine against WT SARS-CoV-2 (FIG. 30a), but also against variants of concern (VOCs) including Omicron BA.2 (FIG. 31a) and Omicron BA.5 (FIG. 22k). These immunized and challenged animals were co-housed with non-immunized contact animals.

Remarkably, OTS-228 immunization resulted in full protection against lethality (FIG. 30b) and body weight loss (FIG. 30c), significantly reduced shedding of virus genome (FIG. 30d) and drastically reduced genome loads in organ samples (FIG. 30e, f). This prevents virus transmission of the WT virus to naïve contact animals (triangles in FIG. 30b, c, d, f), also corroborated by serology (FIG. 30g, h).

After the Omicron BA.2 challenge, there was no lethality (FIG. 31b) or weight loss observed (FIG. 31c). Virus shedding (FIG. 31d) and replication in the lungs was significantly inhibited (FIG. 31e), and no viral genome was detected at 14 days post-challenge (FIG. 31f). Among the contact animals, only one showed evidence of infection through serological analysis (FIG. 31g). The immunized animals exhibited similar neutralizing titers against both the WT D614G and the Omicron BA.2 variant (FIG. 31h).

Following the Omicron BA.5 challenge, the OTS-228-immunized animals did not experience lethality or weight loss, while control animals did, and one control animal died during the sampling procedure (FIG. 22l, m). Viral loads in the nasal washing samples of the immunized group were significantly lower compared to the non-immunized group (FIG. 22n). By 8 days post-challenge, the immunized animals had undetectable levels of viral genome in nasal washing samples, while the non-immunized mock animals still showed viral presence (FIG. 22n). Viral loads in organ samples and conchae were also significantly reduced in the immunized animals (FIG. 220). All lung samples from the immunized animals tested negative for the virus at 14 days post-challenge (FIG. 32). Serological evaluation confirmed the presence of SARS-CoV-2-RBD-specific antibodies in the immunized group (FIG. 22p). Two contact animals of the OTS-228 group tested positive for the Omicron BA.5 challenge virus in nasal washing samples (FIG. 22n), the conchae samples (FIG. 22o) and showed reactivity in the serological test (FIG. 22p), indicating transmission. The immunized animals exhibited comparable neutralization titers against WT D614G, Omicron BA.2 and BA.5, while the control animals only showed neutralization against Omicron BA.5 (FIG. 22r).

Histopathological examination of the lungs showed that the OTS-228 vaccination protected against pneumonia-related atelectasis and SARS-CoV-2 characteristic lesions after challenge with WT, BA.2 or BA.5 (FIG. 35). However, oligofocal SARS-CoV-2-typical lesions were observed depending on the challenge virus.

Overall, the intranasal single-dose application of OTS-228 was safe and highly effective in providing protection against WT and Omicron BA.2 and BA.5 variants. Importantly, transmission of WT from OTS-228-immunized animals to contact animals was completely prevented, demonstrating sterile immunity. Additionally, transmission of the Omicron BA.2 and BA.5 VOCs to contact animals was reduced.

1. A pharmaceutical product comprising a polynucleotide for use in the prevention or treatment of a SARS-CoV-2 virus infection,
wherein said polynucleotide encodes an attenuated human coronavirus or a fragment thereof, wherein the polynucleotide comprises at least 20 one-to-stop codons, wherein a one-to-stop codon is i) a different but synonymous codon compared to the corresponding codon in a natural human coronavirus genome and ii) differs by one nucleotide from a STOP codon, and
wherein said SARS-CoV-2 virus is not a Wuhan wild-type SARS-CoV-2 virus.

2. The pharmaceutical product for use according to claim 1, wherein said SARS-CoV-2 virus is a variant of the Wuhan wild-type SARS-CoV-2 virus.

3. The pharmaceutical product for use according to claim 2, wherein said variant is of lineage B, preferably B.1, more preferably B.1.1 or B.1.617, again more preferably B.1.1.529 or B.1.617.

4. The pharmaceutical product for use according claim 2 or 3, wherein the variant is selected from the group comprising, or preferably consisting of, Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), Mu (lineage B.1.621) and a missense variant of a Wuhan wild-type SARS-CoV-2 virus, wherein the genome of said missense variant comprises at least one missense mutation;
preferably the variant is selected from the group comprising, or preferably consisting of, Alpha (lineage B.1.1.7), B.1.1.7 with E484K, Beta (lineage B.1.351), Delta (lineage B.1.617.2), Omicron (B.1.1.529), Epsilon (lineages B.1.429, B.1.427, CAL.20C), Eta (lineage B.1.525), Iota (lineage B.1.526), Kappa (lineage B.1.617.1), Mu (lineage B.1.621) and a missense variant of a Wuhan wild-type SARS-CoV-2 virus wherein the genome of said missense variant comprises at least one missense mutation;
more preferably the variant is Delta (lineage B.1.617.2), Omicron (B.1.1.529) or a missense variant of a Wuhan wild-type SARS-CoV-2 virus, wherein the genome of said missense variant comprises at least one missense mutation; and
again more preferably the variant is Delta (B.1.617.2), Omicron BA.2, Omicron BA.5 or a missense variant of a Wuhan wild-type SARS-CoV-2 virus, wherein the genome of said missense variant comprises at least one missense mutation.

5. The pharmaceutical product for use according to claim 4, wherein said missense mutation is in an ORF encoding a SARS-CoV-2 spike protein, preferably said missense mutation is D614G.

6. The pharmaceutical product for use according to claims 2-5, wherein the variant is selected from the group comprising, or preferably consisting of, Alpha (lineage B.1.1.7), B.1.1.7 with E484K, Beta (lineage B.1.351), Gamma (lineage P.1), Delta (lineage B.1.617.2), Omicron (B.1.1.529), Epsilon (lineages B.1.429, B.1.427, CAL.20C), Zeta (lineage P.2), Eta (lineage B.1.525), Theta (lineage P.3), Iota (lineage B.1.526), Kappa (lineage B.1.617.1), Lambda (lineage C.37), and Mu (lineage B.1.621);
preferably the variant is selected from the group comprising, or preferably consisting of, Alpha (lineage B.1.1.7), B.1.1.7 with E484K, Beta (lineage B.1.351), Delta (lineage B.1.617.2), Omicron (B.1.1.529), Epsilon (lineages B.1.429, B.1.427, CAL.20C), Eta (lineage B.1.525), Iota (lineage B.1.526), Kappa (lineage B.1.617.1), and Mu (lineage B.1.621);
more preferably the variant is Delta (lineage B.1.617.2) or Omicron (B.1.1.529); and
again more preferably the variant is Delta (B.1.617.2), Omicron BA.2 or Omicron BA.5.

7. The pharmaceutical product for use according to any one of the preceding claims, wherein the pharmaceutical product is administered intranasally or intramuscularly.

8. The pharmaceutical product for use according to any one of the preceding claims, wherein the natural human coronavirus genome is a natural SARS-CoV-2 genome, preferably
a) a SARS-CoV-2 sequence comprised in or consisting of a sequence as defined by SEQ ID NO: 7 or
b) a SARS-CoV-2 sequence being 80% identical to a sequence comprised in or consisting of a sequence as defined by SEQ ID NO: 7, preferably a SARS-CoV-2 sequence being 80% identical to a sequence comprised in or consisting of sequence as defined by SEQ ID NO: 7 which maintains the ability to encode one or more SARS-CoV-2 virus proteins.

9. The pharmaceutical product for use according to any one of the preceding claims, wherein at least one of the one-to-stop codons is in a sequence encoding non-structural proteins; preferably the natural human coronavirus genome is a natural SARS-CoV-2 genome, and at least one of the one-to-stop codons is in a sequence corresponding to ORF1ab in the natural SARS-CoV-2 genome.

10. The pharmaceutical product for use according to claim 9, wherein at least one of the one-to-stop codons is in a sequence corresponding to an Nsp1 to Nsp15, preferably Nsp3 to Nsp15 encoding sequence in the natural SARS-CoV-2 genome.

11. The pharmaceutical product for use according to claim 9 or 10, wherein at least one of the one-to-stop codons is in a sequence corresponding to an Nsp3 to Nsp7 and/or an Nsp12 to Nsp15 encoding sequence in the natural SARS-CoV-2 genome.

12. The pharmaceutical product for use according to any one of the preceding claims, wherein the natural human coronavirus genome is a natural SARS-CoV-2 genome, and wherein at least one of the one-to-stop codons has a CDS codon number corresponding to a CDS codon number as indicated in Table 1 or supplementary Table 3 for SEQ ID NO: 7.

13. The pharmaceutical product for use according to claim 12, wherein at least one of the one-to-stop codons is in a sequence corresponding to an Nsp3 to Nsp7 or an Nsp12 to Nsp15 encoding sequence in the natural SARS-CoV-2 genome and at least one of the one-to-stop codons has a CDS codon number corresponding to a CDS codon number as indicated in Table 1 or supplementary Table 3 for SEQ ID NO: 7.

14. The pharmaceutical product for use according to claim 12 or 13, wherein the one-to-stop codons are defined by CDS codon numbers corresponding each to a CDS codon number from 2023 to 6614 as indicated in Table 1 or supplementary Table 3 for SEQ ID NO: 7;
preferably, the one-to-stop codons are defined by codon changes and CDS codon numbers corresponding each to a CDS codon number from 2023 to 6614 as indicated in Table 1 or supplementary Table 3 for SEQ ID NO: 7.

15. The pharmaceutical product for use according to any one of the preceding claims, wherein the polynucleotide consists of or comprises a sequence as defined in SEQ ID NO: 3-6 or 9-23, preferably SEQ ID NO: 3-6.
